(19) United States
(12) Patent Application Publication
(10) Pub. No.: US 2025/0034172 A1
(43) Pub. Date: Jan. 30, 2025

(54) COMPOUND, LIGHT-EMITTING MATERIAL, AND LIGHT-EMITTING ELEMENT

(71) Applicant: KYULUX, INC., Fukuoka-shi, Fukuoka (JP)

(72) Inventors: Yoshitake SUZUKI, Fukuoka-shi, Fukuoka (JP); Masataka YAMASHITA, Fukuoka-shi, Fukuoka (JP); Takuya HIGA, Fukuoka-shi, Fukuoka (JP); Naomi SHIMAMURA, Fukuoka-shi, Fukuoka (JP); Satoshi OHNO, Fukuoka-shi, Fukuoka (JP); Songhye HWANG, Fukuoka-shi, Fukuoka (JP); Kousei KANAHARA, Fukuoka-shi, Fukuoka (JP); Kei MORIMOTO, Fukuoka-shi, Fukuoka (JP)

(21) Appl. No.: 18/710,751

(22) PCT Filed: Nov. 2, 2022
(86) PCT No.: PCT/JP2022/040962

§ 371 Date: May 16, 2024

(30) Foreign Application Priority Data
Nov. 19, 2021 (JP) 2021-188860

(51) Int. Cl. C07D 519/00; C07B 59/00; H10K 50/12; H10K 85/20; H10K 85/40; H10K 85/60; H10K 101/20

(52) U.S. Cl. CPC C07D 519/00; C07B 59/004; H10K 50/12; H10K 85/20; H10K 85/40; H10K 85/622; H10K 85/623; H10K 85/633; H10K 85/654; H10K 85/656; H10K 85/6572; H10K 85/6574; H10K 85/6576; H10K 85/658; C07B 2200/05; H10K 2101/20

(57) ABSTRACT

An organic light emitting device using a compound of the following general formula has excellent characteristics. R¹ to R⁴ each are a hydrogen atom, a deuterium atom, an alkyl group, an aryl group, or a donor group; two or more of R¹ to R⁴ are donor groups, and at least one thereof is a substituted ring-fused carbazol-9-yl group; X¹ to X³ each are N or C(R), R is a hydrogen atom, a deuterium atom or a substituent; Ar¹ and Ar² each are an aryl group; L¹ is a single bond or a linking group.

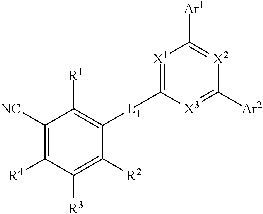

### TECHNICAL FIELD

The present invention relates to a compound useful as a light emitting material, and a light emitting device using the compound.

### BACKGROUND ART

Studies for enhancing the light emission efficiency of light emitting devices such as organic electroluminescent devices (organic EL devices) are being made actively. In particular, various kinds of efforts have been made for increasing light emission efficiency by newly developing and combining an electron transporting material, a hole transporting material, and a light-emitting material to constitute an organic electroluminescent device. Among them, there are seen some reports relating to an organic electroluminescent device that utilizes a delayed fluorescent material.

A delayed fluorescent material is a material which, in an excited state, after having undergone reverse intersystem crossing from an excited triplet state to an excited singlet state, emits fluorescence when returning back from the excited singlet state to a ground state thereof. Fluorescence through the route is observed later than fluorescence from the excited singlet state directly occurring from the ground state (ordinary fluorescence), and is therefore referred to as delayed fluorescence. Here, for example, in the case where a light emitting compound is excited through carrier injection thereinto, the occurring probability of the excited singlet state to the excited triplet state is statistically 25%/75%, and therefore improvement of light emission efficiency by the fluorescence alone from the directly occurring excited singlet state is limited. On the other hand, in a delayed fluorescent material, not only the excited singlet state but also the excited triplet state can be utilized for fluorescent emission through the route via the above-mentioned reverse intersystem crossing, and therefore as compared with an ordinary fluorescent material, a delayed fluorescent material can realize a higher emission efficiency.

Since such a principle has been clarified, various studies have led to the discovery of various delayed fluorescent materials. Among these, many compounds in which cyanobenzene is substituted with a donor group and an acceptor group are included. For example, a compound of cyanobenzene substituted with a benzofurocarbazolyl group as a donor group and with a diphenyltriazinyl group as an acceptor group has been proposed, and one example thereof is a compound having the following structure (see PTL 1).

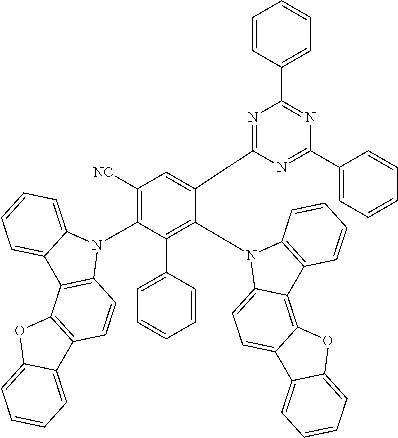 [SMILES: N#Cc1cc(-c2nc(-c3ccccc3)nc(-c3ccccc3)n2)c(-n2c3ccccc3c3c4oc5ccccc5c4ccc32)c(-c2ccccc2)c1-n1c2ccccc2c2c3oc4ccccc4c3ccc21]

### CITATION LIST

### Patent Literature

PTL 1: WO2021/045623A1

### SUMMARY OF INVENTION

### Technical Problem

Even if a material emits delayed fluorescence, one having extremely good characteristics and having no problem in practical use has not been provided. Therefore, for example, it is more useful if it is possible to provide a delayed fluorescent material having a higher light emission efficiency than the delayed fluorescent material proposed in PTL 1. However, the improvement of delayed fluorescent materials is in the stage of trial and error, and it is not easy to generalize the chemical structure of useful light emitting materials.

Under such circumstances, the present inventors have conducted research for the purpose of providing a compound more useful as a light emitting material for a light emitting device. Then, the present inventors have conducted intensive studies for the purpose of deriving and generalizing a general formula of a compound more useful as a light emitting material.

### Solution to Problem

As a result of intensive studies for achieving the above object, the present inventors have found that a cyanobenzene compound having a structure satisfying a specific condition is useful as a light emitting material. The present invention has been proposed based on these findings, and specifically has the following configuration.

[1] A compound represented by the following general formula (1).

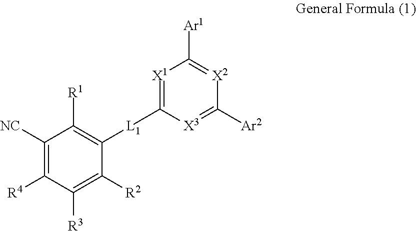

in which R¹ to R⁴ each independently represent a hydrogen atom, a deuterium atom, a substituted or unsubstituted alkyl group, a substituted or unsubstituted aryl group, or a donor group; two or more of R¹ to R⁴ are donor groups, and at least one of the two or more donor groups is a substituted ring-fused carbazol-9-yl group; X¹ to X³ each independently represent N or C(R), and at least one of X¹ to X³ is N; R represents a hydrogen atom, a deuterium atom or a substituent; Ar¹ and Ar² each independently represent a substituted or unsubstituted aryl group; and L¹ represents a single bond or a divalent linking group.

[2] The compound according to [1], in which the ring-fused carbazol-9-yl group is substituted with a substituted or unsubstituted alkyl group, or a substituted or unsubstituted aryl group.

[3] The compound according to [1] or [2], in which the ring-fused carbazol-9-yl group is a carbazol-9-yl group in which a ring having one or more atoms selected from the group consisting of an oxygen atom, a sulfur atom, and a nitrogen atom as a ring skeleton-constituting atom is fused.

[4] The compound according to any one of [1] to [3], in which X¹ to X³ are N.

[5] The compound according to any one of [1] to [4], in which Ar¹ and Ar² each are an aryl group optionally substituted with a deuterium atom.

[6] The compound according to any one of [1] to [5], in which L¹ is a single bond.

[7] The compound according to any one of [1] to [6], in which R¹ is a hydrogen atom.

[8] The compound according to [7], in which R³ is a phenyl group.

[9] The compound according to [8], in which R² and R⁴ are the same substituted ring-fused carbazol-9-yl group.

[10] The compound according to any one of [1] to [9], in which the compound has at least one deuterium atom.

[11] A light emitting material including the compound according to any one of [1] to [10].

[12] A delayed fluorescent material including the compound according to any one of [1] to [10].

[13] A film including the compound according to any one of [1] to [10].

[14] An organic semiconductor device including the compound according to any one of [1] to [10].

[15] An organic light emitting device including the compound according to any one of [1] to [10].

[16] The organic light emitting device according to [15], in which the device has a layer containing the compound, and the layer also contains a host material.

[17] The organic light emitting device according to [16], in which the layer containing the compound further contains a delayed fluorescent material in addition to the compound and the host material, and the delayed fluorescent material has a lowest excited singlet energy lower than that of the host material and higher than that of the compound.

[18] The organic light emitting device according to [16], in which the device has a layer containing the compound, and the layer also contains a light emitting material having a structure different from that of the compound.

[19] The organic light emitting device according to any one of [16] to [18], in which the amount of light emitted from the compound is the largest among materials contained in the device.

[20] The organic light emitting device according to [18], in which the amount of light emitted from the light emitting material is larger than the amount of light emitted from the compound.

[21] The organic light emitting device according to any one of[15] to [20], which is an organic electroluminescent device.

[22] The organic light emitting device according to any one of[15] to [21], which emits delayed fluorescence.

### Advantageous Effects of Invention

The compound of the present invention is useful as a light emitting material. The compound of the present invention includes compounds having a high light emission efficiency. Further, the organic light emitting device using the compound of the present invention also includes excellent devices having a high light emission efficiency.

### DESCRIPTION OF EMBODIMENTS

The contents of the invention will be described in detail below. The constitutional elements may be described below with reference to representative embodiments and specific examples of the invention, but the invention is not limited to the embodiments and the specific examples. In the description herein, a numerical range expressed as “to” means a range that includes the numerical values described before and after “to” as the lower limit and the upper limit. A part or all of hydrogen atoms existing in the molecule of the compound for use in the present invention can be substituted with deuterium atoms (²H, deuterium D). In the chemical structural formulae in the description herein, the hydrogen atom is expressed as H, or the expression thereof is omitted. For example, when expression of the atoms bonding to the ring skeleton-constituting carbon atoms of a benzene ring is omitted, H is considered to bond to the ring skeleton-constituting carbon atom at the site having the omitted expression. In the chemical structural formulae in the present description, a deuterium atom is expressed as D.

[Compound Represented by General Formula (1)]

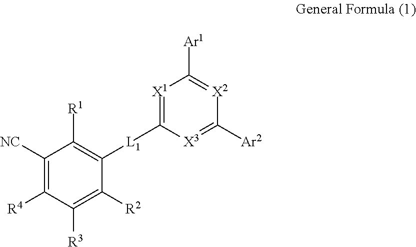

In the general formula (1), R¹ to R⁴ each independently represent a hydrogen atom, a deuterium atom, a substituted or unsubstituted alkyl group, a substituted or unsubstituted aryl group, or a donor group.

Two or more of R¹ to R⁴ are donor groups, and at least one of the two or more donor groups is a substituted ring-fused carbazol-9-yl group. In one preferred aspect of the present invention, at least R² is a substituted ring-fused carbazol-9-yl group. In one preferred aspect of the present invention, at least R⁴ is a substituted ring-fused carbazol-9-yl group. In the present invention, R¹ can be a substituted ring-fused carbazol-9-yl group, and R³ can be a substituted ring-fused carbazol-9-yl group. In one preferred aspect of the present invention, R² and R⁴ are each independently a substituted ring-fused carbazol-9-yl group, and for example, among R¹ to R⁴, only R² and R⁴ are each independently a substituted ring-fused carbazol-9-yl group. Preferably, R² and R⁴ are the same. However, R² and R⁴ can be different. In the present invention, only R² and R³ can be each independently a substituted ring-fused carbazol-9-yl group, and only R³ and R⁴ can be each independently a substituted ring-fused carbazol-9-yl group. Only R¹ and R² can be each independently a substituted ring-fused carbazol-9-yl group, only R¹ and R³ can be each independently a substituted ring-fused carbazol-9-yl group, and only R¹ and R⁴ can be each independently a substituted ring-fused carbazol-9-yl group. In one aspect of the present invention, only R² to R⁴ can be each independently a substituted ring-fused carbazol-9-yl group, all these can be the same, or only one can be different, or all can be different. R¹ to R³ can be each independently a substituted ring-fused carbazol-9-yl group, only R¹, R² and R⁴ can be each independently a substituted ring-fused carbazol-9-yl group, and only R¹, R³ and R⁴ can be each independently a substituted ring-fused carbazol-9-yl group. In one aspect of the present invention, R¹ to R⁴ can be each independently a substituted ring-fused carbazol-9-yl group, all these can be the same, or only one can be different, or all can be different.

The number of rings constituting the fused ring in the substituted ring-fused carbazol-9-yl group is preferably 5 or more, more preferably 5 to 9, even more preferably 5 to 7. In one preferred aspect of the present invention, the number of rings constituting the fused ring is 5. Here, the number of rings includes the number of rings of carbazole to be fused (i.e. 3).

The substituted ring-fused carbazol-9-yl group is a group that bonds via the nitrogen atom constituting the ring skeleton of carbazole, and has a structure in which a ring is fused to at least one of the two benzene rings constituting carbazole. The fused ring can be any of an aromatic hydrocarbon ring, an aromatic heterocyclic ring, an aliphatic hydrocarbon ring, and an aliphatic heterocyclic ring, and can be a ring obtained by further fusing these rings. An aromatic hydrocarbon ring and an aromatic heterocyclic ring are preferable. Examples of the aromatic hydrocarbon ring include a substituted or unsubstituted benzene ring. Another benzene ring can be further fused to the benzene ring, and a heterocyclic ring such as a pyridine ring can be fused to the benzene ring. The aromatic heterocyclic ring means a ring exhibiting aromaticity including a heteroatom as a ring skeleton-constituting atom, and is preferably a 5- to 7-membered ring, and for example, a 5-membered ring or a 6-membered ring can be employed. In one aspect of the present invention, a furan ring, a thiophene ring, or a pyrrole ring can be employed as the aromatic heterocyclic ring. In one aspect of the present invention, the fused ring is a furan ring of a substituted or unsubstituted benzofuran, a thiophene ring of a substituted or unsubstituted benzothiophene, or a pyrrole ring of a substituted or unsubstituted indole. It is preferable that a substituent selected from Substituent Group E bonds to the nitrogen atom of the pyrrole ring, and it is more preferable that an aryl group which can be substituted with an alkyl group or an aryl group is bonded. In the present invention, it is preferable to employ a carbazol-9-yl group in which a ring having one or more atoms selected from the group consisting of an oxygen atom, a sulfur atom, and a nitrogen atom as a ring skeleton-constituting atom is fused. Above all, preferably employed are a benzofuro structure-fused carbazol-9-yl group, a benzothieno structure-fused carbazol-9-yl group, and an indolo structure-fused carbazol-9-yl group. In one aspect of the present invention, the compound has at least one benzofuro structure-fused carbazol-9-yl group, and for example, has two or more such groups. In one aspect of the present invention, the compound has at least one benzothieno structure-fused carbazol-9-yl group, and for example, has two or more such groups.

In the present invention, a substituted benzofuro[2,3-a]carbazol-9-yl group can be employed as the substituted ring-fused carbazol-9-yl group. Also a substituted benzofuro[3,2-a]carbazol-9-yl group can be employed. Also a substituted benzofuro[2,3-b]carbazol-9-yl group can be employed. Also a substituted benzofuro[3,2-b]carbazol-9-yl group can be employed. Also a substituted benzofuro[2,3-c]carbazol-9-yl group can be employed. Also a substituted benzofuro[3,2-c]carbazol-9-yl group can be employed.

A preferred substituted benzofuran-fused carbazol-9-yl group is a carbazol-9-yl group in which only one benzofuran ring is fused at the 2,3-positions and no ring is fused to the other. Specifically, the group includes those having any of the following structures, in which at least one hydrogen atom is substituted.

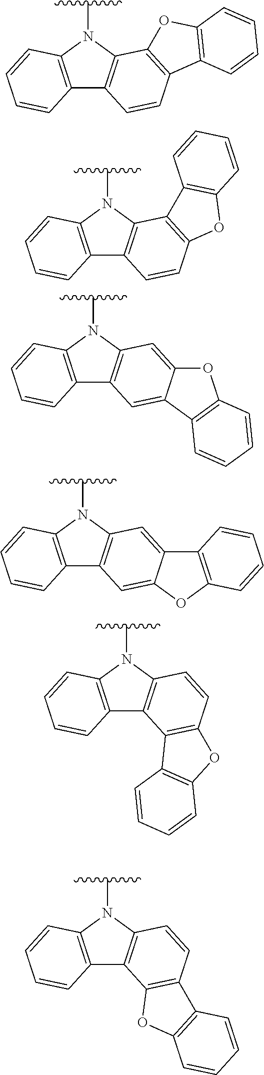

In the present invention, a substituted benzothieno[2,3-a]carbazol-9-yl group can be employed as the substituted ring-fused carbazol-9-yl group. Also a substituted benzothieno[3,2-a]carbazol-9-yl group can be employed. Also a substituted benzothieno[2,3-b]carbazol-9-yl group can be employed. Also a substituted benzothieno[3,2-b]carbazol-9-yl group can be employed. Also a substituted benzothieno[2,3-c]carbazol-9-yl group can be employed. Also a substituted benzothieno[3,2-c]carbazol-9-yl group can be employed.

A preferred substituted benzothiophene-fused carbazol-9-yl group is a carbazol-9-yl group in which only one benzothiophene ring is fused at the 2,3-positions and no ring is fused to the other. Specifically, the group includes those having any of the following structures, in which at least one hydrogen atom is substituted.

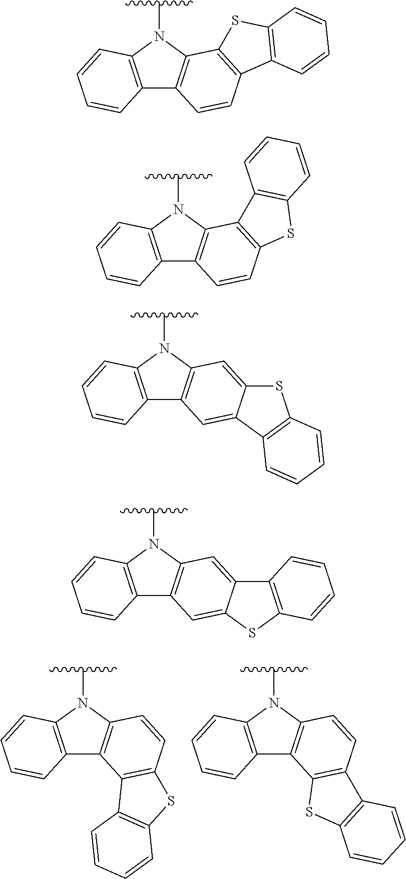

In the present invention, a substituted indolo[2,3-a]carbazol-9-yl group can be employed as the substituted ring-fused carbazol-9-yl group. Also a substituted indolo[3,2-a]carbazol-9-yl group can be employed. Also a substituted indolo[2,3-b]carbazol-9-yl group can be employed. Also a substituted indolo[3,2-b]carbazol-9-yl group can be employed. Also a substituted indolo[2,3-c]carbazol-9-yl group can be employed. Also a substituted indolo[3,2-c]carbazol-9-yl group can be employed. A preferred substituted indole-fused carbazol-9-yl group is a carbazol-9-yl group in which only one indole ring is fused at the 2,3-positions and no ring is fused to the other. Specifically, the group includes those having any of the following structures, in which at least one hydrogen atom is substituted. R¹ in the following structures represents a hydrogen atom, or a substituent. In one aspect of the present invention, R¹ is a substituted or unsubstituted aryl group or a substituted or unsubstituted alkyl group, and is preferably a substituted or unsubstituted aryl group. The substituent for the aryl group and the alkyl group can be selected from Substituent Group A, can be selected from Substituent Group B, can be selected from Substituent Group C, can be selected from Substituent Group D, or can be selected from Substituent Group E. In one preferred aspect of the present invention, the aryl group and the alkyl group are unsubstituted.

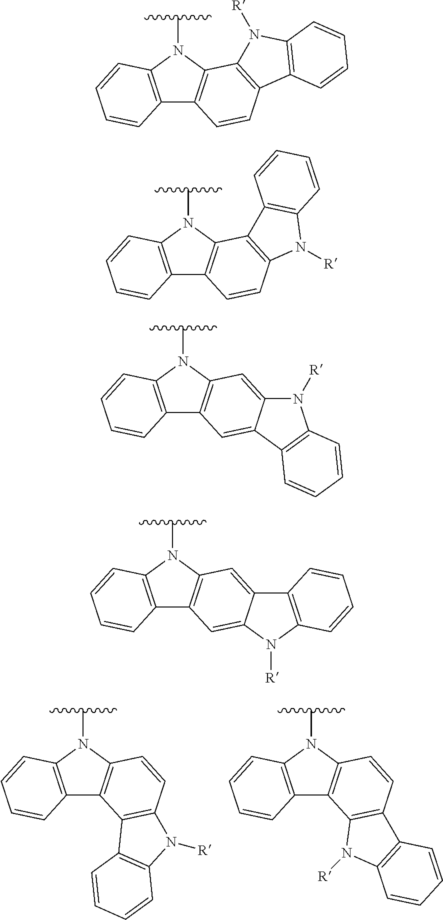

The substituted ring-fused carbazol-9-yl group has a structure in which a substituent bonds to at least one ring skeleton-constituting carbon atom that constitutes the ring-fused carbazol-9-yl group. For example, the substituent for the ring-fused carbazol-9-yl group can be selected from Substituent Group A, can be selected from Substituent Group B, can be selected from Substituent Group C, can be selected from Substituent Group D, or can be selected from Substituent Group E. In one preferred aspect of the present invention, the substituent for the ring-fused carbazol-9-yl group can be selected from a substituted or unsubstituted aryl group and a substituted or unsubstituted alkyl group, and a part or all of the hydrogen atoms of these substituents can be substituted with deuterium atoms. In one preferred aspect of the present invention, the ring-fused carbazol-9-yl group does not have any other substituent than those described herein.

The aryl group can be a monocyclic ring or a fused ring in which two or more rings are fused. In the case of a fused ring, the number of fused rings is preferably 2 to 6, and can be selected from, for example, 2 to 4. Specific examples of the ring include a benzene ring, a naphthalene ring, an anthracene ring, a phenanthrene ring, and a triphenylene ring. In one aspect of the present invention, the aryl group is a substituted or unsubstituted phenyl group, a substituted or unsubstituted naphthalen-1-yl group, or a substituted or unsubstituted naphthalen-2-yl group, and is preferably a substituted or unsubstituted phenyl group. For example, the substituent for the aryl group can be selected from Substituent Group A, can be selected from Substituent Group B, can be selected from Substituent Group C, can be selected from Substituent Group D, or can be selected from Substituent Group E. In one aspect of the present invention, the substituent for the aryl group is at least one selected from the group consisting of an alkyl group, an aryl group and a deuterium atom. In one preferred aspect of the present invention, the aryl group is unsubstituted.

The alkyl group can be linear, branched or cyclic. Two or more of a linear moiety, a cyclic moiety and a branched moiety can be in the group as mixed. The carbon number of the alkyl group can be, for example, 1 or more, 2 or more, or 4 or more. The carbon number can also be 30 or less, 20 or less, 10 or less, 6 or less, or 4 or less. Specific examples of the alkyl group include a methyl group, an ethyl group, an n-propyl group, an isopropyl group, an n-butyl group, an isobutyl group, a tert-butyl group, an n-pentyl group, an isopentyl group, an n-hexyl group, an isohexyl group, a 2-ethylhexyl group, an n-heptyl group, an isoheptyl group, an n-octyl group, an isooctyl group, an n-nonyl group, an isononyl group, an n-decanyl group, an isodecanyl group, a cyclopentyl group, a cyclohexyl group, and a cycloheptyl group. The alkyl group which is the substituent can be further substituted with, for example, a deuterium atom, an aryl group, an alkoxy group, an aryloxy group, and a halogen atom. In one aspect of the present invention, the substituent for the alkyl group is at least one selected from the group consisting of an aryl group and a deuterium atom. In one preferred aspect of the present invention, the alkyl group is unsubstituted.

The number of the substituents substituted on the ring-fused carbazol-9-yl group is preferably 1 to 10, more preferably 1 to 6, even more preferably 1 to 4, and can be, for example 1, or can be, for example 2. In one preferred aspect of the present invention, any of the 3-position or the 6-position of the ring-fused carbazol-9-yl group is substituted. In one preferred aspect of the present invention, the compound has at least one substituent on the para-position of the benzene ring viewed from the heteroatom present in the ring-fused carbazol-9-yl group. In one preferred aspect of the present invention, the compound has at least one substituent only on the para-position of the benzene ring viewed from the heteroatom present in the ring-fused carbazol-9-yl group. In one preferred aspect of the present invention, the compound has substituents on all the substitutable para-positions of the benzene ring viewed from the heteroatom present in the ring-fused carbazol-9-yl group.

Specific examples of the substituted ring-fused carbazol-9-yl group which can be employed in the general formula (1) are shown below. However, the substituted ring-fused carbazol-9-yl group which can be employed in the present invention shall not be construed as being limited by the following specific examples. In the following specific examples, * indicates a bonding site, and Ph represents a phenyl group. A methyl group is not shown. Accordingly, D200 to D223 have a methyl group.

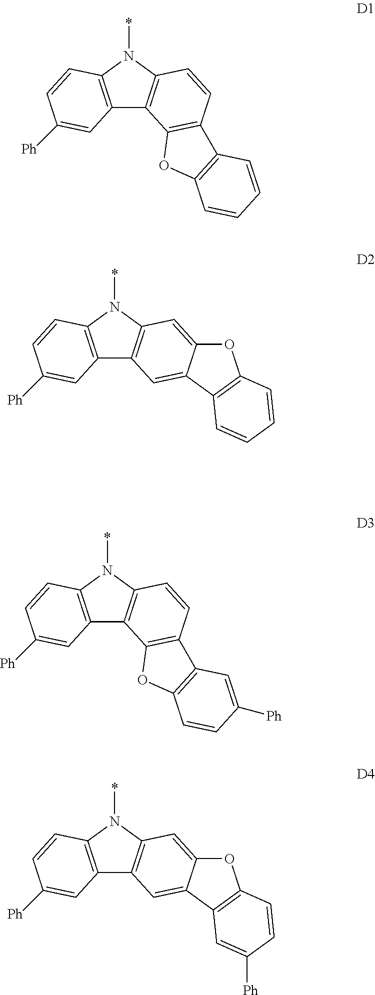 [SMILES: *n1c2ccc(-c3ccccc3)cc2c2c3oc4ccccc4c3ccc21 ; *n1c2ccc(-c3ccccc3)cc2c2cc3c(cc21)oc1ccccc13 ; *n1c2ccc(-c3ccccc3)cc2c2c3oc4ccc(-c5ccccc5)cc4c3ccc21 ; *n1c2ccc(-c3ccccc3)cc2c2cc3c(cc21)oc1ccc(-c2ccccc2)cc13]

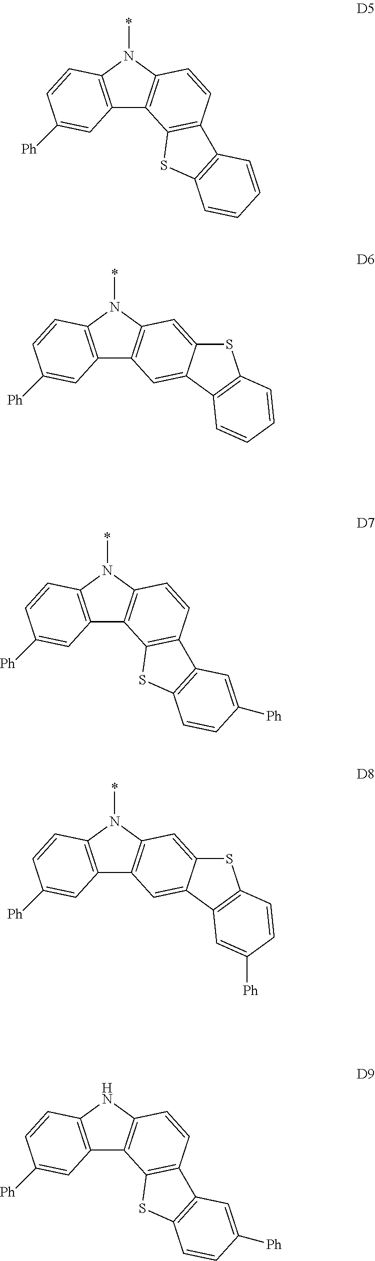 [SMILES: *n1c2ccc(-c3ccccc3)cc2c2c3sc4ccccc4c3ccc21 ; *n1c2ccc(-c3ccccc3)cc2c2cc3c(cc21)sc1ccccc13 ; *n1c2ccc(-c3ccccc3)cc2c2c3sc4ccc(-c5ccccc5)cc4c3ccc21 ; *n1c2ccc(-c3ccccc3)cc2c2cc3c(cc21)sc1ccc(-c2ccccc2)cc13 ; c1ccc(-c2ccc3sc4c(ccc5[nH]c6ccc(-c7ccccc7)cc6c54)c3c2)cc1]

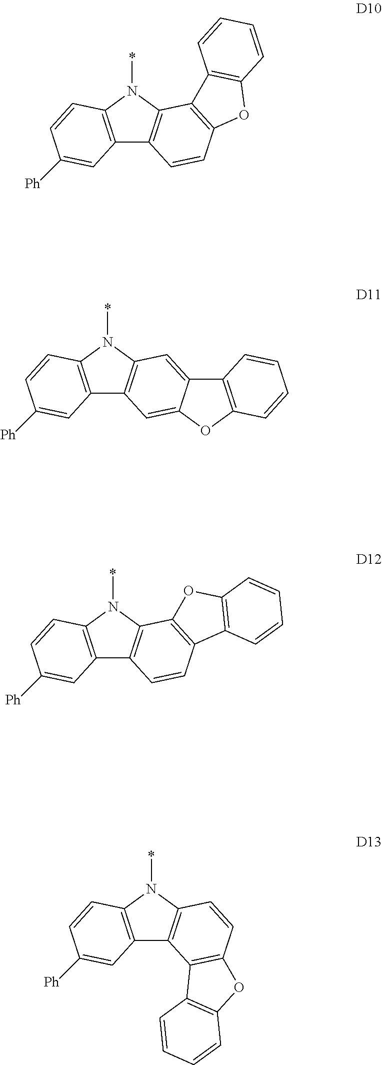 [SMILES: *n1c2ccc(-c3ccccc3)cc2c2ccc3oc4ccccc4c3c21 ; *n1c2ccc(-c3ccccc3)cc2c2cc3oc4ccccc4c3cc21 ; *n1c2ccc(-c3ccccc3)cc2c2ccc3c4ccccc4oc3c21 ; *n1c2ccc(-c3ccccc3)cc2c2c3c(ccc21)oc1ccccc13]

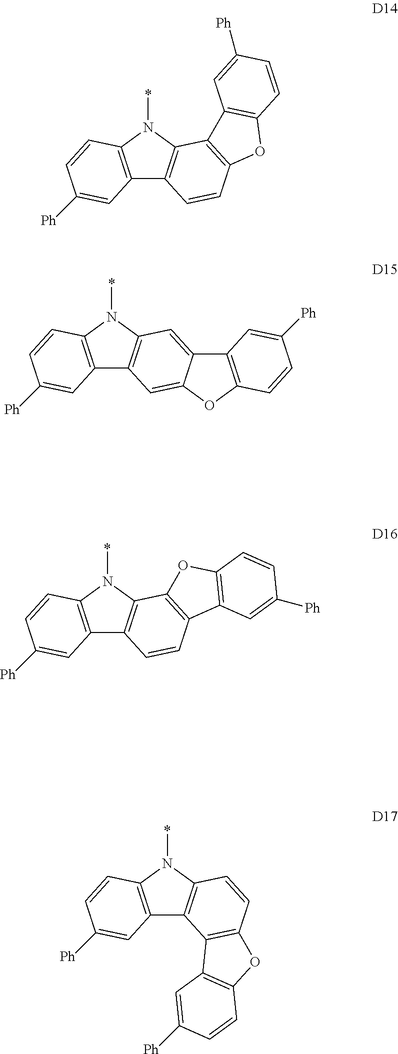 [SMILES: *n1c2ccc(-c3ccccc3)cc2c2ccc3oc4ccc(-c5ccccc5)cc4c3c21 ; *n1c2ccc(-c3ccccc3)cc2c2cc3oc4ccc(-c5ccccc5)cc4c3cc21 ; *n1c2ccc(-c3ccccc3)cc2c2ccc3c4cc(-c5ccccc5)ccc4oc3c21 ; *n1c2ccc(-c3ccccc3)cc2c2c3c(ccc21)oc1ccc(-c2ccccc2)cc13]

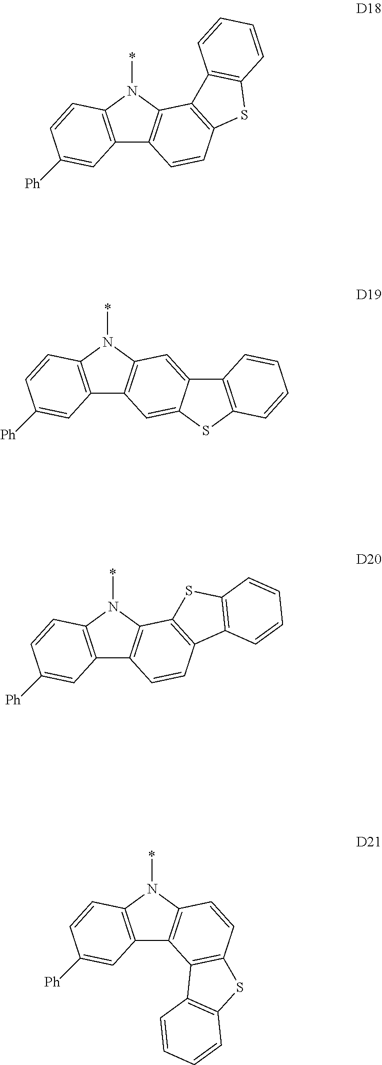 [SMILES: *n1c2ccc(-c3ccccc3)cc2c2ccc3sc4ccccc4c3c21 ; *n1c2ccc(-c3ccccc3)cc2c2cc3sc4ccccc4c3cc21 ; *n1c2ccc(-c3ccccc3)cc2c2ccc3c4ccccc4sc3c21 ; *n1c2ccc(-c3ccccc3)cc2c2c3c(ccc21)sc1ccccc13]

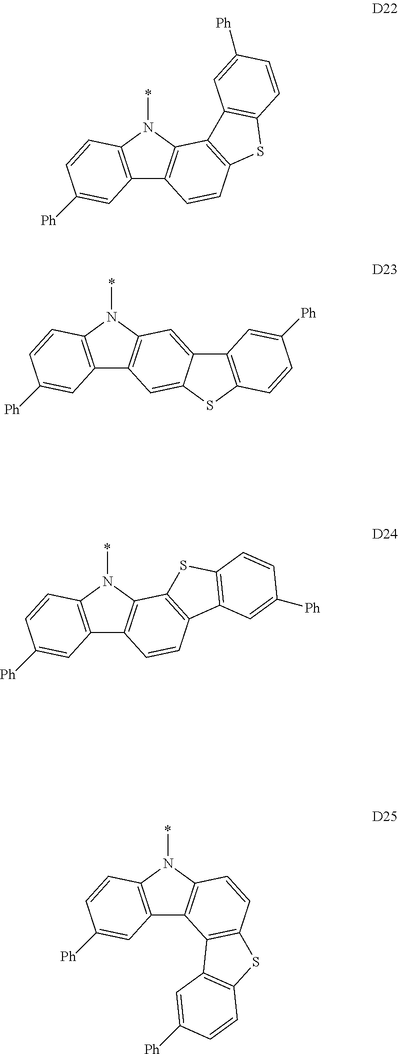 [SMILES: *n1c2ccc(-c3ccccc3)cc2c2ccc3sc4ccc(-c5ccccc5)cc4c3c21 ; *n1c2ccc(-c3ccccc3)cc2c2cc3sc4ccc(-c5ccccc5)cc4c3cc21 ; *n1c2ccc(-c3ccccc3)cc2c2ccc3c4cc(-c5ccccc5)ccc4sc3c21 ; *n1c2ccc(-c3ccccc3)cc2c2c3c(ccc21)sc1ccc(-c2ccccc2)cc13]

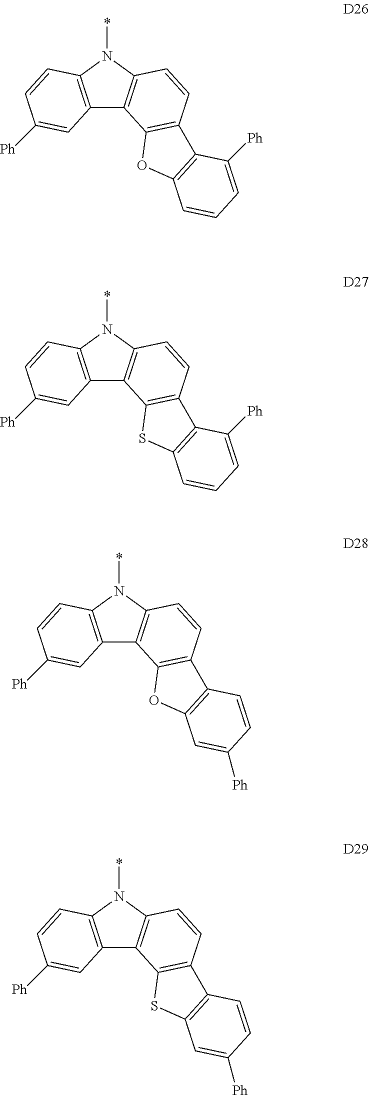 [SMILES: *n1c2ccc(-c3ccccc3)cc2c2c3oc4cccc(-c5ccccc5)c4c3ccc21 ; *n1c2ccc(-c3ccccc3)cc2c2c3sc4cccc(-c5ccccc5)c4c3ccc21 ; *n1c2ccc(-c3ccccc3)cc2c2c3oc4cc(-c5ccccc5)ccc4c3ccc21 ; *n1c2ccc(-c3ccccc3)cc2c2c3sc4cc(-c5ccccc5)ccc4c3ccc21]

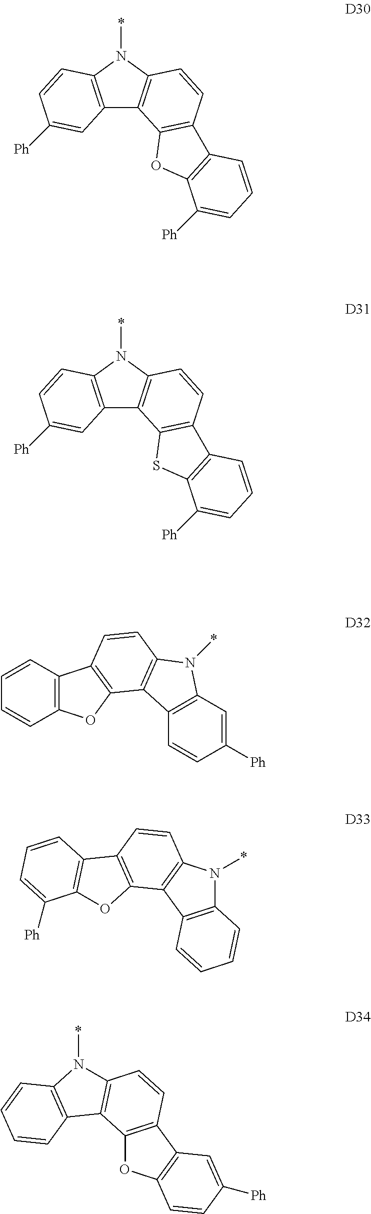 [SMILES: *n1c2ccc(-c3ccccc3)cc2c2c3oc4c(-c5ccccc5)cccc4c3ccc21 ; *n1c2ccc(-c3ccccc3)cc2c2c3sc4c(-c5ccccc5)cccc4c3ccc21 ; *n1c2cc(-c3ccccc3)ccc2c2c3oc4ccccc4c3ccc21 ; *n1c2ccccc2c2c3oc4c(-c5ccccc5)cccc4c3ccc21 ; *n1c2ccccc2c2c3oc4ccc(-c5ccccc5)cc4c3ccc21]

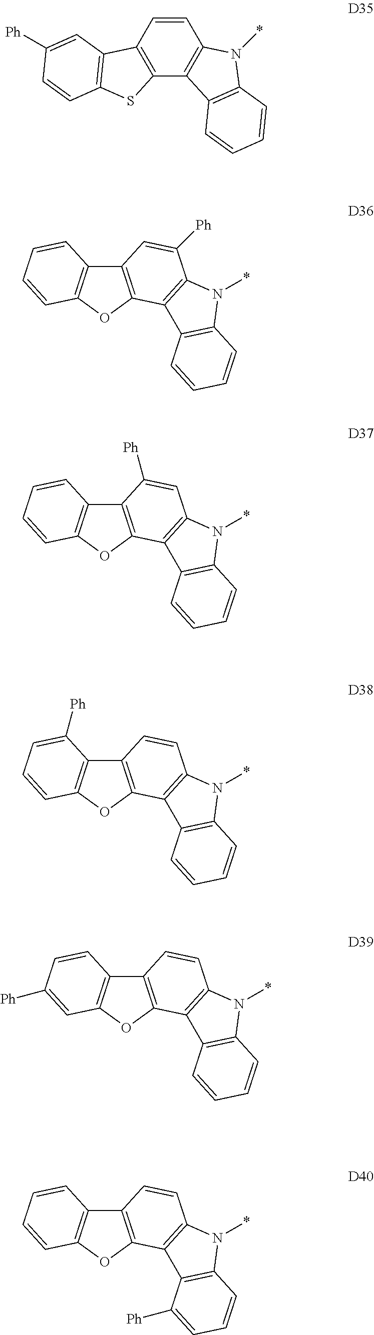 [SMILES: *n1c2ccccc2c2c3sc4ccc(-c5ccccc5)cc4c3ccc21 ; *n1c2ccccc2c2c3oc4ccccc4c3cc(-c3ccccc3)c21 ; *n1c2ccccc2c2c3oc4ccccc4c3c(-c3ccccc3)cc21 ; *n1c2ccccc2c2c3oc4cccc(-c5ccccc5)c4c3ccc21 ; *n1c2ccccc2c2c3oc4cc(-c5ccccc5)ccc4c3ccc21 ; *n1c2cccc(-c3ccccc3)c2c2c3oc4ccccc4c3ccc21]

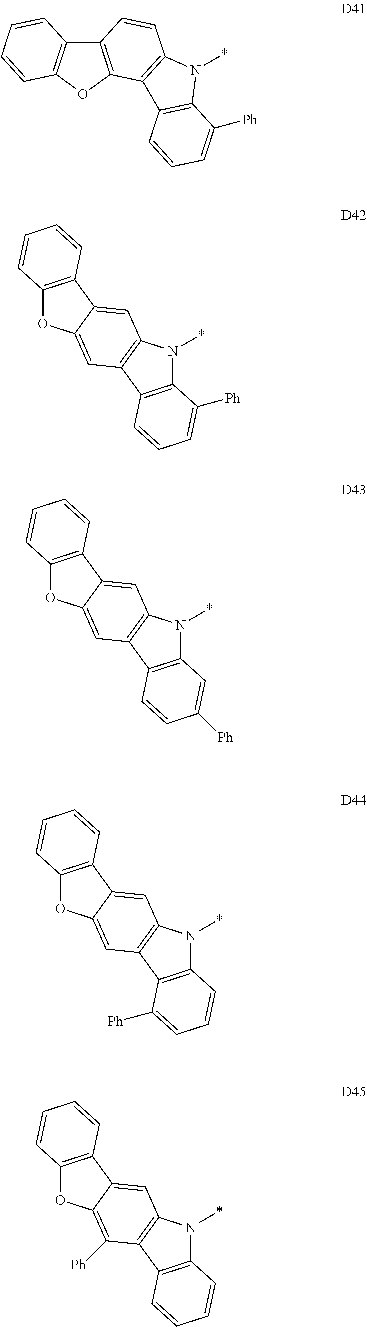 [SMILES: *n1c2ccc3c4ccccc4oc3c2c2cccc(-c3ccccc3)c21 ; *n1c2cc3c(cc2c2cccc(-c4ccccc4)c21)oc1ccccc13 ; *n1c2cc(-c3ccccc3)ccc2c2cc3oc4ccccc4c3cc21 ; *n1c2cc3c(cc2c2c(-c4ccccc4)cccc21)oc1ccccc13 ; *n1c2ccccc2c2c(-c3ccccc3)c3oc4ccccc4c3cc21]

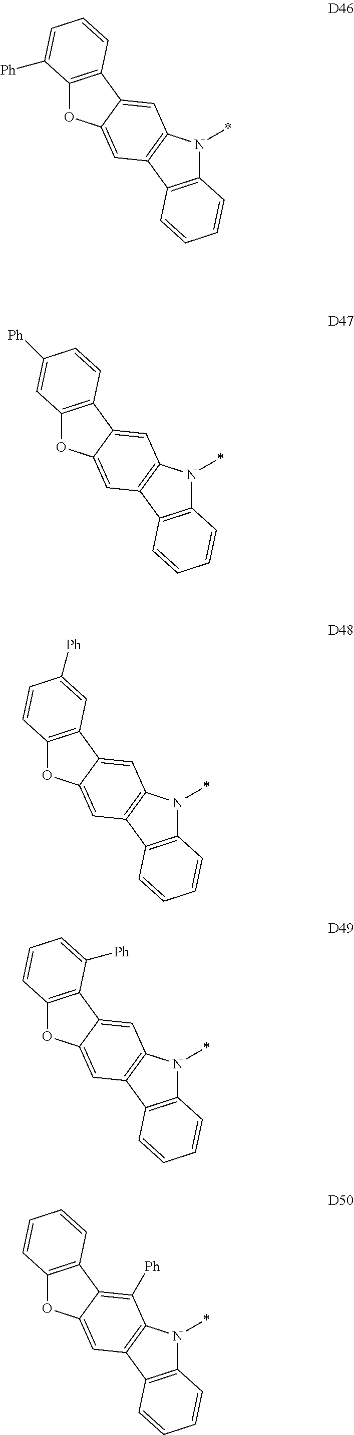 [SMILES: *n1c2ccccc2c2cc3oc4c(-c5ccccc5)cccc4c3cc21 ; *n1c2ccccc2c2cc3oc4cc(-c5ccccc5)ccc4c3cc21 ; *n1c2ccccc2c2cc3oc4ccc(-c5ccccc5)cc4c3cc21 ; *n1c2ccccc2c2cc3oc4cccc(-c5ccccc5)c4c3cc21 ; *n1c2ccccc2c2cc3oc4ccccc4c3c(-c3ccccc3)c21]

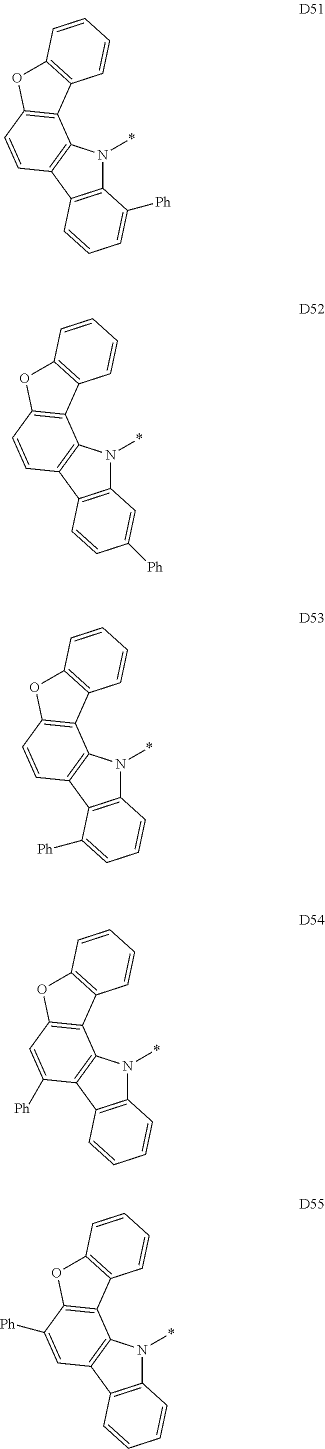 [SMILES: *n1c2c(-c3ccccc3)cccc2c2ccc3oc4ccccc4c3c21 ; *n1c2cc(-c3ccccc3)ccc2c2ccc3oc4ccccc4c3c21 ; *n1c2cccc(-c3ccccc3)c2c2ccc3oc4ccccc4c3c21 ; *n1c2ccccc2c2c(-c3ccccc3)cc3oc4ccccc4c3c21 ; *n1c2ccccc2c2cc(-c3ccccc3)c3oc4ccccc4c3c21]

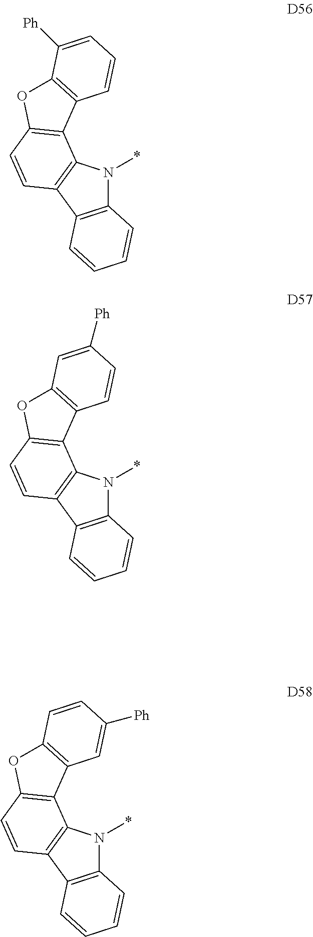

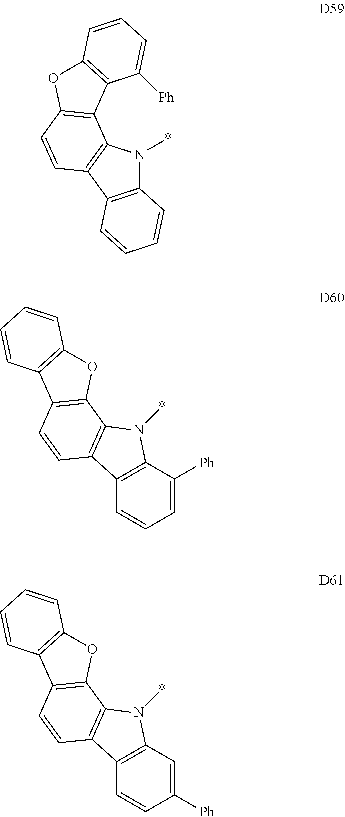 [SMILES: *n1c2ccccc2c2ccc3oc4cccc(-c5ccccc5)c4c3c21 ; *n1c2c(-c3ccccc3)cccc2c2ccc3c4ccccc4oc3c21 ; *n1c2cc(-c3ccccc3)ccc2c2ccc3c4ccccc4oc3c21]

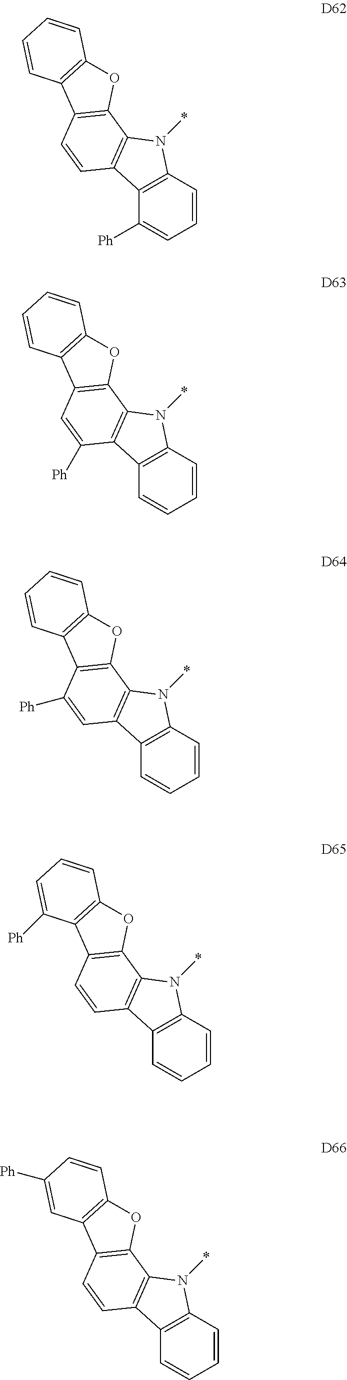 [SMILES: *n1c2cccc(-c3ccccc3)c2c2ccc3c4ccccc4oc3c21 ; *n1c2ccccc2c2c(-c3ccccc3)cc3c4ccccc4oc3c21 ; *n1c2ccccc2c2cc(-c3ccccc3)c3c4ccccc4oc3c21 ; *n1c2ccccc2c2ccc3c(oc4cccc(-c5ccccc5)c43)c21 ; *n1c2ccccc2c2ccc3c4cc(-c5ccccc5)ccc4oc3c21]

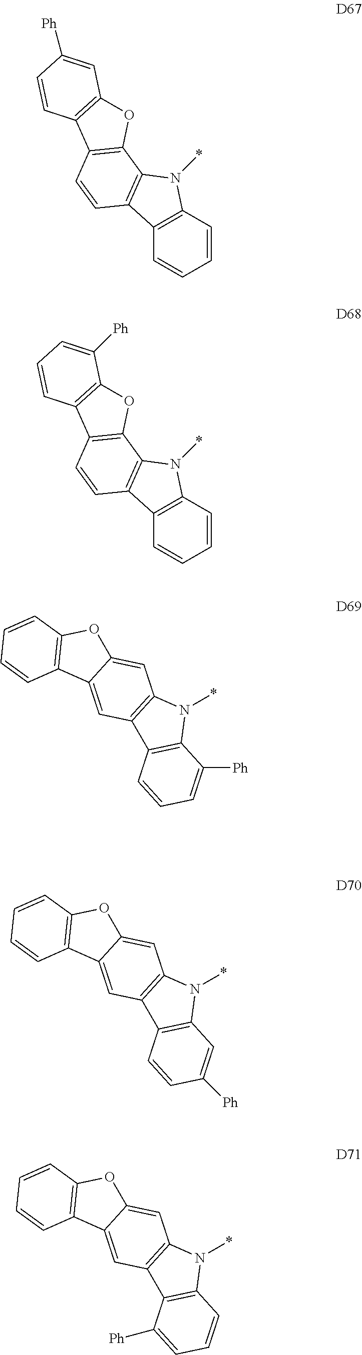 [SMILES: *n1c2ccccc2c2ccc3c4ccc(-c5ccccc5)cc4oc3c21 ; *n1c2ccccc2c2ccc3c4cccc(-c5ccccc5)c4oc3c21 ; *n1c2cc3oc4ccccc4c3cc2c2cccc(-c3ccccc3)c21 ; *n1c2cc(-c3ccccc3)ccc2c2cc3c(cc21)oc1ccccc13 ; *n1c2cc3oc4ccccc4c3cc2c2c(-c3ccccc3)cccc21]

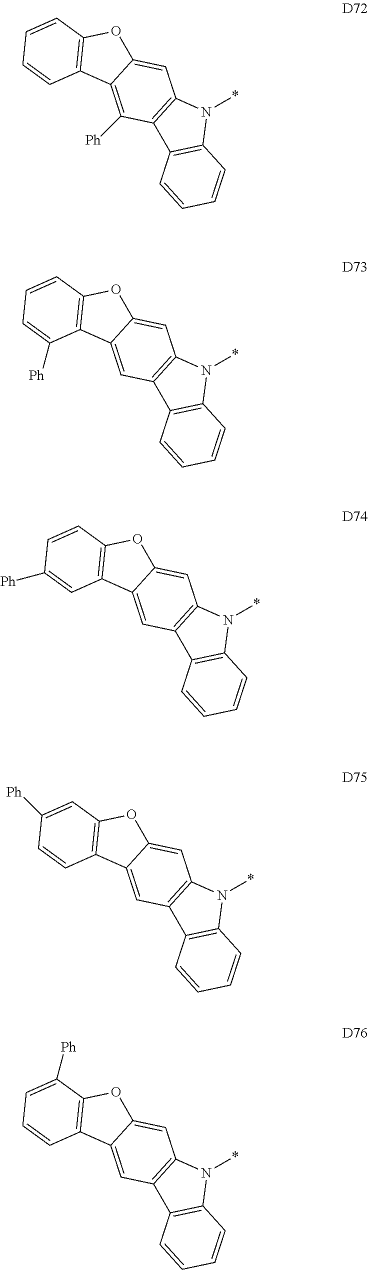 [SMILES: *n1c2ccccc2c2c(-c3ccccc3)c3c(cc21)oc1ccccc13 ; *n1c2ccccc2c2cc3c(cc21)oc1cccc(-c2ccccc2)c13 ; *n1c2ccccc2c2cc3c(cc21)oc1ccc(-c2ccccc2)cc13 ; *n1c2ccccc2c2cc3c(cc21)oc1cc(-c2ccccc2)ccc13 ; *n1c2ccccc2c2cc3c(cc21)oc1c(-c2ccccc2)cccc13]

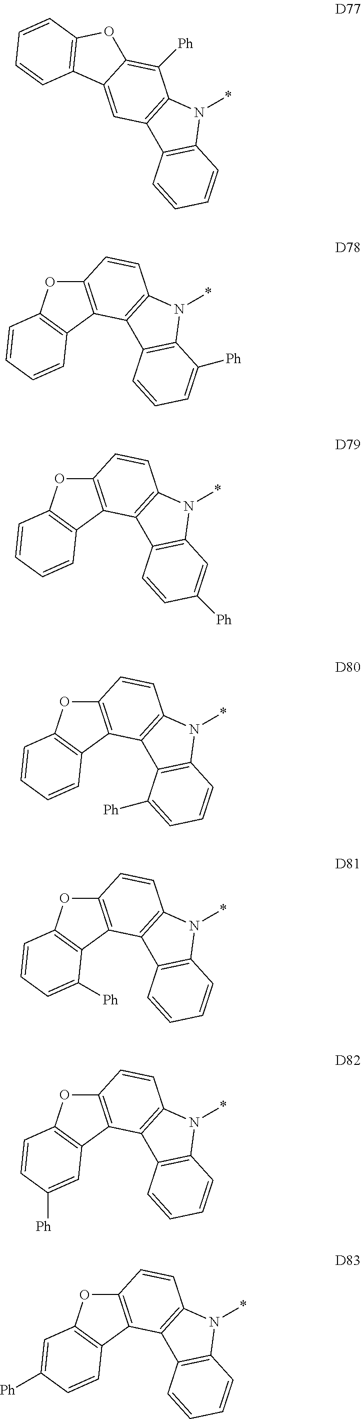

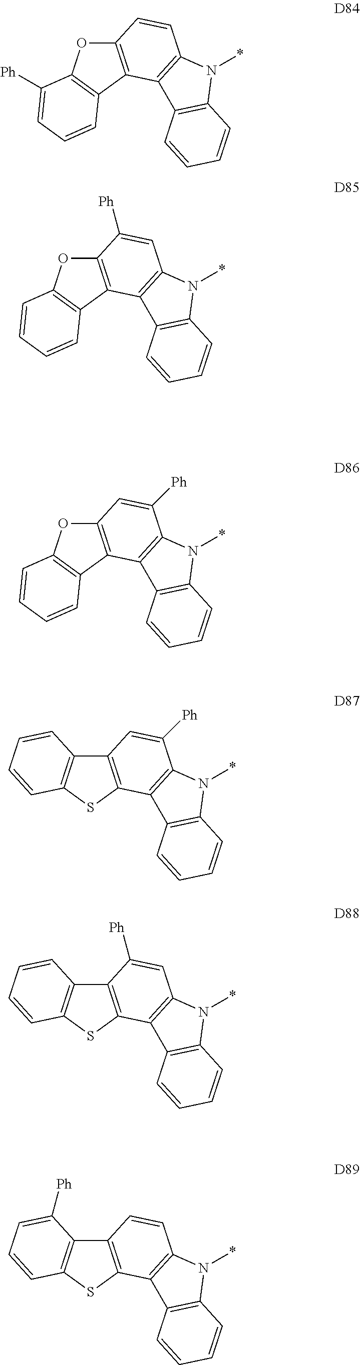 [SMILES: *n1c2ccccc2c2c3c(ccc21)oc1c(-c2ccccc2)cccc13 ; *n1c2ccccc2c2c3c(oc4ccccc43)c(-c3ccccc3)cc21 ; *n1c2ccccc2c2c3c(cc(-c4ccccc4)c21)oc1ccccc13 ; *n1c2ccccc2c2c3sc4ccccc4c3cc(-c3ccccc3)c21 ; *n1c2ccccc2c2c3sc4ccccc4c3c(-c3ccccc3)cc21 ; *n1c2ccccc2c2c3sc4cccc(-c5ccccc5)c4c3ccc21]

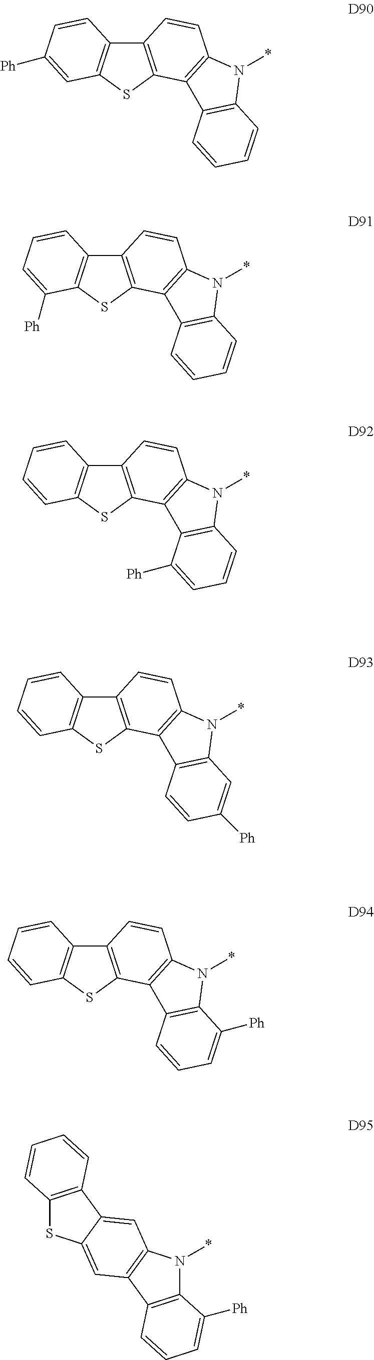 [SMILES: *n1c2ccccc2c2c3sc4cc(-c5ccccc5)ccc4c3ccc21 ; *n1c2ccccc2c2c3sc4c(-c5ccccc5)cccc4c3ccc21 ; *n1c2cccc(-c3ccccc3)c2c2c3sc4ccccc4c3ccc21 ; *n1c2cc(-c3ccccc3)ccc2c2c3sc4ccccc4c3ccc21 ; *n1c2ccc3c4ccccc4sc3c2c2cccc(-c3ccccc3)c21 ; *n1c2cc3c(cc2c2cccc(-c4ccccc4)c21)sc1ccccc13]

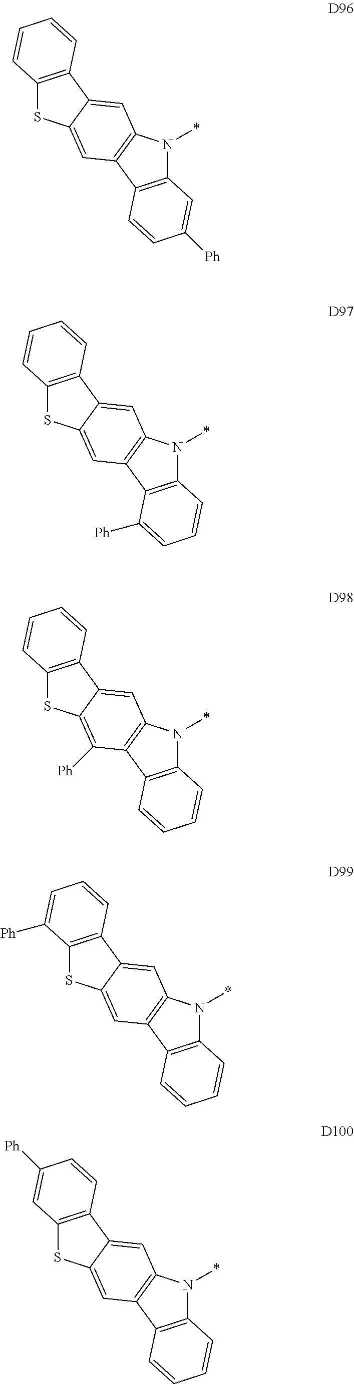 [SMILES: *n1c2cc(-c3ccccc3)ccc2c2cc3sc4ccccc4c3cc21 ; *n1c2cc3c(cc2c2c(-c4ccccc4)cccc21)sc1ccccc13 ; *n1c2ccccc2c2c(-c3ccccc3)c3sc4ccccc4c3cc21 ; *n1c2ccccc2c2cc3sc4c(-c5ccccc5)cccc4c3cc21 ; *n1c2ccccc2c2cc3sc4cc(-c5ccccc5)ccc4c3cc21]

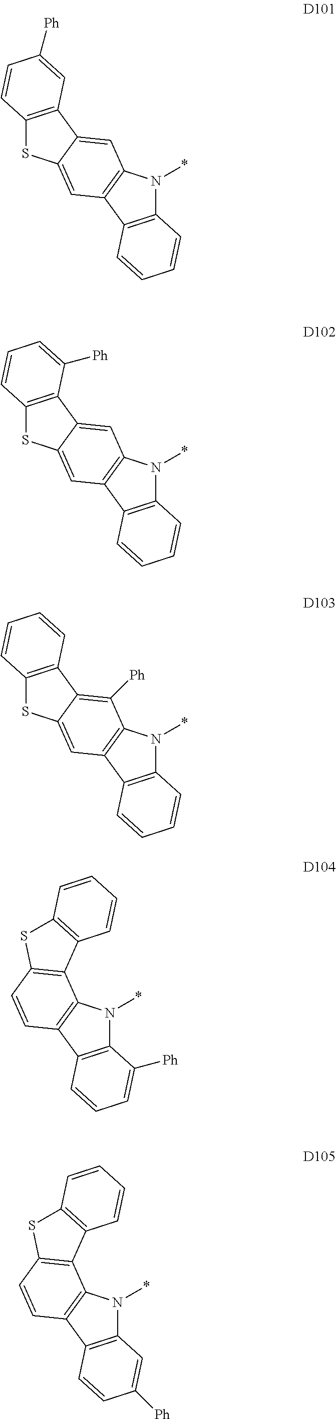 [SMILES: *n1c2ccccc2c2cc3sc4ccc(-c5ccccc5)cc4c3cc21 ; *n1c2ccccc2c2cc3sc4cccc(-c5ccccc5)c4c3cc21 ; *n1c2ccccc2c2cc3sc4ccccc4c3c(-c3ccccc3)c21 ; *n1c2c(-c3ccccc3)cccc2c2ccc3sc4ccccc4c3c21 ; *n1c2cc(-c3ccccc3)ccc2c2ccc3sc4ccccc4c3c21]

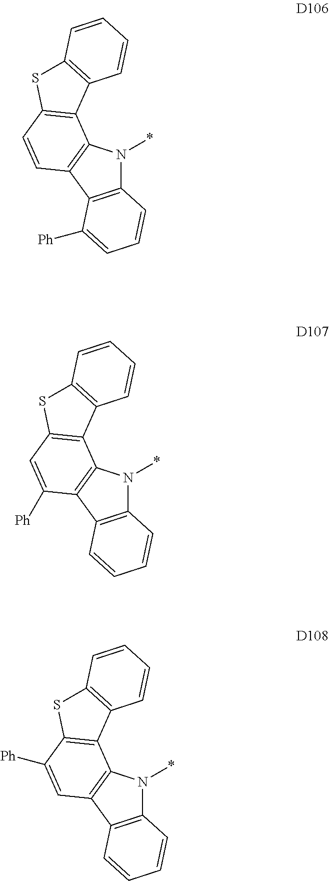 [SMILES: *n1c2cccc(-c3ccccc3)c2c2ccc3sc4ccccc4c3c21 ; *n1c2ccccc2c2c(-c3ccccc3)cc3sc4ccccc4c3c21 ; *n1c2ccccc2c2cc(-c3ccccc3)c3sc4ccccc4c3c21]

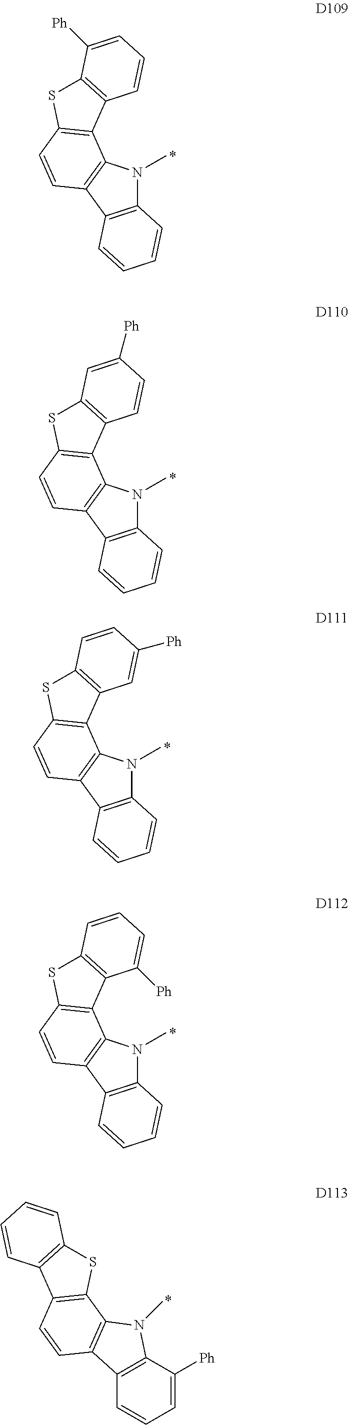

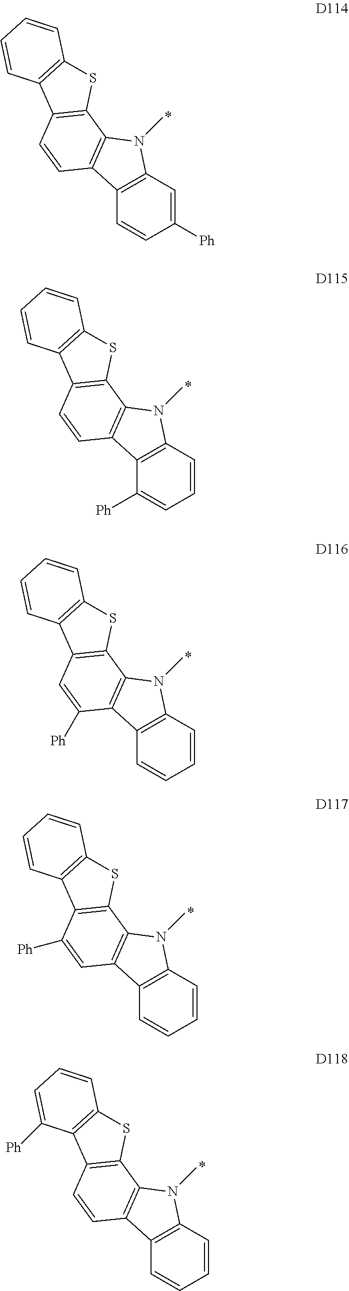 [SMILES: Cn1c2cc(-c3ccccc3)ccc2c2ccc3c4ccccc4sc3c21 ; Cn1c2cccc(-c3ccccc3)c2c2ccc3c4ccccc4sc3c21 ; Cn1c2ccccc2c2c(-c3ccccc3)cc3c4ccccc4sc3c21 ; Cn1c2ccccc2c2cc(-c3ccccc3)c3c4ccccc4sc3c21 ; Cn1c2ccccc2c2ccc3c(sc4cccc(-c5ccccc5)c43)c21]

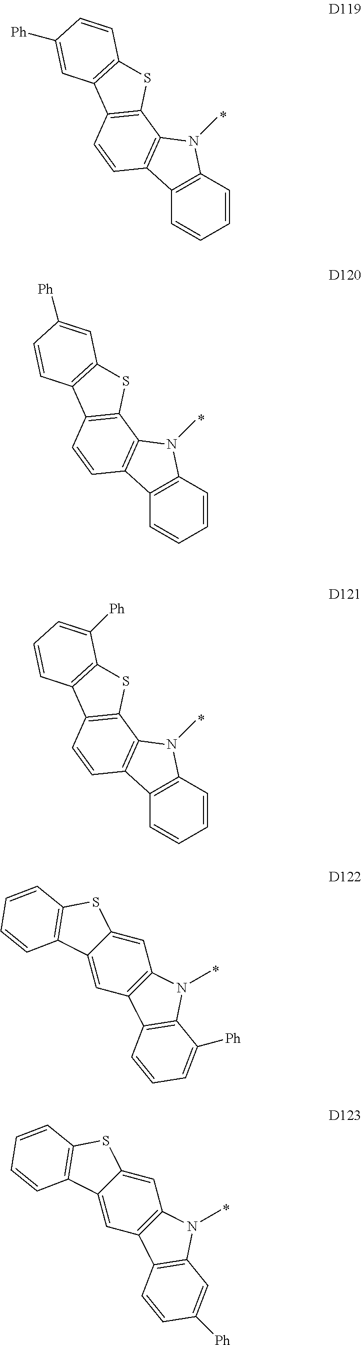 [SMILES: Cn1c2ccccc2c2ccc3c4cc(-c5ccccc5)ccc4sc3c21 ; Cn1c2ccccc2c2ccc3c4ccc(-c5ccccc5)cc4sc3c21 ; Cn1c2ccccc2c2ccc3c4cccc(-c5ccccc5)c4sc3c21 ; Cn1c2cc3sc4ccccc4c3cc2c2cccc(-c3ccccc3)c21 ; Cn1c2cc(-c3ccccc3)ccc2c2cc3c(cc21)sc1ccccc13]

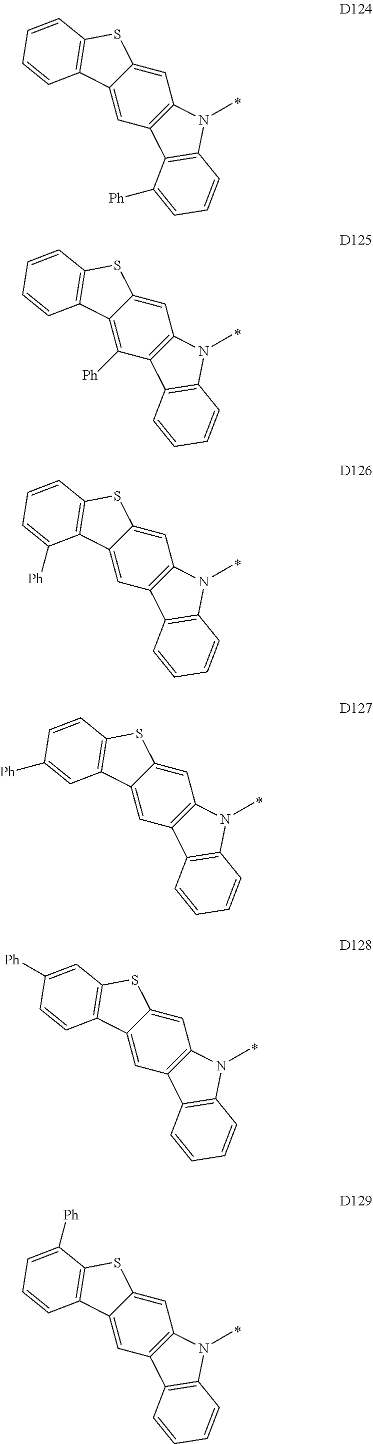 [SMILES: Cn1c2cc3sc4ccccc4c3cc2c2c(-c3ccccc3)cccc21 ; Cn1c2ccccc2c2c(-c3ccccc3)c3c(cc21)sc1ccccc13 ; Cn1c2ccccc2c2cc3c(cc21)sc1cccc(-c2ccccc2)c13 ; Cn1c2ccccc2c2cc3c(cc21)sc1ccc(-c2ccccc2)cc13 ; Cn1c2ccccc2c2cc3c(cc21)sc1cc(-c2ccccc2)ccc13 ; Cn1c2ccccc2c2cc3c(cc21)sc1c(-c2ccccc2)cccc13]

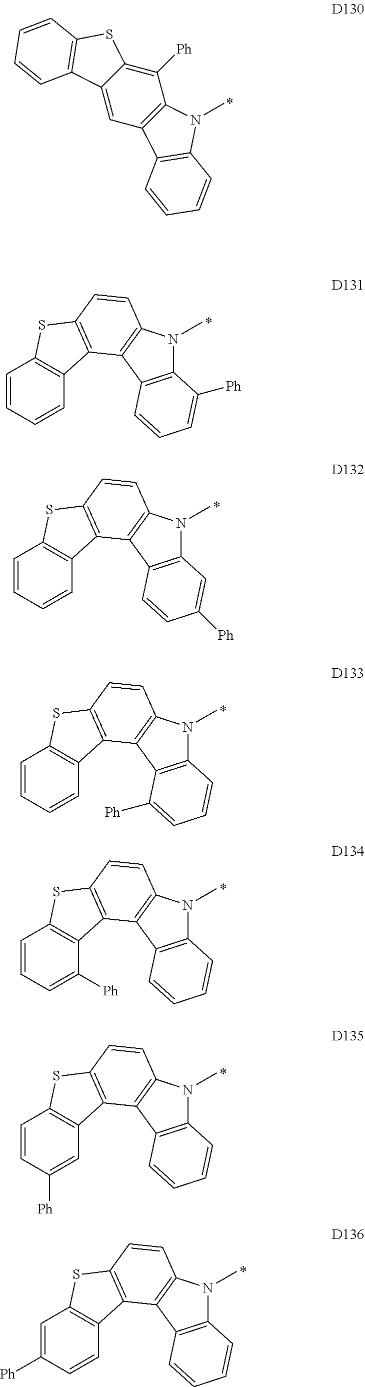

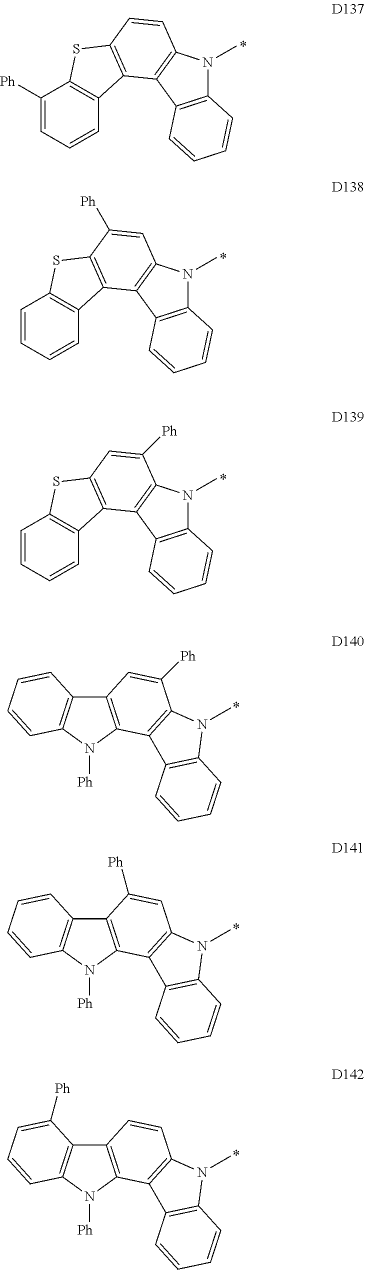

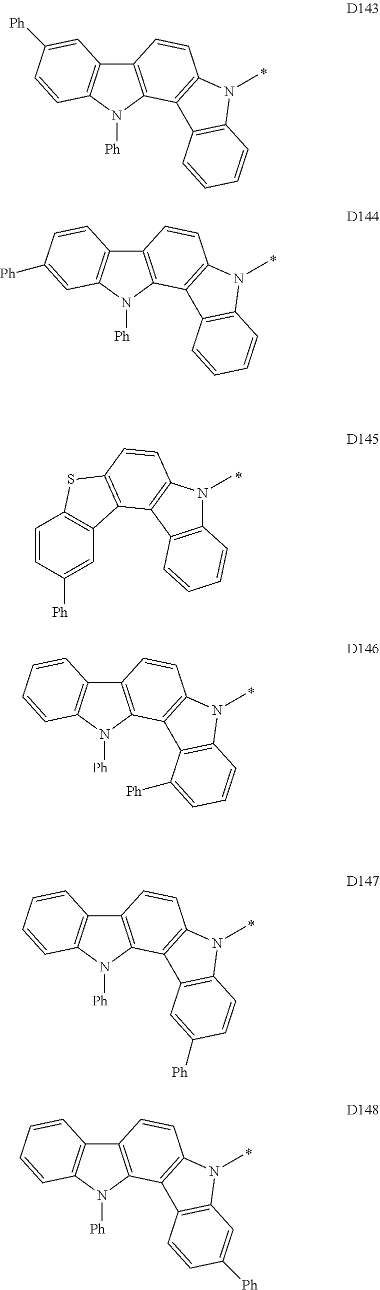

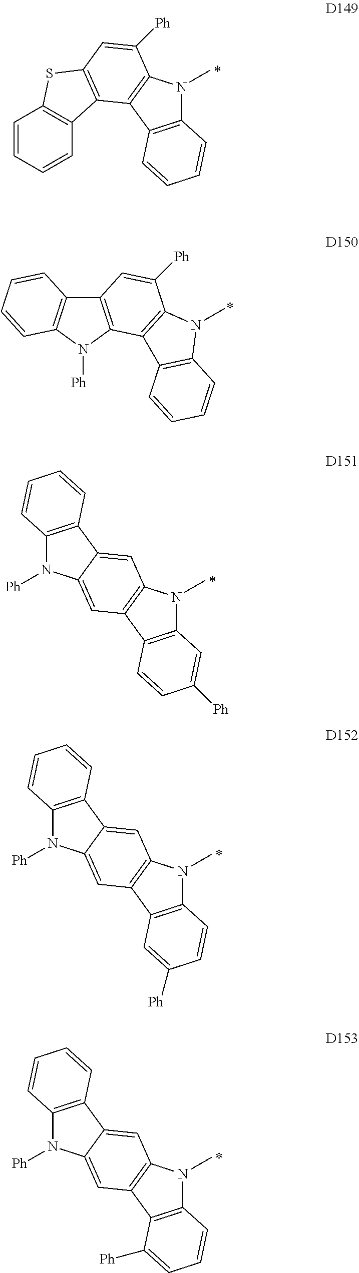 [SMILES: Cn1c2ccccc2c2c3c(cc(-c4ccccc4)c21)sc1ccccc13 ; Cn1c2ccccc2c2c1c(-c1ccccc1)cc1c3ccccc3n(-c3ccccc3)c12 ; Cn1c2cc(-c3ccccc3)ccc2c2cc3c(cc21)c1ccccc1n3-c1ccccc1 ; Cn1c2ccc(-c3ccccc3)cc2c2cc3c(cc21)c1ccccc1n3-c1ccccc1 ; Cn1c2cc3c4ccccc4n(-c4ccccc4)c3cc2c2c(-c3ccccc3)cccc21]

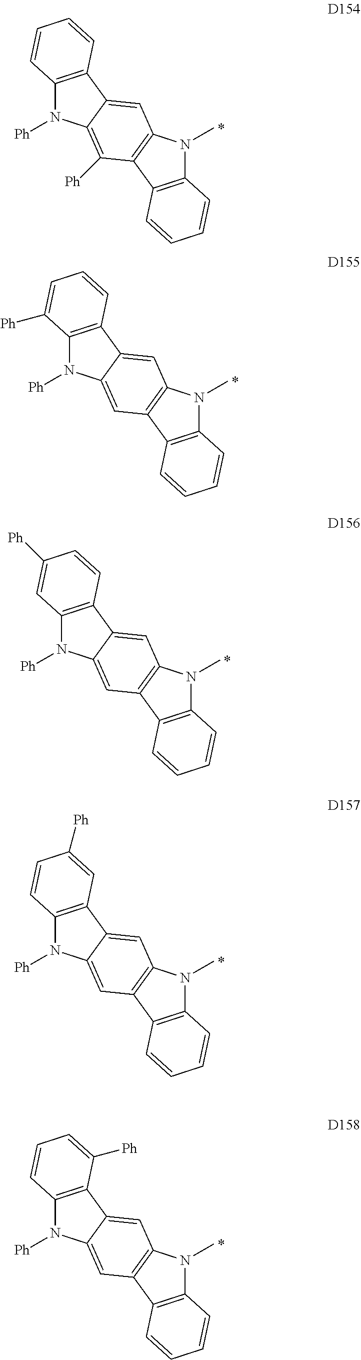 [SMILES: Cn1c2ccccc2c2c(-c3ccccc3)c3c(cc21)c1ccccc1n3-c1ccccc1 ; Cn1c2ccccc2c2cc3c(cc21)c1cccc(-c2ccccc2)c1n3-c1ccccc1 ; Cn1c2ccccc2c2cc3c(cc21)c1ccc(-c2ccccc2)cc1n3-c1ccccc1 ; Cn1c2ccccc2c2cc3c(cc21)c1cc(-c2ccccc2)ccc1n3-c1ccccc1 ; Cn1c2ccccc2c2cc3c(cc21)c1c(-c2ccccc2)cccc1n3-c1ccccc1]

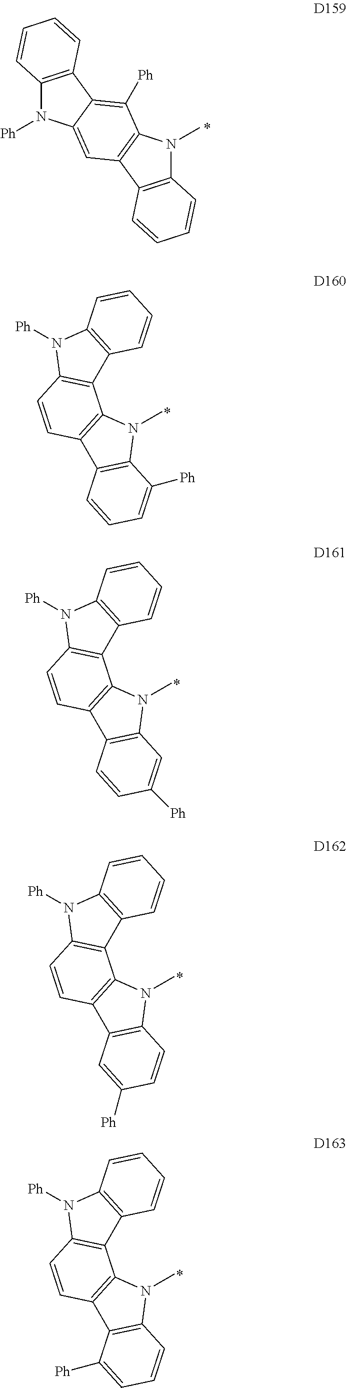

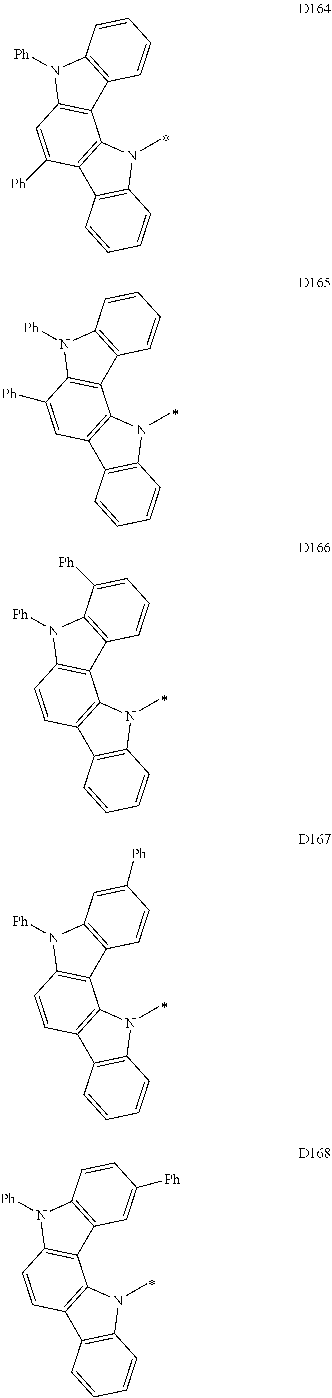

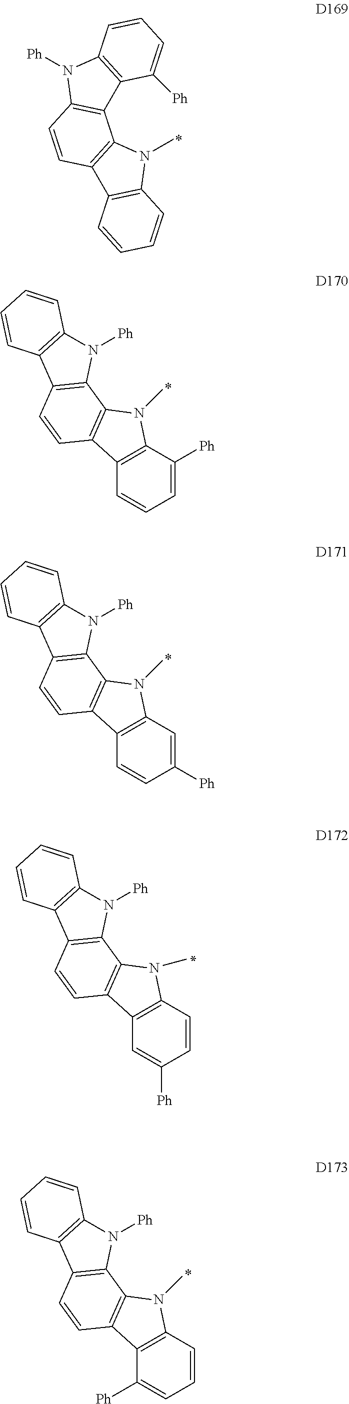 [SMILES: Cn1c2ccccc2c2ccc3c(c4c(-c5ccccc5)cccc4n3-c3ccccc3)c21 ; Cn1c2c(-c3ccccc3)cccc2c2ccc3c4ccccc4n(-c4ccccc4)c3c21 ; Cn1c2cc(-c3ccccc3)ccc2c2ccc3c4ccccc4n(-c4ccccc4)c3c21 ; Cn1c2ccc(-c3ccccc3)cc2c2ccc3c4ccccc4n(-c4ccccc4)c3c21 ; Cn1c2cccc(-c3ccccc3)c2c2ccc3c4ccccc4n(-c4ccccc4)c3c21]

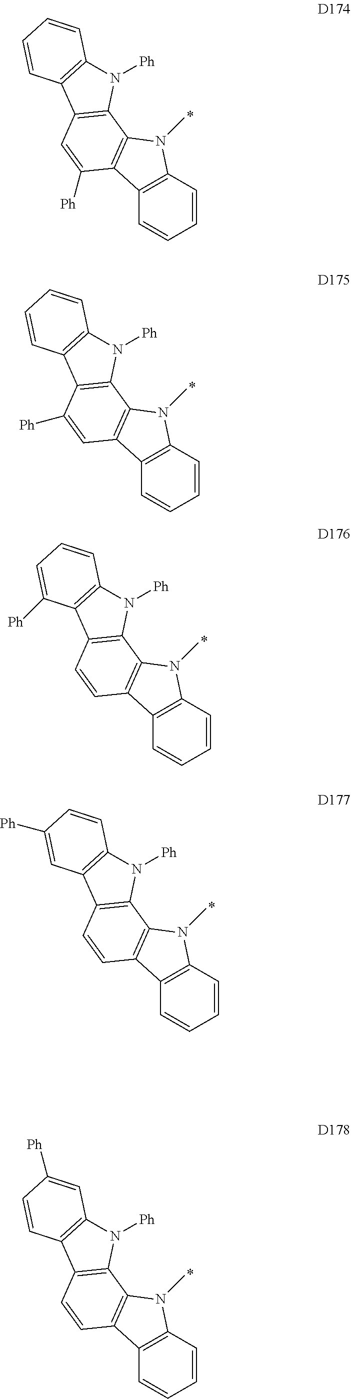 [SMILES: Cn1c2ccccc2c2c(-c3ccccc3)cc3c4ccccc4n(-c4ccccc4)c3c21 ; Cn1c2ccccc2c2cc(-c3ccccc3)c3c4ccccc4n(-c4ccccc4)c3c21 ; Cn1c2ccccc2c2ccc3c4c(-c5ccccc5)cccc4n(-c4ccccc4)c3c21 ; Cn1c2ccccc2c2ccc3c4cc(-c5ccccc5)ccc4n(-c4ccccc4)c3c21 ; Cn1c2ccccc2c2ccc3c4ccc(-c5ccccc5)cc4n(-c4ccccc4)c3c21]

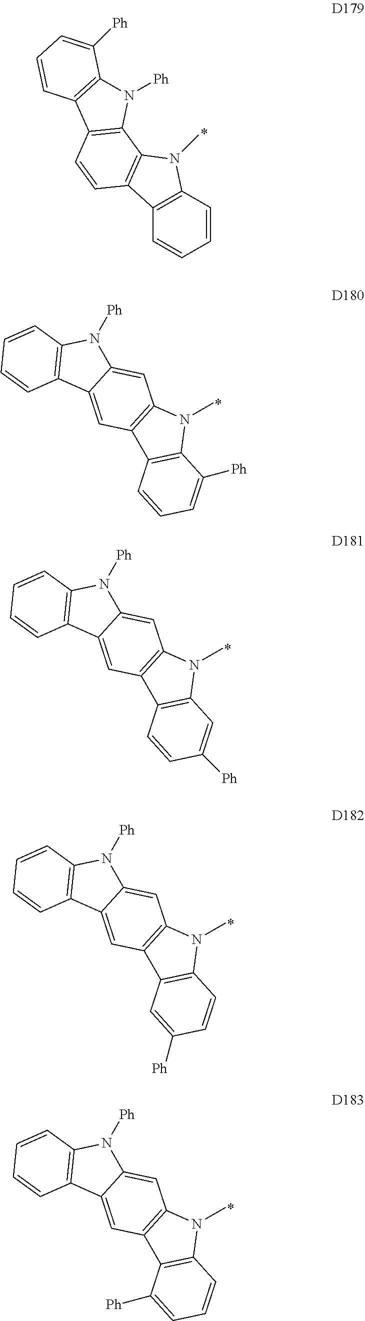 [SMILES: Cn1c2ccccc2c2ccc3c4cccc(-c5ccccc5)c4n(-c4ccccc4)c3c21 ; Cn1c2cc3c(cc2c2cccc(-c4ccccc4)c21)c1ccccc1n3-c1ccccc1 ; Cn1c2cc(-c3ccccc3)ccc2c2cc3c4ccccc4n(-c4ccccc4)c3cc21 ; Cn1c2ccc(-c3ccccc3)cc2c2cc3c4ccccc4n(-c4ccccc4)c3cc21 ; Cn1c2cc3c(cc2c2c(-c4ccccc4)cccc21)c1ccccc1n3-c1ccccc1]

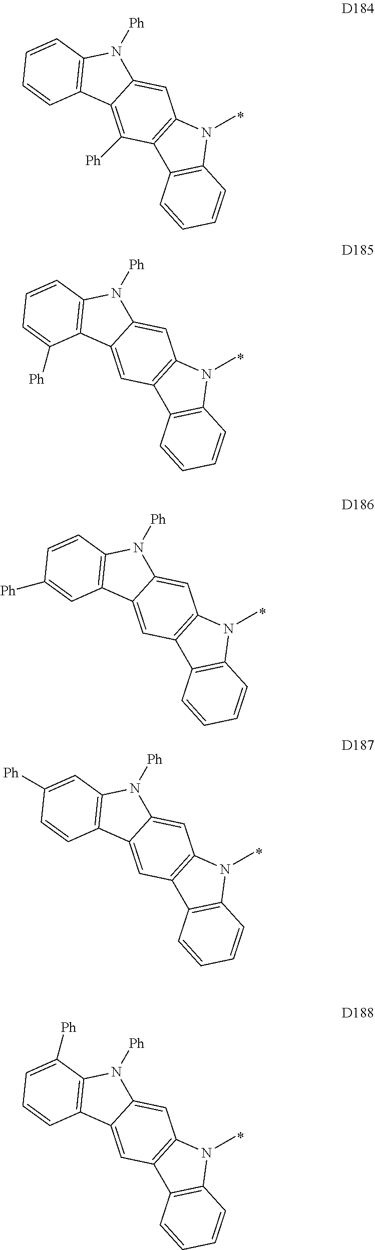 [SMILES: Cn1c2ccccc2c2c(-c3ccccc3)c3c4ccccc4n(-c4ccccc4)c3cc21 ; Cn1c2ccccc2c2cc3c4c(-c5ccccc5)cccc4n(-c4ccccc4)c3cc21 ; Cn1c2ccccc2c2cc3c4cc(-c5ccccc5)ccc4n(-c4ccccc4)c3cc21 ; Cn1c2ccccc2c2cc3c4ccc(-c5ccccc5)cc4n(-c4ccccc4)c3cc21 ; Cn1c2ccccc2c2cc3c4cccc(-c5ccccc5)c4n(-c4ccccc4)c3cc21]

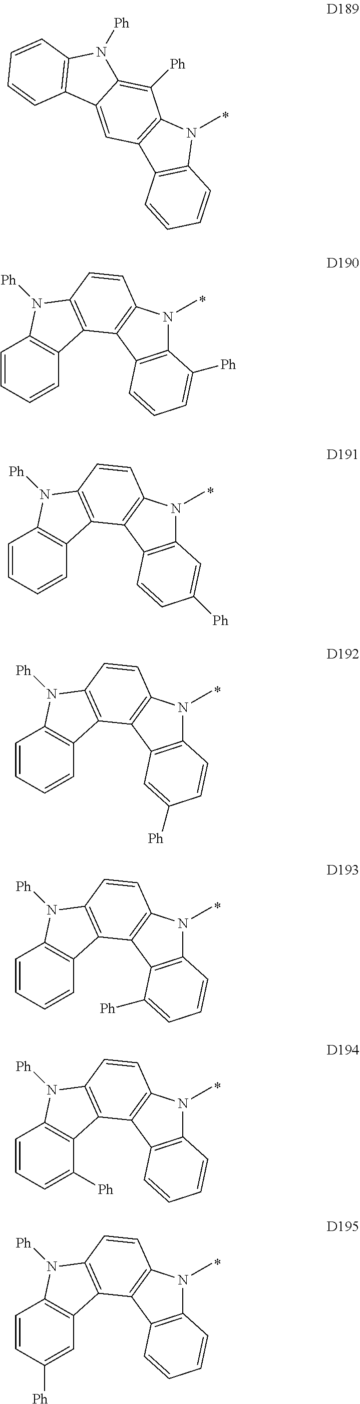

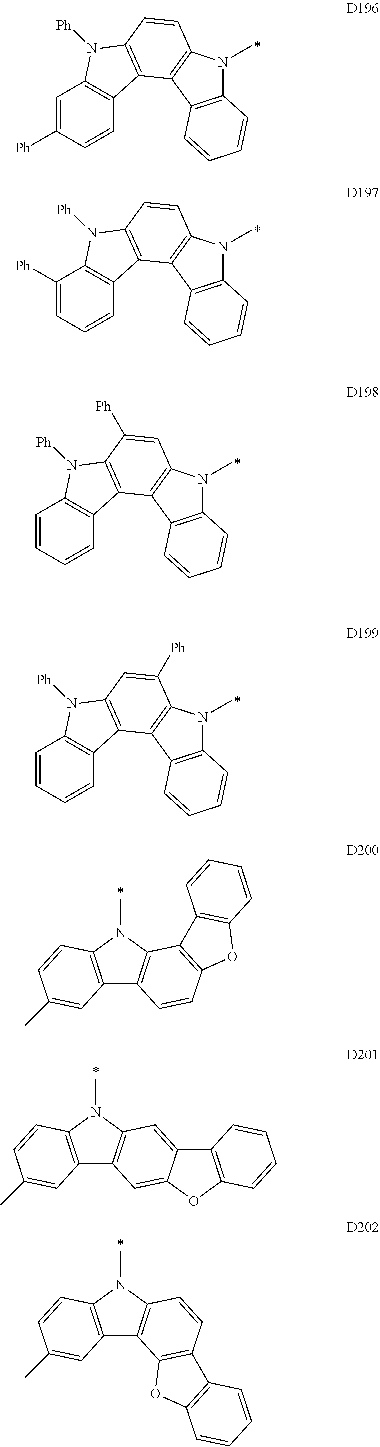

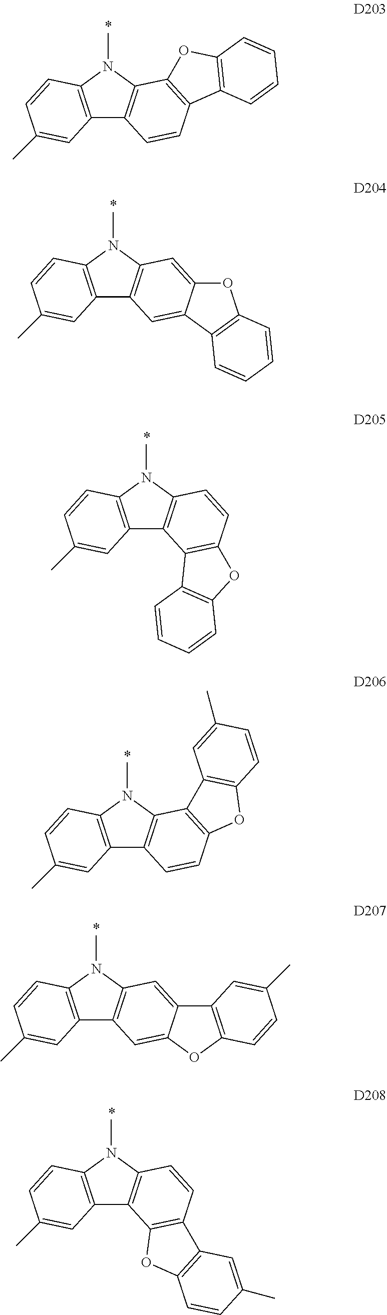

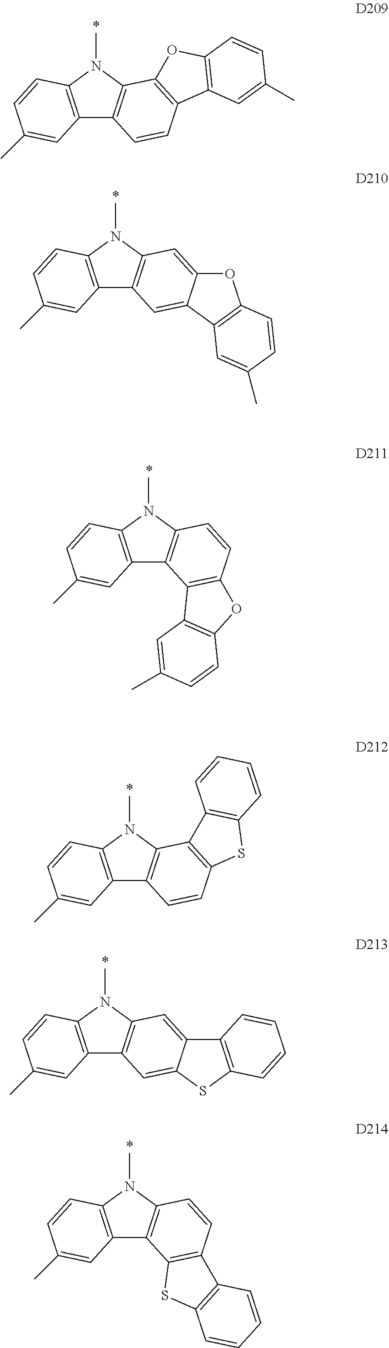

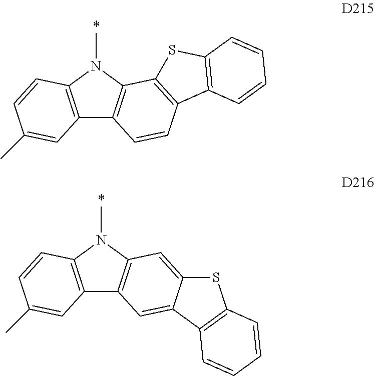 [SMILES: Cc1ccc2c(c1)c1ccc3c4ccccc4sc3c1n2C ; Cc1ccc2c(c1)c1cc3c(cc1n2C)sc1ccccc13]

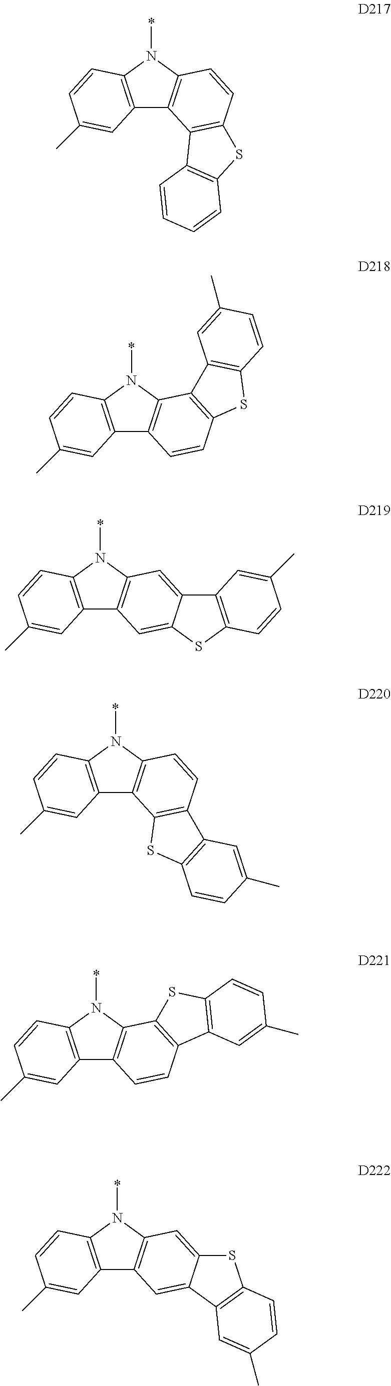

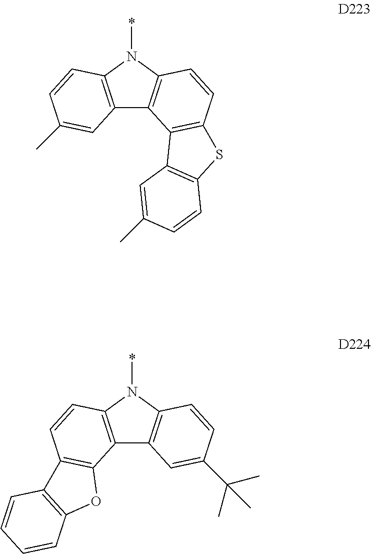 [SMILES: *n1c2ccc(C)cc2c2c3c(ccc21)sc1ccc(C)cc13 ; *n1c2ccc(C(C)(C)C)cc2c2c3oc4ccccc4c3ccc21]

Groups obtained by substituting all hydrogen atoms present in the above D1 to D224 with deuterium atoms are disclosed as D1(Da) to D224(Da). Groups obtained by substituting all hydrogen atoms present in the phenyl group or the alkyl group which is the substituent in the above D1 to D224 with deuterium atoms are disclosed as D1(Db) to D224(Db).

In one aspect of the present invention, the compounds represented by the general formula (1) have a group selected from the group consisting of D1 to D224, D1(Da) to D224(Da), and D1(Db) to D224(Db). For example, the compounds have only a group selected from the group consisting of D1 to D224, D1(Da) to D224(Da), and D1(Db) to D224(Db), as the substituted ring-fused carbazol-9-yl group.

In one preferred aspect of the present invention, the compounds represented by the general formula (1) have a group selected from the group consisting of D1 to D31, D1(Da) to D31(Da), and D1(Db) to D31(Db). For example, the compounds have only a group selected from the group consisting of D1 to D31, D1(Da) to D31(Da), and D1(Db) to D31(Db), as the substituted ring-fused carbazol-9-yl group.

In a more preferred aspect of the present invention, the compounds represented by the general formula (1) have a group selected from the group consisting of D1 to D9, D1(Da) to D9(Da), and D1(Db) to D9(Db). For example, the compounds have only a group selected from the group consisting of D1 to D9, D1(Da) to D9(Da), and D1(Db) to D9(Db), as the substituted ring-fused carbazol-9-yl group.

In one preferred aspect of the present invention, the compounds represented by the general formula (1) have a group selected from the group consisting of D10 to D31, D10(Da) to D31(Da), and D10(Db) to D31(Db). For example, the compounds have only a group selected from the group consisting of D10 to D31, D10(Da) to D31(Da), and D10(Db) to D31(Db), as the substituted ring-fused carbazol-9-yl group.

R¹ to R⁴ in the general formula (1) can be any other donor group than the substituted ring-fused carbazol-9-yl group. Here, the other donor group than the substituted ring-fused carbazol-9-yl group is referred to as “the other donor group”.

The donor group can be selected from groups having a negative Hammett's up value. The Hammett's up value is proposed by L. P. Hammett and quantifies the influence of a substituent on the reaction rate or equilibrium of a para-substituted benzene derivative. Specifically, the value is a constant (σp) peculiar to the substituent in the following equation that is established between a substituent and a reaction rate constant or an equilibrium constant in a para-substituted benzene derivative:

$$log ( k / {k}_{0} ) = ρσ p$$

$$or$$

$$log ( K / {K}_{0} ) = ρσ p$$

In the above equations, k₀ represents a rate constant of a benzene derivative not having a substituent; k represents a rate constant of a benzene derivative substituted with a substituent; K₀ represents an equilibrium constant of a benzene derivative not having a substituent; K represents an equilibrium constant of a benzene derivative substituted with a substituent; and ρ represents a reaction constant to be determined by the kind and the condition of reaction. Regarding the description relating to the “Hammett's op value” and the numerical value of each substituent in the present invention, reference can be made to the description relating to op value in Hansch, C. et. al., Chem. Rev., 91, 165-195 (1991).

The number of the other donor groups among R¹ to R⁴ is 0 to 3, preferably 0 to 2, more preferably 0 or 1. In the case where the number of the other donor groups is 2 or more, they can be the same as or different from each other. In one aspect of the present invention, the number of the other donor groups is 0. In one aspect of the present invention, the number of the other donor groups is 1. In one aspect of the present invention, R¹ is the other donor group. In one aspect of the present invention, R² is the other donor group. In one aspect of the present invention, R³ is the other donor group. In one aspect of the present invention, R⁴ is the other donor group. In one aspect of the present invention, only R¹ is the other donor group. In one aspect of the present invention, only R² is the other donor group. In one aspect of the present invention, only R³ is the other donor group. In one aspect of the present invention, only R⁴ is the other donor group.

The other donor group is preferably a substituted or unsubstituted diarylamino group, a substituted or unsubstituted dialkylamino group, or a substituted or unsubstituted alkylarylamino group, and more preferably a substituted or unsubstituted diarylamino group. The two aryl groups constituting the diarylamino group as referred to herein can bond to each other to form, for example, a cyclic structure such as a carbazole ring. Regarding the description and the preferred range of the aryl group and the alkyl group to constitute the diarylamino group, the dialkylamino group and the alkylarylamino group, reference can be made to the description and the preferred range of the aryl group and the alkyl group in the description section of the substituted ring-fused carbazol-9-yl group given hereinabove.

The other donor group can be a ring-fused indol-1-yl group. The number of the rings constituting the ring-fused indol-1-yl group is preferably 4 or more, more preferably 4 to 9, even more preferably 4 to 7.

In one preferred aspect of the present invention, the other donor group is a substituted or unsubstituted non-fused carbazol-9-yl group. The other donor group can be an unsubstituted ring-fused carbazol-9-yl group. In that case, the number of the rings constituting a fused ring of the ring-fused carbazol-9-yl group is preferably 4 or more, more preferably 5 to 9, even more preferably 5 to 7. In one aspect of the present invention, the number of the rings constituting the fused ring is 6. In one aspect of the present invention, the number of rings constituting the fused ring is 7. In one aspect of the present invention, the number of rings constituting the fused ring is 5. In any case, no substituent bonds to the ring skeleton-constituting carbon atom of the fused ring. At least one ring skeleton-constituting carbon atom of the two benzene rings that constitute the carbazol-9-yl group as referred to herein can be substituted with a nitrogen atom.

Specific examples of the other donor group which can be employed in the general formula (1) are shown below. However, the other donor group which can be employed in the present invention shall not be construed as being limited by the following specific examples. In the following specific examples, * indicates a bonding site, and Ph represents a phenyl group. A methyl group is not shown. Accordingly, for example, Z2 and Z3 have a methyl group.

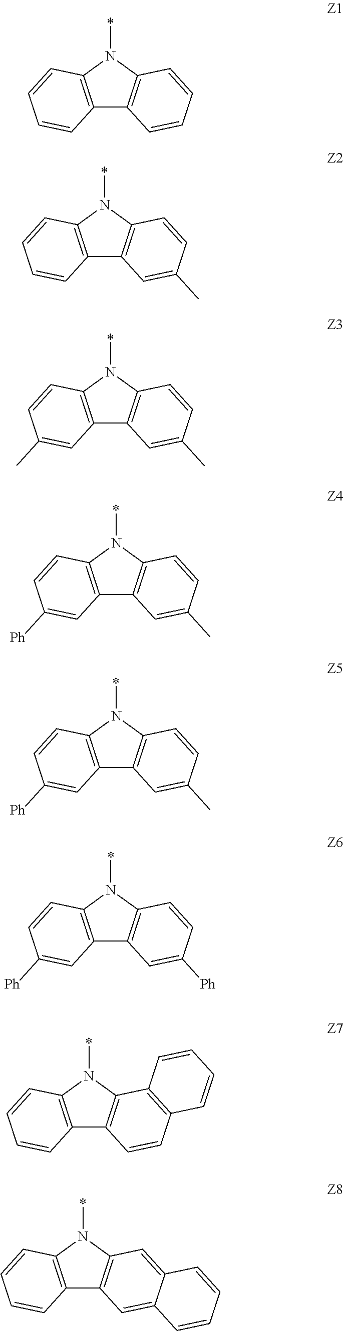

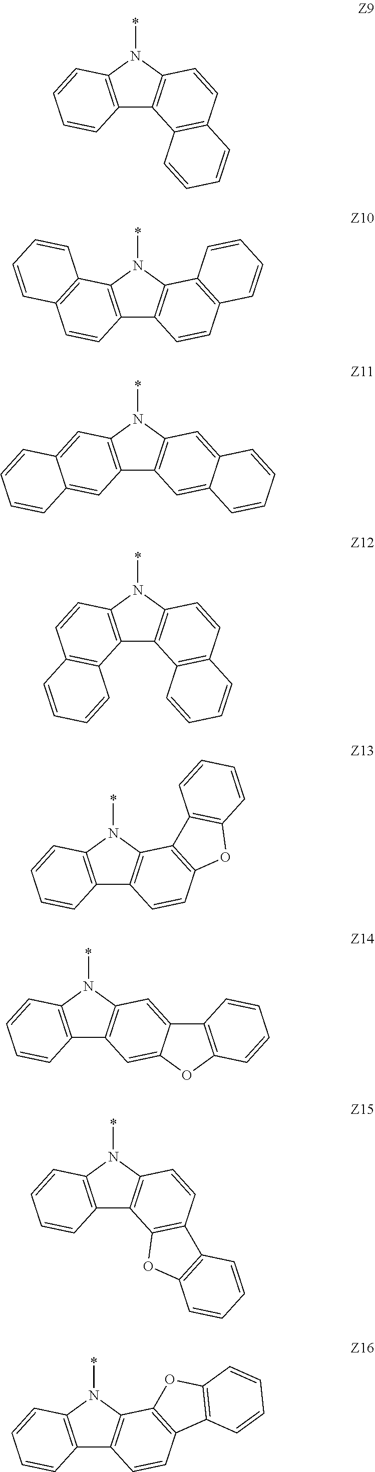

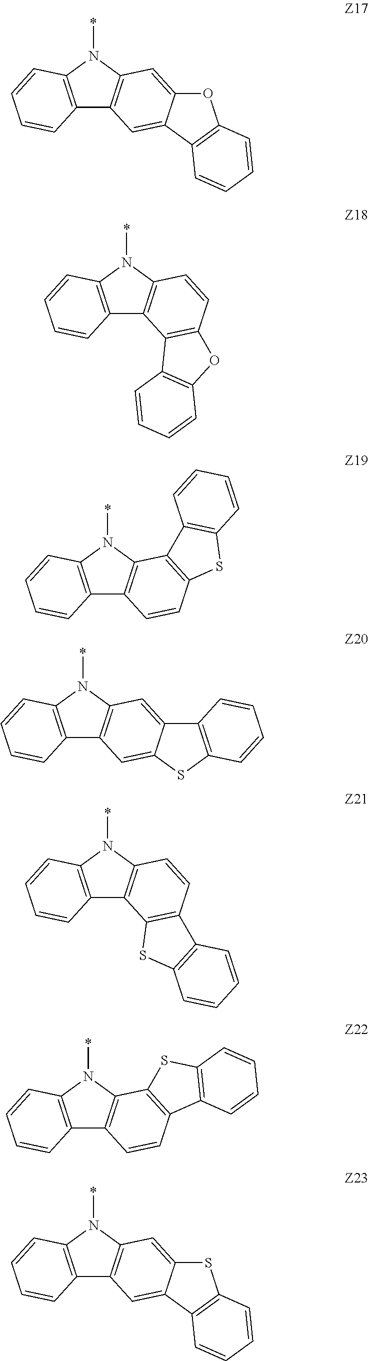

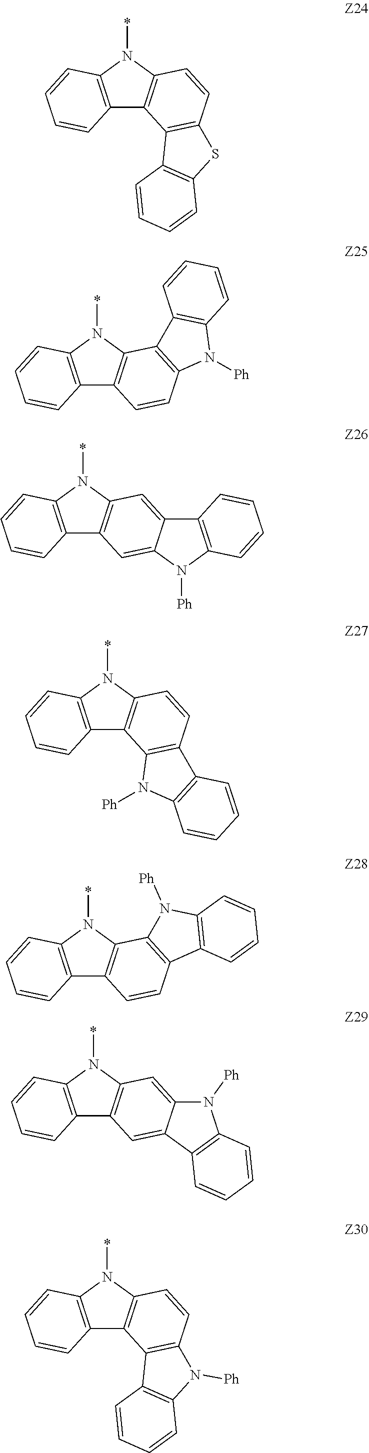

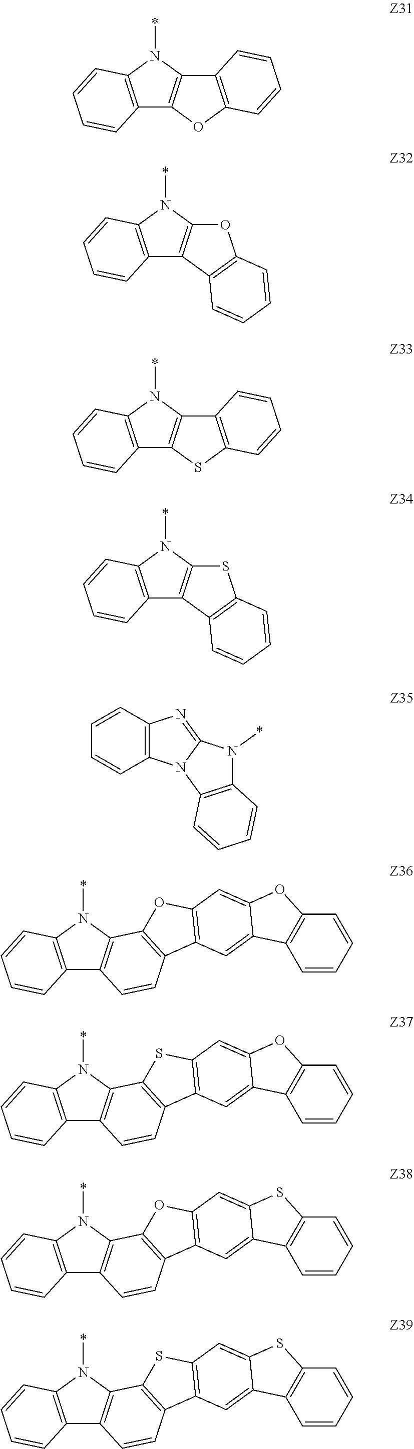

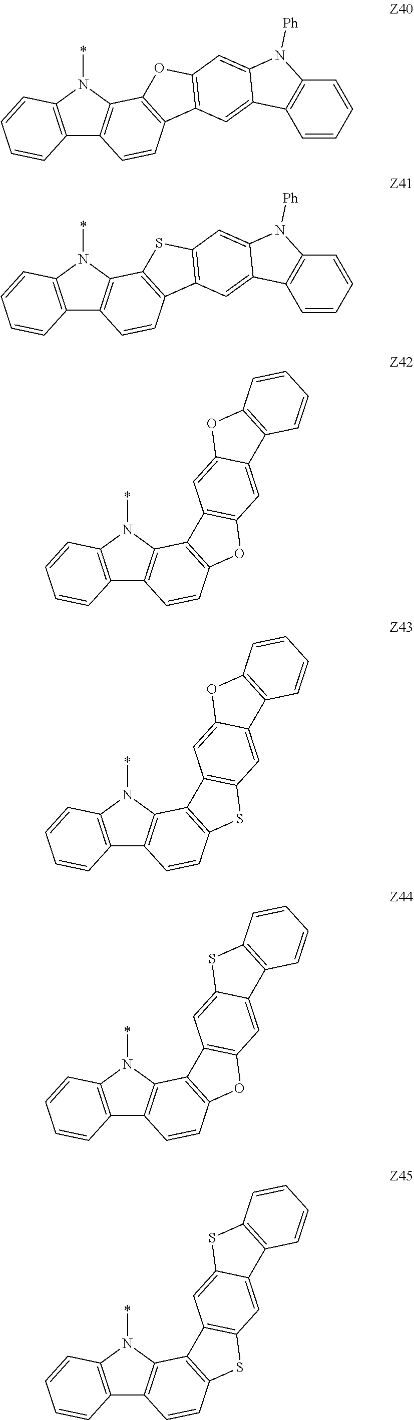 [SMILES: *n1c2ccccc2c2ccc3c4cc5c6ccccc6n(-c6ccccc6)c5cc4oc3c21 ; *n1c2ccccc2c2ccc3c4cc5c6ccccc6n(-c6ccccc6)c5cc4sc3c21 ; *n1c2ccccc2c2ccc3oc4cc5c(cc4c3c21)oc1ccccc15 ; *n1c2ccccc2c2ccc3sc4cc5c(cc4c3c21)oc1ccccc15 ; *n1c2ccccc2c2ccc3oc4cc5c(cc4c3c21)sc1ccccc15 ; *n1c2ccccc2c2ccc3sc4cc5c(cc4c3c21)sc1ccccc15]

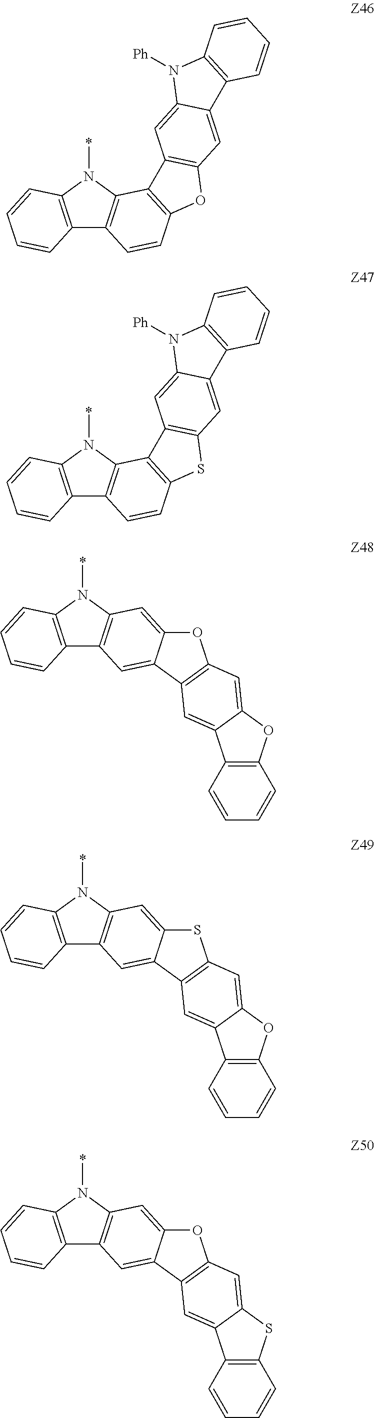 [SMILES: *n1c2ccccc2c2ccc3oc4cc5c6ccccc6n(-c6ccccc6)c5cc4c3c21 ; *n1c2ccccc2c2ccc3sc4cc5c6ccccc6n(-c6ccccc6)c5cc4c3c21 ; *n1c2ccccc2c2cc3c(cc21)oc1cc2oc4ccccc4c2cc13 ; *n1c2ccccc2c2cc3c(cc21)sc1cc2oc4ccccc4c2cc13 ; *n1c2ccccc2c2cc3c(cc21)oc1cc2sc4ccccc4c2cc13]

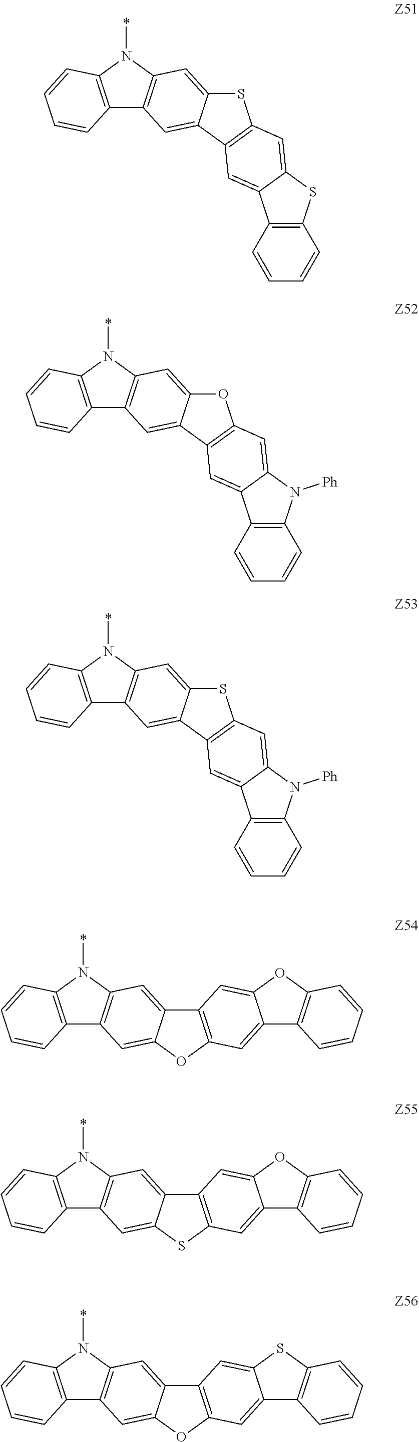 [SMILES: *n1c2ccccc2c2cc3c(cc21)sc1cc2sc4ccccc4c2cc13 ; *n1c2ccccc2c2cc3c(cc21)oc1cc2c(cc13)c1ccccc1n2-c1ccccc1 ; *n1c2ccccc2c2cc3c(cc21)sc1cc2c(cc13)c1ccccc1n2-c1ccccc1 ; *n1c2ccccc2c2cc3oc4cc5c(cc4c3cc21)oc1ccccc15 ; *n1c2ccccc2c2cc3sc4cc5c(cc4c3cc21)oc1ccccc15 ; *n1c2ccccc2c2cc3oc4cc5c(cc4c3cc21)sc1ccccc15]

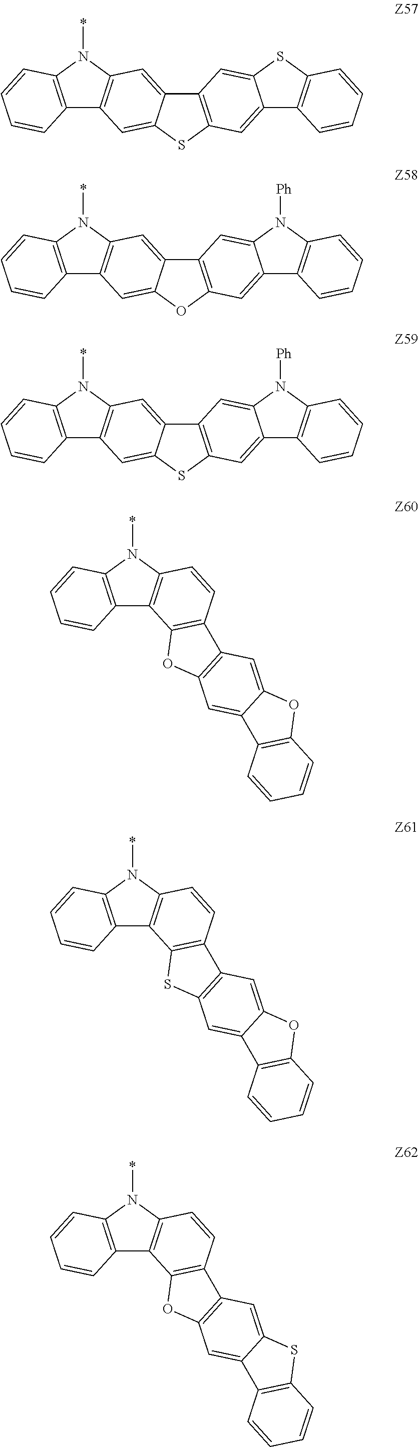 [SMILES: *n1c2ccccc2c2cc3sc4cc5c(cc4c3cc21)sc1ccccc15 ; *n1c2ccccc2c2cc3oc4cc5c6ccccc6n(-c6ccccc6)c5cc4c3cc21 ; *n1c2ccccc2c2cc3sc4cc5c6ccccc6n(-c6ccccc6)c5cc4c3cc21 ; *n1c2ccccc2c2c3oc4cc5c(cc4c3ccc21)oc1ccccc15 ; *n1c2ccccc2c2c3sc4cc5c(cc4c3ccc21)oc1ccccc15 ; *n1c2ccccc2c2c3oc4cc5c(cc4c3ccc21)sc1ccccc15]

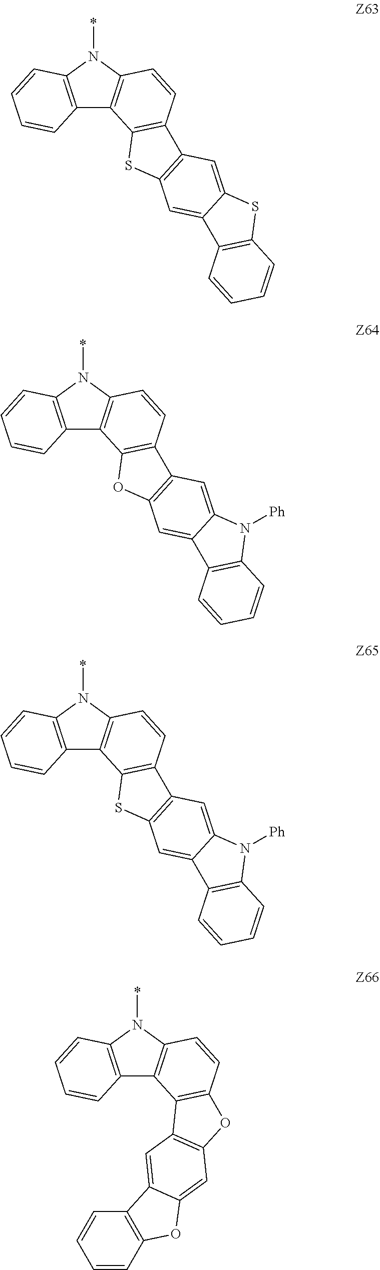 [SMILES: *n1c2ccccc2c2c3sc4cc5c(cc4c3ccc21)sc1ccccc15 ; *n1c2ccccc2c2c3oc4cc5c6ccccc6n(-c6ccccc6)c5cc4c3ccc21 ; *n1c2ccccc2c2c3sc4cc5c6ccccc6n(-c6ccccc6)c5cc4c3ccc21 ; *n1c2ccccc2c2c3c(ccc21)oc1cc2oc4ccccc4c2cc13]

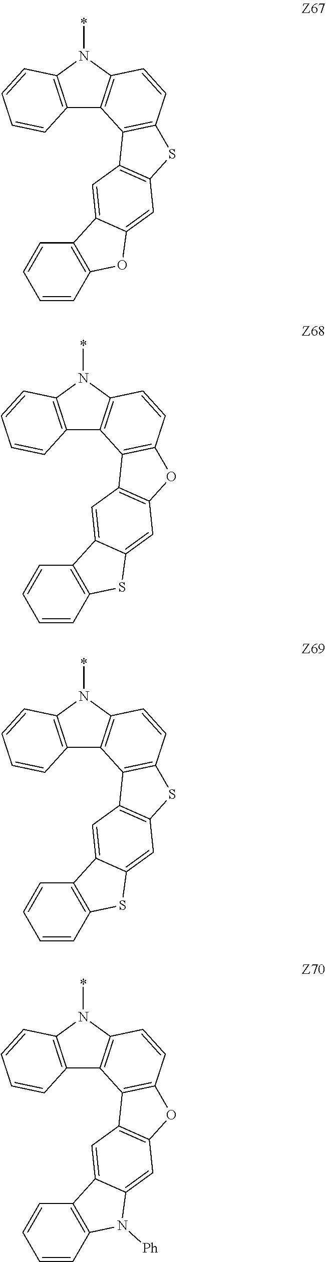

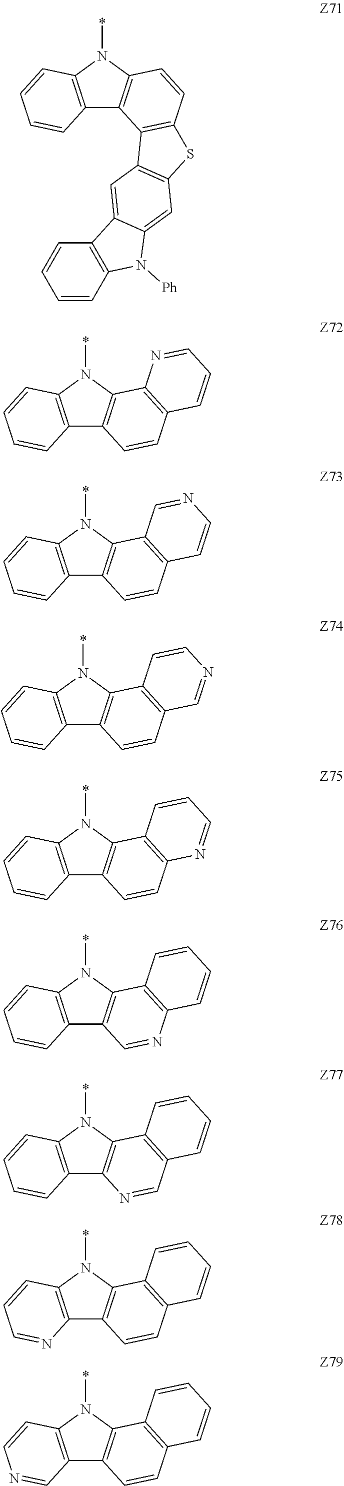

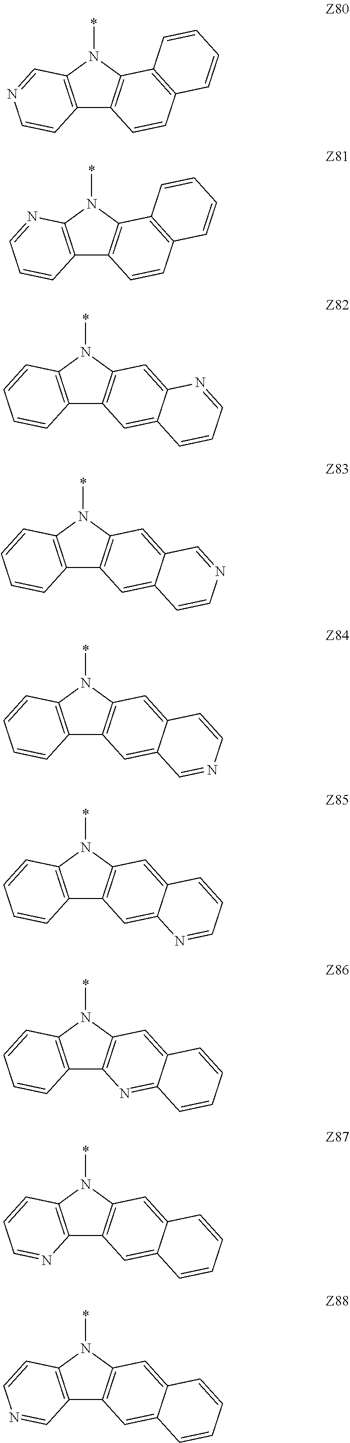

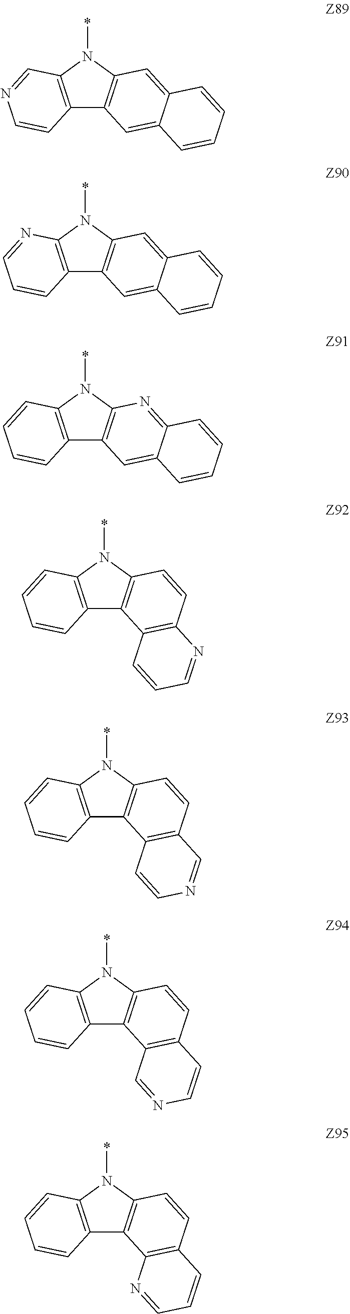

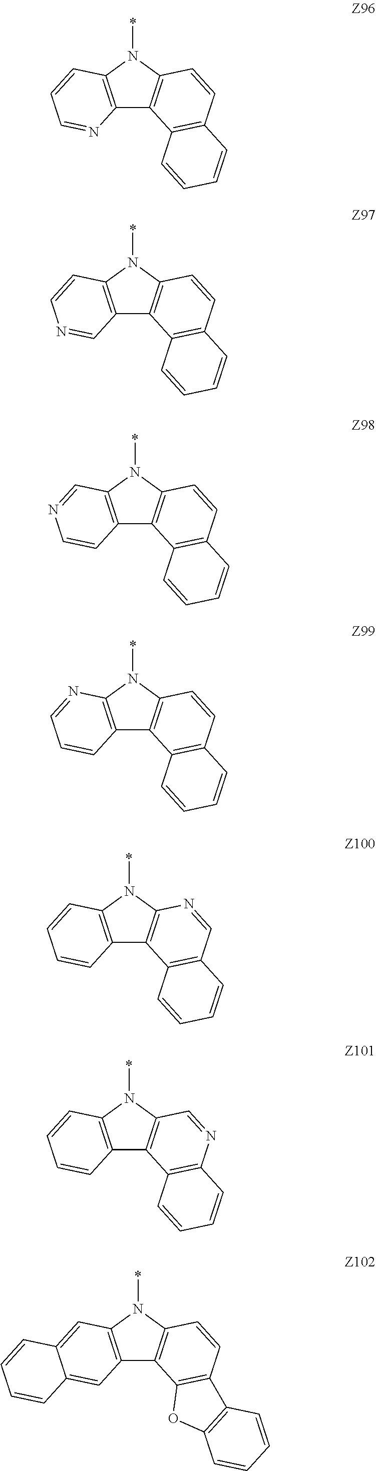

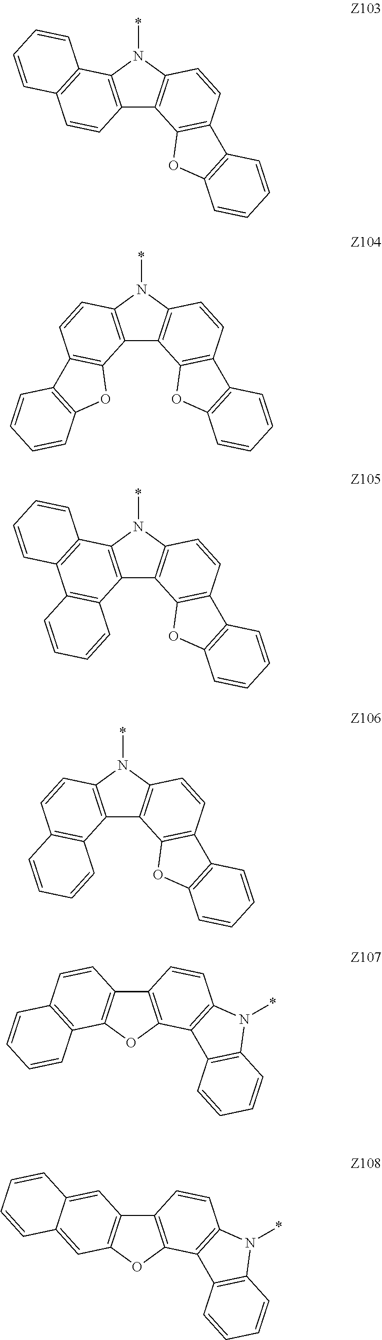 [SMILES: *n1c2ccc3c4ccccc4oc3c2c2ccc3ccccc3c21 ; *n1c2ccc3c4ccccc4oc3c2c2c3oc4ccccc4c3ccc21 ; *n1c2ccc3c4ccccc4oc3c2c2c3ccccc3c3ccccc3c21 ; *n1c2ccc3ccccc3c2c2c3oc4ccccc4c3ccc21 ; *n1c2ccccc2c2c3oc4c5ccccc5ccc4c3ccc21 ; *n1c2ccccc2c2c3oc4cc5ccccc5cc4c3ccc21]

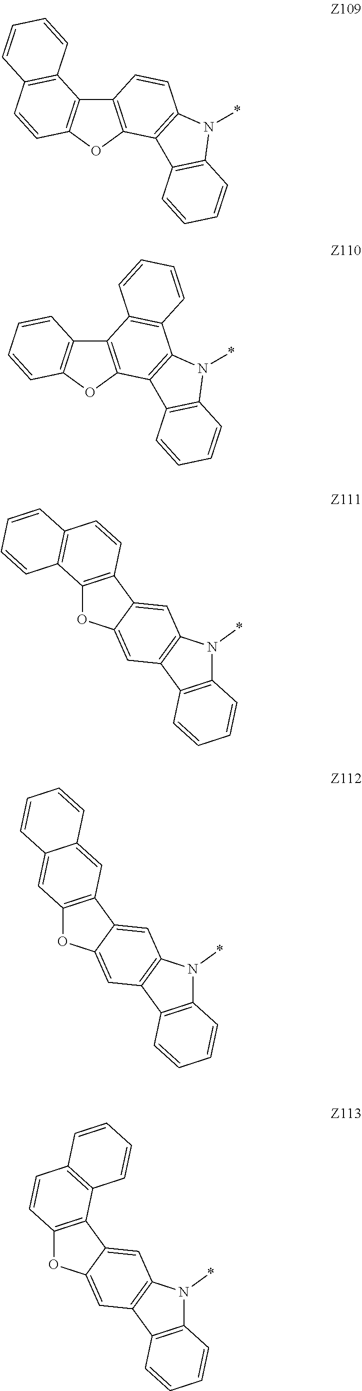

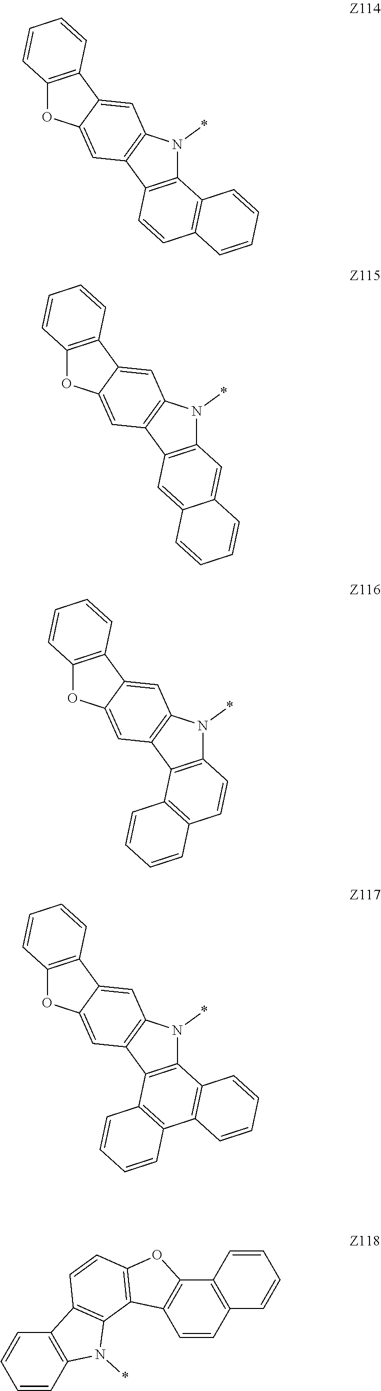 [SMILES: *n1c2cc3c(cc2c2ccc4ccccc4c21)oc1ccccc13 ; *n1c2cc3ccccc3cc2c2cc3oc4ccccc4c3cc21 ; *n1c2cc3c(cc2c2c4ccccc4ccc21)oc1ccccc13 ; *n1c2cc3c(cc2c2c4ccccc4c4ccccc4c21)oc1ccccc13 ; *n1c2ccccc2c2ccc3oc4c5ccccc5ccc4c3c21]

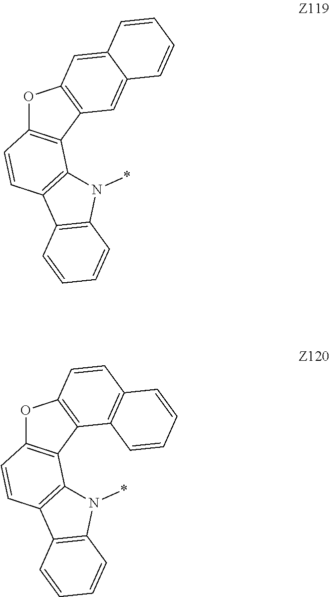 [SMILES: *n1c2ccccc2c2ccc3oc4cc5ccccc5cc4c3c21 ; *n1c2ccccc2c2ccc3oc4ccc5ccccc5c4c3c21]

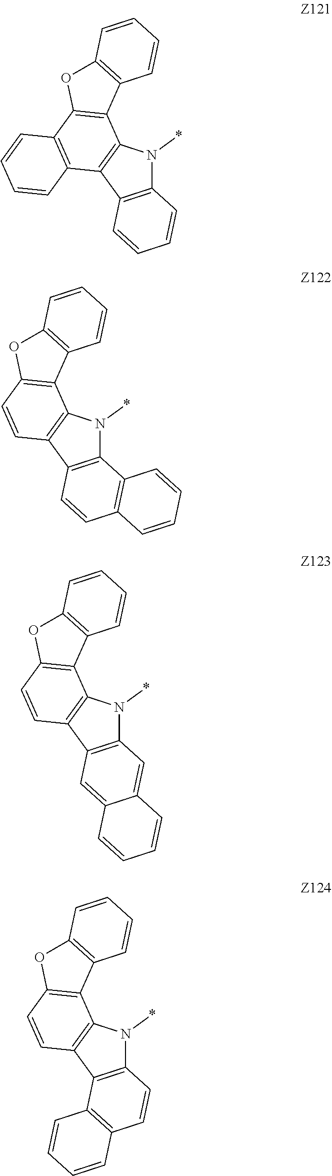

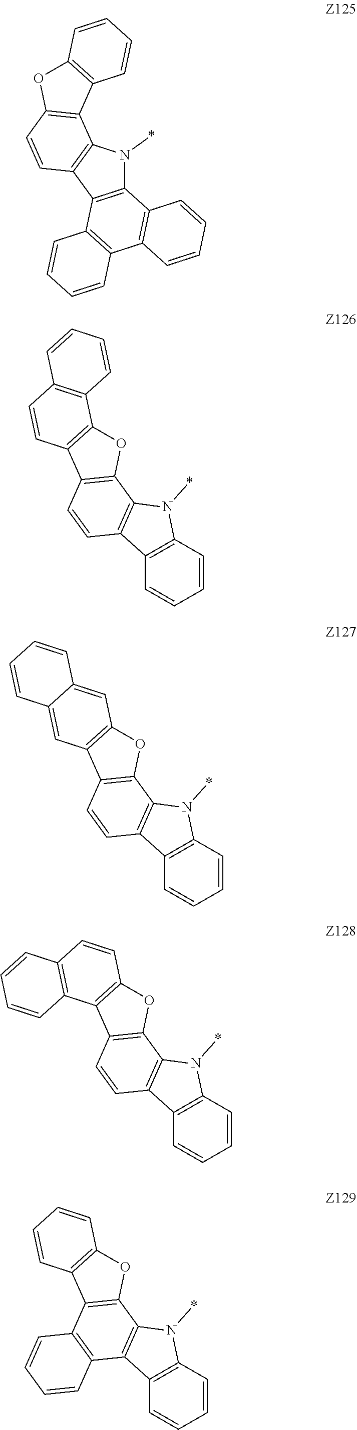

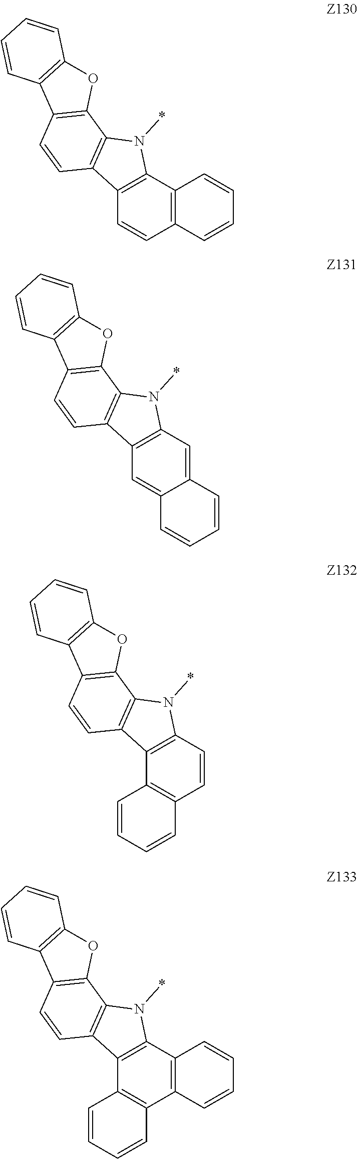 [SMILES: *n1c2c3ccccc3ccc2c2ccc3c4ccccc4oc3c21 ; *n1c2cc3ccccc3cc2c2ccc3c4ccccc4oc3c21 ; *n1c2ccc3ccccc3c2c2ccc3c4ccccc4oc3c21 ; *n1c2c(ccc3c4ccccc4oc32)c2c3ccccc3c3ccccc3c21]

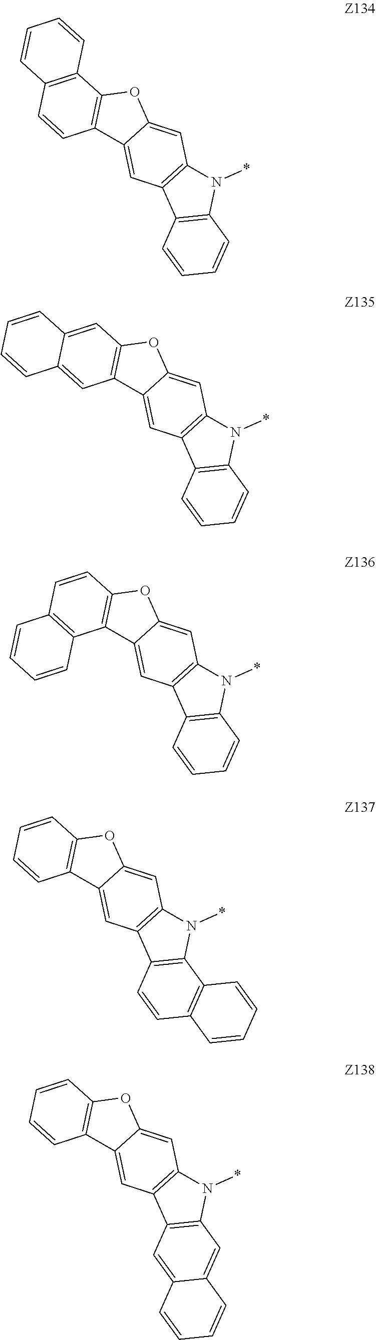 [SMILES: *n1c2ccccc2c2cc3c(cc21)oc1c2ccccc2ccc31 ; *n1c2ccccc2c2cc3c(cc21)oc1cc2ccccc2cc13 ; *n1c2ccccc2c2cc3c(cc21)oc1ccc2ccccc2c13 ; *n1c2cc3oc4ccccc4c3cc2c2ccc3ccccc3c21 ; *n1c2cc3ccccc3cc2c2cc3c(cc21)oc1ccccc13]

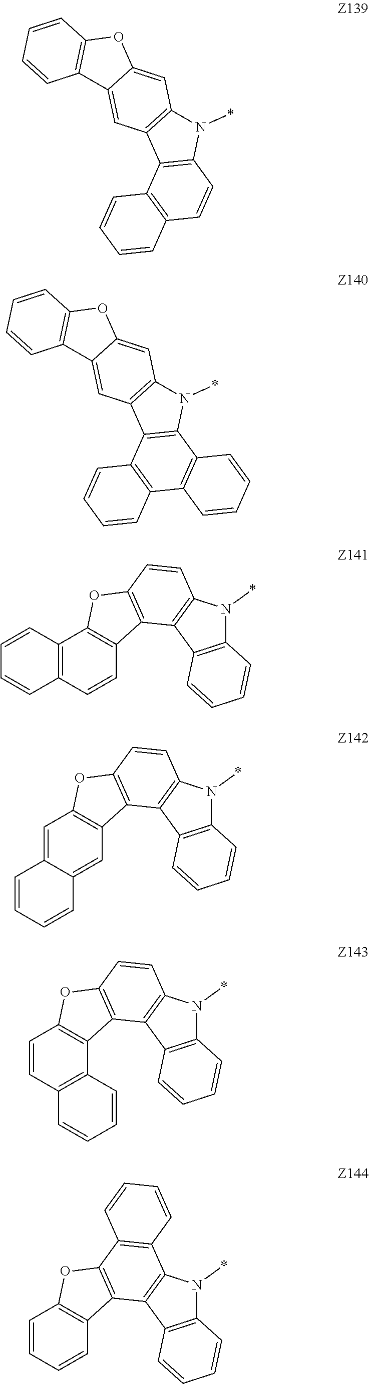

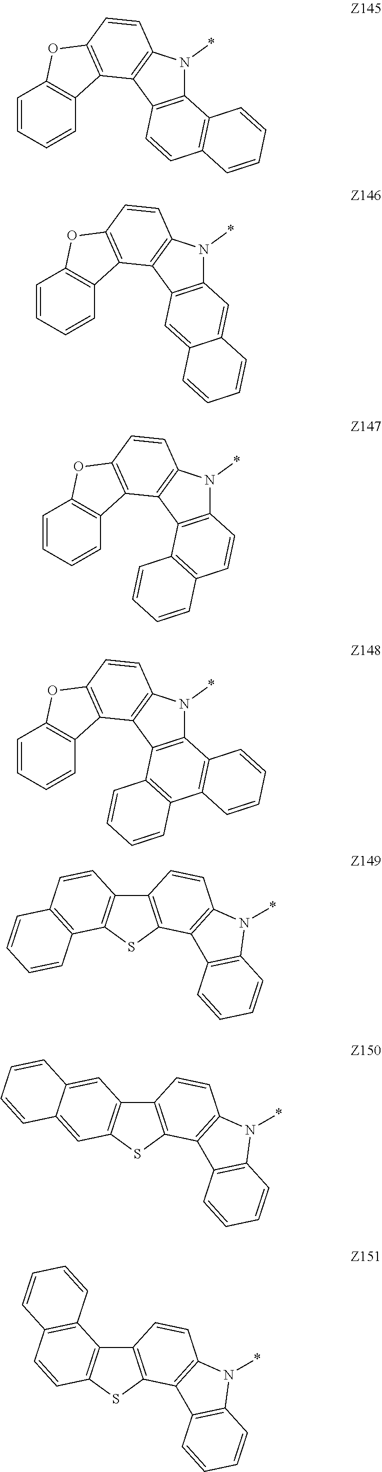

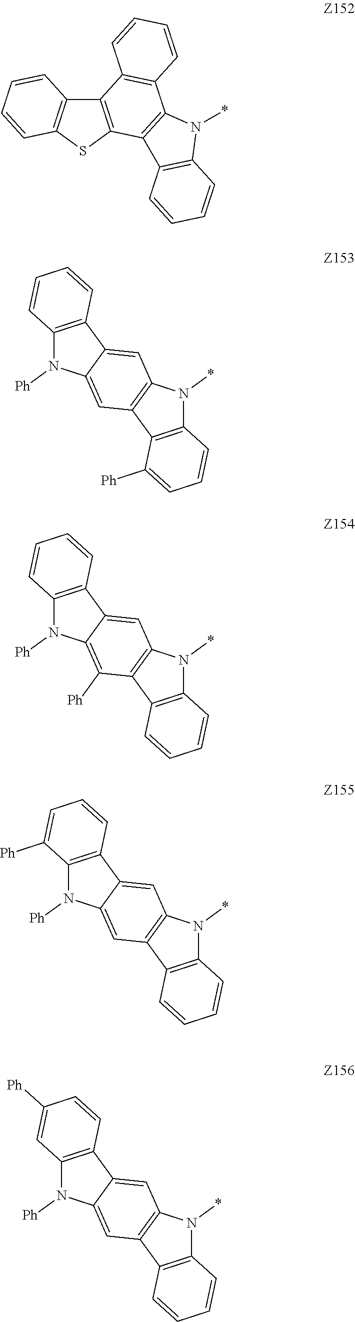 [SMILES: *n1c2ccccc2c2c3sc4ccccc4c3c3ccccc3c21 ; *n1c2cc3c4ccccc4n(-c4ccccc4)c3cc2c2c(-c3ccccc3)cccc21 ; *n1c2ccccc2c2c(-c3ccccc3)c3c(cc21)c1ccccc1n3-c1ccccc1 ; *n1c2ccccc2c2cc3c(cc21)c1cccc(-c2ccccc2)c1n3-c1ccccc1 ; *n1c2ccccc2c2cc3c(cc21)c1ccc(-c2ccccc2)cc1n3-c1ccccc1]

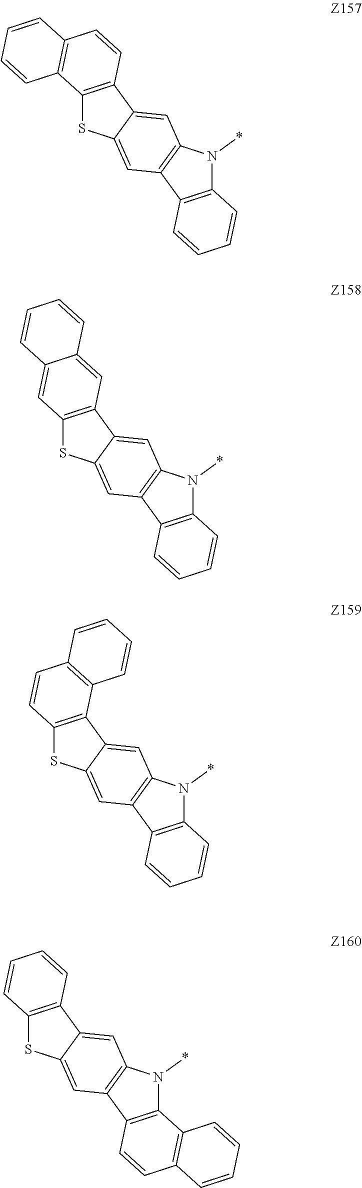 [SMILES: *n1c2ccccc2c2cc3sc4c5ccccc5ccc4c3cc21 ; *n1c2ccccc2c2cc3sc4cc5ccccc5cc4c3cc21 ; *n1c2ccccc2c2cc3sc4ccc5ccccc5c4c3cc21 ; *n1c2cc3c(cc2c2ccc4ccccc4c21)sc1ccccc13]

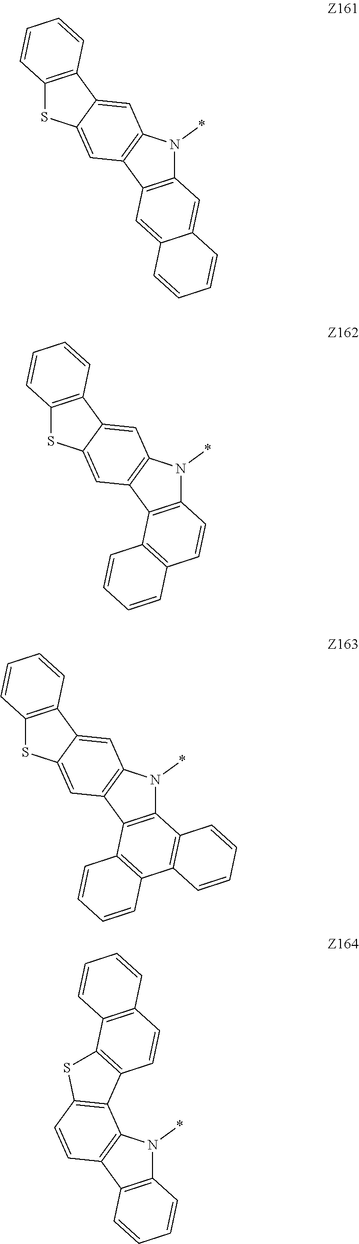

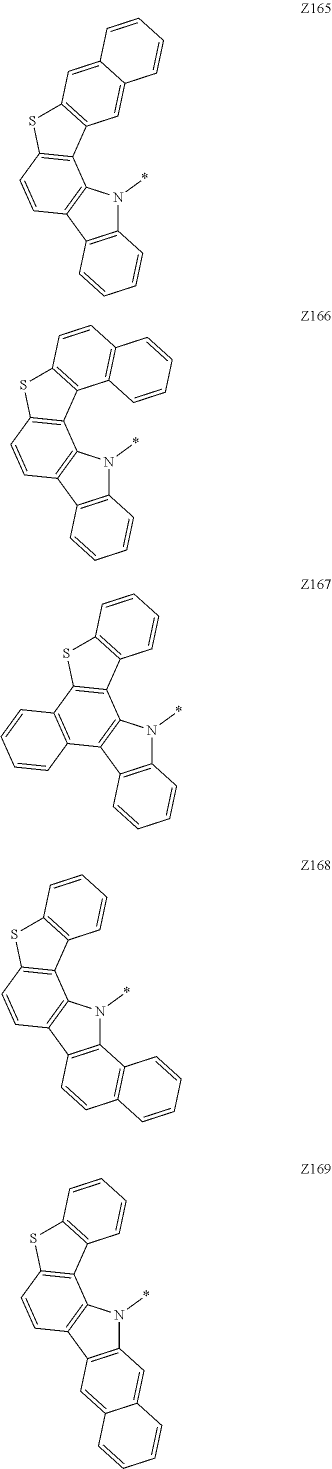

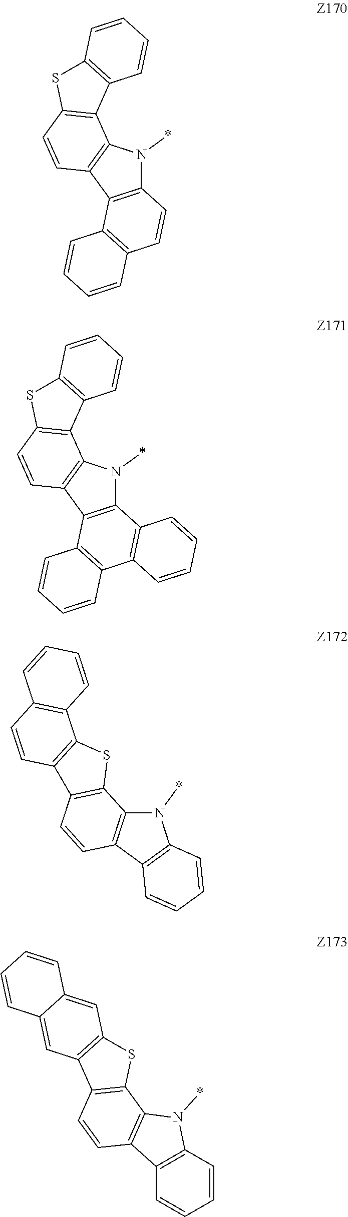

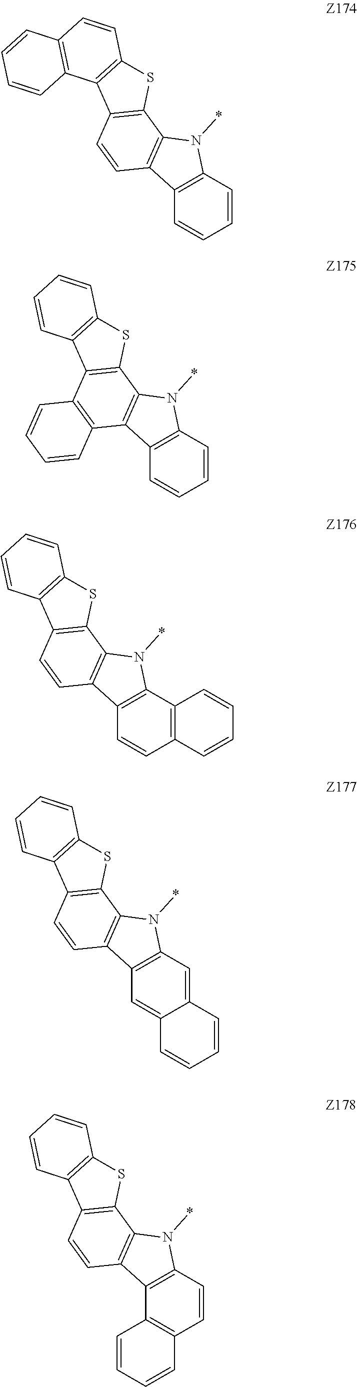 [SMILES: *n1c2ccccc2c2ccc3c(sc4ccc5ccccc5c43)c21 ; *n1c2ccccc2c2c3ccccc3c3c4ccccc4sc3c21 ; *n1c2c3ccccc3ccc2c2ccc3c4ccccc4sc3c21 ; *n1c2cc3ccccc3cc2c2ccc3c4ccccc4sc3c21 ; *n1c2ccc3ccccc3c2c2ccc3c4ccccc4sc3c21]

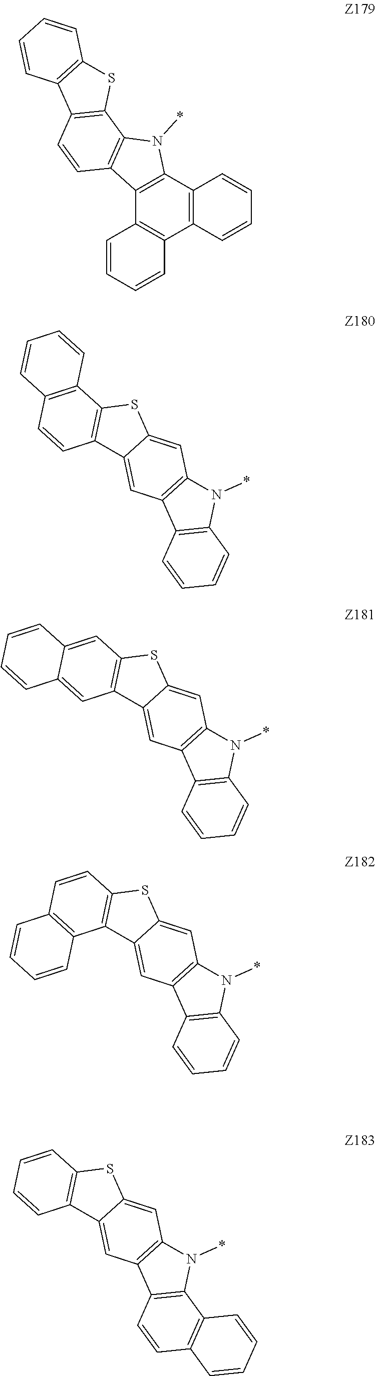 [SMILES: *n1c2c(ccc3c4ccccc4sc32)c2c3ccccc3c3ccccc3c21 ; *n1c2ccccc2c2cc3c(cc21)sc1c2ccccc2ccc31 ; *n1c2ccccc2c2cc3c(cc21)sc1cc2ccccc2cc13 ; *n1c2ccccc2c2cc3c(cc21)sc1ccc2ccccc2c13 ; *n1c2cc3sc4ccccc4c3cc2c2ccc3ccccc3c21]

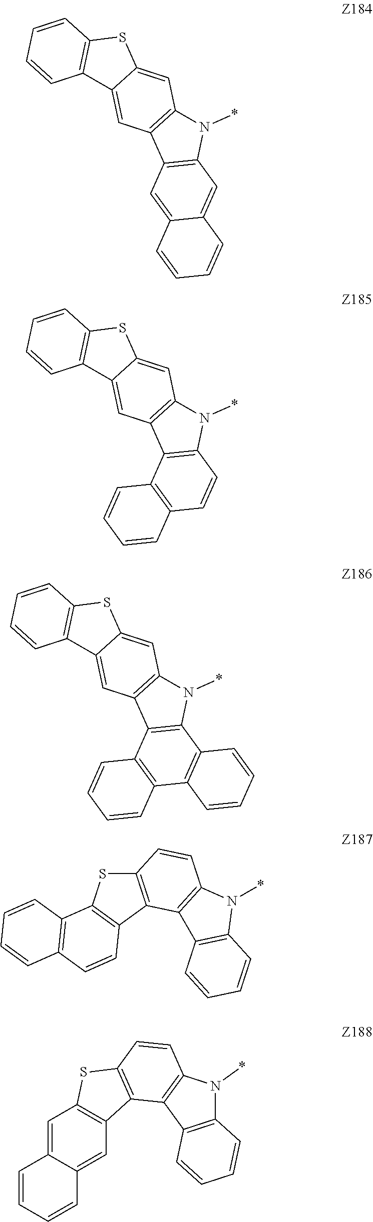

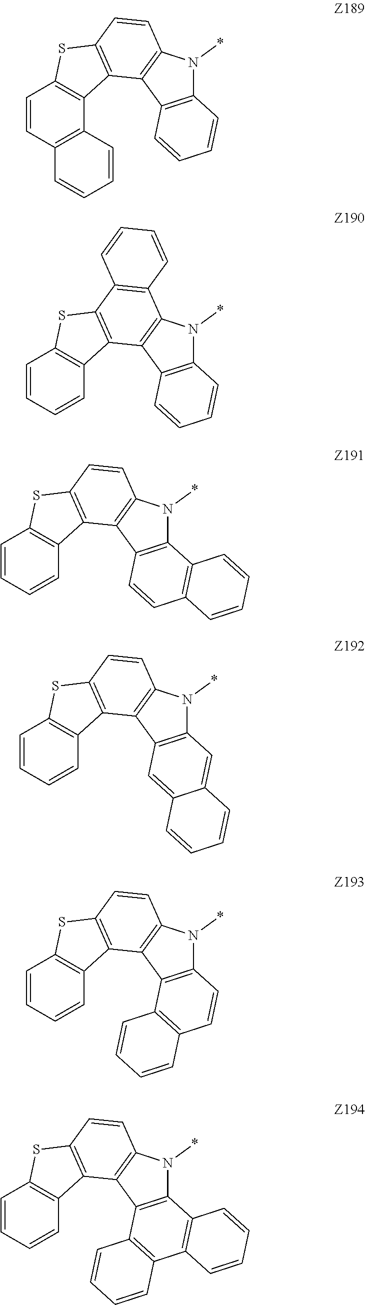 [SMILES: *n1c2ccccc2c2c3c(ccc21)sc1ccc2ccccc2c13 ; *n1c2ccccc2c2c3c4ccccc4sc3c3ccccc3c21 ; *n1c2ccc3sc4ccccc4c3c2c2ccc3ccccc3c21 ; *n1c2cc3ccccc3cc2c2c3c(ccc21)sc1ccccc13 ; *n1c2ccc3ccccc3c2c2c3c(ccc21)sc1ccccc13 ; *n1c2ccc3sc4ccccc4c3c2c2c3ccccc3c3ccccc3c21]

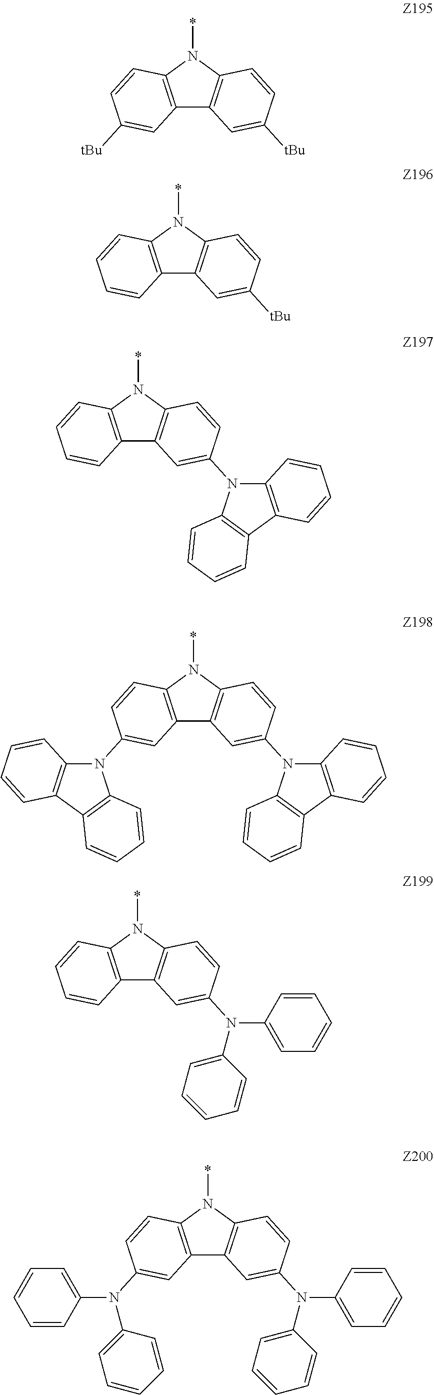

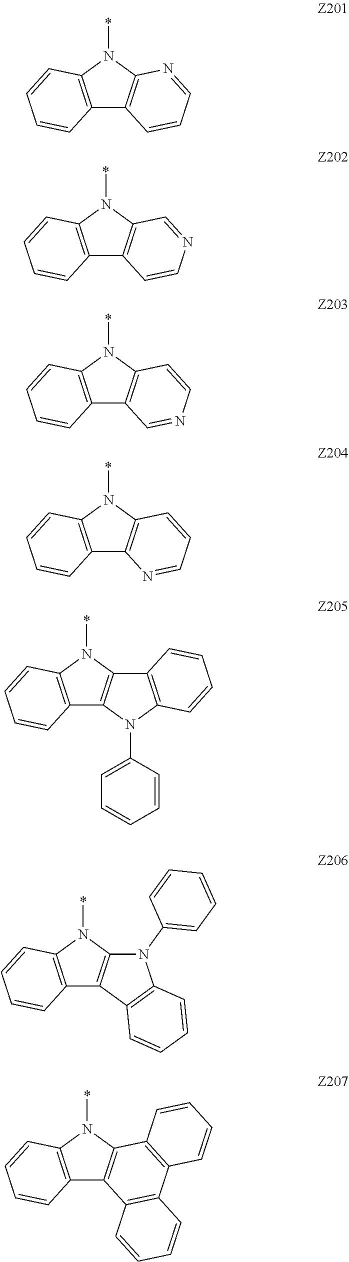

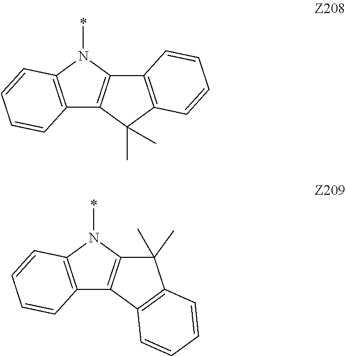 [SMILES: *n1c2c(c3ccccc31)C(C)(C)c1ccccc1-2 ; *n1c2c(c3ccccc31)-c1ccccc1C2(C)C]

Groups obtained by substituting all hydrogen atoms present in the above Z1 to Z209 with deuterium atoms are disclosed as Z1(Da) to Z209(Da).

In one aspect of the present invention, the compounds represented by the general formula (1) have a group selected from the group consisting of Z1 to Z209 and Z1(Da) to Z209(Da). For example, the compounds have only a group selected from the group consisting of Z1 to Z209 and Z1(Da) to Z209(Da), as the other donor group.

In one aspect of the present invention, the compounds represented by the general formula (1) have a group selected from the group consisting of Z1 to Z6, Z195 to Z209, Z1(Da) to Z6(Da), and Z195(Da) to Z209(Da). For example, the compounds have only a group selected from the group consisting of Z1 to Z6, Z195 to Z209, Z1(Da) to Z6(Da), and Z195(Da) to Z209(Da), as the other donor group.

In one aspect of the present invention, the compounds represented by the general formula (1) have a group selected from the group consisting of Z7 to Z194, and Z7(Da) to Z194 (Da). For example, the compounds have only a group selected from the group consisting of Z7 to Z194, and Z7(Da) to Z194 (Da), as the other donor group.

In the general formula (1), R¹ to R⁴ each can be a hydrogen atom, a deuterium atom, a substituted or unsubstituted alkyl group, or a substituted or unsubstituted aryl group.

Regarding the description and the preferred range of the aryl group and the alkyl group, reference can be made to the description and the preferred range of the aryl group and the alkyl group in the description section of the substituted ring-fused carbazol-9-yl group given hereinabove. In one aspect of the present invention, the hydrogen atom of the aryl group and the alkyl group can be substituted with a deuterium atom and a group selected from Substituent Group E. In one aspect of the present invention, the aryl group and the alkyl group are unsubstituted.

Specific examples of the alkyl group include a methyl group, an ethyl group, an isopropyl group, an n-propyl group, and a tert-butyl group. Specific examples of the substituted or unsubstituted aryl group further include the following groups. However, the substituted or unsubstituted alkyl group and the substituted or unsubstituted aryl group that can be employed in the present invention are not to be construed as being limited by these specific examples. In the following specific examples, t-Bu represents a tert-butyl group, and * indicates a bonding site.

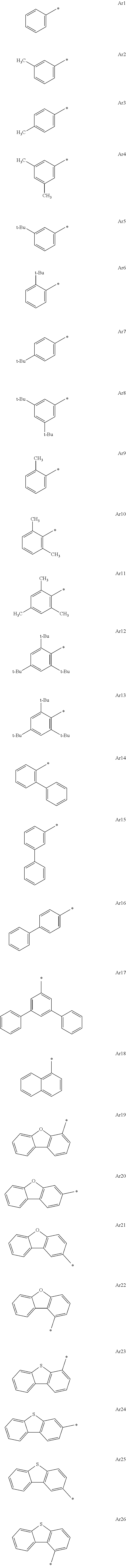

Groups obtained by substituting all hydrogen atoms present in the above Ar1 to Ar26 with deuterium atoms are disclosed as Ar1(Da) to Ar26(Da). Groups obtained by substituting all hydrogen atoms present in the alkyl group or the phenyl group of the substituent in the above Ar2 to Ar18 with deuterium atoms are disclosed as Ar2(Db) to Ar18(Db).

In one aspect of the present invention, at least one of R¹ to R⁴ is a hydrogen atom or a deuterium atom. In one preferred aspect of the present invention, only one of R¹ to R⁴ is a hydrogen atom or a deuterium atom. In one preferred aspect of the present invention, R¹ is a hydrogen atom or a deuterium atom. In one aspect of the present invention, R² is a hydrogen atom or a deuterium atom. In one aspect of the present invention, R³ is a hydrogen atom or a deuterium atom. In one aspect of the present invention, R⁴ is a hydrogen atom or a deuterium atom. In one preferred aspect of the present invention, only R¹ is a hydrogen atom or a deuterium atom. In one aspect of the present invention, only R² is a hydrogen atom or a deuterium atom. In one aspect of the present invention, only R³ is a hydrogen atom or a deuterium atom. In one aspect of the present invention, only R⁴ is a hydrogen atom or a deuterium atom.

In one aspect of the present invention, R¹ is a substituted or unsubstituted alkyl group. In one aspect of the present invention, R² is a substituted or unsubstituted alkyl group. In one aspect of the present invention, R³ is a substituted or unsubstituted alkyl group. In one aspect of the present invention, R⁴ is a substituted or unsubstituted alkyl group.

In one aspect of the present invention, R¹ is a substituted or unsubstituted aryl group. In one aspect of the present invention, R² is a substituted or unsubstituted aryl group. In one preferred aspect of the present invention, R³ is a substituted or unsubstituted aryl group. In one aspect of the present invention, R⁴ is a substituted or unsubstituted aryl group.

In one preferred aspect of the present invention, two of R¹ to R⁴ are donor groups, one is a hydrogen atom or a deuterium atom, and one is a substituted or unsubstituted aryl group. More preferably, two of R¹ to R⁴ are substituted ring-fused carbazol-9-yl groups, one is a hydrogen atom or a deuterium atom, and one is an unsubstituted aryl group. Further preferably, two of R¹ to R⁴ are ring-fused carbazol-9-yl groups substituted with an alkyl group or an aryl group, one is a hydrogen atom or a deuterium atom, and one is an unsubstituted phenyl group.

In one aspect of the present invention, three of R¹ to R⁴ are donor groups, and one is a hydrogen atom or a deuterium atom. In one aspect of the present invention, three of R¹ to R⁴ are donor groups, and one is a substituted or unsubstituted aryl group (preferably an unsubstituted aryl group). In one aspect of the present invention, three of R¹ to R⁴ are ring-fused carbazol-9-yl groups substituted with an alkyl group or an aryl group, and one is a hydrogen atom or a deuterium atom. In one aspect of the present invention, three of R¹ to R⁴ are ring-fused carbazol-9-yl groups substituted with an alkyl group or an aryl group, and one is a substituted or unsubstituted aryl group (preferably an unsubstituted aryl group).

In one aspect of the present invention, R¹ to R⁴ are all donor groups. In one aspect of the present invention, R¹ to R⁴ are all ring-fused carbazol-9-yl groups substituted with an alkyl group or an aryl group.

In one aspect of the present invention, R¹ and R² are donor groups. In one aspect of the present invention, R¹ and R³ are donor groups. In one aspect of the present invention, R¹ and R⁴ are donor groups. In one aspect of the present invention, R² and R₃ are donor groups. In one aspect of the present invention, R³ and R⁴ are donor groups. In one aspect of the present invention, R¹, R² and R³ are donor groups. In one aspect of the present invention, R¹, R² and R⁴ are donor groups. In one aspect of the present invention, R¹, R³ and R⁴ are donor groups. In one aspect of the present invention, R², R³ and R⁴ are donor groups.

In one preferred aspect of the present invention, R¹ is a hydrogen atom or a deuterium atom, R² and R⁴ are donor groups (preferably ring-fused carbazol-9-yl groups substituted with an alkyl group or an aryl group), and R³ is a substituted or unsubstituted aryl group (preferably an unsubstituted aryl group). In one aspect of the present invention, R¹ is a hydrogen atom or a deuterium atom, R² and R⁴ are donor groups (preferably ring-fused carbazol-9-yl groups substituted with an alkyl group or an aryl group), and R³ is a substituted or unsubstituted alkyl group (preferably an unsubstituted alkyl group). In one aspect of the present invention, R¹ is a hydrogen atom or a deuterium atom, R² and R⁴ are donor groups (preferably ring-fused carbazol-9-yl groups substituted with an alkyl group or an aryl group), and R³ is a hydrogen atom or a deuterium atom. In one aspect of the present invention, R¹ is a substituted or unsubstituted aryl group (preferably an unsubstituted aryl group), R² and R⁴ are donor groups (preferably ring-fused carbazol-9-yl groups substituted with an alkyl group or an aryl group), and R³ is a hydrogen atom or a deuterium atom.

In one aspect of the present invention, R¹ is a hydrogen atom or a deuterium atom, and R² to R⁴ are donor groups. In one aspect of the present invention, R¹ is a hydrogen atom or a deuterium atom, and R² to R⁴ are ring-fused carbazol-9-yl groups substituted with an alkyl group or an aryl group. In one aspect of the present invention, R¹ is a hydrogen atom or a deuterium atom, R² and R⁴ are ring-fused carbazol-9-yl groups substituted with an alkyl group or an aryl group, and R³ is the other donor group.

In one preferred aspect of the present invention, in the general formula (1) of the present invention, none of R¹ and R², R² and R³, and R³ and R⁴ bonds to each other to form a cyclic structure.

In the general formula (1), X¹ to X³ each independently represent N or C(R). However, at least one of X¹ to X³ is N. R represents a hydrogen atom, a deuterium atom or a substituent. As referred to herein, the substituent can be selected from Substituent Group A, can be selected from Substituent Group B, can be selected from Substituent Group C, can be selected from Substituent Group D, or can be selected from Substituent Group E. In one preferred aspect of the present invention, X¹ to X³ are N. In one aspect of the present invention, X¹ and X³ are N, and X² is C(R). In one aspect of the present invention, X¹ and X² are N, and X³ is C(R). In one aspect of the present invention, X¹ is N, and X² and X³ are C(R). In one aspect of the present invention, X² is N, and X¹ and X³ are C(R).

In the general formula (1), Ar¹ and Ar² each independently represent a substituted or unsubstituted aryl group. Regarding the description and the preferred range of the substituted or unsubstituted aryl group, reference can be made to the description and the preferred range of the alkyl group and the aryl group in the description section of the substituted ring-fused carbazol-9-yl group given hereinabove. Specific examples of Ar¹ and Ar² include the above Ar1 to Ar26, Ar1(Da) to Ar26(Da), and Ar1(Db) to Ar18(Db). In one preferred aspect of the present invention, Ar¹ and Ar² are unsubstituted aryl groups, and more preferably unsubstituted phenyl groups.

In the general formula (1), L¹ represents a single bond or a divalent linking group. The divalent linking group includes a substituted or unsubstituted arylene group, and a substituted or unsubstituted heteroarylene group. In one preferred aspect of the present invention, L¹ is a single bond. In one aspect of the present invention, L¹ is a substituted or unsubstituted arylene group. In one aspect of the present invention, L¹ is a substituted or unsubstituted heteroarylene group. Regarding the aryl moiety constituting the arylene group, reference can be made to the description and the preferred range of the aryl group and the alkyl group in the description section of the substituted ring-fused carbazol-9-yl group given hereinabove. The heteroarylene group includes a linking group formed by substituting at least one ring skeleton carbon atom constituting the arylene group with a nitrogen atom.

Specific examples of L¹ are shown below. However, L¹ which can be employed in the present invention shall not be construed as being limited by the following specific examples. In the following specific examples, expression of a methyl group is omitted. Consequently, for example, L3 to L5 are substituted with a methyl group.* indicates a bonding site. L1 is a single bond.

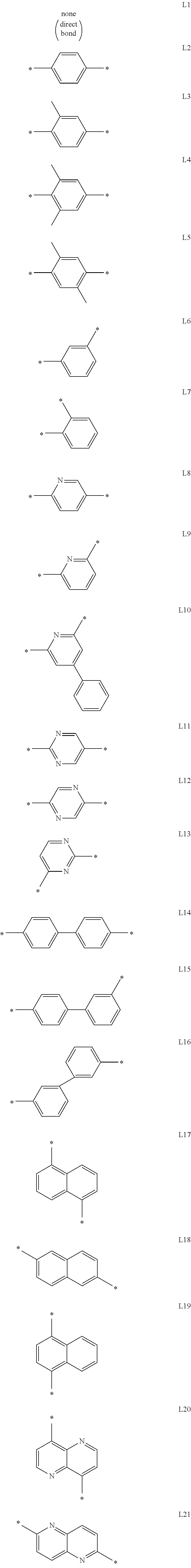

In one preferred aspect of the present invention, X¹ to X³ are N, Ar¹ and Ar² are substituted or unsubstituted aryl groups (preferably substituted or unsubstituted phenyl groups, more preferably phenyl groups), and L¹ is a single bond.

The compound represented by the general formula (1) preferably does not contain a metal atom, and can be a compound composed only of atoms selected from the group consisting of a carbon atom, a hydrogen atom, a deuterium atom, a nitrogen atom, an oxygen atom, and a sulfur atom. In one preferred aspect of the present invention, the compound represented by the general formula (1) is composed only of atoms selected from the group consisting of a carbon atom, a hydrogen atom, a deuterium atom, a nitrogen atom, and an oxygen atom. In addition, the compound represented by the general formula (1) can be a compound composed only of atoms selected from the group consisting of a carbon atom, a hydrogen atom, a deuterium atom, a nitrogen atom, and a sulfur atom. The compound represented by the general formula (1) can be a compound composed only of atoms selected from the group consisting of a carbon atom, a hydrogen atom, a deuterium atom, and a nitrogen atom. The compound represented by the general formula (1) can be a compound composed only of atoms selected from the group consisting of a carbon atom, a hydrogen atom, and a nitrogen atom. Further, the compound represented by the general formula (1) can be a compound which does not contain a hydrogen atom but contains a deuterium atom.

In the description herein, the term “Substituent Group A” means one group or a combination of two or more groups selected from the group consisting of a hydroxyl group, a halogen atom (for example, a fluorine atom, a chlorine atom, a bromine atom, and an iodine atom), an alkyl group (for example, having 1 to 40 carbon atoms), an alkoxy group (for example, having 1 to 40 carbon atoms), an alkylthio group (for example, having 1 to 40 carbon atoms), an aryl group (for example, having 6 to 30 carbon atoms), an aryloxy group (for example, having 6 to 30 carbon atoms), an arylthio group (for example, having 6 to 30 carbon atoms), a heteroaryl group (for example, having 5 to 30 ring skeleton-constituting atoms), a heteroaryloxy group (for example, having 5 to 30 ring skeleton-constituting atoms), a heteroarylthio group (for example, having 5 to 30 ring skeleton-constituting atoms), an acyl group (for example, having 1 to 40 carbon atoms), an alkenyl group (for example, having 1 to 40 carbon atoms), an alkynyl group (for example, having 1 to 40 carbon atoms), an alkoxycarbonyl group (for example, having 1 to 40 carbon atoms), an aryloxycarbonyl group (for example, having 1 to 40 carbon atoms), a heteroaryloxycarbonyl group (for example, having 1 to 40 carbon atoms), a silyl group (for example, a trialkylsilyl group having 1 to 40 carbon atoms), and a nitro group.

In the description herein, the term “Substituent Group B” means one group or a combination of two or more groups selected from the group consisting of an alkyl group (for example, having 1 to 40 carbon atoms), an alkoxy group (for example, having 1 to 40 carbon atoms), an aryl group (for example, having 6 to 30 carbon atoms), an aryloxy group (for example, having 6 to 30 carbon atoms), a heteroaryl group (for example, having 5 to 30 ring skeleton-constituting atoms), a heteroaryloxy group (for example, having 5 to 30 ring skeleton-constituting atoms), and a diarylaminoamino group (for example, having 0 to 20 carbon atoms).

In the description herein, the term “Substituent Group C” means one group or a combination of two or more groups selected from the group consisting of an alkyl group (for example, having 1 to 20 carbon atoms), an aryl group (for example, having 6 to 22 carbon atoms), a heteroaryl group (for example, having 5 to 20 ring skeleton-constituting atoms), and a diarylamino group (for example, having 12 to 20 carbon atoms). In the description herein, the term “Substituent Group D” means one group or a combination of two or more groups selected from the group consisting of an alkyl group (for example, having 1 to 20 carbon atoms), an aryl group (for example, having 6 to 22 carbon atoms), and a heteroaryl group (for example, having 5 to 20 ring skeleton-constituting atoms).

In the description herein, the term “Substituent Group E” means one group or a combination of two or more groups selected from the group consisting of an alkyl group (for example, having 1 to 20 carbon atoms), and an aryl group (for example, having 6 to 22 carbon atoms).

In the description herein, in the case where “substituent” or “substituted or unsubstituted” is described, for example, the substituent can be selected from Substituent Group A, can be selected from Substituent Group B, can be selected from Substituent Group C, can be selected from Substituent Group D, or can be selected from Substituent Group E.

In Table 1 and Table 2 below, specific examples of the compound represented by the general formula (1) are shown. However, the compound represented by the general formula (1) that can be used in the present invention should not be construed as being limited by these specific examples.

In Table 1 and Table 2, structures of Compound 1 to Compound 1700384 are individually shown by specifying R¹ to R⁴ of the following general formula for each compound. Specifically, structures where Ar¹ and Ar² are phenyl groups (Ar1), X¹ to X³ are nitrogen atoms (N), L¹ is a single bond, and R¹ to R⁴ are groups specified as in Table 1 and Table 2 are individually shown as structures of Compounds 1 to 1700384.

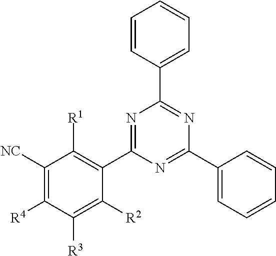

In Table 2, structures of Compounds 1 to 1700384 are shown by collectively displaying R¹ to R⁴ of a plurality of compounds in each row. For example, in the row of Compounds 1 to 224 in Table 2, compounds in which R¹ is a hydrogen atom (H), R³ is fixed to a phenyl group (Ph), and R² and R⁴ are D1 to D224 are referred to as Compounds 1 to 224 in that order. R² and R⁴ are the same. That is, the row of Compounds 1 to 224 in Table 2 collectively represents Compounds 1 to 224 specified in Table 1. Similarly, in the row of Compounds 225 to 448 in Table 2, those in which R¹ is a hydrogen atom (H), R³ is fixed to a methyl group (Me), and R² and R⁴ are D1 to D224 are referred to as Compounds 225 to 448 in that order. In the same manner, Compounds 449 to 12096 in Table 2 are also specified. Compounds 12097 to 14784 in Table 2 specify structures in which three of R¹ to R⁴ are the same and any of D1 to D224. Compounds 14785 to 15008 in Table 2 specify structures in which all of R¹ to R⁴ are the same and any of D1 to D224.

Compounds 15009 to 858696 in Table 2 specify structures in which one of R¹ to R⁴ is any of D1 to D224, and the other one of R¹ to R⁴ is any of Z1 to Z209. Here, D1 to D224 are first fixed to one while Z1 to Z209 are changed in order to specify the compounds, and thereafter D1 to D224 are fixed to the next one while Z1 to Z209 are changed in order to specify the compounds. Therefore, compound numbers are assigned in such a manner that compounds in which R¹ is a hydrogen atom (H), R³ is fixed to a phenyl group (Ph), R² is D1 and R⁴ is Z1 to Z209 are sequentially assigned as Compounds 15009 to 15232, compounds in which R² is D2 and R⁴ is Z1 to Z209 are sequentially assigned as Compounds 15233 to 15456, compounds in which R³ is D3 and R⁴ is Z1 to Z209 are sequentially assigned as Compounds 15457 to 15680, and compounds in which R³ is D224 and R⁴ is Z1 to Z209 are sequentially assigned as Compounds 61601 to 61824. In the same manner, Compounds 61825 to 857696 in Table 2 are also specified.

Compounds 857697 to 1700384 in Table 2 specify structures in which two of R¹ to R⁴ are the same and any of D1 to D224, and the other one of R¹ to R⁴ is any of Z1 to Z209, and structures in which one of R¹ to R⁴ is any of D1 to D224, and the other two of R¹ to R⁴ are the same and any of Z1 to Z209. Here, also D1 to D224 are first fixed to one while Z1 to Z209 are changed in order to specify the compounds, and thereafter D1 to D224 are fixed to the next one while Z1 to Z209 are changed in order to specify the compounds. In the same manner as above, Compounds 1 to 1700384 are specified.

TABLE 1

| No. | R¹ | R² | R³ | R⁴ |
|---|---|---|---|---|
| 1 | H | D1 | Ph | D1 |
| 2 | H | D2 | Ph | D2 |
| 3 | H | D3 | Ph | D3 |
| 4 | H | D4 | Ph | D4 |
| 5 | H | D5 | Ph | D5 |
| 6 | H | D6 | Ph | D6 |
| 7 | H | D7 | Ph | D7 |
| 8 | H | D8 | Ph | D8 |
| 9 | H | D9 | Ph | D9 |
| 10 | H | D10 | Ph | D10 |
| 11 | H | D11 | Ph | D11 |
| 12 | H | D12 | Ph | D12 |
| 13 | H | D13 | Ph | D13 |
| 14 | H | D14 | Ph | D14 |
| 15 | H | D15 | Ph | D15 |
| 16 | H | D16 | Ph | D16 |
| 17 | H | D17 | Ph | D17 |
| 18 | H | D18 | Ph | D18 |
| 19 | H | D19 | Ph | D19 |
| 20 | H | D20 | Ph | D20 |
| 21 | H | D21 | Ph | D21 |
| 22 | H | D22 | Ph | D22 |
| 23 | H | D23 | Ph | D23 |
| 24 | H | D24 | Ph | D24 |
| 25 | H | D25 | Ph | D25 |
| 26 | H | D26 | Ph | D26 |
| 27 | H | D27 | Ph | D27 |
| 28 | H | D28 | Ph | D28 |
| 29 | H | D29 | Ph | D29 |
| 30 | H | D30 | Ph | D30 |
| 31 | H | D31 | Ph | D31 |
| 32 | H | D32 | Ph | D32 |
| 33 | H | D33 | Ph | D33 |
| 34 | H | D34 | Ph | D34 |
| 35 | H | D35 | Ph | D35 |
| 36 | H | D36 | Ph | D36 |
| 37 | H | D37 | Ph | D37 |
| 38 | H | D38 | Ph | D38 |
| 39 | H | D39 | Ph | D39 |
| 40 | H | D40 | Ph | D40 |
| 41 | H | D41 | Ph | D41 |
| 42 | H | D42 | Ph | D42 |
| 43 | H | D43 | Ph | D43 |
| 44 | H | D44 | Ph | D44 |
| 45 | H | D45 | Ph | D45 |
| 46 | H | D46 | Ph | D46 |
| 47 | H | D47 | Ph | D47 |
| 48 | H | D48 | Ph | D48 |
| 49 | H | D49 | Ph | D49 |
| 50 | H | D50 | Ph | D50 |
| 51 | H | D51 | Ph | D51 |
| 52 | H | D52 | Ph | D52 |
| 53 | H | D53 | Ph | D53 |
| 54 | H | D54 | Ph | D54 |
| 55 | H | D55 | Ph | D55 |
| 56 | H | D56 | Ph | D56 |
| 57 | H | D57 | Ph | D57 |
| 58 | H | D58 | Ph | D58 |
| 59 | H | D59 | Ph | D59 |
| 60 | H | D60 | Ph | D60 |
| 61 | H | D61 | Ph | D61 |
| 62 | H | D62 | Ph | D62 |
| 63 | H | D63 | Ph | D63 |
| 64 | H | D64 | Ph | D64 |
| 65 | H | D65 | Ph | D65 |
| 66 | H | D66 | Ph | D66 |
| 67 | H | D67 | Ph | D67 |
| 68 | H | D68 | Ph | D68 |
| 69 | H | D69 | Ph | D69 |
| 70 | H | D70 | Ph | D70 |
| 71 | H | D71 | Ph | D71 |
| 72 | H | D72 | Ph | D72 |
| 73 | H | D73 | Ph | D73 |
| 74 | H | D74 | Ph | D74 |
| 75 | H | D75 | Ph | D75 |
| 76 | H | D76 | Ph | D76 |
| 77 | H | D77 | Ph | D77 |
| 78 | H | D78 | Ph | D78 |
| 79 | H | D79 | Ph | D79 |
| 80 | H | D80 | Ph | D80 |
| 81 | H | D81 | Ph | D81 |
| 82 | H | D82 | Ph | D82 |
| 83 | H | D83 | Ph | D83 |
| 84 | H | D84 | Ph | D84 |
| 85 | H | D85 | Ph | D85 |
| 86 | H | D86 | Ph | D86 |
| 87 | H | D87 | Ph | D87 |
| 88 | H | D88 | Ph | D88 |
| 89 | H | D89 | Ph | D89 |
| 90 | H | D90 | Ph | D90 |
| 91 | H | D91 | Ph | D91 |
| 92 | H | D92 | Ph | D92 |
| 93 | H | D93 | Ph | D93 |
| 94 | H | D94 | Ph | D94 |
| 95 | H | D95 | Ph | D95 |
| 96 | H | D96 | Ph | D96 |
| 97 | H | D97 | Ph | D97 |
| 98 | H | D98 | Ph | D98 |
| 99 | H | D99 | Ph | D99 |
| 100 | H | D100 | Ph | D100 |
| 101 | H | D101 | Ph | D101 |
| 102 | H | D102 | Ph | D102 |
| 103 | H | D103 | Ph | D103 |
| 104 | H | D104 | Ph | D104 |
| 105 | H | D105 | Ph | D105 |
| 106 | H | D106 | Ph | D106 |
| 107 | H | D107 | Ph | D107 |
| 108 | H | D108 | Ph | D108 |
| 109 | H | D109 | Ph | D109 |
| 110 | H | D110 | Ph | D110 |
| 111 | H | D111 | Ph | D111 |
| 112 | H | D112 | Ph | D112 |
| 113 | H | D113 | Ph | D113 |
| 114 | H | D114 | Ph | D114 |
| 115 | H | D115 | Ph | D115 |
| 116 | H | D116 | Ph | D116 |
| 117 | H | D117 | Ph | D117 |
| 118 | H | D118 | Ph | D118 |
| 119 | H | D119 | Ph | D119 |
| 120 | H | D120 | Ph | D120 |
| 121 | H | D121 | Ph | D121 |
| 122 | H | D122 | Ph | D122 |
| 123 | H | D123 | Ph | D123 |
| 124 | H | D124 | Ph | D124 |
| 125 | H | D125 | Ph | D125 |
| 126 | H | D126 | Ph | D126 |
| 127 | H | D127 | Ph | D127 |
| 128 | H | D128 | Ph | D128 |
| 129 | H | D129 | Ph | D129 |
| 130 | H | D130 | Ph | D130 |
| 131 | H | D131 | Ph | D131 |
| 132 | H | D132 | Ph | D132 |
| 133 | H | D133 | Ph | D133 |
| 134 | H | D134 | Ph | D134 |
| 135 | H | D135 | Ph | D135 |
| 136 | H | D136 | Ph | D136 |
| 137 | H | D137 | Ph | D137 |
| 138 | H | D138 | Ph | D138 |
| 139 | H | D139 | Ph | D139 |
| 140 | H | D140 | Ph | D140 |
| 141 | H | D141 | Ph | D141 |
| 142 | H | D142 | Ph | D142 |
| 143 | H | D143 | Ph | D143 |
| 144 | H | D144 | Ph | D144 |
| 145 | H | D145 | Ph | D145 |
| 146 | H | D146 | Ph | D146 |
| 147 | H | D147 | Ph | D147 |
| 148 | H | D148 | Ph | D148 |
| 149 | H | D149 | Ph | D149 |
| 150 | H | D150 | Ph | D150 |
| 151 | H | D151 | Ph | D151 |
| 152 | H | D152 | Ph | D152 |
| 153 | H | D153 | Ph | D153 |
| 154 | H | D154 | Ph | D154 |
| 155 | H | D155 | Ph | D155 |
| 156 | H | D156 | Ph | D156 |
| 157 | H | D157 | Ph | D157 |
| 158 | H | D158 | Ph | D158 |
| 159 | H | D159 | Ph | D159 |
| 160 | H | D160 | Ph | D160 |
| 161 | H | D161 | Ph | D161 |
| 162 | H | D162 | Ph | D162 |
| 163 | H | D163 | Ph | D163 |
| 164 | H | D164 | Ph | D164 |
| 165 | H | D165 | Ph | D165 |
| 166 | H | D166 | Ph | D166 |
| 167 | H | D167 | Ph | D167 |
| 168 | H | D168 | Ph | D168 |
| 169 | H | D169 | Ph | D169 |
| 170 | H | D170 | Ph | D170 |
| 171 | H | D171 | Ph | D171 |
| 172 | H | D172 | Ph | D172 |
| 173 | H | D173 | Ph | D173 |
| 174 | H | D174 | Ph | D174 |
| 175 | H | D175 | Ph | D 175 |
| 176 | H | D176 | Ph | D176 |
| 177 | H | D177 | Ph | D177 |
| 178 | H | D178 | Ph | D178 |
| 179 | H | D179 | Ph | D179 |
| 180 | H | D180 | Ph | D180 |
| 181 | H | D181 | Ph | D181 |
| 182 | H | D182 | Ph | D182 |
| 183 | H | D183 | Ph | D183 |
| 184 | H | D184 | Ph | D184 |
| 185 | H | D185 | Ph | D185 |
| 186 | H | D186 | Ph | D186 |
| 187 | H | D187 | Ph | D187 |
| 188 | H | D188 | Ph | D188 |
| 189 | H | D189 | Ph | D189 |
| 190 | H | D190 | Ph | D190 |
| 191 | H | D191 | Ph | D191 |
| 192 | H | D192 | Ph | D192 |
| 193 | H | D193 | Ph | D193 |
| 194 | H | D194 | Ph | D194 |
| 195 | H | D 195 | Ph | D 195 |
| 196 | H | D196 | Ph | D196 |
| 197 | H | D197 | Ph | D197 |
| 198 | H | D198 | Ph | D198 |
| 199 | H | D199 | Ph | D199 |
| 200 | H | D200 | Ph | D200 |
| 201 | H | D201 | Ph | D201 |
| 202 | H | D202 | Ph | D202 |
| 203 | H | D203 | Ph | D203 |
| 204 | H | D204 | Ph | D204 |
| 205 | H | D205 | Ph | D205 |
| 206 | H | D206 | Ph | D206 |
| 207 | H | D207 | Ph | D207 |
| 208 | H | D208 | Ph | D208 |
| 209 | H | D209 | Ph | D209 |
| 210 | H | D210 | Ph | D210 |
| 211 | H | D211 | Ph | D211 |
| 212 | H | D212 | Ph | D212 |
| 213 | H | D213 | Ph | D213 |
| 214 | H | D214 | Ph | D214 |
| 215 | H | D215 | Ph | D215 |
| 216 | H | D216 | Ph | D216 |
| 217 | H | D217 | Ph | D217 |
| 218 | H | D218 | Ph | D218 |
| 219 | H | D219 | Ph | D219 |
| 220 | H | D220 | Ph | D220 |
| 221 | H | D221 | Ph | D221 |
| 222 | H | D222 | Ph | D222 |
| 223 | H | D223 | Ph | D223 |
| 224 | H | D224 | Ph | D224 |
| 225 | H | D1 | Me | D1 |
| 226 | H | D2 | Me | D2 |
| 227 | H | D3 | Me | D3 |
| 228 | H | D4 | Me | D4 |
| 229 | H | D5 | Me | D5 |
| 230 | H | D6 | Me | D6 |
| 231 | H | D7 | Me | D7 |
| 232 | H | D8 | Me | D8 |
| 233 | H | D9 | Me | D9 |
| 234 | H | D10 | Me | D10 |
| 235 | H | D11 | Me | D11 |
| 236 | H | D12 | Me | D12 |
| 237 | H | D13 | Me | D13 |
| 238 | H | D14 | Me | D14 |
| 239 | H | D15 | Me | D15 |
| 240 | H | D16 | Me | D16 |
| 241 | H | D17 | Me | D17 |
| 242 | H | D18 | Me | D18 |
| 243 | H | D19 | Me | D19 |
| 244 | H | D20 | Me | D20 |
| 245 | H | D21 | Me | D21 |
| 246 | H | D22 | Me | D22 |
| 247 | H | D23 | Me | D23 |
| 248 | H | D24 | Me | D24 |
| 249 | H | D25 | Me | D25 |
| 250 | H | D26 | Me | D26 |
| 251 | H | D27 | Me | D27 |
| 252 | H | D28 | Me | D28 |
| 253 | H | D29 | Me | D29 |
| 254 | H | D30 | Me | D30 |
| 255 | H | D31 | Me | D31 |
| 256 | H | D32 | Me | D32 |
| 257 | H | D33 | Me | D33 |
| 258 | H | D34 | Me | D34 |
| 259 | H | D35 | Me | D35 |
| 260 | H | D36 | Me | D36 |
| 261 | H | D37 | Me | D37 |
| 262 | H | D38 | Me | D38 |
| 263 | H | D39 | Me | D39 |
| 264 | H | D40 | Me | D40 |
| 265 | H | D41 | Me | D41 |
| 266 | H | D42 | Me | D42 |
| 267 | H | D43 | Me | D43 |
| 268 | H | D44 | Me | D44 |
| 269 | H | D45 | Me | D45 |
| 270 | H | D46 | Me | D46 |
| 271 | H | D47 | Me | D47 |
| 272 | H | D48 | Me | D48 |
| 273 | H | D49 | Me | D49 |
| 274 | H | D50 | Me | D50 |
| 275 | H | D51 | Me | D51 |
| 276 | H | D52 | Me | D52 |
| 277 | H | D53 | Me | D53 |
| 278 | H | D54 | Me | D54 |
| 279 | H | D55 | Me | D55 |
| 280 | H | D56 | Me | D56 |
| 281 | H | D57 | Me | D57 |
| 282 | H | D58 | Me | D58 |
| 283 | H | D59 | Me | D59 |
| 284 | H | D60 | Me | D60 |
| 285 | H | D61 | Me | D61 |
| 286 | H | D62 | Me | D62 |
| 287 | H | D63 | Me | D63 |
| 288 | H | D64 | Me | D64 |
| 289 | H | D65 | Me | D65 |
| 290 | H | D66 | Me | D66 |
| 291 | H | D67 | Me | D67 |
| 292 | H | D68 | Me | D68 |
| 293 | H | D69 | Me | D69 |
| 294 | H | D70 | Me | D70 |
| 295 | H | D71 | Me | D71 |
| 296 | H | D72 | Me | D72 |
| 297 | H | D73 | Me | D73 |
| 298 | H | D74 | Me | D74 |
| 299 | H | D75 | Me | D75 |
| 300 | H | D76 | Me | D76 |
| 301 | H | D77 | Me | D77 |
| 302 | H | D78 | Me | D78 |
| 303 | H | D79 | Me | D79 |
| 304 | H | D80 | Me | D80 |
| 305 | H | D81 | Me | D81 |
| 306 | H | D82 | Me | D82 |
| 307 | H | D83 | Me | D83 |
| 308 | H | D84 | Me | D84 |
| 309 | H | D85 | Me | D85 |
| 310 | H | D86 | Me | D86 |
| 311 | H | D87 | Me | D87 |
| 312 | H | D88 | Me | D88 |
| 313 | H | D89 | Me | D89 |
| 314 | H | D90 | Me | D90 |
| 315 | H | D91 | Me | D91 |
| 316 | H | D92 | Me | D92 |
| 317 | H | D93 | Me | D93 |
| 318 | H | D94 | Me | D94 |
| 319 | H | D95 | Me | D95 |
| 320 | H | D96 | Me | D96 |
| 321 | H | D97 | Me | D97 |
| 322 | H | D98 | Me | D98 |
| 323 | H | D99 | Me | D99 |
| 324 | H | D100 | Me | D100 |
| 325 | H | D101 | Me | D101 |
| 326 | H | D102 | Me | D102 |
| 327 | H | D103 | Me | D103 |
| 328 | H | D104 | Me | D104 |
| 329 | H | D105 | Me | D105 |
| 330 | H | D106 | Me | D106 |
| 331 | H | D107 | Me | D107 |
| 332 | H | D108 | Me | D108 |
| 333 | H | D109 | Me | D109 |
| 334 | H | D110 | Me | D110 |
| 335 | H | D111 | Me | D111 |
| 336 | H | D112 | Me | D112 |
| 337 | H | D113 | Me | D113 |
| 338 | H | D114 | Me | D114 |
| 339 | H | D115 | Me | D115 |
| 340 | H | D116 | M? | D116 |
| 341 | H | D117 | Me | D117 |
| 342 | H | D118 | Me | D118 |
| 343 | H | D119 | Me | D119 |
| 344 | H | D120 | Me | D120 |
| 345 | H | D121 | Me | D121 |
| 346 | H | D122 | Me | D122 |
| 347 | H | D123 | Me | D123 |
| 348 | H | D124 | Me | D124 |
| 349 | H | D125 | Me | D125 |
| 350 | H | D126 | Me | D126 |
| 351 | H | D127 | Me | D127 |
| 352 | H | D128 | Me | D128 |
| 353 | H | D129 | Me | D129 |
| 354 | H | D130 | Me | D130 |
| 355 | H | D131 | Me | D131 |
| 356 | H | D132 | Me | D132 |
| 357 | H | D133 | Me | D133 |
| 358 | H | D134 | Me | D134 |
| 359 | H | D135 | Me | D135 |
| 360 | H | D136 | Me | D136 |
| 361 | H | D137 | Me | D137 |
| 362 | H | D138 | Me | D138 |
| 363 | H | D139 | Me | D139 |
| 364 | H | D140 | Me | D140 |
| 365 | H | D141 | Me | D141 |
| 366 | H | D 142 | Me | D142 |
| 367 | H | D143 | Me | D143 |
| 368 | H | D144 | Me | D144 |
| 369 | H | D145 | Me | D145 |
| 370 | H | D146 | Me | D146 |
| 371 | H | D147 | Me | D147 |
| 372 | H | D148 | Me | D148 |
| 373 | Ph | D149 | Me | D149 |
| 374 | Ph | D150 | Me | D150 |
| 375 | Ph | D151 | Me | D151 |
| 376 | Ph | D152 | Me | D152 |
| 377 | Ph | D153 | Me | D153 |
| 378 | Ph | D154 | Me | D154 |
| 379 | Ph | D155 | Me | D155 |
| 380 | Ph | D156 | Me | D156 |
| 381 | Ph | D157 | Me | D157 |
| 382 | Ph | D158 | Me | D158 |
| 383 | Ph | D159 | Me | D159 |
| 384 | Ph | D160 | Me | D160 |
| 385 | Ph | D161 | Me | D161 |
| 386 | Ph | D162 | Me | D162 |
| 387 | Ph | D163 | Me | D163 |
| 388 | Ph | D164 | Me | D164 |
| 389 | Ph | D165 | Me | D165 |
| 390 | Ph | D166 | M? | D166 |
| 391 | Ph | D167 | Me | D167 |
| 392 | Ph | D168 | Me | D168 |
| 393 | Ph | D169 | Me | D169 |
| 394 | Ph | D170 | Me | D170 |
| 395 | Ph | D171 | Me | D171 |
| 396 | Ph | D172 | Me | D172 |
| 397 | Ph | D173 | Me | D173 |
| 398 | Ph | D174 | Me | D174 |
| 399 | Ph | D175 | Me | D175 |
| 400 | Ph | D176 | Me | D176 |
| 401 | Ph | D177 | Me | D177 |
| 402 | Ph | D178 | Me | D178 |
| 403 | Ph | D179 | Me | D179 |
| 404 | Ph | D180 | Me | D180 |
| 405 | Ph | D181 | Me | D181 |
| 406 | Ph | D182 | Me | D182 |
| 407 | Ph | D183 | Me | D183 |
| 408 | Ph | D184 | Me | D184 |
| 409 | Ph | D185 | Me | D185 |
| 410 | Ph | D186 | Me | D186 |
| 411 | Ph | D187 | Me | D187 |
| 412 | Ph | D188 | Me | D188 |
| 413 | Ph | D189 | Me | D189 |
| 414 | Ph | D190 | Me | D190 |
| 415 | Ph | D191 | Me | D191 |
| 416 | Ph | D192 | Me | D192 |
| 417 | Ph | D193 | Me | D193 |
| 418 | Ph | D194 | Me | D194 |
| 419 | Ph | D195 | Me | D195 |
| 420 | Ph | D196 | Me | D196 |
| 421 | Ph | D197 | Me | D197 |
| 422 | Ph | D198 | Me | D198 |
| 423 | Ph | D199 | Me | D199 |
| 424 | Ph | D200 | Me | D200 |
| 425 | Ph | D201 | Me | D201 |
| 426 | Ph | D202 | Me | D202 |
| 427 | Ph | D203 | Me | D203 |
| 428 | Ph | D204 | Me | D204 |
| 429 | Ph | D205 | Me | D205 |
| 430 | Ph | D206 | Me | D206 |
| 431 | Ph | D207 | Me | D207 |
| 432 | Ph | D208 | Me | D208 |
| 433 | Ph | D209 | Me | D209 |
| 434 | Ph | D210 | Me | D210 |
| 435 | Ph | D211 | Me | D211 |
| 436 | Ph | D212 | Me | D212 |
| 437 | Ph | D213 | Me | D213 |
| 438 | Ph | D214 | Me | D214 |
| 439 | Ph | D215 | Me | D215 |
| 440 | Ph | D216 | Me | D216 |
| 441 | Ph | D217 | Me | D217 |
| 442 | Ph | D218 | Me | D218 |
| 443 | Ph | D219 | Me | D219 |
| 444 | Ph | D220 | Me | D220 |
| 445 | Ph | D221 | Me | D221 |
| 446 | Ph | D222 | Me | D222 |
| 447 | Ph | D223 | Me | D223 |
| 448 | Ph | D224 | Me | D224 |

TABLE 2

| No. | R¹ | R² | R³ | R⁴ | Note |
|---|---|---|---|---|---|
| 1~224 | H | D1~D224 | Ph | D1~D224 | R² = |
| 225~448 | H | D1~D224 | Me | D1~D224 | R⁴ |
| 449~672 | H | D1~D224 | H | D1~D224 | |
| 673~896 | Ph | D1~D224 | Ph | D1~D224 | |
| 897~1120 | Ph | D1~D224 | Me | D1~D224 | |
| 1121~1344 | Ph | D1~D224 | H | D1~D224 | |
| 1345~1568 | Me | D1~D224 | Ph | D1~D224 | |
| 1569~1792 | Me | D1~D224 | Me | D1~D224 | |
| 1793~2016 | Me | D1~D224 | H | D1~D224 | |
| 2017~2240 | D1~D224 | D1~D224 | H | Ph | R¹ = |
| 2241~2464 | D1~D224 | D1~D224 | H | Me | R² |
| 2465~2688 | D1~D224 | D1~D224 | H | H | |
| 2689~2912 | D1~D224 | D1~D224 | Ph | Ph | |
| 2913~3136 | D1~D224 | D1~D224 | Ph | Me | |
| 3137~3360 | D1~D224 | D1~D224 | Ph | H | |
| 3361~3584 | D1~D224 | D1~D224 | Me | Ph | |
| 3585~3808 | D1~D224 | D1~D224 | Me | Me | |
| 3809~4032 | D1~D224 | D1~D224 | Me | H | |
| 4033~4256 | D1~D224 | H | D1~D224 | Ph | R¹ = |
| 4257~4480 | D1~D224 | H | D1~D224 | Me | R³ |
| 4481~4704 | D1~D224 | H | D1~D224 | H | |
| 4705~4928 | D1~D224 | Ph | D1~D224 | Ph | |
| 4929~5152 | D1~D224 | Ph | D1~D224 | Me | |
| 5153~5376 | D1~D224 | Ph | D1~D224 | H | |
| 5377~5600 | D1~D224 | Me | D1~D224 | Ph | |
| 5601~5824 | D1~D224 | Me | D1~D224 | Me | |
| 5825~6048 | D1~D224 | Me | D1~D224 | H | |
| 6049~6272 | D1~D224 | H | Ph | D1~D224 | R¹ = |
| 6273~6496 | D1~D224 | H | Me | D1~D224 | R⁴ |
| 6497~6720 | D1~D224 | H | H | D1~D224 | |
| 6721~6944 | D1~D224 | Ph | Ph | D1~D224 | |
| 6945~7168 | D1~D224 | Ph | Me | D1~D224 | |
| 7169~7392 | D1~D224 | Ph | H | D1~D224 | |
| 7393~7616 | D1~D224 | Me | Ph | D1~D224 | |
| 7617~7840 | D1~D224 | Me | Me | D1~D224 | |
| 7841~8064 | D1~D224 | Me | H | D1~D224 | |
| 8065~8288 | H | D1~D224 | D1~D224 | Ph | R² = |
| 8289~8512 | H | D1~D224 | D1~D224 | Me | R³ |
| 8513~8736 | H | D1~D224 | D1~D224 | H | |
| 8737~8960 | Ph | D1~D224 | D1~D224 | Ph | |
| 8961~9184 | Ph | D1~D224 | D1~D224 | Me | |
| 9185~9408 | Ph | D1~D224 | D1~D224 | H | |
| 9409~9632 | Me | D1~D224 | D1~D224 | Ph | |
| 9633~9856 | Me | D1~D224 | D1~D224 | Me | |
| 9857~10080 | Me | D1~D224 | D1~D224 | H | |
| 10081~10304 | H | Ph | D1~D224 | D1~D224 | R³ = |
| 10305~10528 | H | Me | D1~D224 | D1~D224 | R⁴ |
| 10529~10752 | H | H | D1~D224 | D1~D224 | |
| 10753~10976 | Ph | Ph | D1~D224 | D1~D224 | |
| 10977~11200 | Ph | Me | D1~D224 | D1~D224 | |
| 11201~11424 | Ph | H | D1~D224 | D1~D224 | |
| 11425~11648 | Me | Ph | D1~D224 | D1~D224 | |
| 11649~11872 | Me | Me | D1~D224 | D1~D224 | |
| 11873~12096 | Me | H | D1~D224 | D1~D224 | |
| 12097~12320 | D1~D224 | D1~D224 | D1~D224 | Ph | R¹ = |
| 12321~12544 | D1~D224 | D1~D224 | D1~D224 | Me | R² = |
| 12545~12768 | D1~D224 | D1~D224 | D1~D224 | H | R³ |
| 12769~12992 | D1~D224 | D1~D224 | Ph | D1~D224 | R¹ = |
| 12993~13216 | D1~D224 | D1~D224 | Me | D1~D224 | R² = |
| 13217~13440 | D1~D224 | D1~D224 | H | D1~D224 | R⁴ |
| 13441~13664 | D1~D224 | Ph | D1~D224 | D1~D224 | R¹ = |
| 13665~13888 | D1~D224 | Me | D1~D224 | D1~D224 | R³ = |
| 13889~14112 | D1~D224 | H | D1~D224 | D1~D224 | R⁴ |
| 14113~14336 | Ph | D1~D224 | D1~D224 | D1~D224 | R² = |
| 14337~14560 | Me | D1~D224 | D1~D224 | D1~D224 | R³ = |
| 14561~14784 | H | D1~D224 | D1~D224 | D1~D224 | R⁴ |
| 14785~15008 | D1~D224 | D1~D224 | D1~D224 | D1~D224 | R¹ = |
| | | | | | R² = |
| | | | | | R³ = |
| | | | | | R⁴ |
| 15009~61824 | H | D1~D224 | Ph | Z1~Z209 | R² ≠ |
| 61825~108640 | H | D1~D224 | Me | Z1~Z209 | R⁴ |
| 108641~155456 | H | D1~D224 | H | Z1~Z209 | |
| 155457~202272 | Ph | D1~D224 | Ph | Z1~Z209 | |
| 202273~249088 | Ph | D1~D224 | Me | Z1~Z209 | |
| 249089~295904 | Ph | D1~D224 | H | Z1~Z209 | |
| 295905~342720 | Me | D1~D224 | Ph | Z1~Z209 | |
| 342721~389536 | Me | D1~D224 | Me | Z1~Z209 | |
| 389537~436352 | Me | D1~D224 | H | Z1~Z209 | |
| 436353~483168 | H | Z1~Z209 | Ph | D1~D224 | |
| 483169~529984 | H | Z1~Z209 | Me | D1~D224 | |
| 529985~576800 | H | Z1~Z209 | H | D1~D224 | |
| 576801~623616 | Ph | Z1Z209 | Ph | D1~D224 | |
| 623617~670432 | Ph | Z1~Z209 | Me | D1~D224 | |
| 670433~717248 | Ph | Z1~Z209 | H | D1~D224 | |
| 717249~764064 | Me | Z1~Z209 | Ph | D1~D224 | |
| 764065~810880 | Me | Z1~Z209 | Me | D1~D224 | |
| 810881~857696 | Me | Z1~Z209 | H | D1~D224 | |
| 857697~904512 | Ph | Z1~Z209 | D1~D224 | D1~D224 | R² = |
| 904513~951328 | Me | Z1~Z209 | D1~D224 | D1~D224 | R⁴ |
| 951329~998144 | H | Z1~Z209 | D1~D224 | D1~D224 | |
| 998145~1044960 | Ph | D1~D224 | Z1~Z209 | D1~D224 | R² = |
| 1044961~1091776 | Me | D1~D224 | Z1~Z209 | D1~D224 | R⁴ |
| 1091777~1138592 | H | D1~D224 | Z1~Z209 | D1~D224 | |
| 1138593~1185408 | Ph | D1~D224 | D1~D224 | Z1~Z209 | R² = |
| 1185409~1232224 | Me | D1~D224 | D1~D224 | Z1~Z209 | R³ |
| 1232225~1279040 | H | D1~D224 | D1~D224 | Z1~Z209 | |
| 1279041~1325856 | Ph | D1~D224 | Z1~Z209 | Z1~Z209 | R³ = |
| 1325857~1372672 | Me | D1~D224 | Z1~Z209 | Z1~Z209 | R⁴ |
| 1372673~1419488 | H | D1~D224 | Z1~Z209 | Z1~Z209 | |
| 1419489~1466304 | Ph | Z1~Z209 | D1~D224 | Z1~Z209 | R² = |
| 1466305~1513120 | Me | Z1~Z209 | D1~D224 | Z1~2209 | R⁴ |
| 1513121~1559936 | H | Z1~Z209 | D1~D224 | Z1~Z209 | |
| 1559937~1606752 | Ph | Z1~Z209 | Z1~Z209 | D1~D224 | R² = |
| 1606753~1653588 | Me | Z1~Z209 | Z1~Z209 | D1~D224 | R³ |
| 1653569~1700384 | H | Z1~Z209 | Z1~Z209 | D1~D224 | |

Compounds obtained by substituting all hydrogen atoms present in the molecules of the above Compounds 1 to 1700384 with deuterium atoms are disclosed as Compounds 1(Da) to 1700384(Da). Compounds obtained by substituting all hydrogen atoms bonding to the two phenyl groups substituting on the triazine ring present in the molecules of the above Compounds 1 to 1700384 with deuterium atoms are disclosed as Compounds 1(Db) to 1700384(Db). Compounds obtained by substituting all hydrogen atoms of the phenyl group or the alkyl group bonding to the benzene ring of D1 to D224 present in the molecules of the above Compounds 1 to 1700384 with deuterium atoms are disclosed as Compounds 1(Dc) to 1700384(Dc). Compounds obtained by substituting all hydrogen atoms bonding to the phenyl group present as one of R¹ to R⁴ in the above Compounds 1 to 224, 673 to 1568, 2017 to 2240, 2689 to 3584, 4033 to 4256, 4705 to 5600, 6049 to 6272, 6721 to 7616, 8065 to 8288, 8737 to 9632, 10081 to 10304, 10753 to 11648, 12097 to 12320, 12769 to 12992, 13441 to 13664, 14113 to 14336, 15009 to 61824, 155457 to 342720, 436353 to 483168, 576801 to 764064, 857697 to 904512, 998145 to U.S. Pat. Nos. 1,044,960, 1,138,593 to U.S. Pat. Nos. 1,185,408, 1,279,041 to U.S. Pat. Nos. 1,325,856, 1,419,489 to U.S. Pat. Nos. 1,466,304, and 1,559,937 to 1606752 (hereinafter the group of these Compounds is referred to as “Compound Group a”) with deuterium atoms are disclosed as Compounds 1(Dd) to 224(Dd), 673(Dd) to 1568(Dd), 2017(Dd) to 2240(Dd), 2689(Dd) to 3584(Dd), 4033(Dd) to 4256(Dd), 4705(Dd) to 5600(Dd), 6049(Dd) to 6272(Dd), 6721(Dd) to 7616(Dd), 8065(Dd) to 8288(Dd), 8737(Dd) to 9632(Dd), 10081(Dd) to 10304(Dd), 10753(Dd) to 11648(Dd), 12097(Dd) to 12320(Dd), 12769(Dd) to 12992(Dd), 13441(Dd) to 13664(Dd), 14113(Dd) to 14336(Dd), 15009(Dd) to 61824(Dd), 155457(Dd) to 342720(Dd), 436353(Dd) to 483168(Dd), 576801(Dd) to 764064(Dd), 857697(Dd) to 904512(Dd), 998145(Dd) to 1044960(Dd), 1138593(Dd) to 1185408(Dd), 1279041(Dd) to 1325856(Dd), 1419489(Dd) to 1466304(Dd), and 1559937(Dd) to 1606752(Dd). Compounds obtained by substituting all hydrogen atoms bonding to the phenyl group present as one of R¹ to R⁴ in the Compound Group a, and all hydrogen atoms of the phenyl group or the alkyl group bonding to the benzene ring of D1 to D224 present in the molecule with deuterium atoms are disclosed as Compounds 1(De) to 224(De), 673(De) to 1568(De), 2017(De) to 2240(De), 2689(De) to 3584(De), 4033(De) to 4256(Dc), 4705(De) to 5600(De), 6049(De) to 6272(Dc), 6721(De) to 7616(De), 8065(De) to 8288(De), 8737(De) to 9632(De), 10081(De) to 10304(De), 10753(De) to 11648(De), 12097(De) to 12320(De), 12769(De) to 12992(De), 13441(De) to 13664(De), 14113(De) to 14336(De), 15009(De) to 61824(De), 155457(De) to 342720(De), 436353(De) to 483168(De), 576801(De) to 764064(De), 857697(De) to 904512(De), 998145(De) to 1044960(De), 1138593(De) to 1185408(De), 1279041(De) to 1325856(De), 1419489(De) to 1466304(De), and 1559937(De) to 1606752(De). Compounds obtained by substituting all hydrogen atoms bonding to the phenyl group present as one of R¹ to R⁴ in the Compound Group a with deuterium atoms, all hydrogen atoms of the phenyl group or the alkyl group bonding to the benzene ring of D1 to D224 present in the molecules with deuterium atoms, and all hydrogen atoms bonding to the two phenyl groups substituting on the triazine ring present in the molecules with deuterium atoms are disclosed as Compounds 1(Df) to 224(Df), 673(Df) to 1568(Df), 2017(Df) to 2240(Df), 2689(Df) to 3584(Df), 4033(Df) to 4256(Df), 4705(Df) to 5600(Df), 6049(Df) to 6272(Df), 6721(Df) to 7616(Df), 8065(Df) to 8288(Df), 8737(Df) to 9632(Df), 10081(Df) to 10304(Df), 10753(Df) to 11648(Df), 12097(Df) to 12320(Df), 12769(Df) to 12992(Df), 13441(Df) to 13664(Df), 14113(Df) to 14336(Df), 15009(Df) to 61824(Df), 155457(Df) to 342720(Df), 436353(Df) to 483168(Df), 576801(Df) to 764064(Df), 857697(Df) to 904512(Df), 998145(Df) to 1044960(Df), 1138593(Df) to 1185408(Df), 1279041(Df) to 1325856(Df), 1419489(Df) to 1466304(Df), and 1559937(Df) to 1606752(Df). Compounds obtained by substituting all hydrogen atoms bonding to the phenyl group present as one of R¹ to R⁴ in the Compound Group a with deuterium atoms, and all hydrogen atoms bonding to the two phenyl groups substituting on the triazine ring present in the molecules with deuterium atoms are disclosed as 1(Dg) to 224(Dg), 673(Dg) to 1568(Dg), 2017(Dg) to 2240(Dg), 2689(Dg) to 3584(Dg), 4033(Dg) to 4256(Dg), 4705(Dg) to 5600(Dg), 6049(Dg) to 6272(Dg), 6721(Dg) to 7616(Dg), 8065(Dg) to 8288(Dg), 8737(Dg) to 9632(Dg), 10081(Dg) to 10304(Dg), 10753(Dg) to 11648(Dg), 12097(Dg) to 12320(Dg), 12769(Dg) to 12992(Dg), 13441(Dg) to 13664(Dg), 14113(Dg) to 14336(Dg), 15009(Dg) to 61824(Dg), 155457(Dg) to 342720(Dg), 436353(Dg) to 483168(Dg), 576801(Dg) to 764064(Dg), 857697(Dg) to 904512(Dg), 998145(Dg) to 1044960(Dg), 1138593(Dg) to 1185408(Dg), 1279041(Dg) to 1325856(Dg), 1419489(Dg) to 1466304(Dg), and 1559937(Dg) to 1606752(Dg).

The compounds specified by the above numbers are all individually disclosed. In addition, among the specific examples of the compounds, in the case where a rotamer is present, a mixture of rotamers and each separated rotamer are also disclosed in the description herein.

In one aspect of the present invention, compounds are selected from Compounds 1 to 12096. In one aspect of the present invention, compounds are selected from Compounds 1 to 2016. In one aspect of the present invention, compounds are selected from Compounds 2017 to 4032. In one aspect of the present invention, compounds are selected from Compounds 4033 to 6048. In one aspect of the present invention, compounds are selected from Compounds 6049 to 8064. In one aspect of the present invention, compounds are selected from Compounds 8065 to 10080. In one aspect of the present invention, compounds are selected from Compounds 10081 to 12096. In one aspect of the present invention, compounds are selected from Compounds 1 to 224, 1121 to 1344, 2017 to 2240, 3137 to 3360, 4033 to 4256, 5153 to 5376, 6049 to 6272, 7169 to 7392, 8065 to 8288, 9185 to 9008, 10081 to 10304, and 11201 to 11424. In one aspect of the present invention, compounds are selected from Compounds 673 to 896, 2689 to 2912, 4705 to 4928, 6721 to 6944, 8737 to 8960, and 10753 to 10976.

In one aspect of the present invention, compounds are selected from Compounds 12097 to 14784. In one aspect of the present invention, compounds are selected from Compounds 12097 to 12768. In one aspect of the present invention, compounds are selected from Compounds 12769 to 13440. In one aspect of the present invention, compounds are selected from Compounds 13441 to 14112. In one aspect of the present invention, compounds are selected from Compounds 14113 to 14784. In one aspect of the present invention, compounds are selected from Compounds 12097 to 12320, 12769 to 12992, 13441 to 13664, and 14113 to 14336.

In one aspect of the present invention, compounds are selected from Compounds 14785 to 15008.

In one aspect of the present invention, compounds are selected from Compounds 15009 to 857696. In one aspect of the present invention, compounds are selected from Compounds 15009 to 155456. In one aspect of the present invention, compounds are selected from Compounds 155457 to 295904. In one aspect of the present invention, compounds are selected from Compounds 295905 to 436352, 436353 to 576800, 576801 to 717248, and 717249 to 857696. In one aspect of the present invention, compounds are selected from Compounds 15009 to 61824, and 436353 to 483168.

In one aspect of the present invention, compounds are selected from Compounds 857697 to 1700384. In one aspect of the present invention, compounds are selected from Compounds 857697 to 998144. In one aspect of the present invention, compounds are selected from Compounds 998145 to 1138592. In one aspect of the present invention, compounds are selected from Compounds 1138593 to 1279040. In one aspect of the present invention, compounds are selected from Compounds 1279041 to 1419488. In one aspect of the present invention, compounds are selected from Compounds 1419489 to 1559936. In one aspect of the present invention, compounds are selected from Compounds 1559937 to 1700384. In one aspect of the present invention, compounds are selected from Compounds 857697 to 904512, 998145 to U.S. Pat. Nos. 1,044,960, 1,138,593 to U.S. Pat. Nos. 1,185,408, 1,279,041 to U.S. Pat. Nos. 1,325,856, 1,419,489 to U.S. Pat. Nos. 1,466,304, and 1,559,937 to 1606752. In one aspect of the present invention, compounds are selected from Compounds 951329 to 998144, 1091777 to U.S. Pat. Nos. 1,138,592, 1,232,225 to U.S. Pat. Nos. 1,279,040, 1,372,673 to U.S. Pat. Nos. 1,419,488, 1,513,121 to U.S. Pat. Nos. 1,559,936, and 1,653,569 to 1700384.

In the above, structures where, in the general formula (1), Ar¹ and Ar² are phenyl groups (Ar1), X¹ to X³ are nitrogen atoms (N), L¹ is a single bond, and R¹ to R⁴ are groups specified as in Table 1 and Table 2 are specified as structures of Compounds 1 to 1700384. Ar¹ and Ar2 in Compounds 1 to 1700384 were changed as in Table 3, in which the resultant compounds were sequentially displayed in a table format. In Table 3, Compounds 1 to 1700384 are also shown for clarifying the correspondence relationship. For example, Compound 1a indicates a compound having a structure in which Ar¹ and Ar² of Compound 1 are changed to Ar19. Compound 2a indicates a compound having a structure in which Ar¹ and Ar² of Compound 2 are changed to Ar19. Compound 1700384a indicates a compound having a structure in which Ar¹ and Ar2 of Compound 1700384 are changed to Ar19. Compounds 1b to 1700384b and the subsequent compounds are specified in the same manner. X¹ to X³ of the compounds specified in Table 3 are all nitrogen atoms (N) and L¹ is a single bond.

TABLE 3

| No. | Ar¹ | Ar² |
|---|---|---|
| 1~1700384 | Ar1 | Ar1 |
| 1a~1700384a | Ar19 | Ar1 |
| 1b~1700384b | Ar20 | Ar1 |
| 1c~1700384c | Ar21 | Ar1 |
| 1d~1700384d | Ar22 | Ar1 |
| 1e~1700384e | Ar23 | Ar1 |
| 1f~1700384f | Ar24 | Ar1 |
| 1g~1700384g | Ar25 | Ar1 |
| 1h~1700384h | Ar26 | Ar1 |
| 1i~1700384i | Ar19 | Ar19 |
| 1j~1700384j | Ar20 | Ar20 |
| 1k~1700384k | Ar21 | Ar21 |
| 1l~1700384l | Ar22 | Ar22 |
| 1m~1700384m | Ar23 | Ar23 |
| 1n~1700384n | Ar24 | Ar24 |
| 1o~1700384o | Ar25 | Ar25 |
| 1p~1700384p | Ar26 | Ar26 |

In one preferred aspect of the present invention, the compound represented by the general formula (1) is selected from the following group of compounds. Compounds can be selected from Group 1, or can be selected from Group 2, or can be selected from Group 3, or can be selected from Group 4, or can be selected from Group 5, or can be selected from Group 6, or can be selected from Group 7, or can be selected from Group 8, or can be selected from Group 9, or can be selected from Group 10, or can be selected from Group 11, or can be selected from Group 12, or can be selected from Group 13, or can be selected from Group 14.

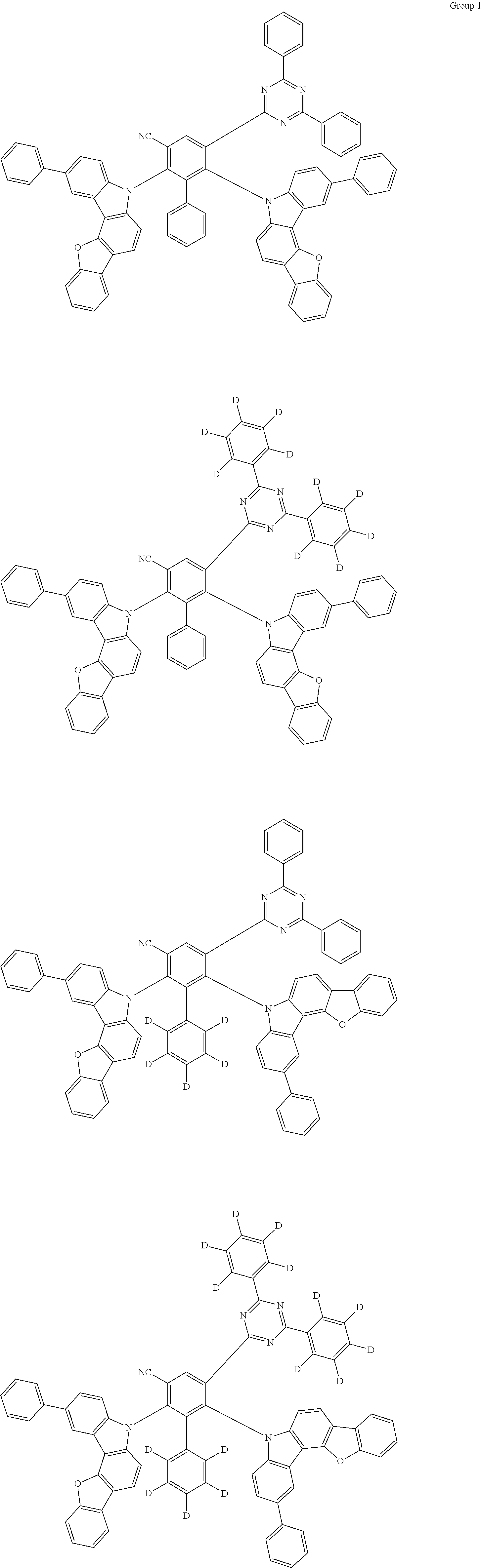

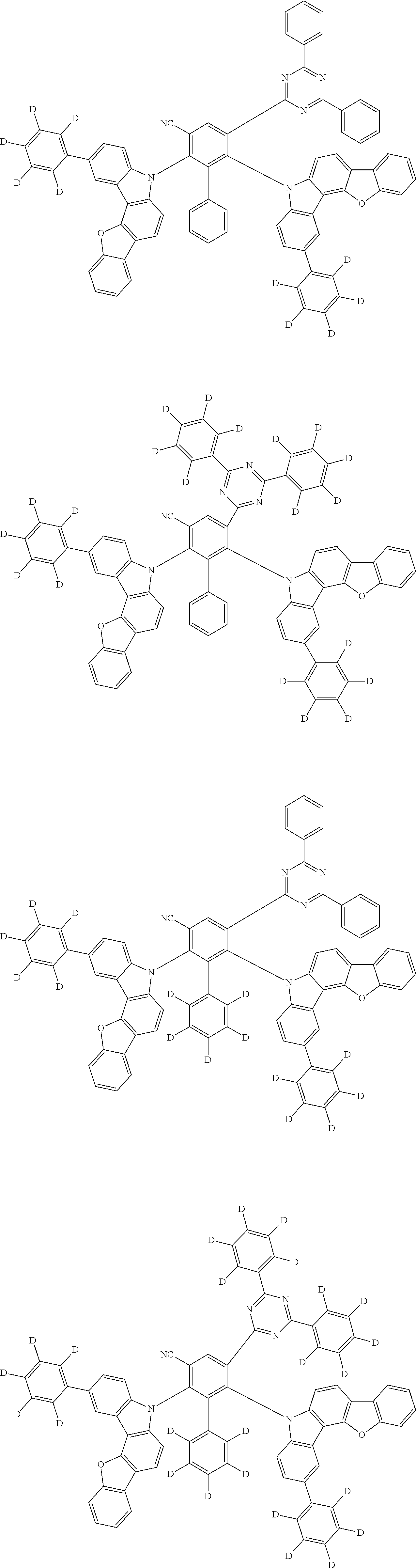

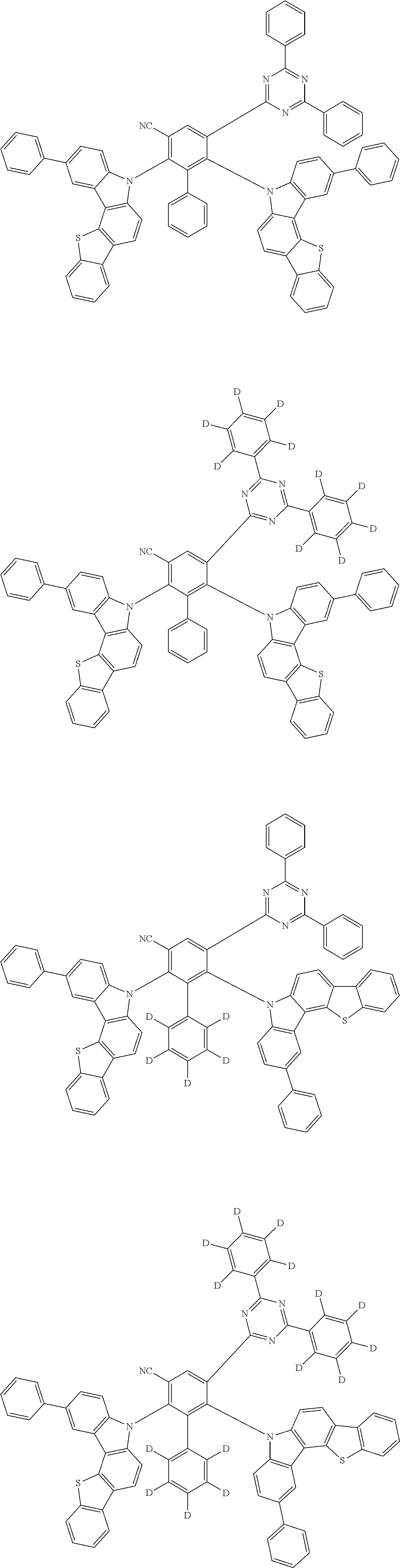

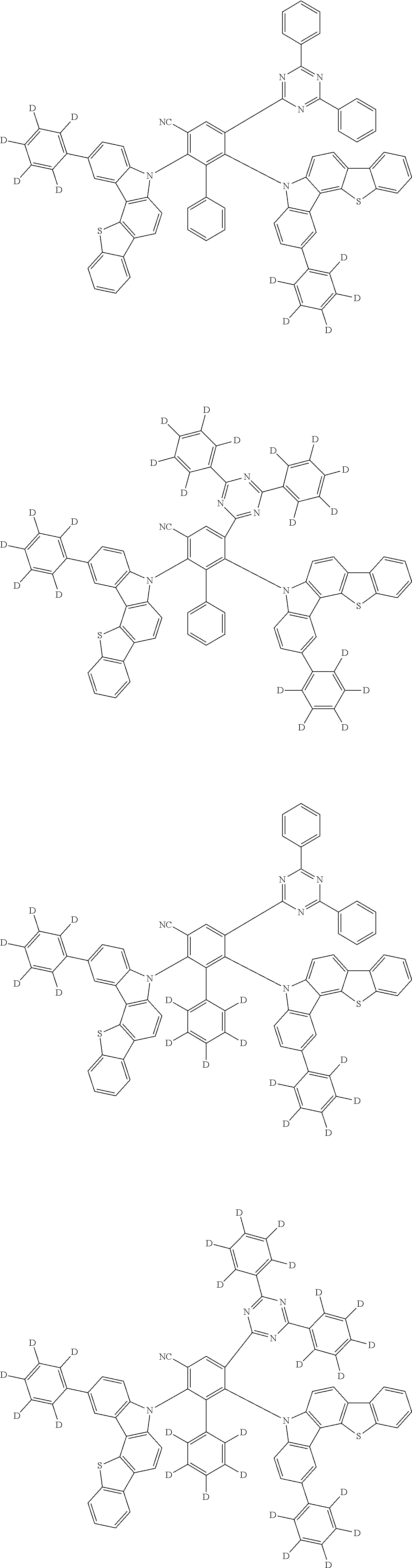

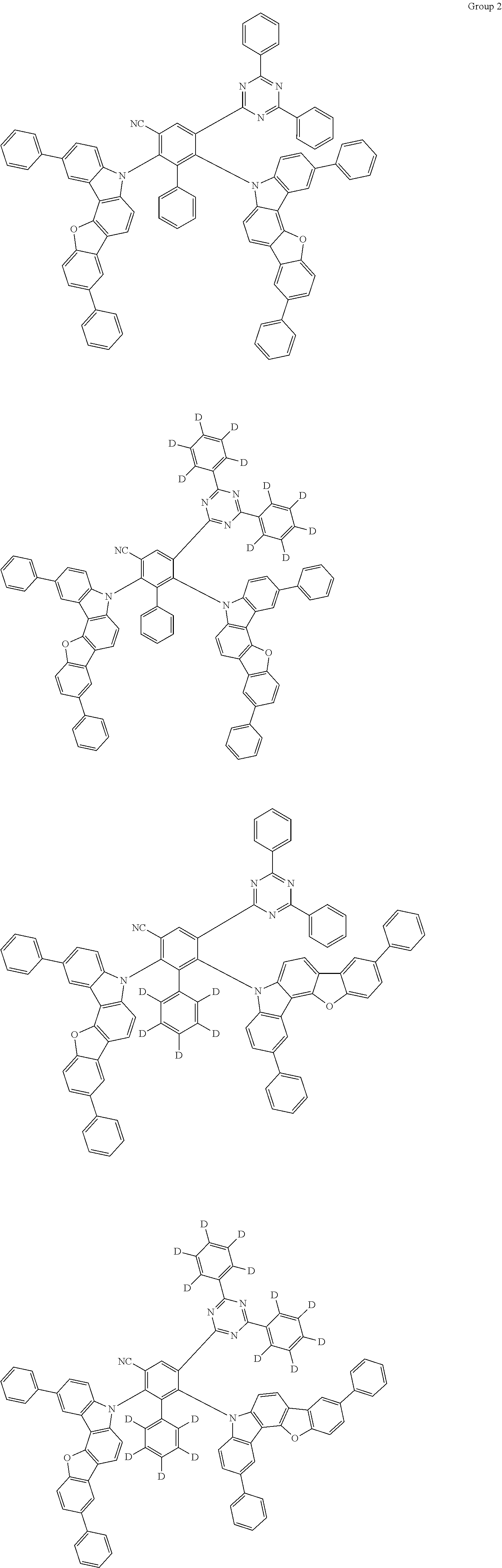

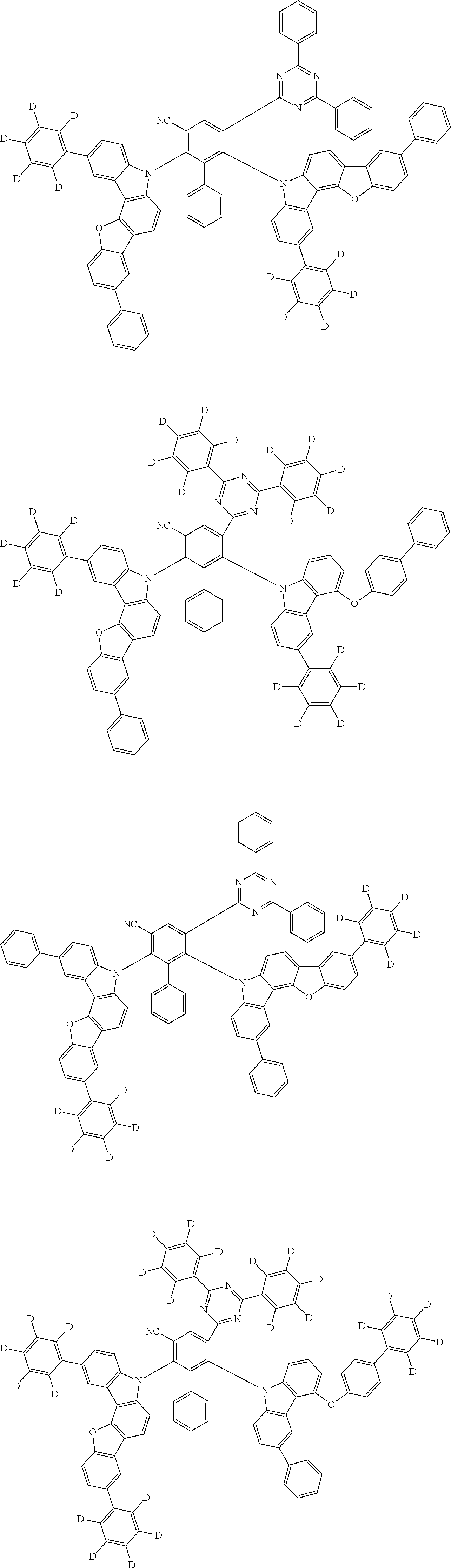

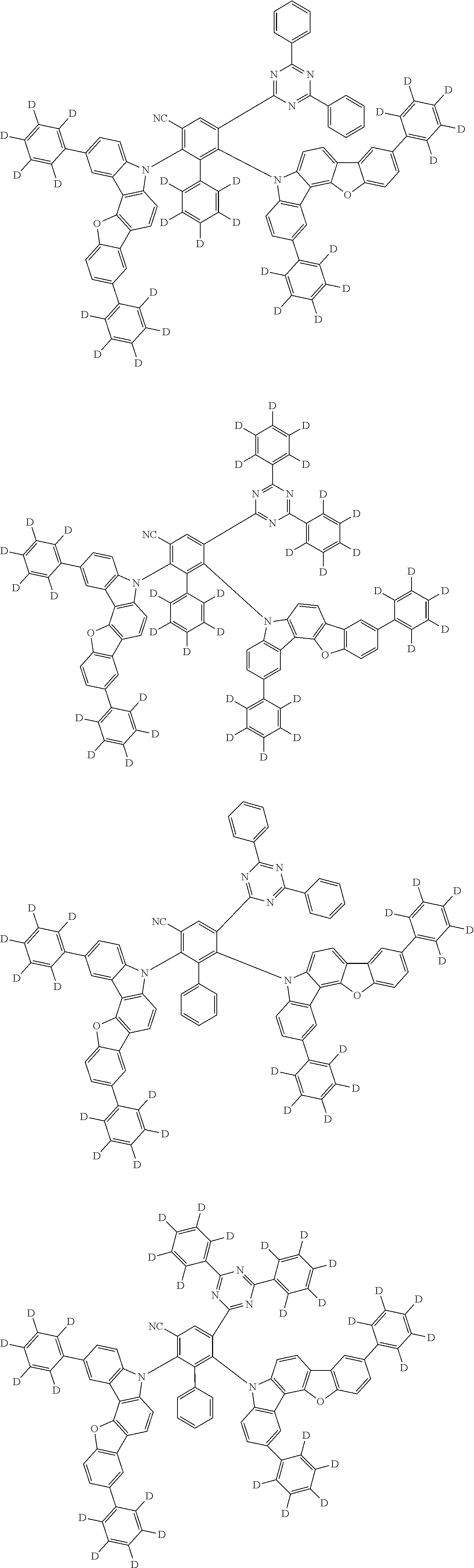

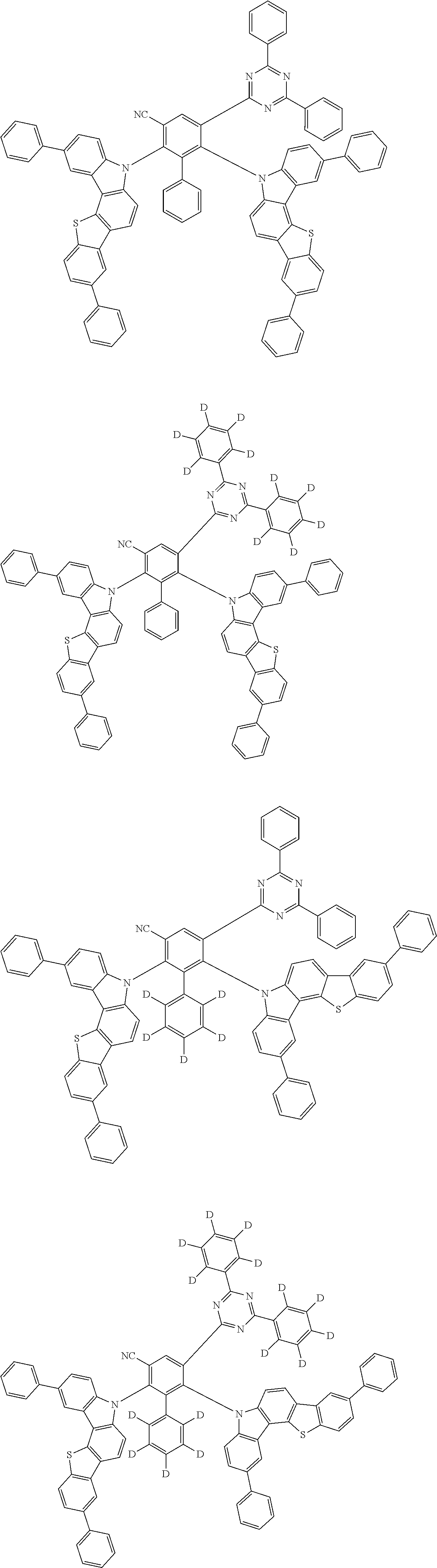

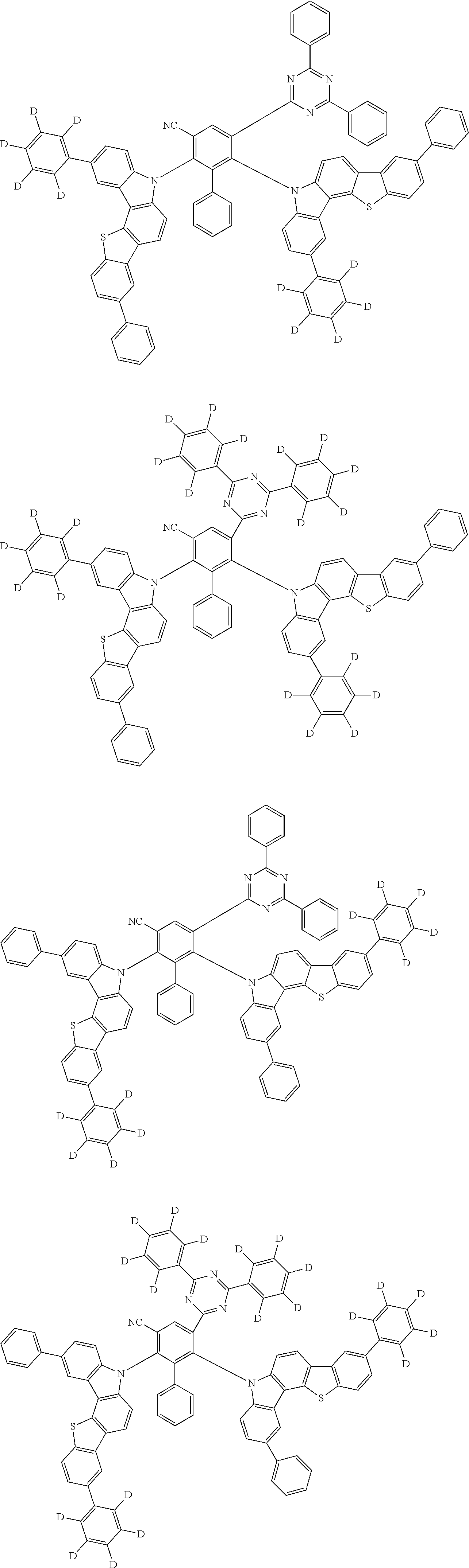

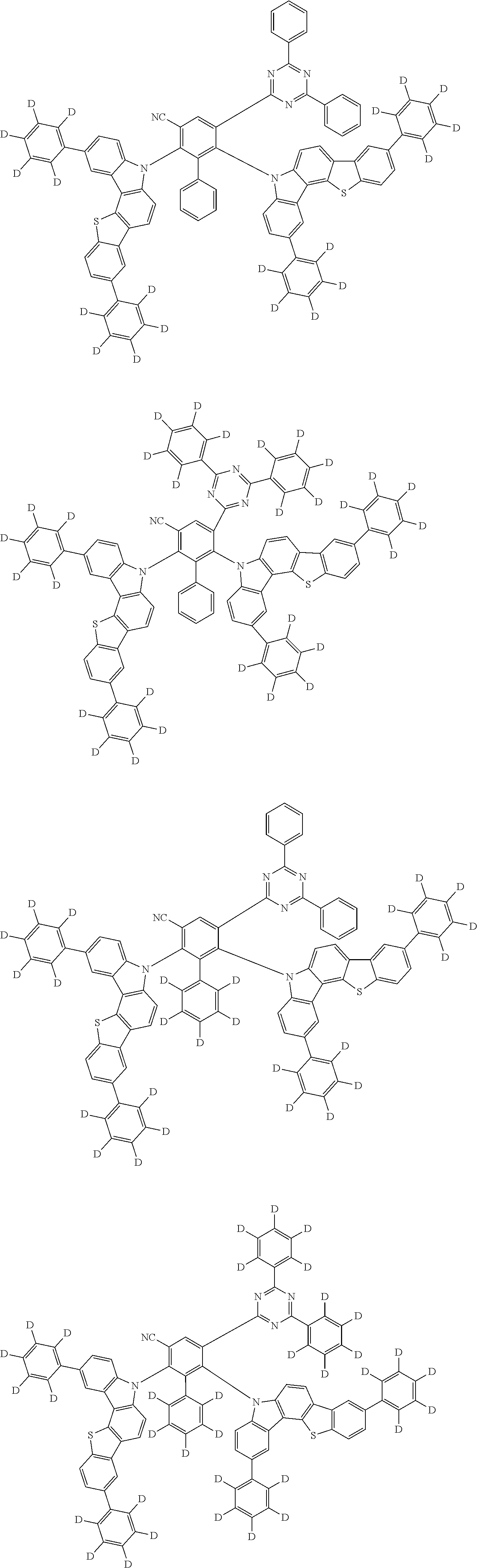

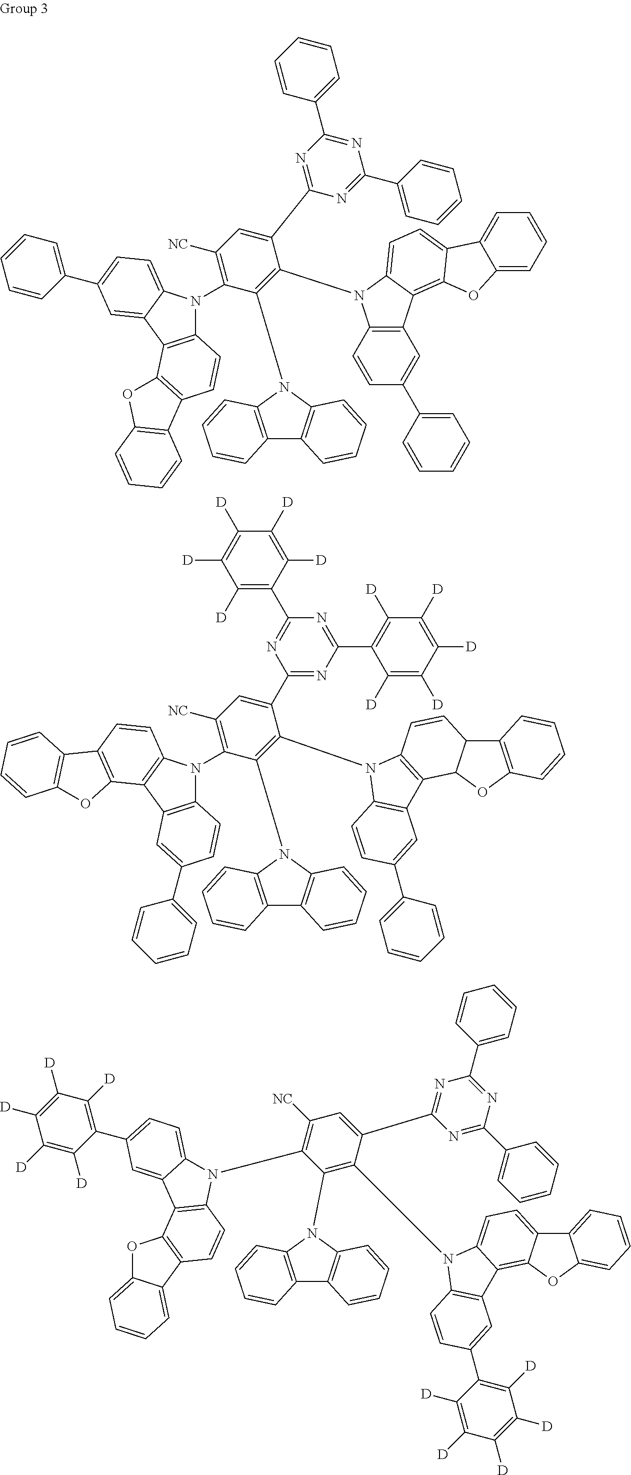

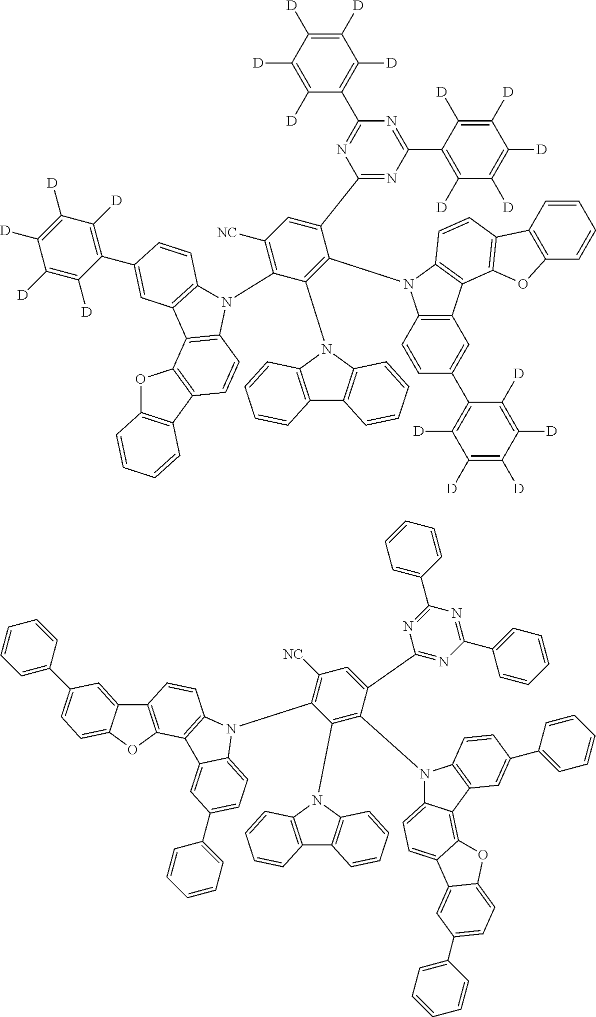

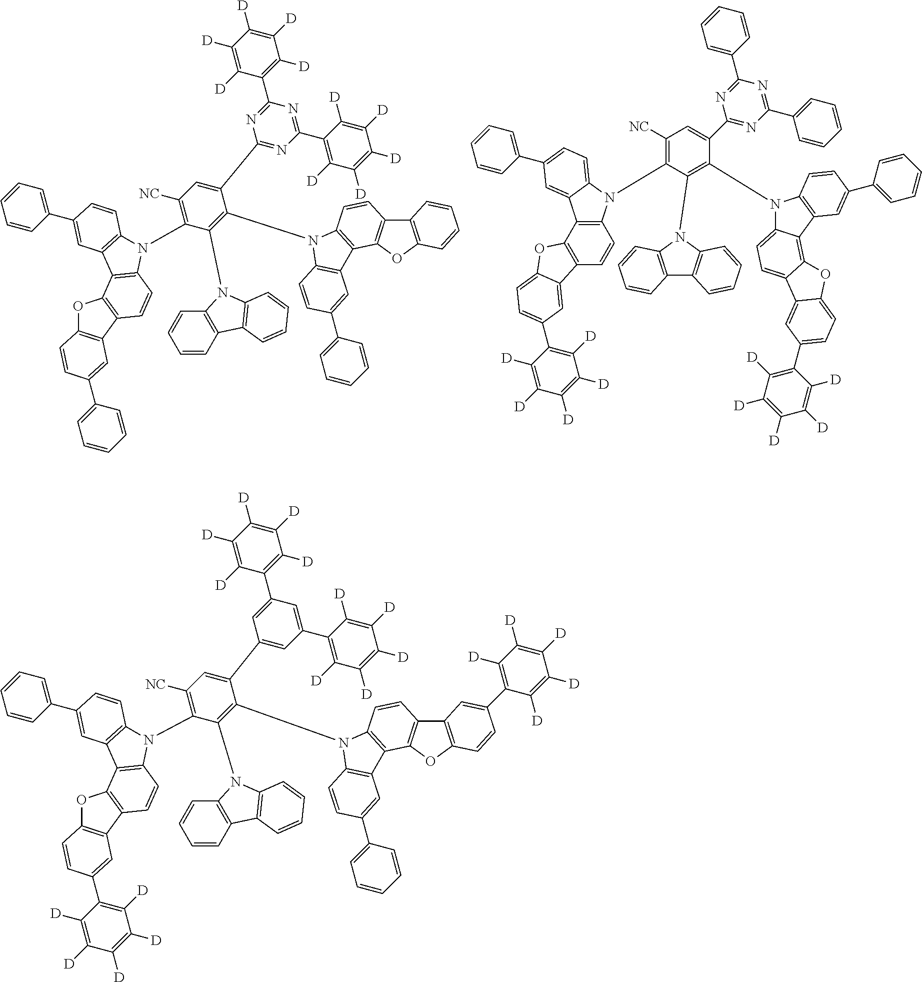

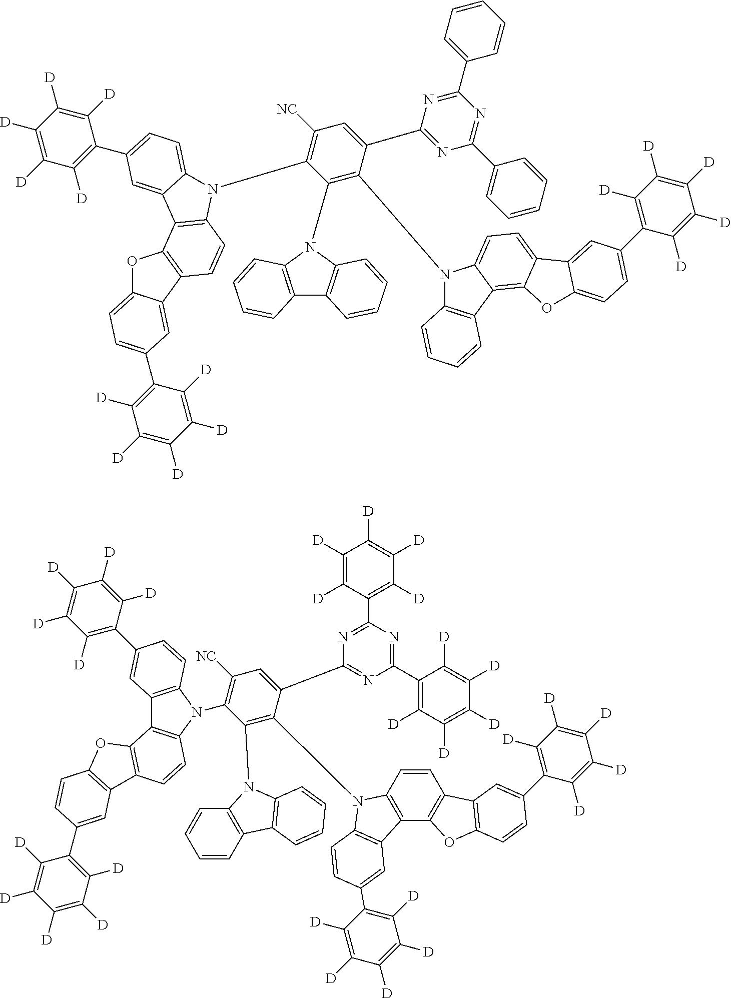

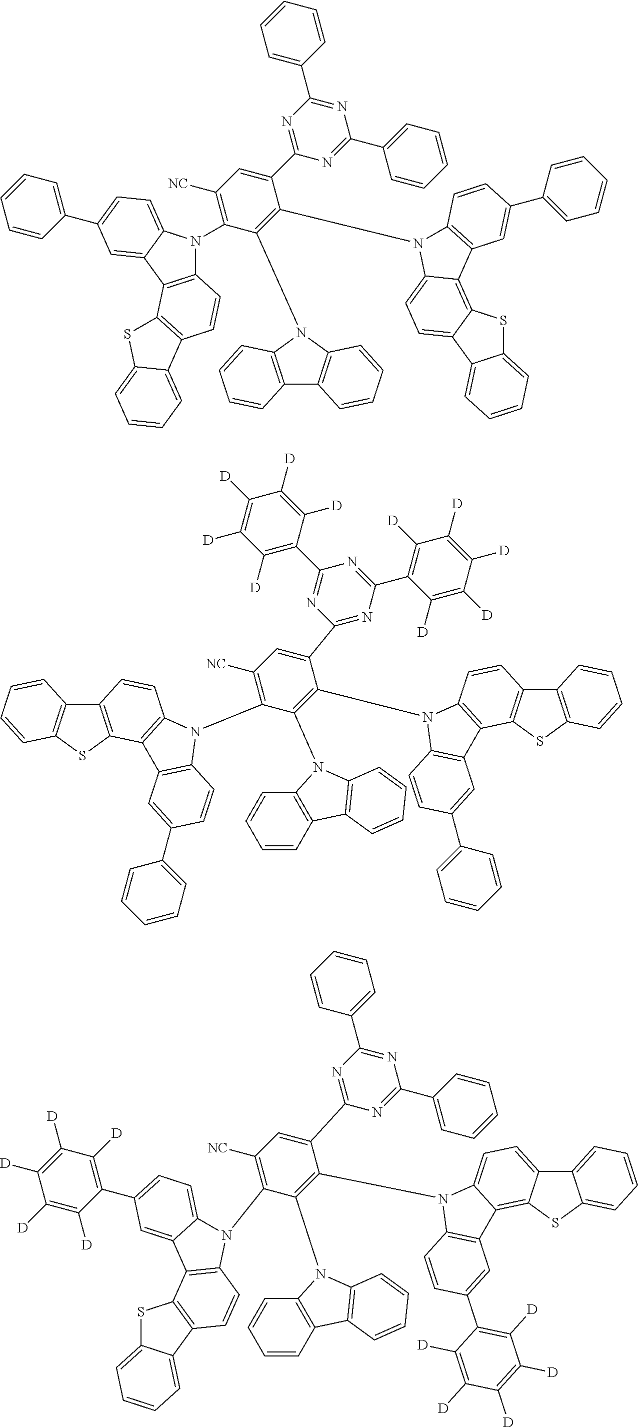

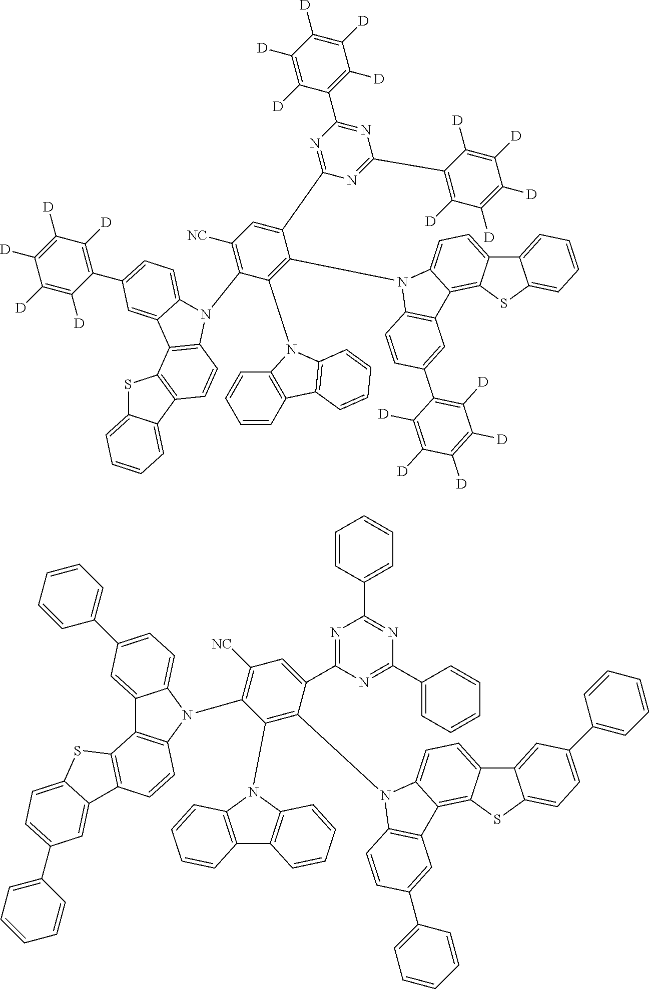 [SMILES: [2H]c1c([2H])c([2H])c(-c2ccc3c(c2)c2c4sc5ccccc5c4ccc2n3-c2c(C#N)cc(-c3nc(-c4c([2H])c([2H])c([2H])c([2H])c4[2H])nc(-c4c([2H])c([2H])c([2H])c([2H])c4[2H])n3)c(-n3c4ccc(-c5c([2H])c([2H])c([2H])c([2H])c5[2H])cc4c4c5sc6ccccc6c5ccc43)c2-n2c3ccccc3c3ccccc32)c([2H])c1[2H] ; N#Cc1cc(-c2nc(-c3ccccc3)nc(-c3ccccc3)n2)c(-n2c3ccc(-c4ccccc4)cc3c3c4sc5ccc(-c6ccccc6)cc5c4ccc32)c(-n2c3ccccc3c3ccccc32)c1-n1c2ccc(-c3ccccc3)cc2c2c3sc4ccc(-c5ccccc5)cc4c3ccc21]

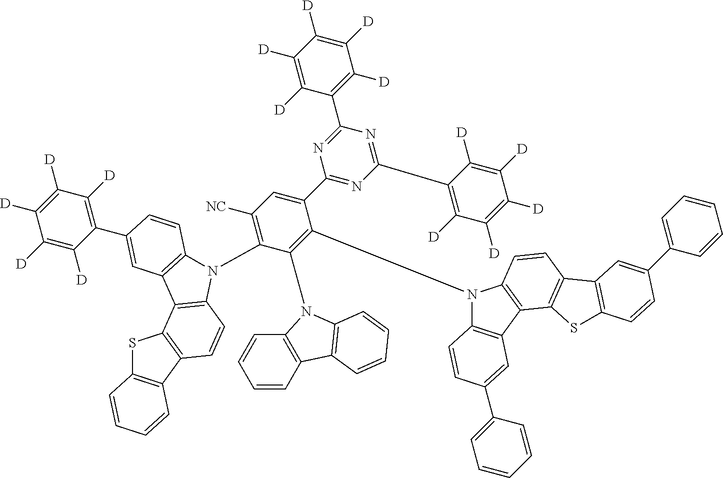 [SMILES: [2H]c1c([2H])c([2H])c(-c2ccc3c(c2)c2c4sc5ccccc5c4ccc2n3-c2c(C#N)cc(-c3nc(-c4c([2H])c([2H])c([2H])c([2H])c4[2H])nc(-c4c([2H])c([2H])c([2H])c([2H])c4[2H])n3)c(-n3c4ccc(-c5ccccc5)cc4c4c5sc6ccc(-c7ccccc7)cc6c5ccc43)c2-n2c3ccccc3c3ccccc32)c([2H])c1[2H]]

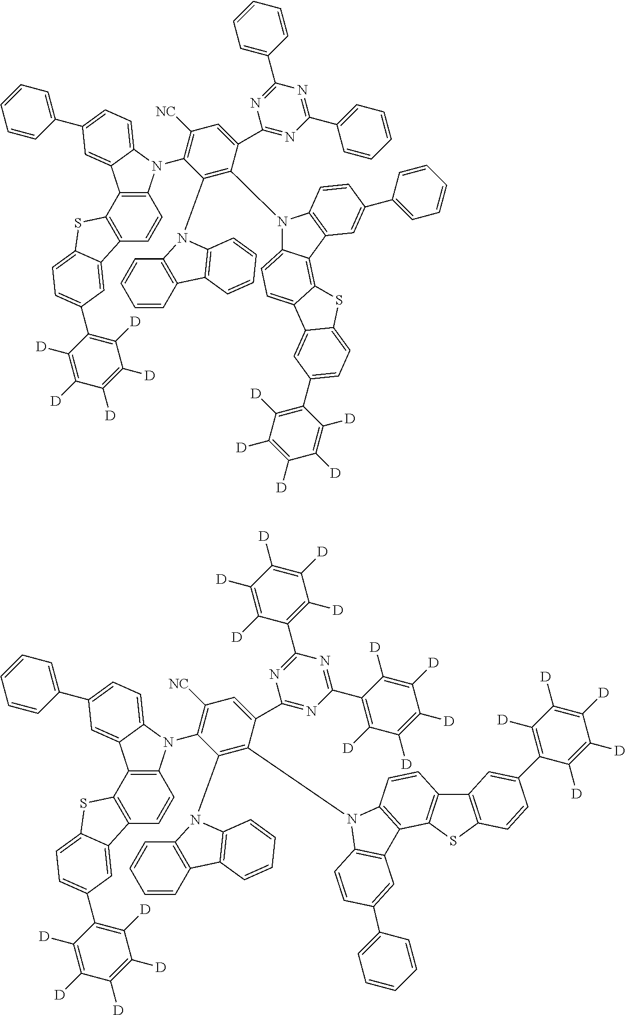

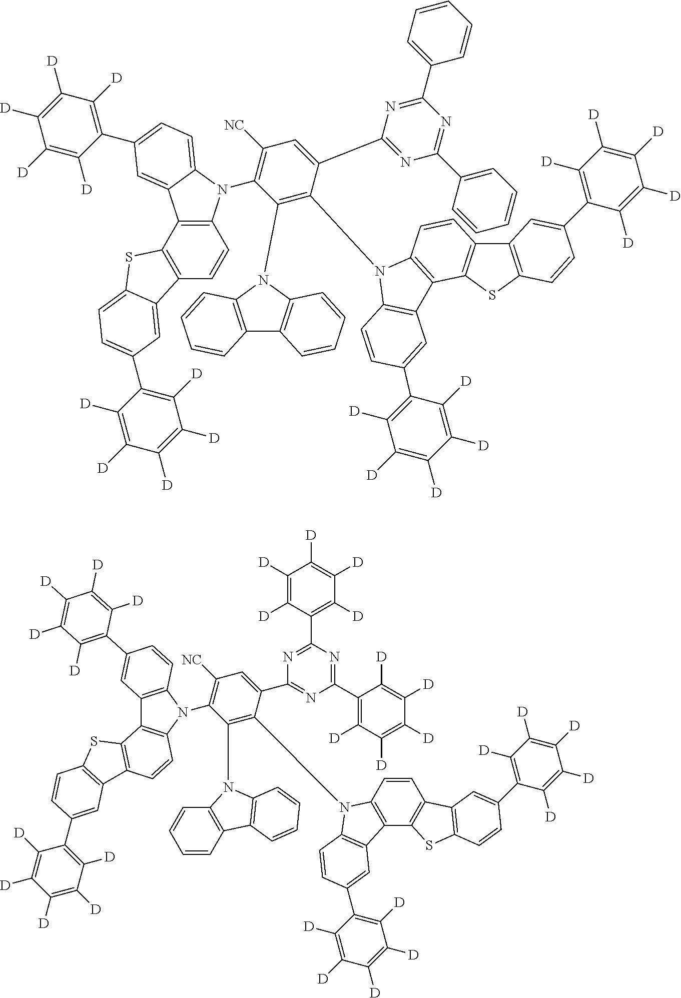

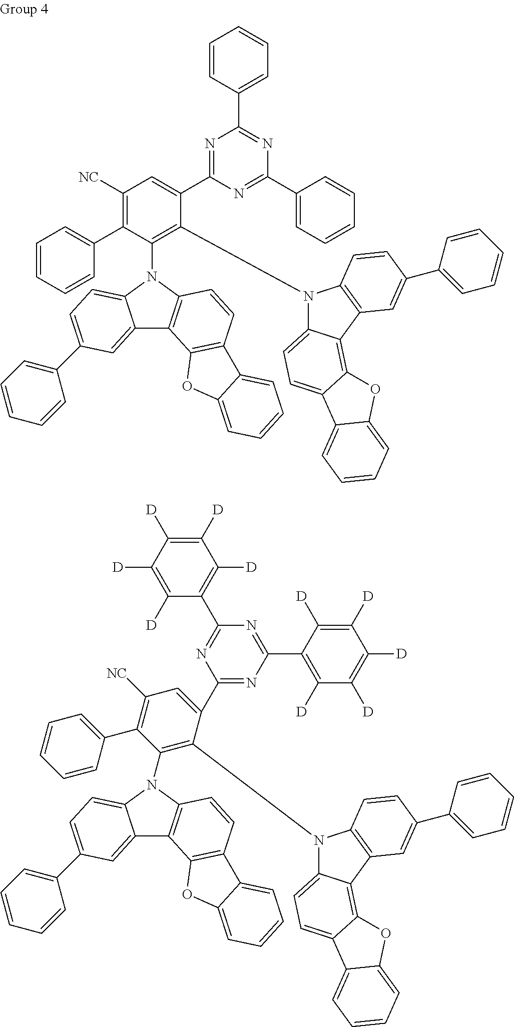 [SMILES: N#Cc1cc(-c2nc(-c3ccccc3)nc(-c3ccccc3)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; [2H]c1c([2H])c([2H])c(-c2nc(-c3cc(C#N)c(-c4ccccc4)c(-n4c5ccc(-c6ccccc6)cc5c5c6oc7ccccc7c6ccc54)c3-n3c4ccc(-c5ccccc5)cc4c4c5oc6ccccc6c5ccc43)nc(-c3c([2H])c([2H])c([2H])c([2H])c3[2H])n2)c([2H])c1[2H]]

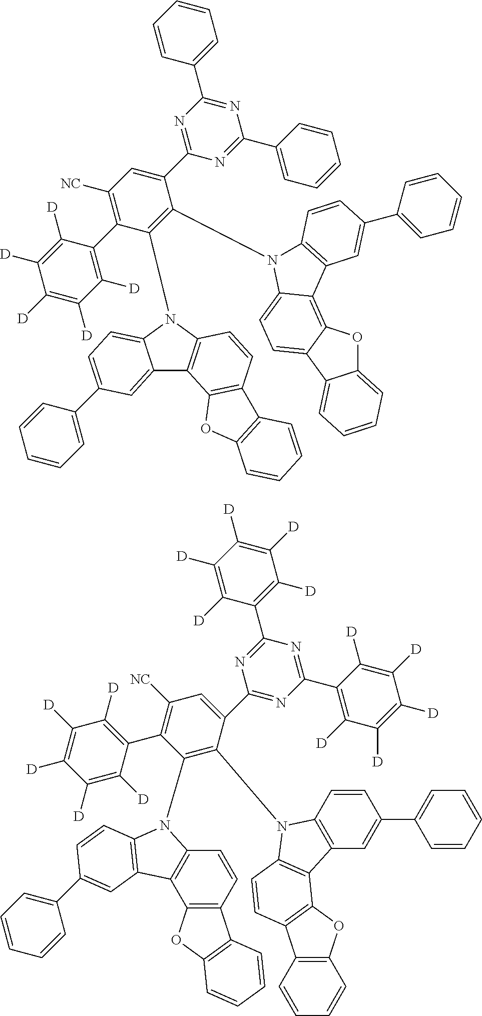

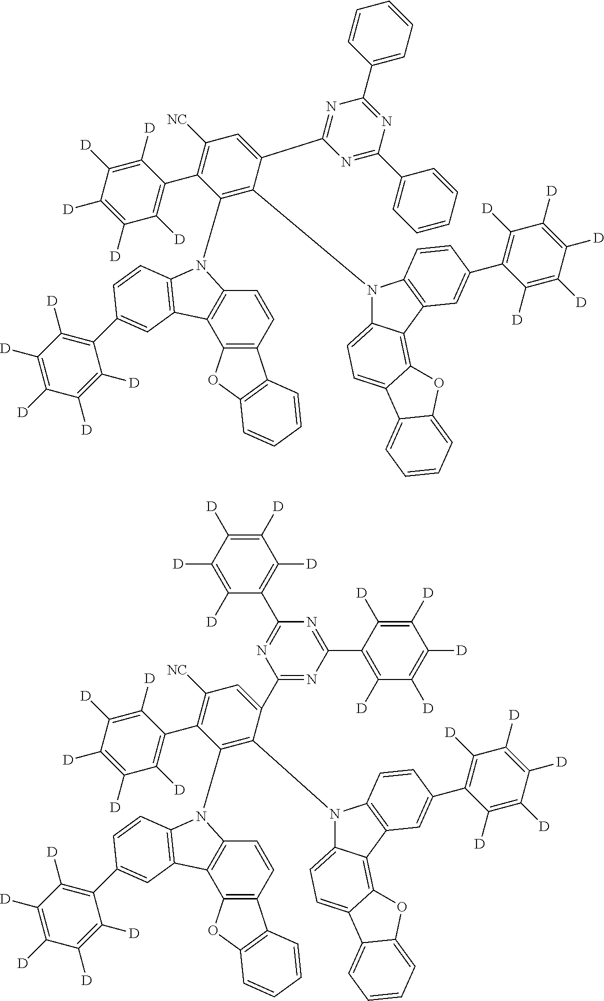

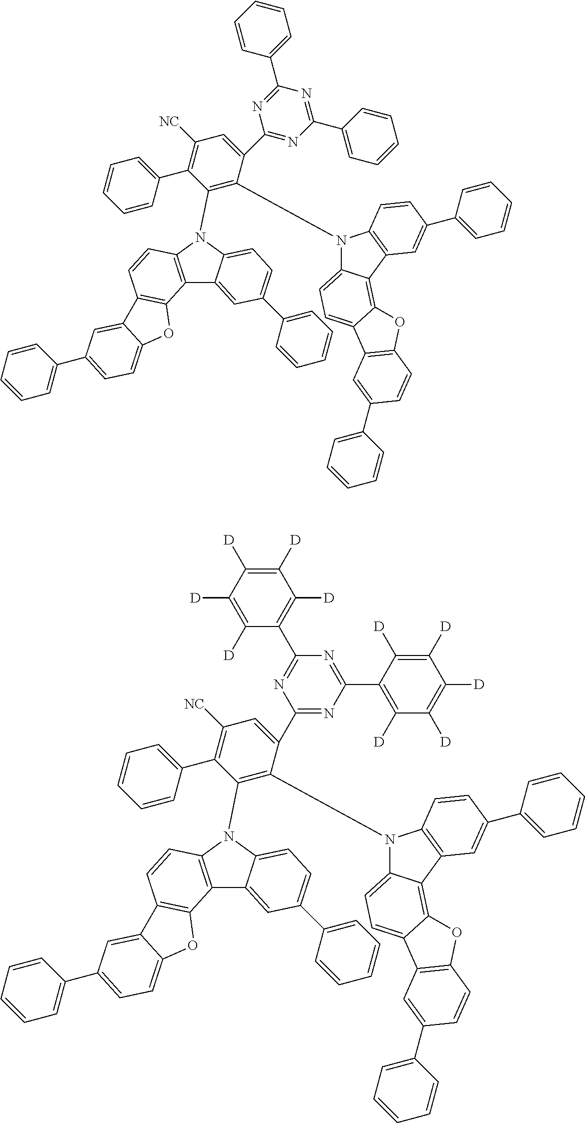 [SMILES: N#Cc1cc(-c2nc(-c3ccccc3)nc(-c3ccccc3)n2)c(-n2c3ccc(-c4ccccc4)cc3c3c4oc5ccc(-c6ccccc6)cc5c4ccc32)c(-n2c3ccc(-c4ccccc4)cc3c3c4oc5ccc(-c6ccccc6)cc5c4ccc32)c1-c1ccccc1 ; [2H]c1c([2H])c([2H])c(-c2nc(-c3cc(C#N)c(-c4ccccc4)c(-n4c5ccc(-c6ccccc6)cc5c5c6oc7ccc(-c8ccccc8)cc7c6ccc54)c3-n3c4ccc(-c5ccccc5)cc4c4c5oc6ccc(-c7ccccc7)cc6c5ccc43)nc(-c3c([2H])c([2H])c([2H])c([2H])c3[2H])n2)c([2H])c1[2H]]

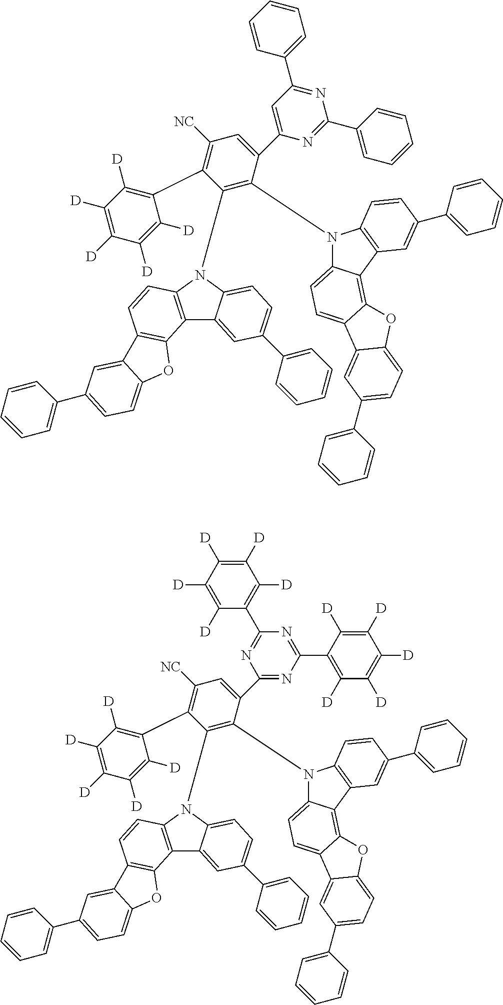 [SMILES: [2H]c1c([2H])c([2H])c(-c2c(C#N)cc(-c3cc(-c4ccccc4)nc(-c4ccccc4)n3)c(-n3c4ccc(-c5ccccc5)cc4c4c5oc6ccc(-c7ccccc7)cc6c5ccc43)c2-n2c3ccc(-c4ccccc4)cc3c3c4oc5ccc(-c6ccccc6)cc5c4ccc32)c([2H])c1[2H] ; [2H]c1c([2H])c([2H])c(-c2nc(-c3cc(C#N)c(-c4c([2H])c([2H])c([2H])c([2H])c4[2H])c(-n4c5ccc(-c6ccccc6)cc5c5c6oc7ccc(-c8ccccc8)cc7c6ccc54)c3-n3c4ccc(-c5ccccc5)cc4c4c5oc6ccc(-c7ccccc7)cc6c5ccc43)nc(-c3c([2H])c([2H])c([2H])c([2H])c3[2H])n2)c([2H])c1[2H]]

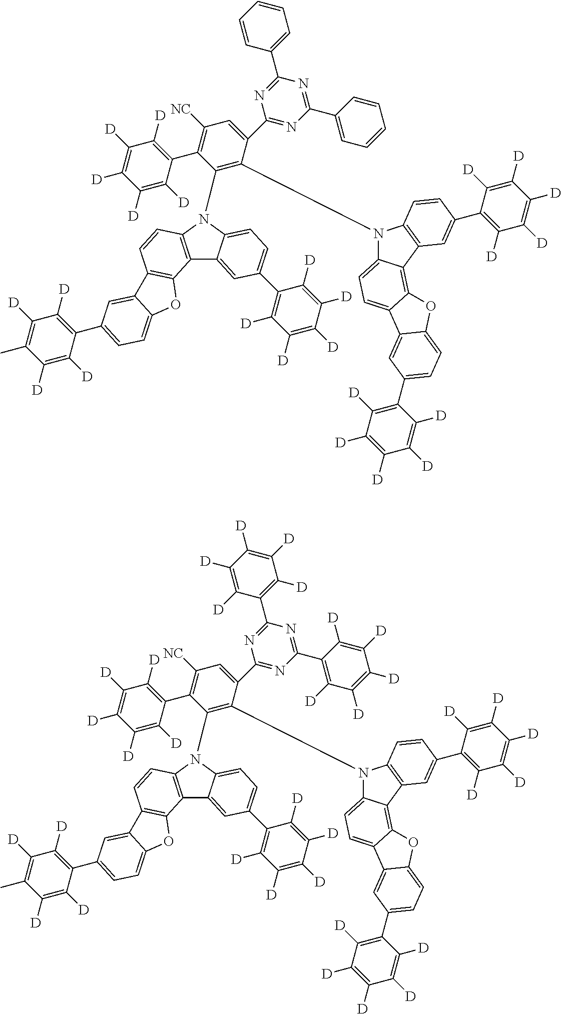

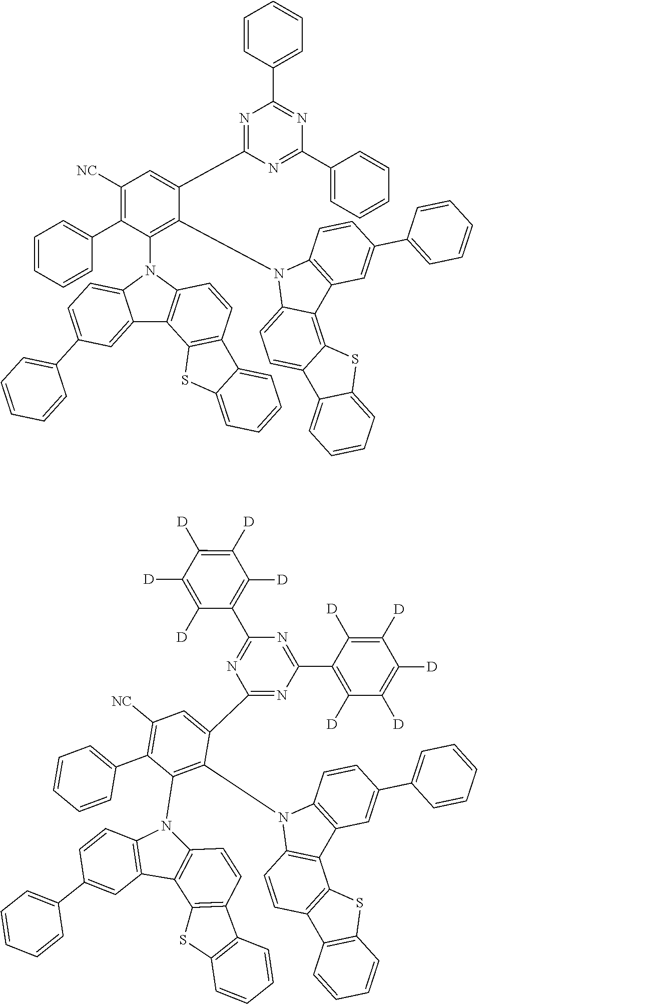 [SMILES: N#Cc1cc(-c2nc(-c3ccccc3)nc(-c3ccccc3)n2)c(-n2c3ccc(-c4ccccc4)cc3c3c4sc5ccccc5c4ccc32)c(-n2c3ccc(-c4ccccc4)cc3c3c4sc5ccccc5c4ccc32)c1-c1ccccc1 ; [2H]c1c([2H])c([2H])c(-c2nc(-c3cc(C#N)c(-c4ccccc4)c(-n4c5ccc(-c6ccccc6)cc5c5c6sc7ccccc7c6ccc54)c3-n3c4ccc(-c5ccccc5)cc4c4c5sc6ccccc6c5ccc43)nc(-c3c([2H])c([2H])c([2H])c([2H])c3[2H])n2)c([2H])c1[2H]]

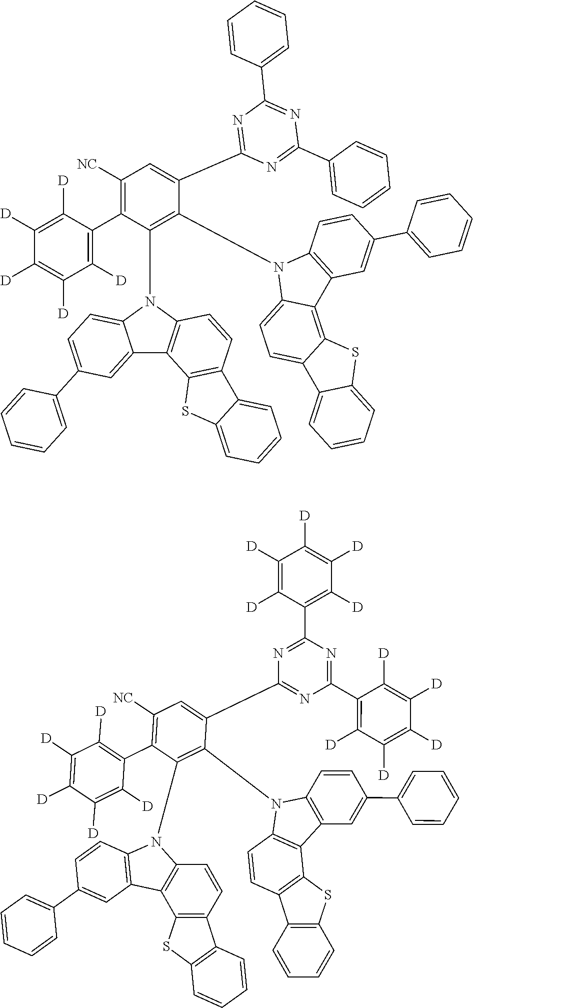 [SMILES: [2H]c1c([2H])c([2H])c(-c2c(C#N)cc(-c3nc(-c4ccccc4)nc(-c4ccccc4)n3)c(-n3c4ccc(-c5ccccc5)cc4c4c5sc6ccccc6c5ccc43)c2-n2c3ccc(-c4ccccc4)cc3c3c4sc5ccccc5c4ccc32)c([2H])c1[2H] ; [2H]c1c([2H])c([2H])c(-c2nc(-c3cc(C#N)c(-c4c([2H])c([2H])c([2H])c([2H])c4[2H])c(-n4c5ccc(-c6ccccc6)cc5c5c6sc7ccccc7c6ccc54)c3-n3c4ccc(-c5ccccc5)cc4c4c5sc6ccccc6c5ccc43)nc(-c3c([2H])c([2H])c([2H])c([2H])c3[2H])n2)c([2H])c1[2H]]

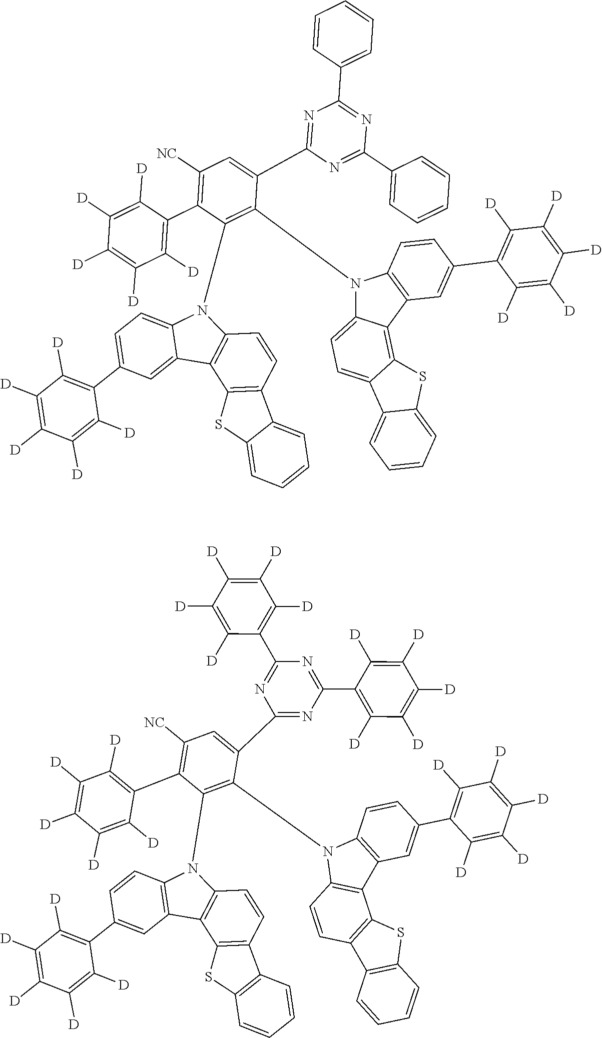

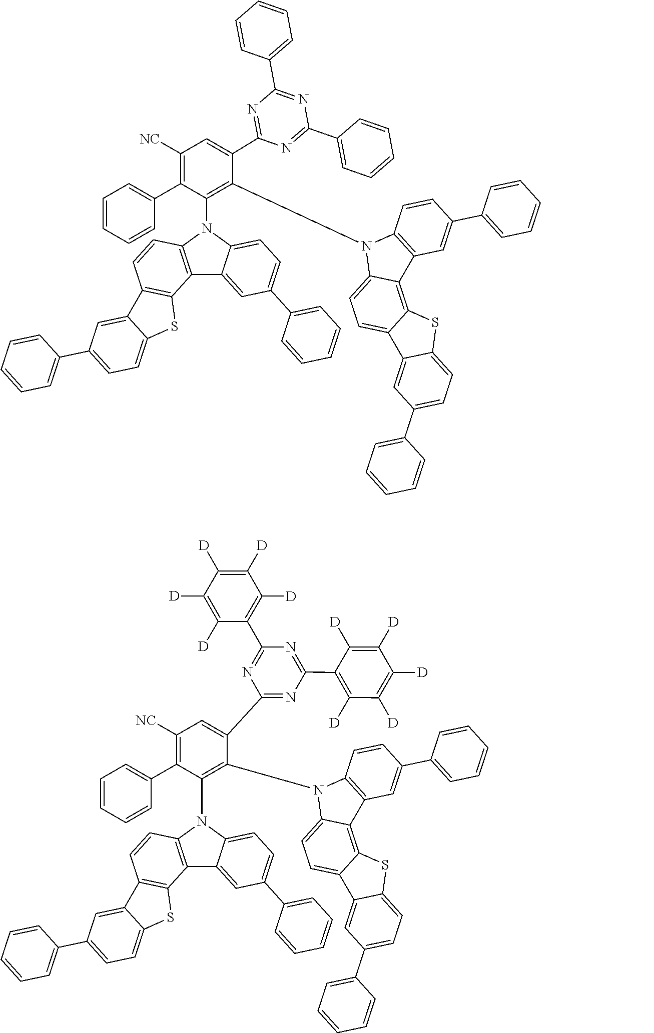 [SMILES: N#Cc1cc(-c2nc(-c3ccccc3)nc(-c3ccccc3)n2)c(-n2c3ccc(-c4ccccc4)cc3c3c4sc5ccc(-c6ccccc6)cc5c4ccc32)c(-n2c3ccc(-c4ccccc4)cc3c3c4sc5ccc(-c6ccccc6)cc5c4ccc32)c1-c1ccccc1 ; [2H]c1c([2H])c([2H])c(-c2nc(-c3cc(C#N)c(-c4ccccc4)c(-n4c5ccc(-c6ccccc6)cc5c5c6sc7ccc(-c8ccccc8)cc7c6ccc54)c3-n3c4ccc(-c5ccccc5)cc4c4c5sc6ccc(-c7ccccc7)cc6c5ccc43)nc(-c3c([2H])c([2H])c([2H])c([2H])c3[2H])n2)c([2H])c1[2H]]

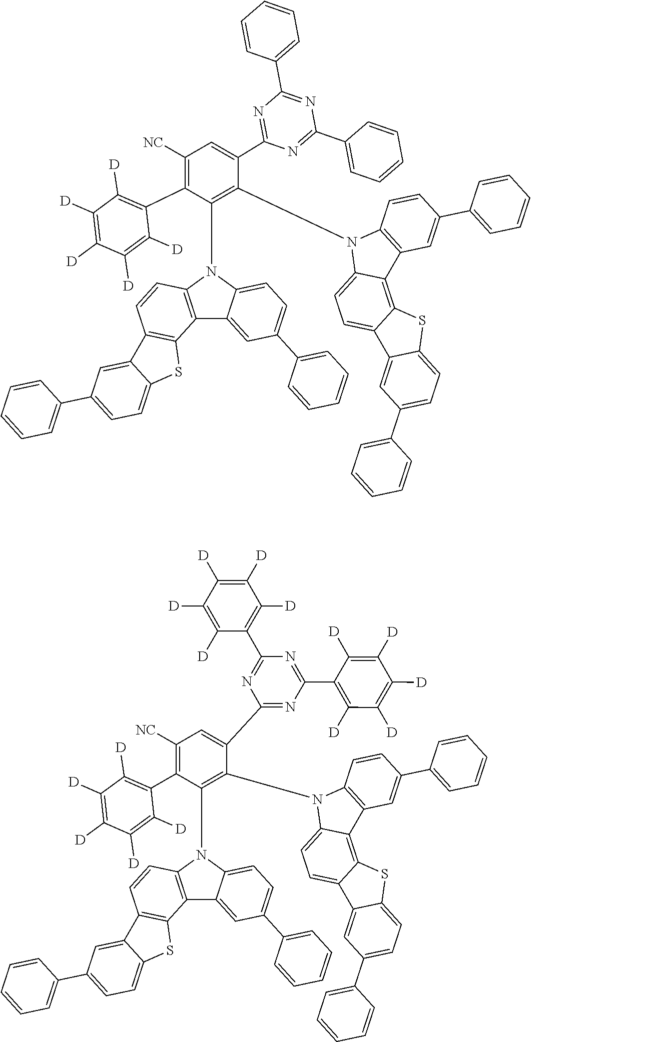 [SMILES: [2H]c1c([2H])c([2H])c(-c2c(C#N)cc(-c3nc(-c4ccccc4)nc(-c4ccccc4)n3)c(-n3c4ccc(-c5ccccc5)cc4c4c5sc6ccc(-c7ccccc7)cc6c5ccc43)c2-n2c3ccc(-c4ccccc4)cc3c3c4sc5ccc(-c6ccccc6)cc5c4ccc32)c([2H])c1[2H] ; [2H]c1c([2H])c([2H])c(-c2nc(-c3cc(C#N)c(-c4c([2H])c([2H])c([2H])c([2H])c4[2H])c(-n4c5ccc(-c6ccccc6)cc5c5c6sc7ccc(-c8ccccc8)cc7c6ccc54)c3-n3c4ccc(-c5ccccc5)cc4c4c5sc6ccc(-c7ccccc7)cc6c5ccc43)nc(-c3c([2H])c([2H])c([2H])c([2H])c3[2H])n2)c([2H])c1[2H]]

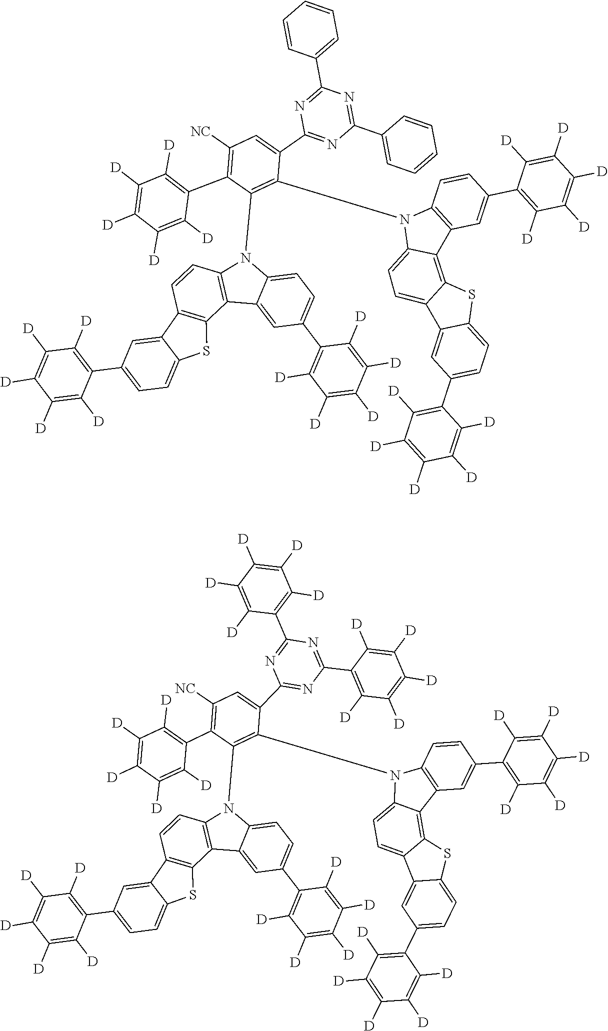

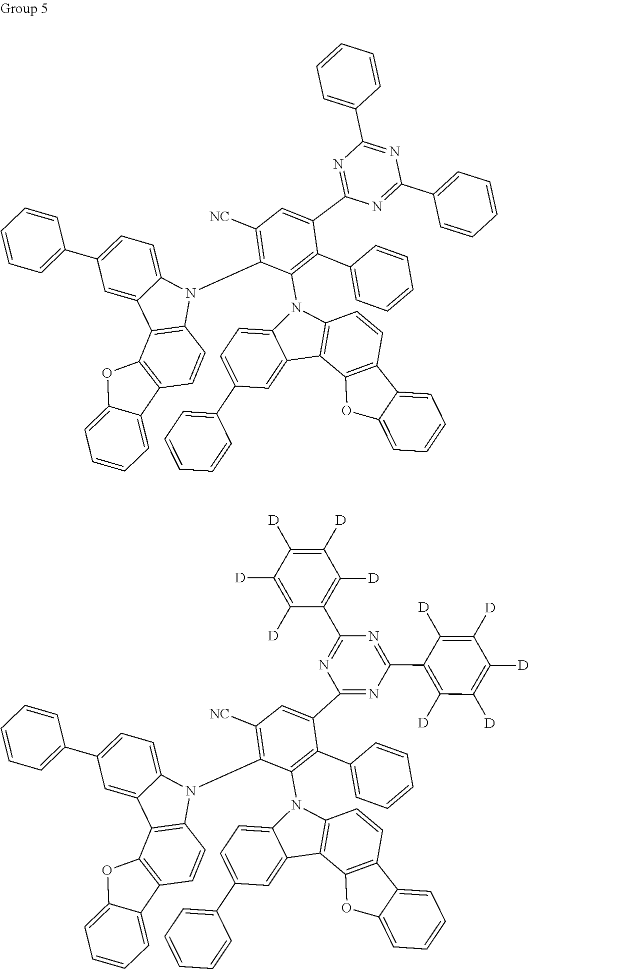 [SMILES: N#Cc1cc(-c2nc(-c3ccccc3)nc(-c3ccccc3)n2)c(-c2ccccc2)c(-n2c3ccc(-c4ccccc4)cc3c3c4oc5ccccc5c4ccc32)c1-n1c2ccc(-c3ccccc3)cc2c2c3oc4ccccc4c3ccc21 ; [2H]c1c([2H])c([2H])c(-c2nc(-c3cc(C#N)c(-n4c5ccc(-c6ccccc6)cc5c5c6oc7ccccc7c6ccc54)c(-n4c5ccc(-c6ccccc6)cc5c5c6oc7ccccc7c6ccc54)c3-c3ccccc3)nc(-c3c([2H])c([2H])c([2H])c([2H])c3[2H])n2)c([2H])c1[2H]]

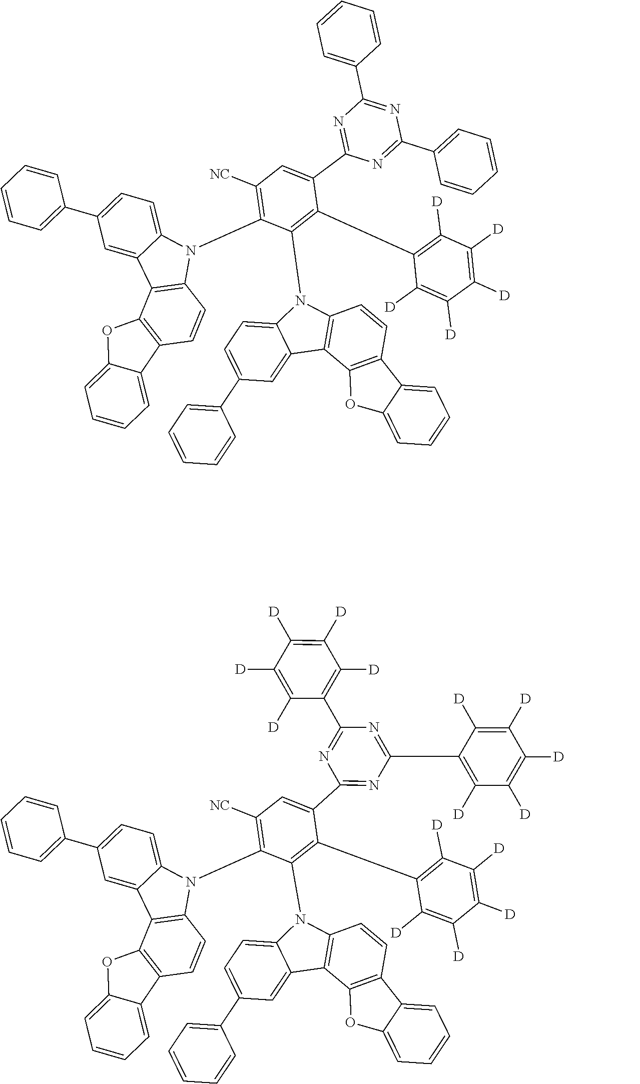 [SMILES: [2H]c1c([2H])c([2H])c(-c2c(-c3nc(-c4ccccc4)nc(-c4ccccc4)n3)cc(C#N)c(-n3c4ccc(-c5ccccc5)cc4c4c5oc6ccccc6c5ccc43)c2-n2c3ccc(-c4ccccc4)cc3c3c4oc5ccccc5c4ccc32)c([2H])c1[2H] ; [2H]c1c([2H])c([2H])c(-c2nc(-c3cc(C#N)c(-n4c5ccc(-c6ccccc6)cc5c5c6oc7ccccc7c6ccc54)c(-n4c5ccc(-c6ccccc6)cc5c5c6oc7ccccc7c6ccc54)c3-c3c([2H])c([2H])c([2H])c([2H])c3[2H])nc(-c3c([2H])c([2H])c([2H])c([2H])c3[2H])n2)c([2H])c1[2H]]

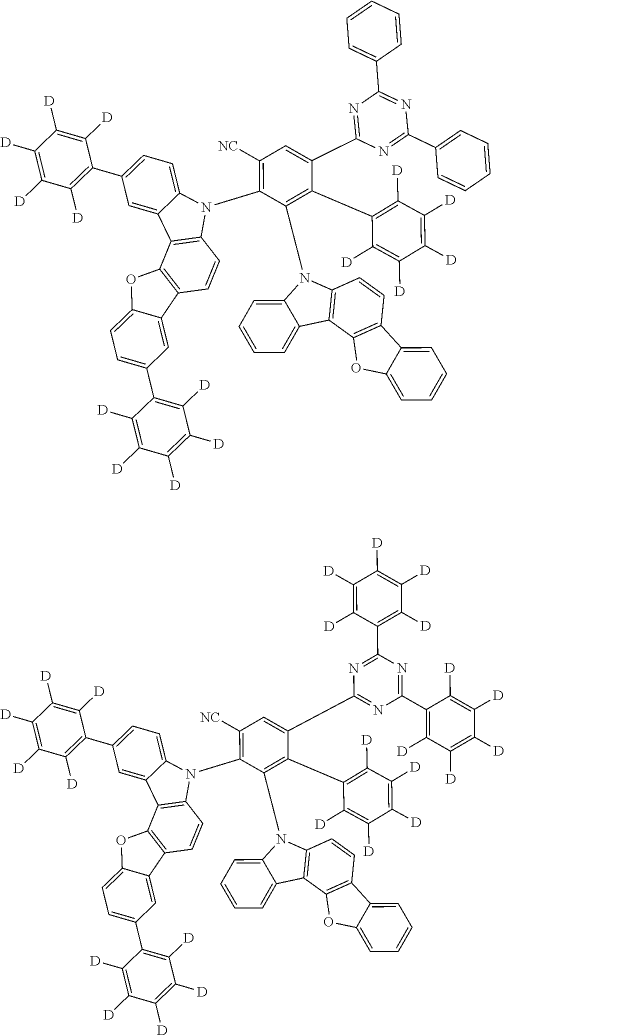

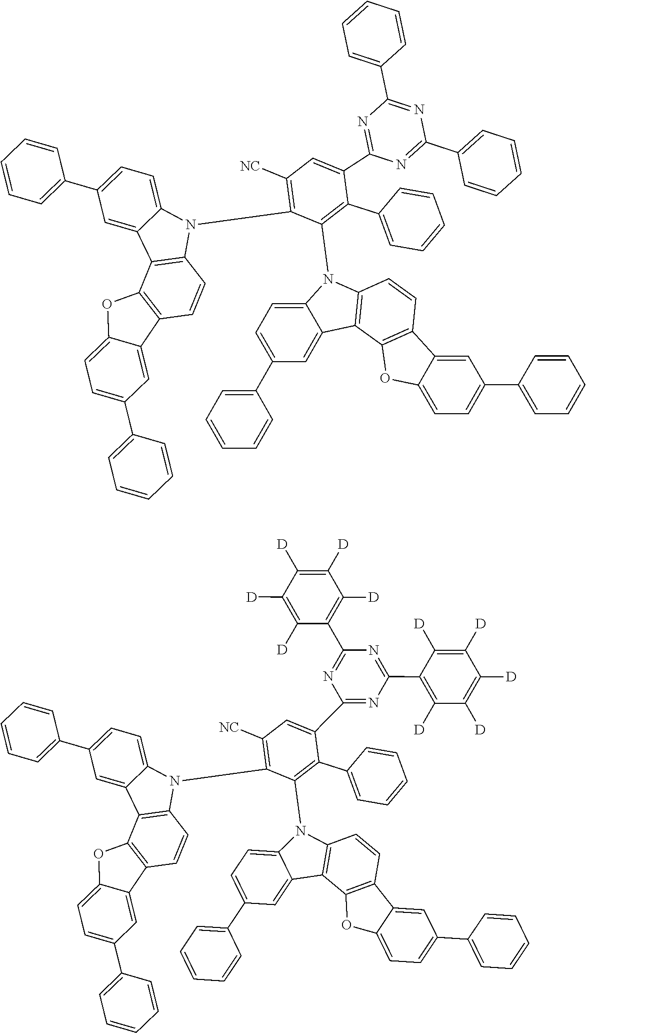 [SMILES: N#Cc1cc(-c2nc(-c3ccccc3)nc(-c3ccccc3)n2)c(-c2ccccc2)c(-n2c3ccc(-c4ccccc4)cc3c3c4oc5ccc(-c6ccccc6)cc5c4ccc32)c1-n1c2ccc(-c3ccccc3)cc2c2c3oc4ccc(-c5ccccc5)cc4c3ccc21 ; [2H]c1c([2H])c([2H])c(-c2nc(-c3cc(C#N)c(-n4c5ccc(-c6ccccc6)cc5c5c6oc7ccc(-c8ccccc8)cc7c6ccc54)c(-n4c5ccc(-c6ccccc6)cc5c5c6oc7ccc(-c8ccccc8)cc7c6ccc54)c3-c3ccccc3)nc(-c3c([2H])c([2H])c([2H])c([2H])c3[2H])n2)c([2H])c1[2H]]

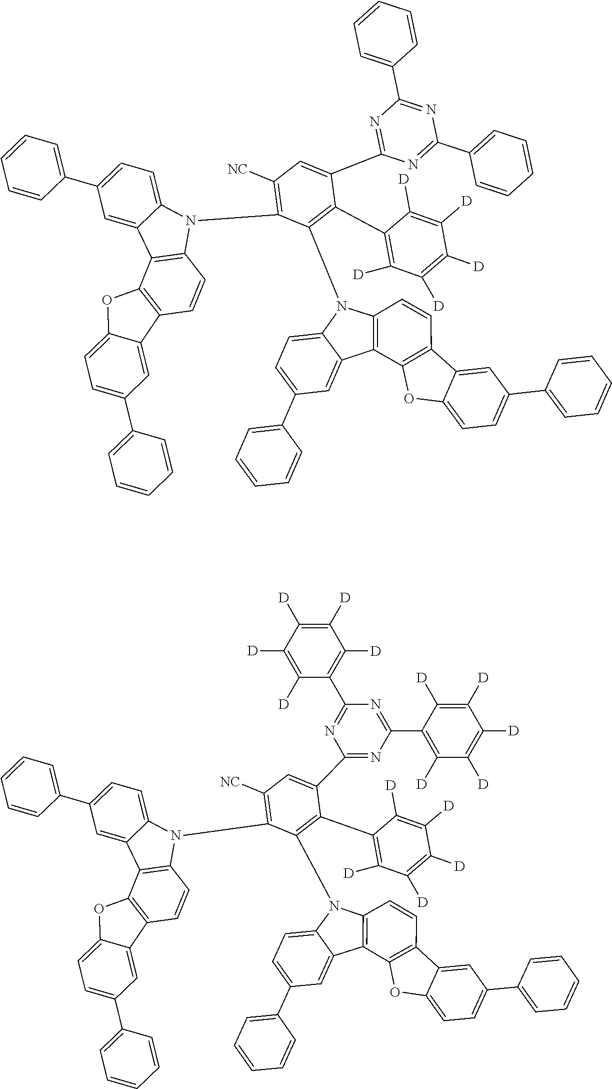 [SMILES: [2H]c1c([2H])c([2H])c(-c2c(-c3nc(-c4ccccc4)nc(-c4ccccc4)n3)cc(C#N)c(-n3c4ccc(-c5ccccc5)cc4c4c5oc6ccc(-c7ccccc7)cc6c5ccc43)c2-n2c3ccc(-c4ccccc4)cc3c3c4oc5ccc(-c6ccccc6)cc5c4ccc32)c([2H])c1[2H] ; [2H]c1c([2H])c([2H])c(-c2nc(-c3cc(C#N)c(-n4c5ccc(-c6ccccc6)cc5c5c6oc7ccc(-c8ccccc8)cc7c6ccc54)c(-n4c5ccc(-c6ccccc6)cc5c5c6oc7ccc(-c8ccccc8)cc7c6ccc54)c3-c3c([2H])c([2H])c([2H])c([2H])c3[2H])nc(-c3c([2H])c([2H])c([2H])c([2H])c3[2H])n2)c([2H])c1[2H]]

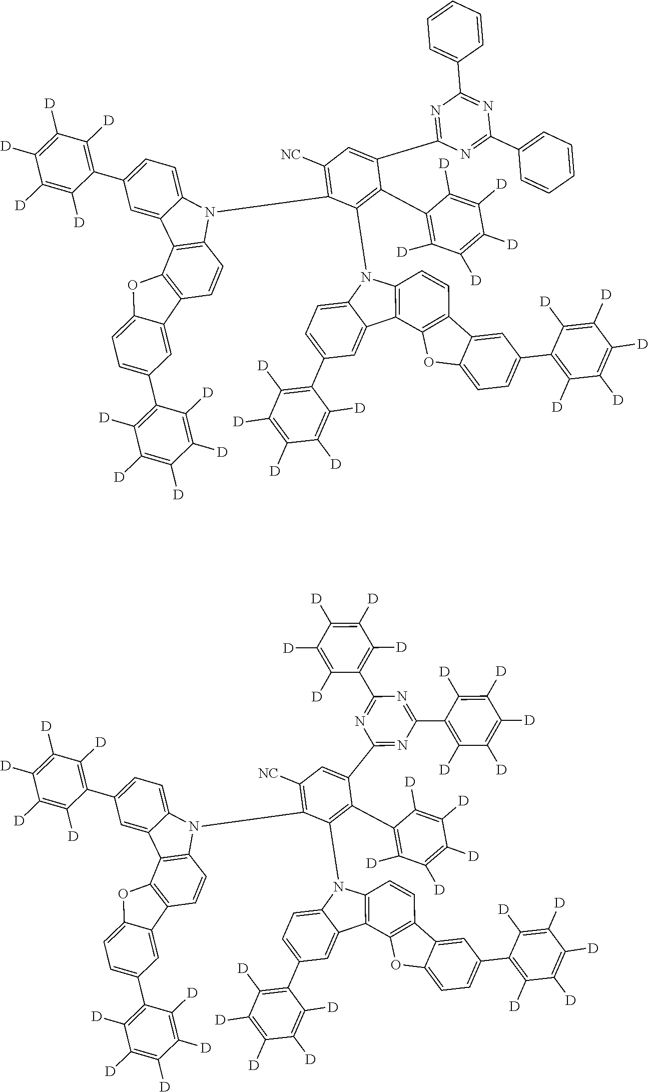

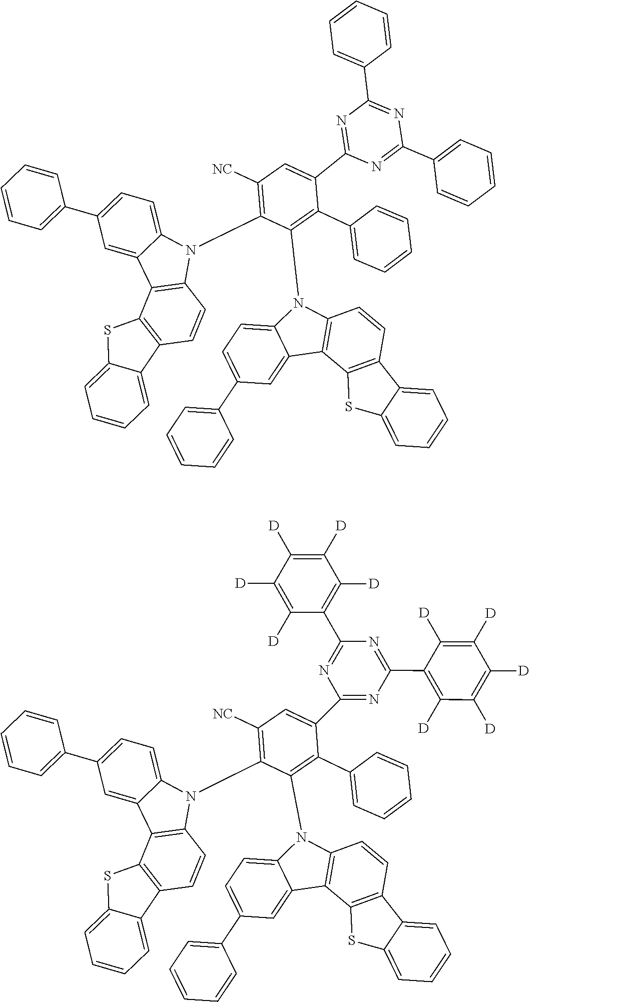 [SMILES: N#Cc1cc(-c2nc(-c3ccccc3)nc(-c3ccccc3)n2)c(-c2ccccc2)c(-n2c3ccc(-c4ccccc4)cc3c3c4sc5ccccc5c4ccc32)c1-n1c2ccc(-c3ccccc3)cc2c2c3sc4ccccc4c3ccc21 ; [2H]c1c([2H])c([2H])c(-c2nc(-c3cc(C#N)c(-n4c5ccc(-c6ccccc6)cc5c5c6sc7ccccc7c6ccc54)c(-n4c5ccc(-c6ccccc6)cc5c5c6sc7ccccc7c6ccc54)c3-c3ccccc3)nc(-c3c([2H])c([2H])c([2H])c([2H])c3[2H])n2)c([2H])c1[2H]]

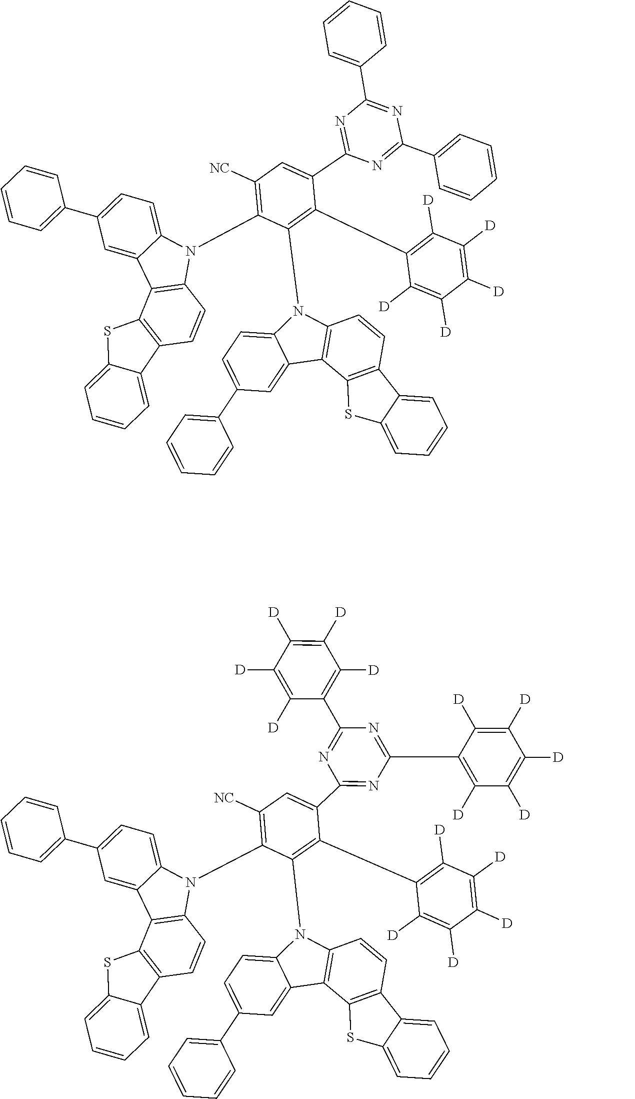 [SMILES: [2H]c1c([2H])c([2H])c(-c2c(-c3nc(-c4ccccc4)nc(-c4ccccc4)n3)cc(C#N)c(-n3c4ccc(-c5ccccc5)cc4c4c5sc6ccccc6c5ccc43)c2-n2c3ccc(-c4ccccc4)cc3c3c4sc5ccccc5c4ccc32)c([2H])c1[2H] ; [2H]c1c([2H])c([2H])c(-c2nc(-c3cc(C#N)c(-n4c5ccc(-c6ccccc6)cc5c5c6sc7ccccc7c6ccc54)c(-n4c5ccc(-c6ccccc6)cc5c5c6sc7ccccc7c6ccc54)c3-c3c([2H])c([2H])c([2H])c([2H])c3[2H])nc(-c3c([2H])c([2H])c([2H])c([2H])c3[2H])n2)c([2H])c1[2H]]

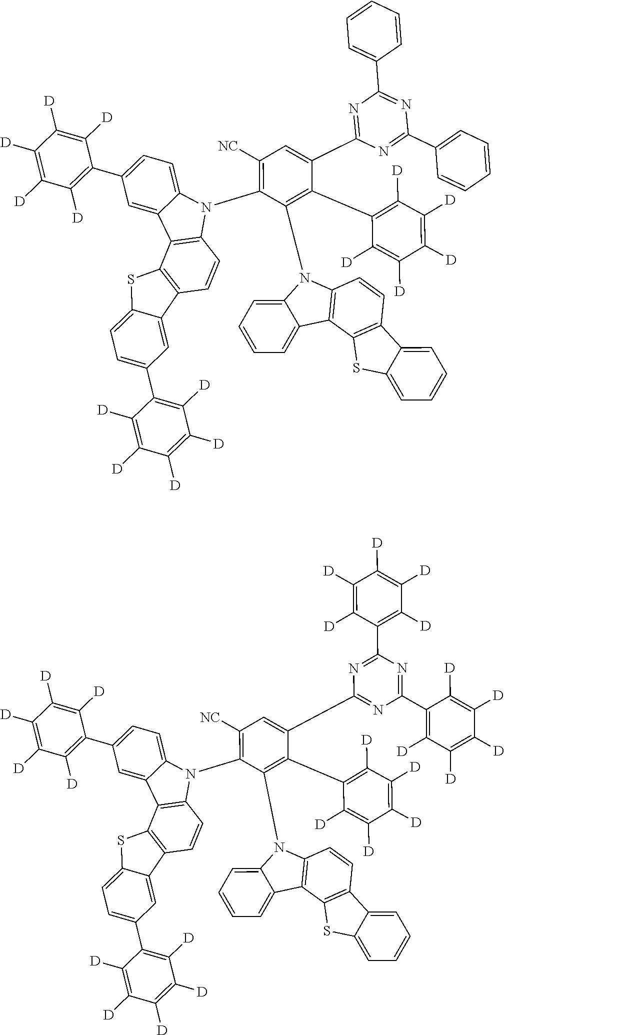

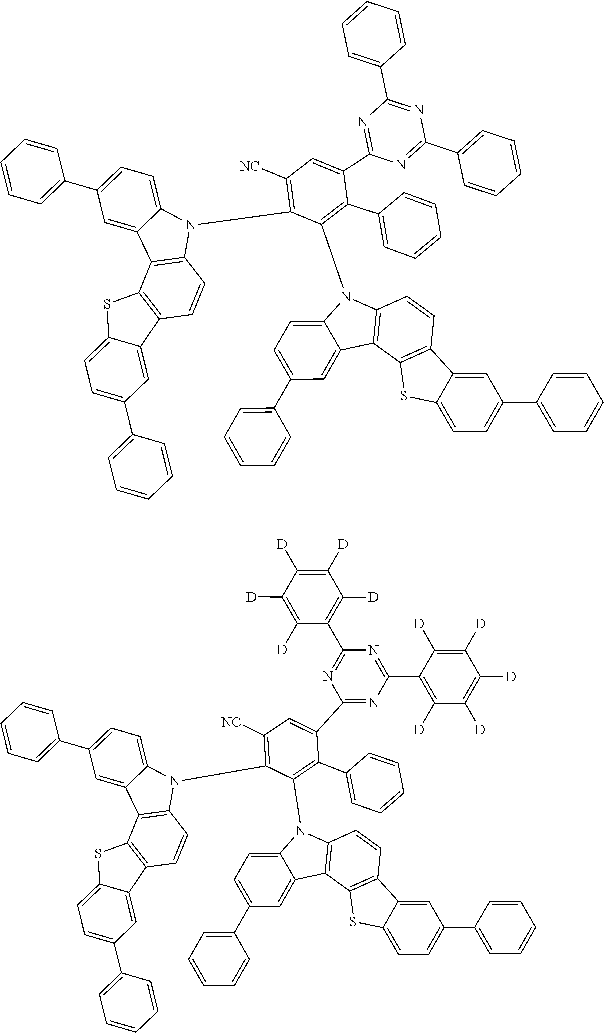 [SMILES: N#Cc1cc(-c2nc(-c3ccccc3)nc(-c3ccccc3)n2)c(-c2ccccc2)c(-n2c3ccc(-c4ccccc4)cc3c3c4sc5ccc(-c6ccccc6)cc5c4ccc32)c1-n1c2ccc(-c3ccccc3)cc2c2c3sc4ccc(-c5ccccc5)cc4c3ccc21 ; [2H]c1c([2H])c([2H])c(-c2nc(-c3cc(C#N)c(-n4c5ccc(-c6ccccc6)cc5c5c6sc7ccc(-c8ccccc8)cc7c6ccc54)c(-n4c5ccc(-c6ccccc6)cc5c5c6sc7ccc(-c8ccccc8)cc7c6ccc54)c3-c3ccccc3)nc(-c3c([2H])c([2H])c([2H])c([2H])c3[2H])n2)c([2H])c1[2H]]

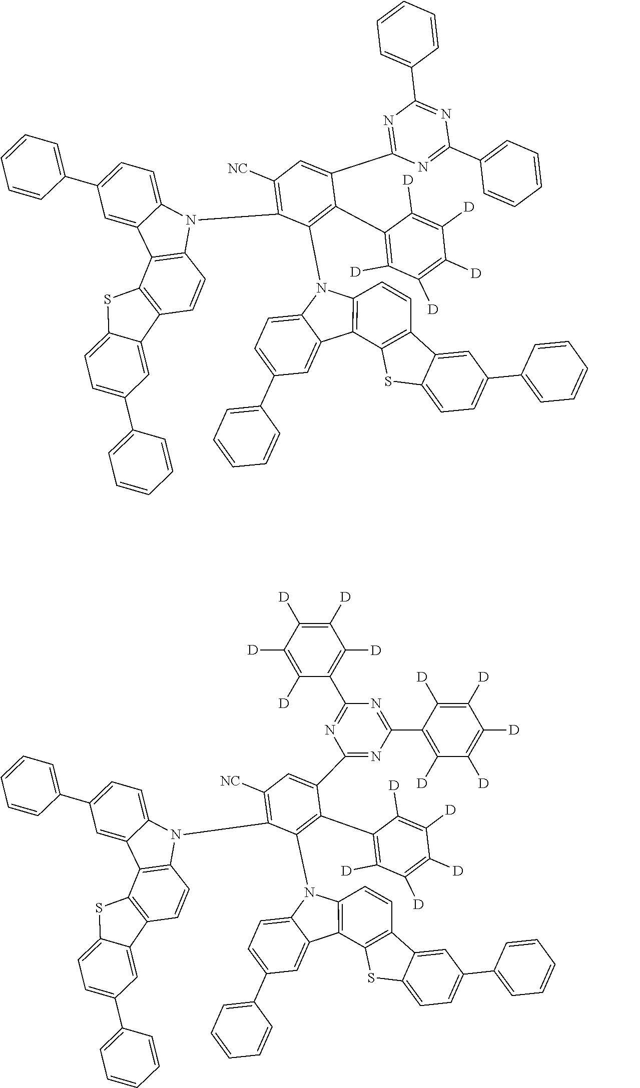 [SMILES: [2H]c1c([2H])c([2H])c(-c2c(-c3nc(-c4ccccc4)nc(-c4ccccc4)n3)cc(C#N)c(-n3c4ccc(-c5ccccc5)cc4c4c5sc6ccc(-c7ccccc7)cc6c5ccc43)c2-n2c3ccc(-c4ccccc4)cc3c3c4sc5ccc(-c6ccccc6)cc5c4ccc32)c([2H])c1[2H] ; [2H]c1c([2H])c([2H])c(-c2nc(-c3cc(C#N)c(-n4c5ccc(-c6ccccc6)cc5c5c6sc7ccc(-c8ccccc8)cc7c6ccc54)c(-n4c5ccc(-c6ccccc6)cc5c5c6sc7ccc(-c8ccccc8)cc7c6ccc54)c3-c3c([2H])c([2H])c([2H])c([2H])c3[2H])nc(-c3c([2H])c([2H])c([2H])c([2H])c3[2H])n2)c([2H])c1[2H]]

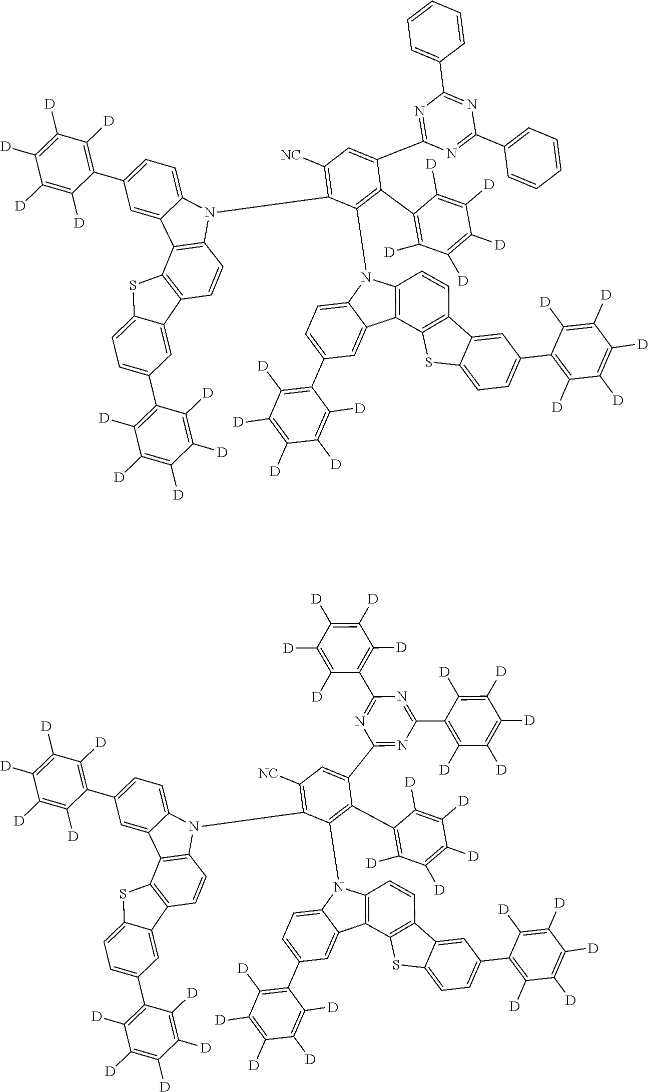

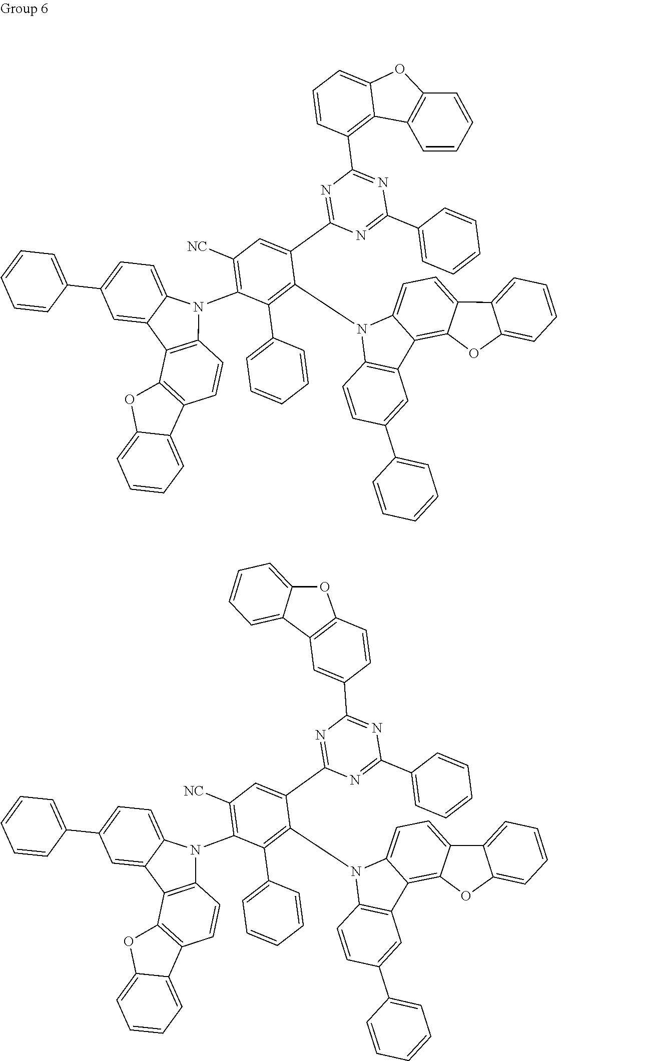 [SMILES: N#Cc1cc(-c2nc(-c3ccccc3)nc(-c3cccc4oc5ccccc5c34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21 ; N#Cc1cc(-c2nc(-c3ccccc3)nc(-c3ccc4oc5ccccc5c4c3)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21]

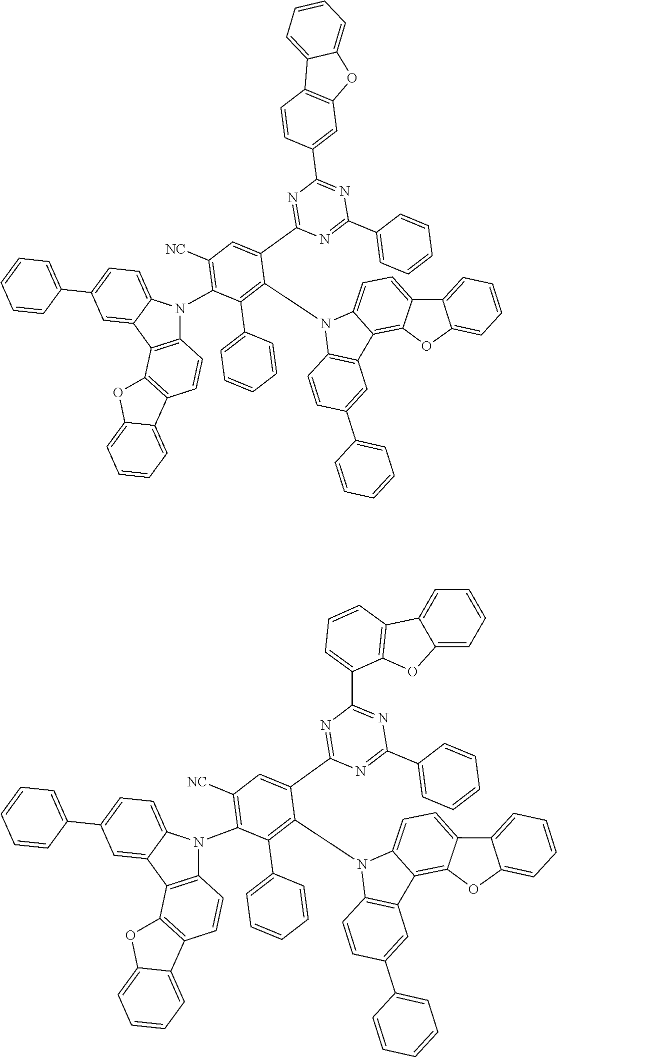 [SMILES: N#Cc1cc(-c2nc(-c3ccccc3)nc(-c3ccc4c(c3)oc3ccccc34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21 ; N#Cc1cc(-c2nc(-c3ccccc3)nc(-c3cccc4c3oc3ccccc34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21]

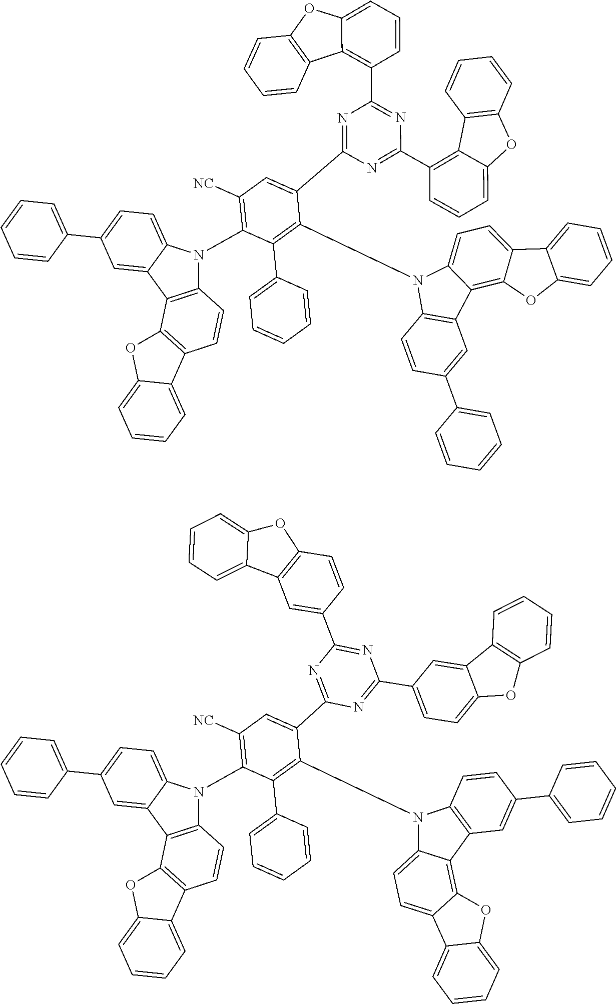 [SMILES: N#Cc1cc(-c2nc(-c3cccc4oc5ccccc5c34)nc(-c3cccc4oc5ccccc5c34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21 ; N#Cc1cc(-c2nc(-c3ccc4oc5ccccc5c4c3)nc(-c3ccc4oc5ccccc5c4c3)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21]

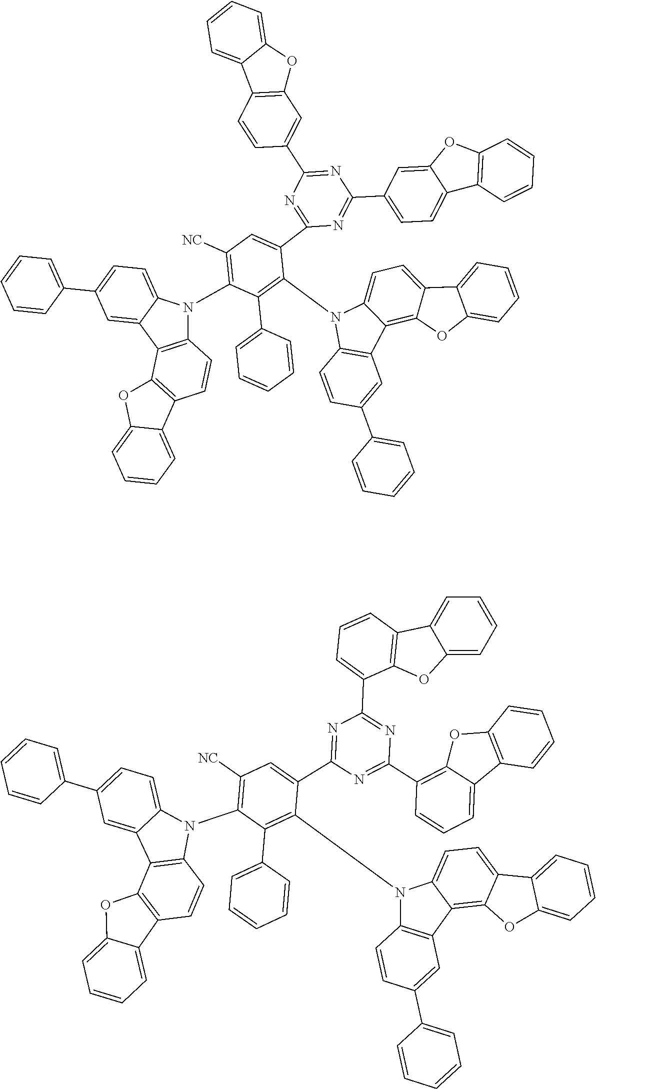 [SMILES: N#Cc1cc(-c2nc(-c3ccc4c(c3)oc3ccccc34)nc(-c3ccc4c(c3)oc3ccccc34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21 ; N#Cc1cc(-c2nc(-c3cccc4c3oc3ccccc34)nc(-c3cccc4c3oc3ccccc34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21]

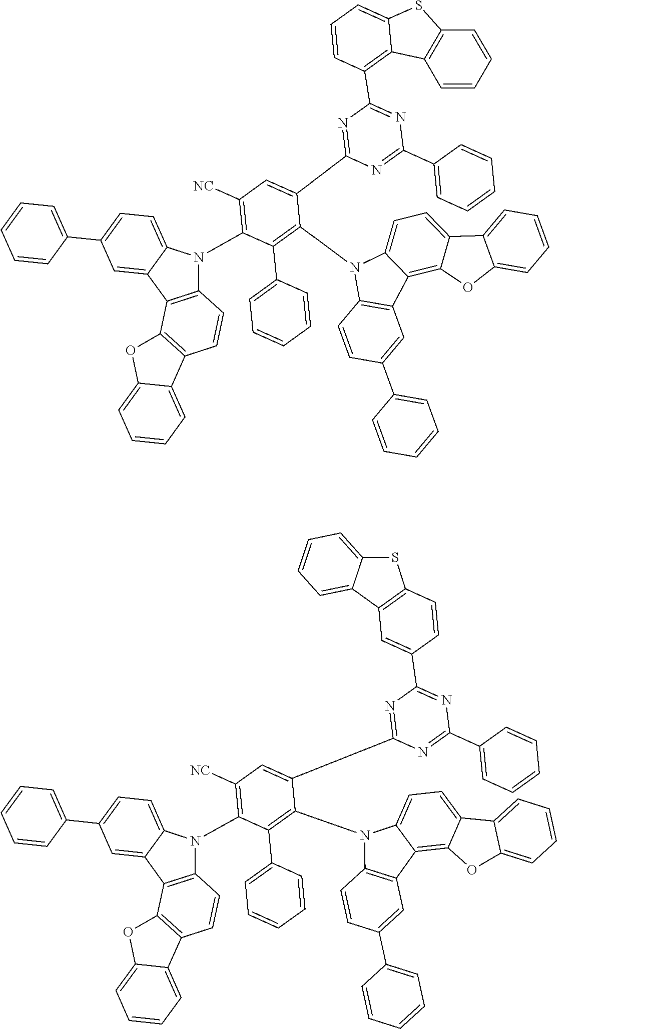 [SMILES: N#Cc1cc(-c2nc(-c3ccccc3)nc(-c3cccc4sc5ccccc5c34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21 ; N#Cc1cc(-c2nc(-c3ccccc3)nc(-c3ccc4sc5ccccc5c4c3)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21]

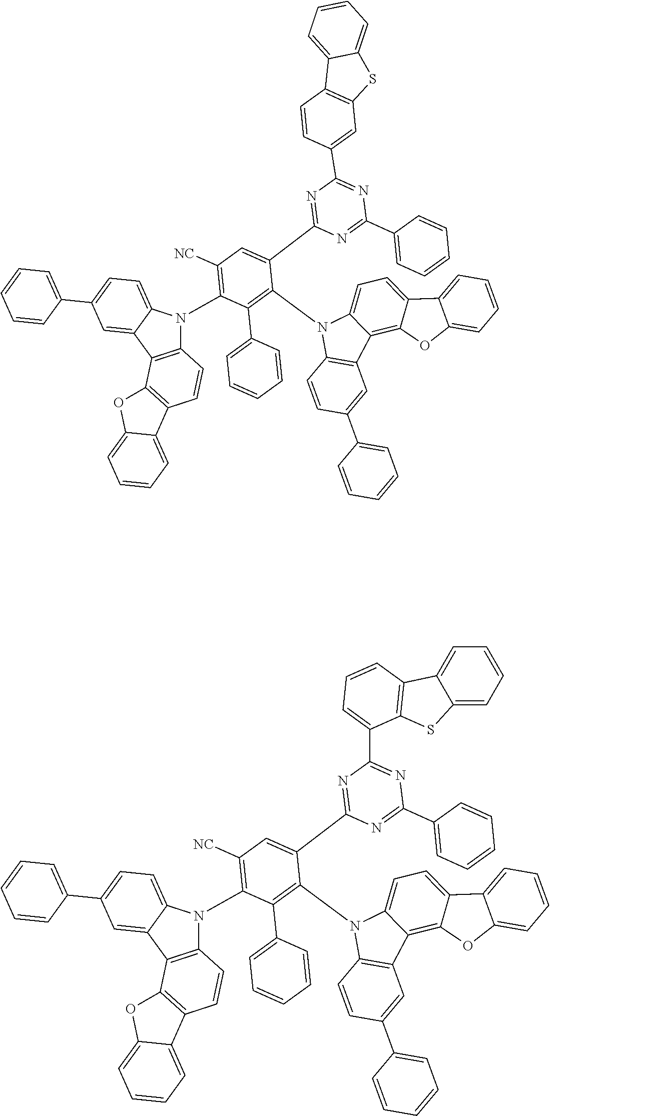 [SMILES: N#Cc1cc(-c2nc(-c3ccccc3)nc(-c3ccc4c(c3)sc3ccccc34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21 ; N#Cc1cc(-c2nc(-c3ccccc3)nc(-c3cccc4c3sc3ccccc34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21]

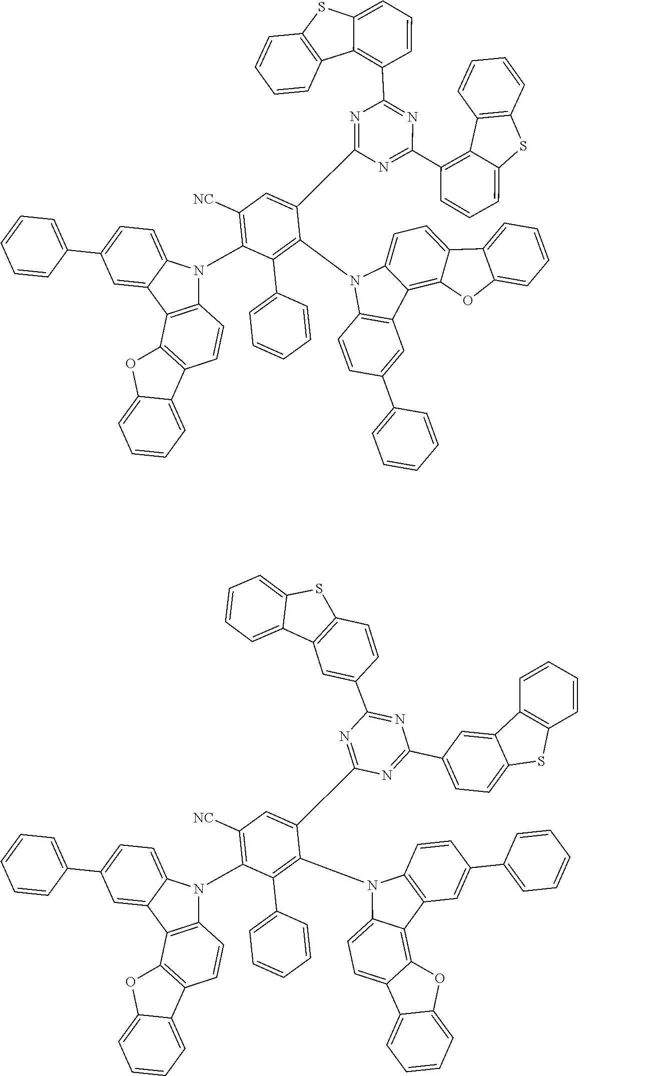 [SMILES: N#Cc1cc(-c2nc(-c3cccc4sc5ccccc5c34)nc(-c3cccc4sc5ccccc5c34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21 ; N#Cc1cc(-c2nc(-c3ccc4sc5ccccc5c4c3)nc(-c3ccc4sc5ccccc5c4c3)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21]

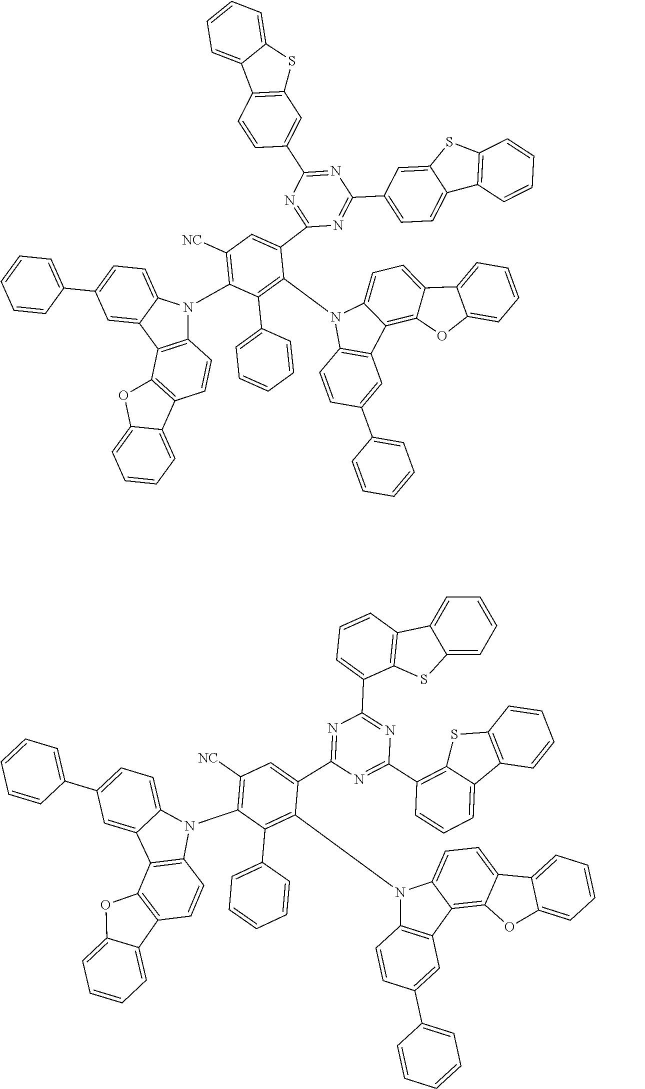 [SMILES: N#Cc1cc(-c2nc(-c3ccc4c(c3)sc3ccccc34)nc(-c3ccc4c(c3)sc3ccccc34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21 ; N#Cc1cc(-c2nc(-c3cccc4c3sc3ccccc34)nc(-c3cccc4c3sc3ccccc34)n2)c(-n2c3ccc(-c4ccccc4)cc3c3c4oc5ccccc5c4ccc32)c(-c2ccccc2)c1-n1c2ccc(-c3ccccc3)cc2c2c3oc4ccccc4c3ccc21]

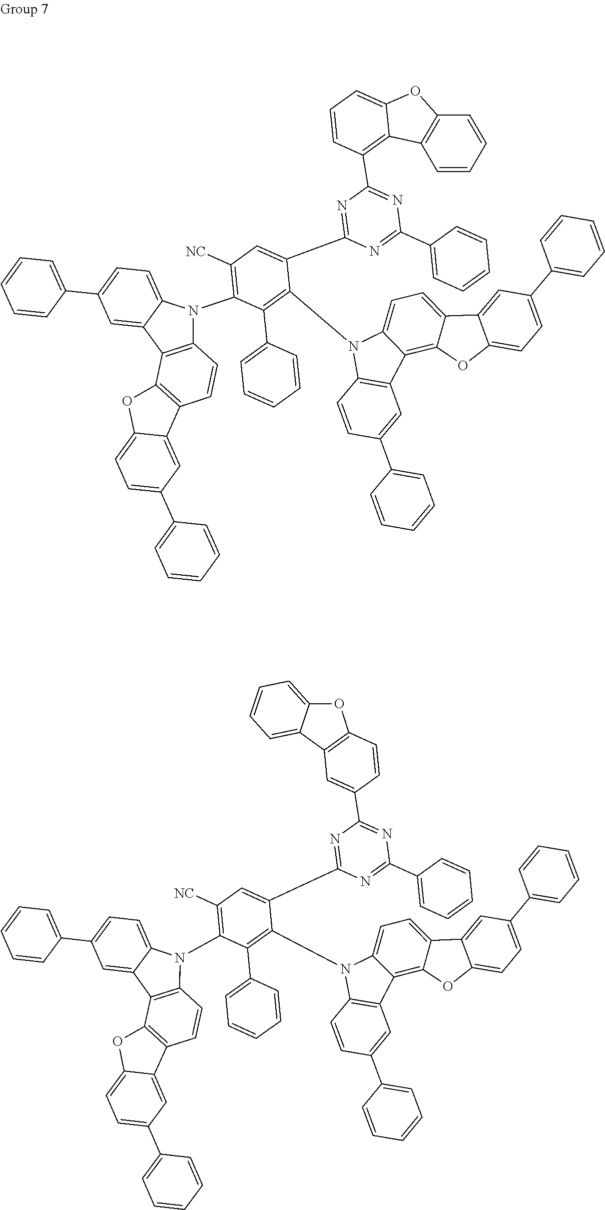 [SMILES: N#Cc1cc(-c2nc(-c3ccccc3)nc(-c3cccc4oc5ccccc5c34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21 ; N#Cc1cc(-c2nc(-c3ccccc3)nc(-c3ccc4oc5ccccc5c4c3)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

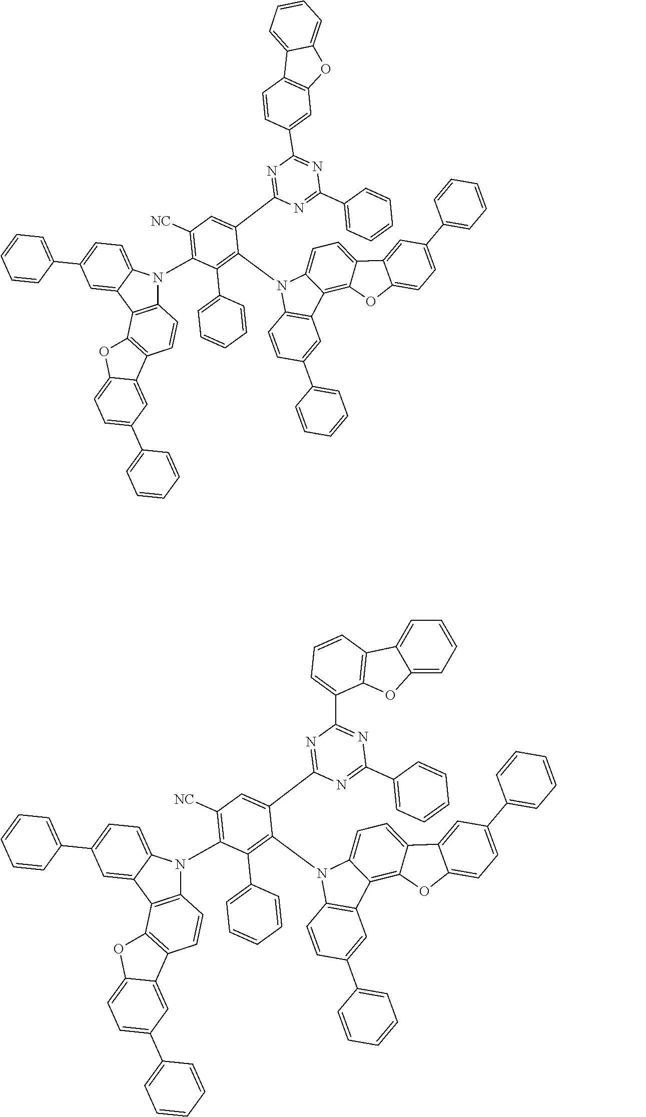 [SMILES: N#Cc1cc(-c2nc(-c3ccccc3)nc(-c3ccc4c(c3)oc3ccccc34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21 ; N#Cc1cc(-c2nc(-c3ccccc3)nc(-c3cccc4c3oc3ccccc34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

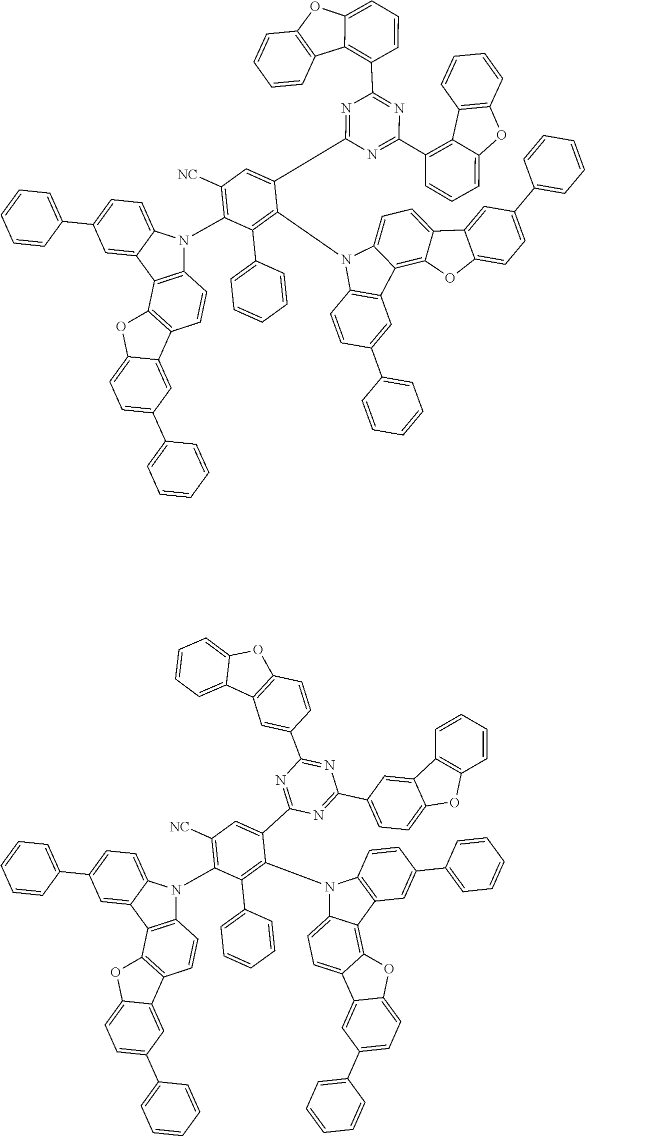 [SMILES: N#Cc1cc(-c2nc(-c3cccc4oc5ccccc5c34)nc(-c3cccc4oc5ccccc5c34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21 ; N#Cc1cc(-c2nc(-c3ccc4oc5ccccc5c4c3)nc(-c3ccc4oc5ccccc5c4c3)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

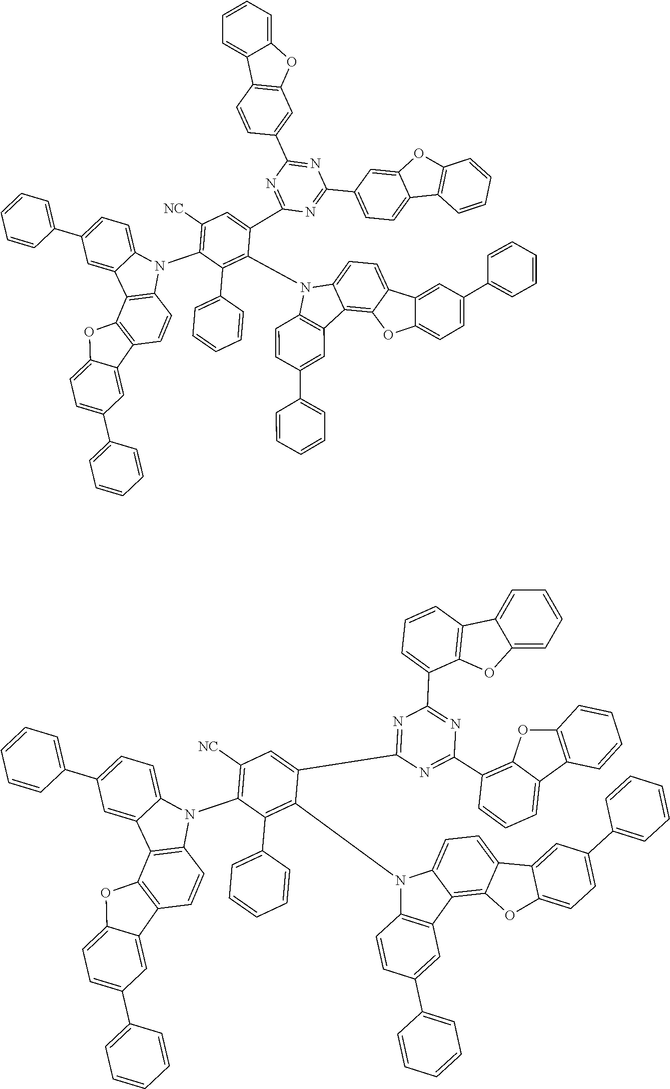 [SMILES: N#Cc1cc(-c2nc(-c3ccc4c(c3)oc3ccccc34)nc(-c3ccc4c(c3)oc3ccccc34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21 ; N#Cc1cc(-c2nc(-c3cccc4c3oc3ccccc34)nc(-c3cccc4c3oc3ccccc34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

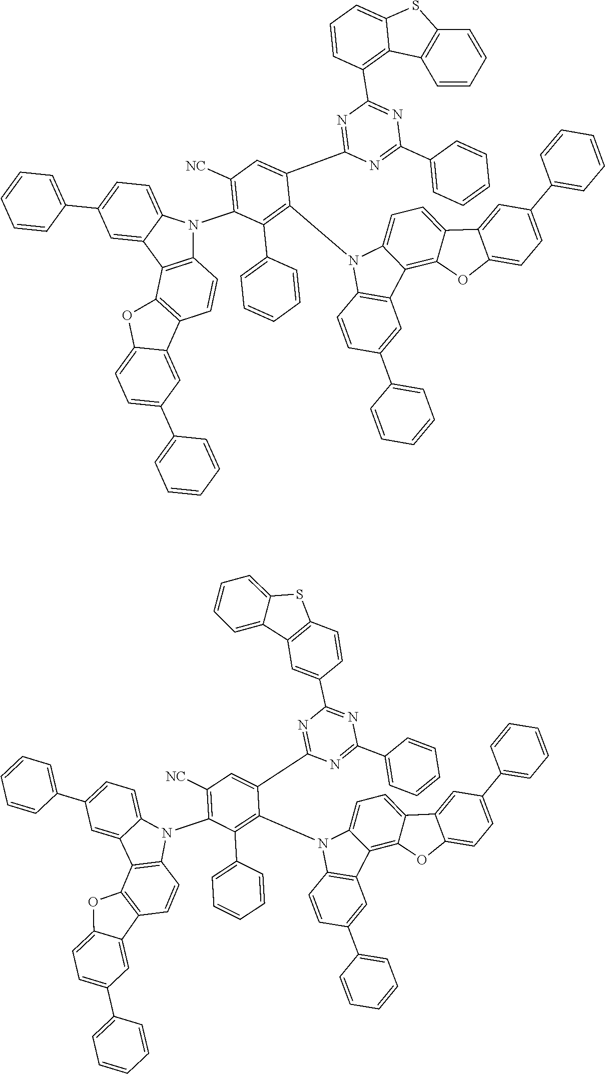 [SMILES: N#Cc1cc(-c2nc(-c3ccccc3)nc(-c3cccc4sc5ccccc5c34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21 ; N#Cc1cc(-c2nc(-c3ccccc3)nc(-c3ccc4sc5ccccc5c4c3)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

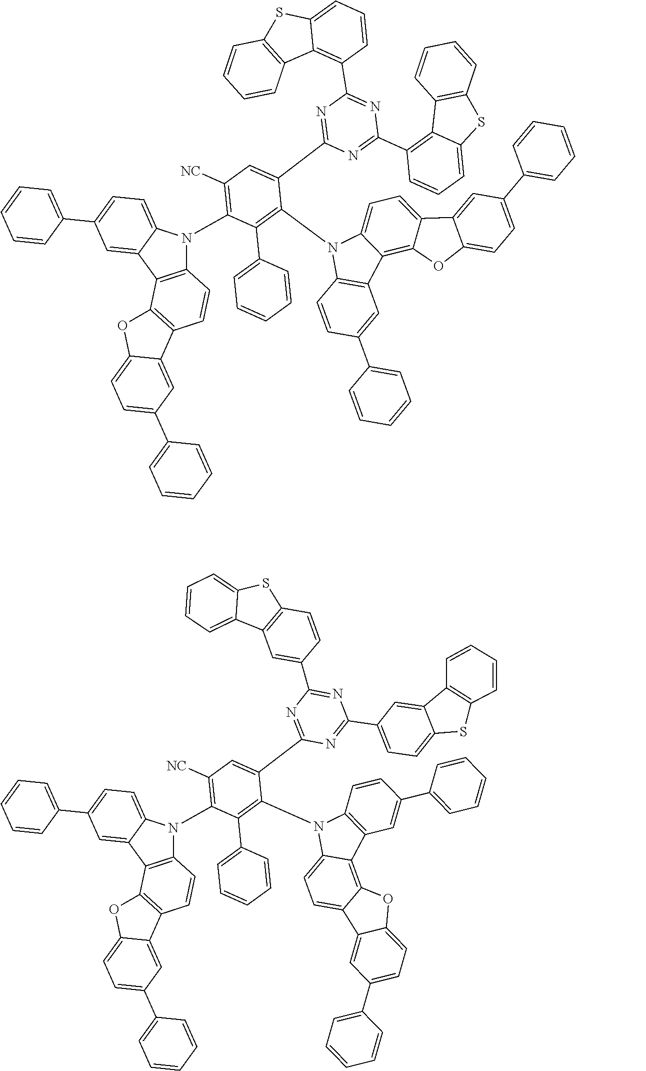 [SMILES: N#Cc1cc(-c2nc(-c3cccc4sc5ccccc5c34)nc(-c3cccc4sc5ccccc5c34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21 ; N#Cc1cc(-c2nc(-c3ccc4sc5ccccc5c4c3)nc(-c3ccc4sc5ccccc5c4c3)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

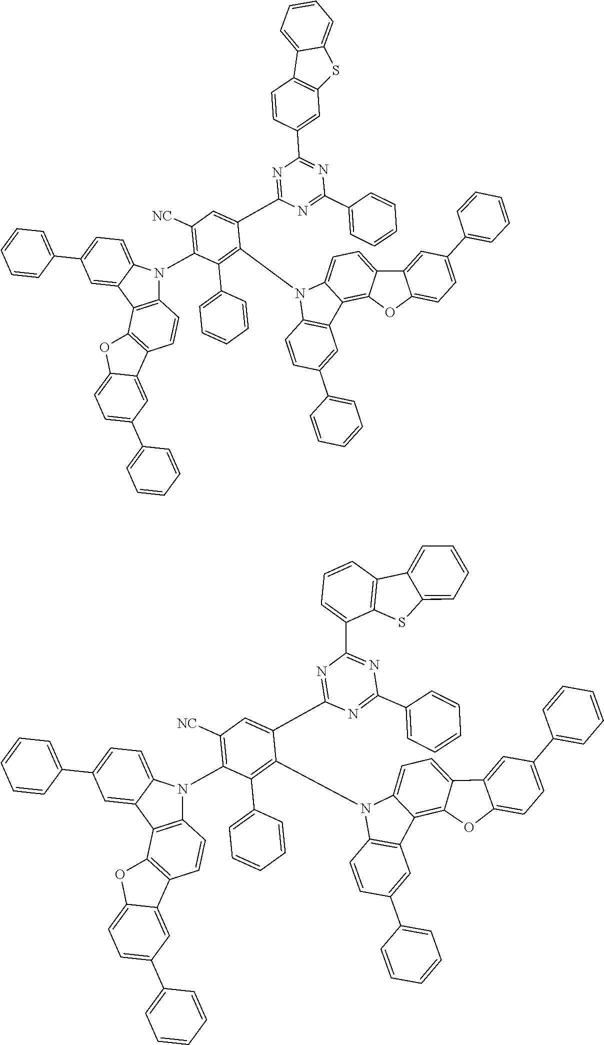 [SMILES: N#Cc1cc(-c2nc(-c3ccccc3)nc(-c3ccc4c(c3)sc3ccccc34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21 ; N#Cc1cc(-c2nc(-c3ccccc3)nc(-c3cccc4c3sc3ccccc34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

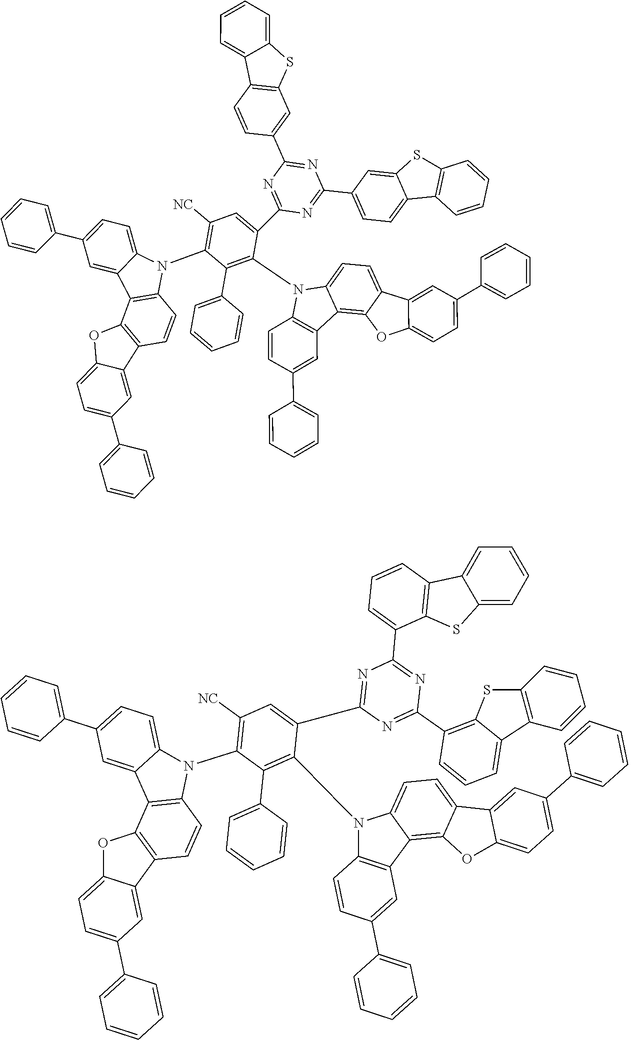 [SMILES: N#Cc1cc(-c2nc(-c3ccc4c(c3)sc3ccccc34)nc(-c3ccc4c(c3)sc3ccccc34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21 ; N#Cc1cc(-c2nc(-c3cccc4c3sc3ccccc34)nc(-c3cccc4c3sc3ccccc34)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

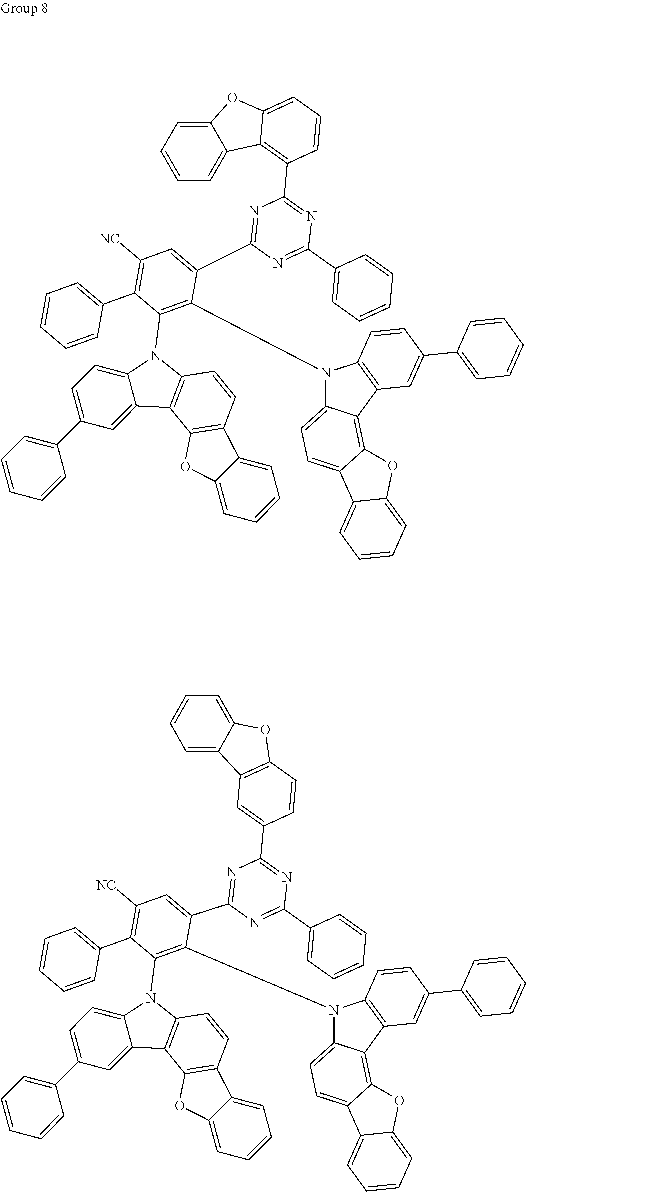 [SMILES: N#Cc1cc(-c2nc(-c3ccccc3)nc(-c3cccc4oc5ccccc5c34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; N#Cc1cc(-c2nc(-c3ccccc3)nc(-c3ccc4oc5ccccc5c4c3)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1]

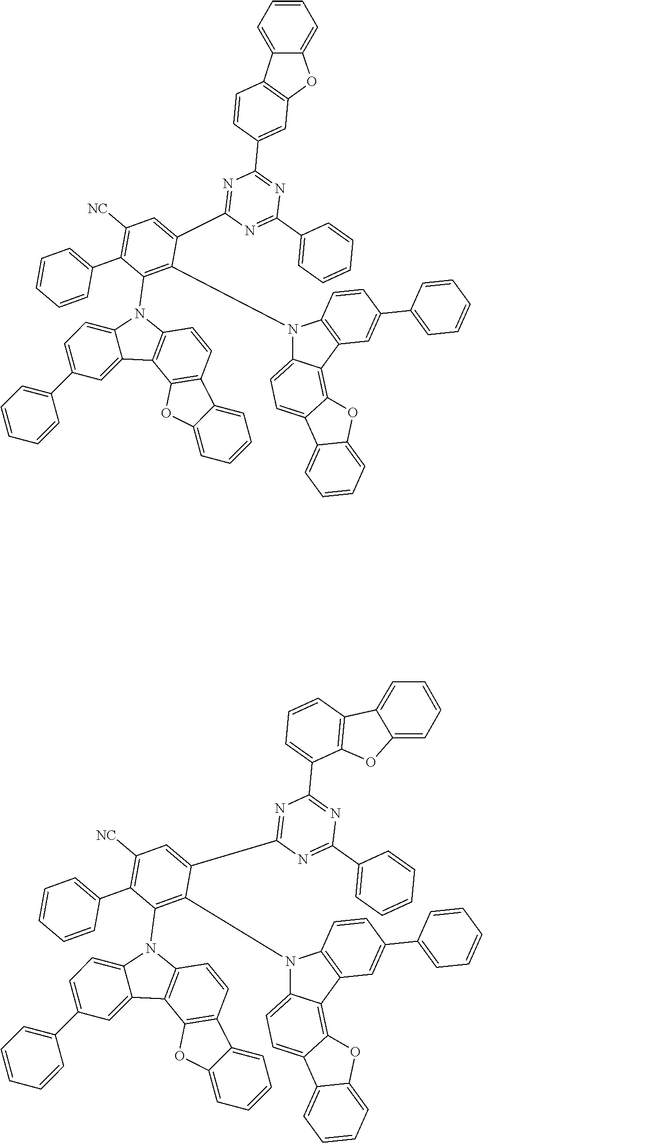 [SMILES: N#Cc1cc(-c2nc(-c3ccccc3)nc(-c3ccc4c(c3)oc3ccccc34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; N#Cc1cc(-c2nc(-c3ccccc3)nc(-c3cccc4c3oc3ccccc34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1]

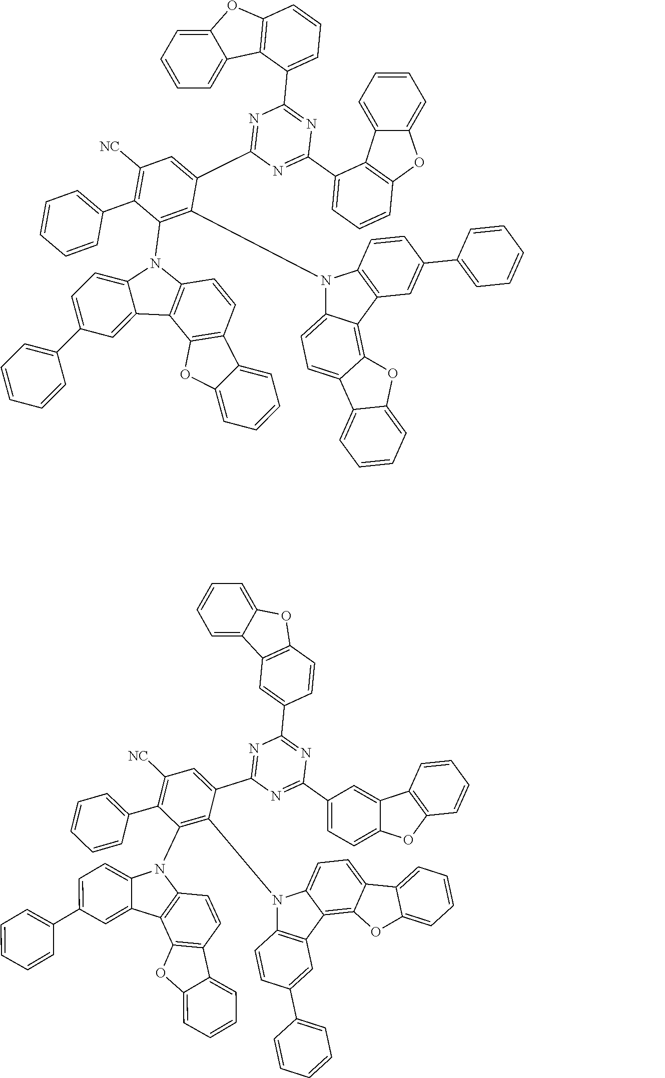 [SMILES: N#Cc1cc(-c2nc(-c3cccc4oc5ccccc5c34)nc(-c3cccc4oc5ccccc5c34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; N#Cc1cc(-c2nc(-c3ccc4oc5ccccc5c4c3)nc(-c3ccc4oc5ccccc5c4c3)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1]

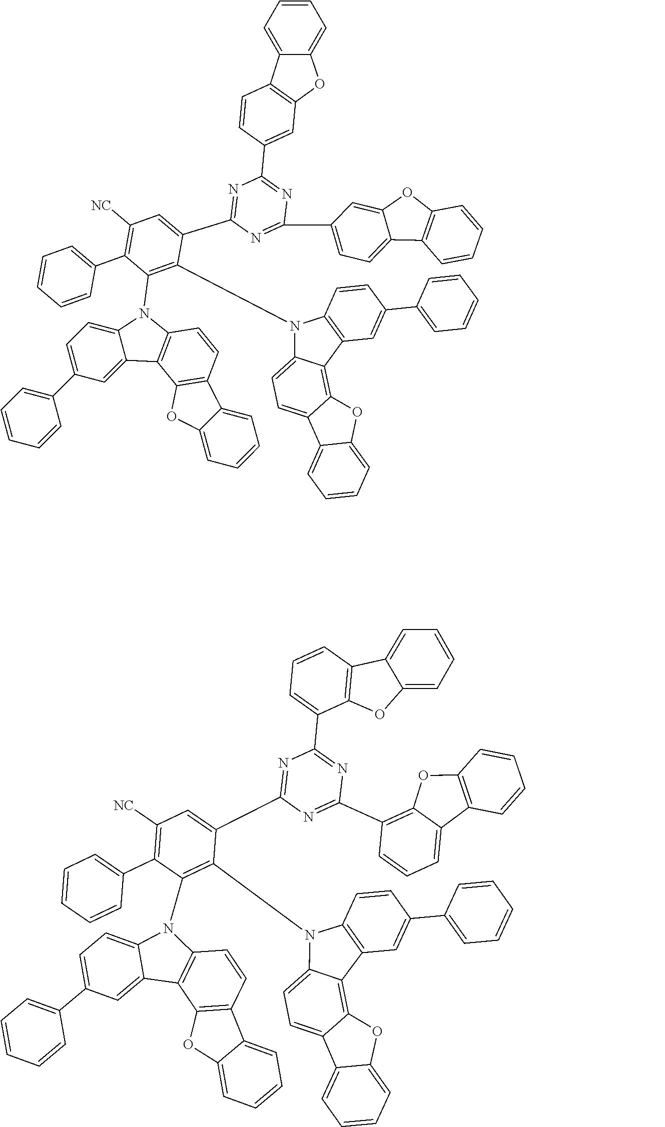 [SMILES: N#Cc1cc(-c2nc(-c3ccc4c(c3)oc3ccccc34)nc(-c3ccc4c(c3)oc3ccccc34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; N#Cc1cc(-c2nc(-c3cccc4c3oc3ccccc34)nc(-c3cccc4c3oc3ccccc34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1]

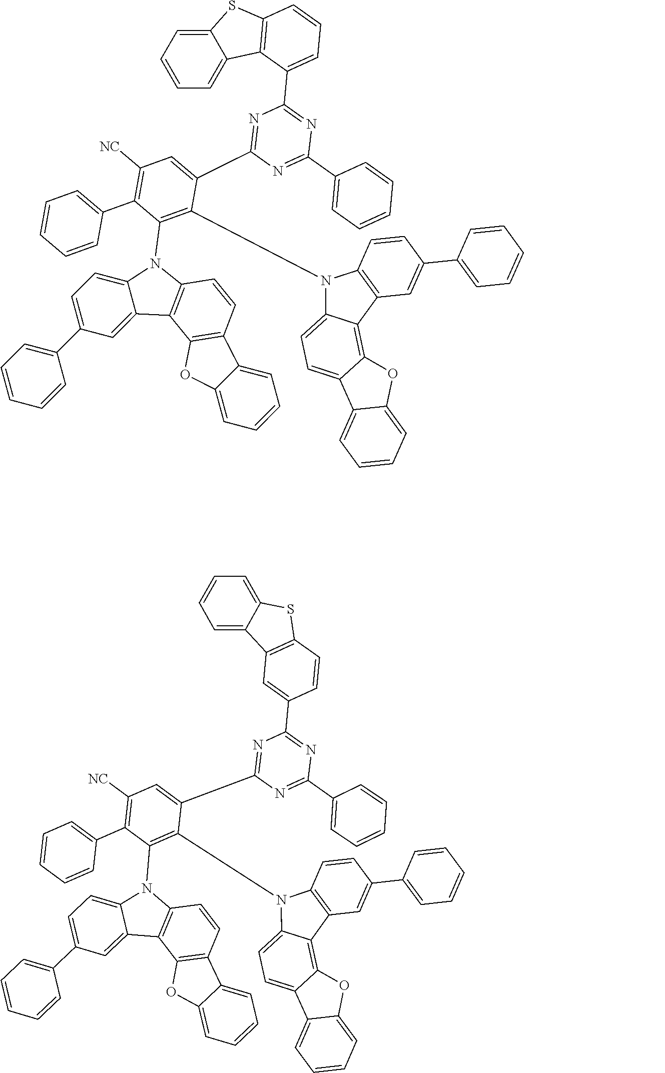 [SMILES: N#Cc1cc(-c2nc(-c3ccccc3)nc(-c3cccc4sc5ccccc5c34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; N#Cc1cc(-c2nc(-c3ccccc3)nc(-c3ccc4sc5ccccc5c4c3)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1]

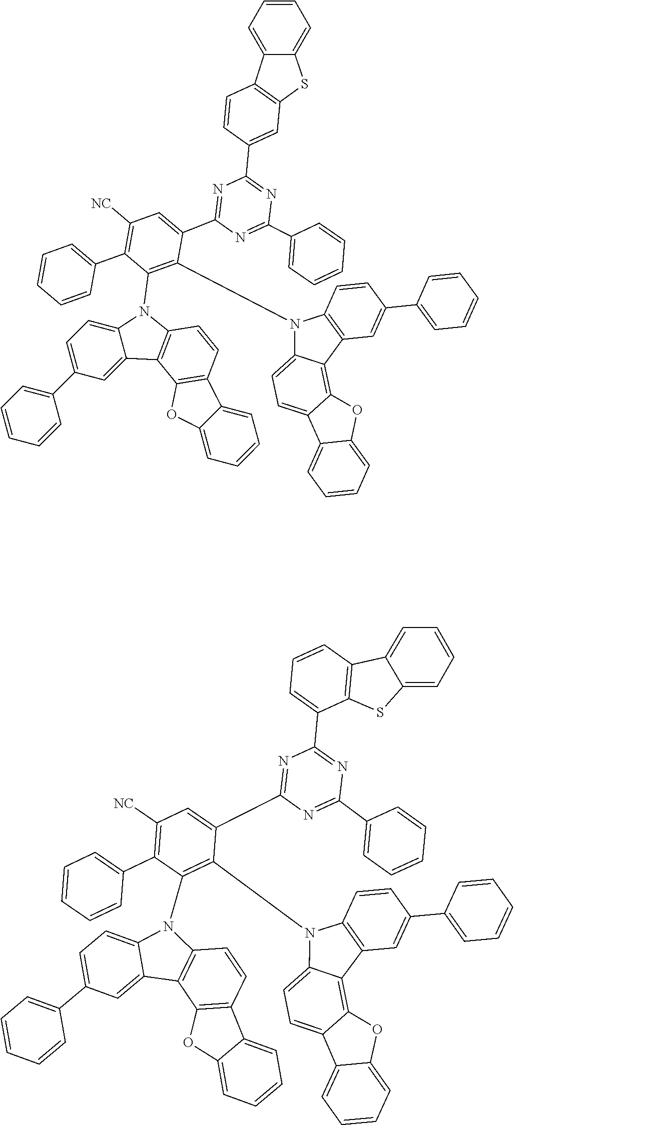 [SMILES: N#Cc1cc(-c2nc(-c3ccccc3)nc(-c3ccc4c(c3)sc3ccccc34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; N#Cc1cc(-c2nc(-c3ccccc3)nc(-c3cccc4c3sc3ccccc34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1]

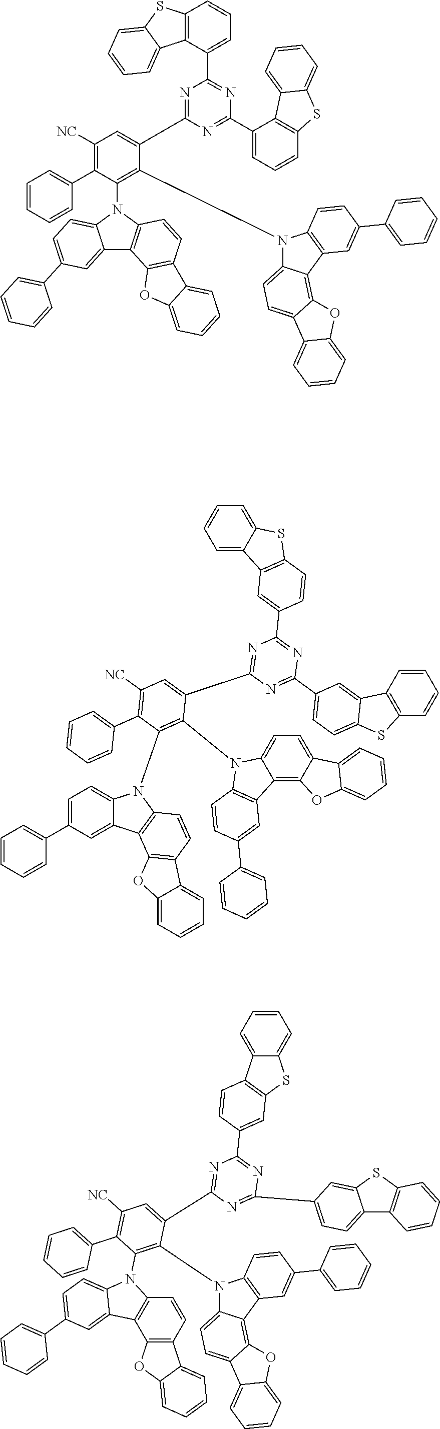 [SMILES: N#Cc1cc(-c2nc(-c3cccc4sc5ccccc5c34)nc(-c3cccc4sc5ccccc5c34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; N#Cc1cc(-c2nc(-c3ccc4sc5ccccc5c4c3)nc(-c3ccc4sc5ccccc5c4c3)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1 ; N#Cc1cc(-c2nc(-c3ccc4c(c3)sc3ccccc34)nc(-c3ccc4c(c3)sc3ccccc34)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-c1ccccc1]

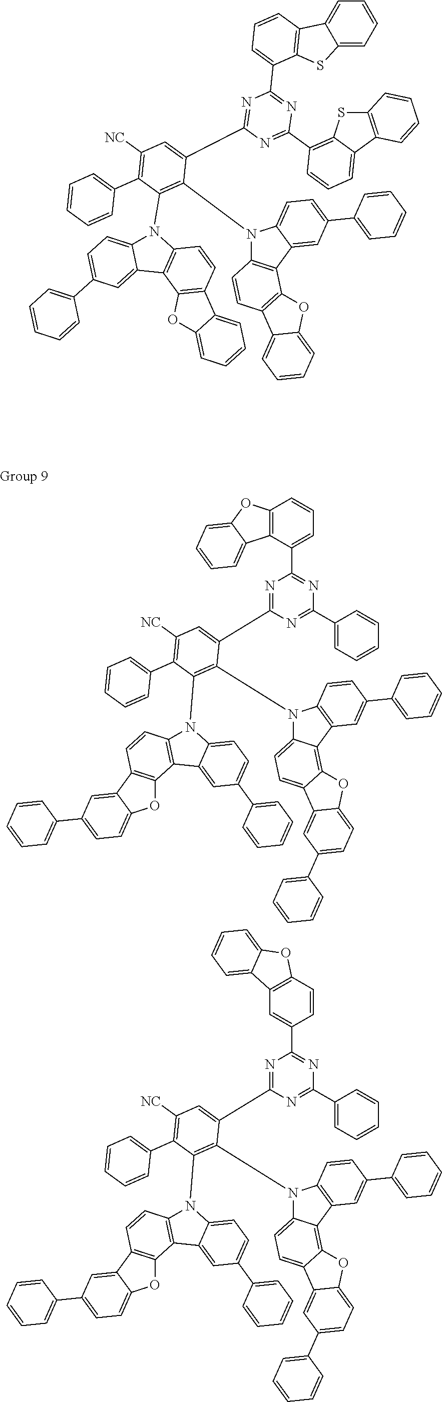

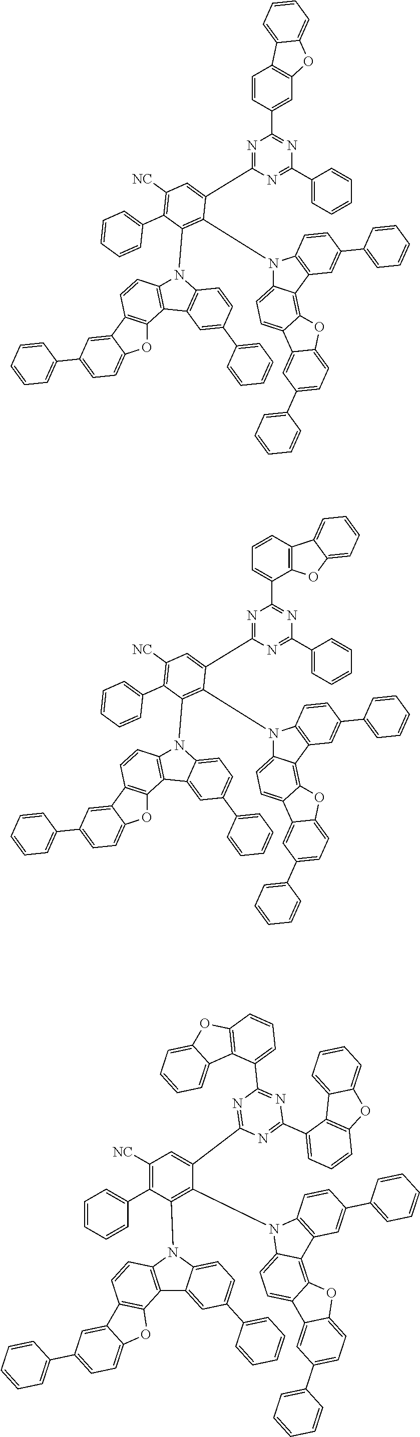

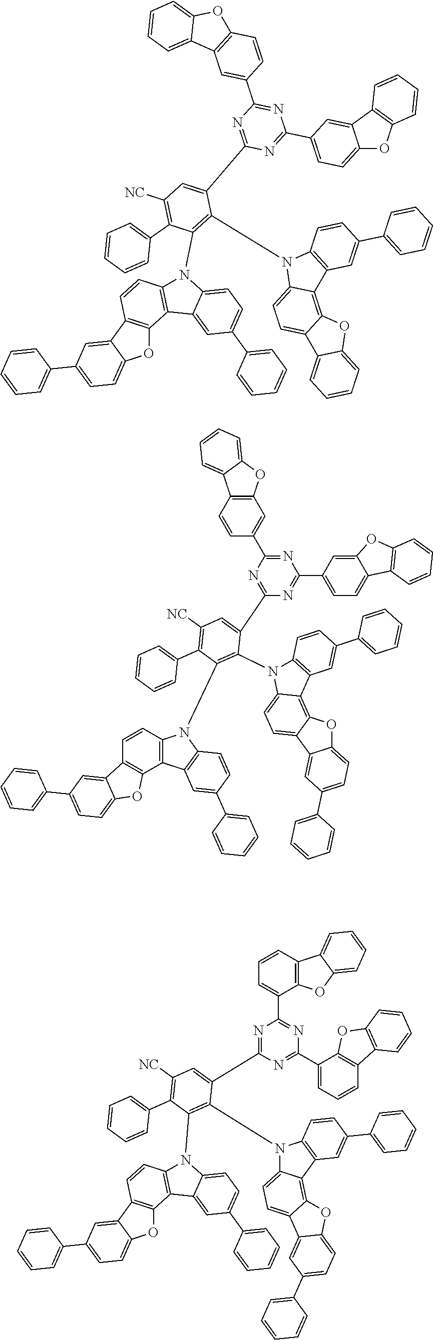

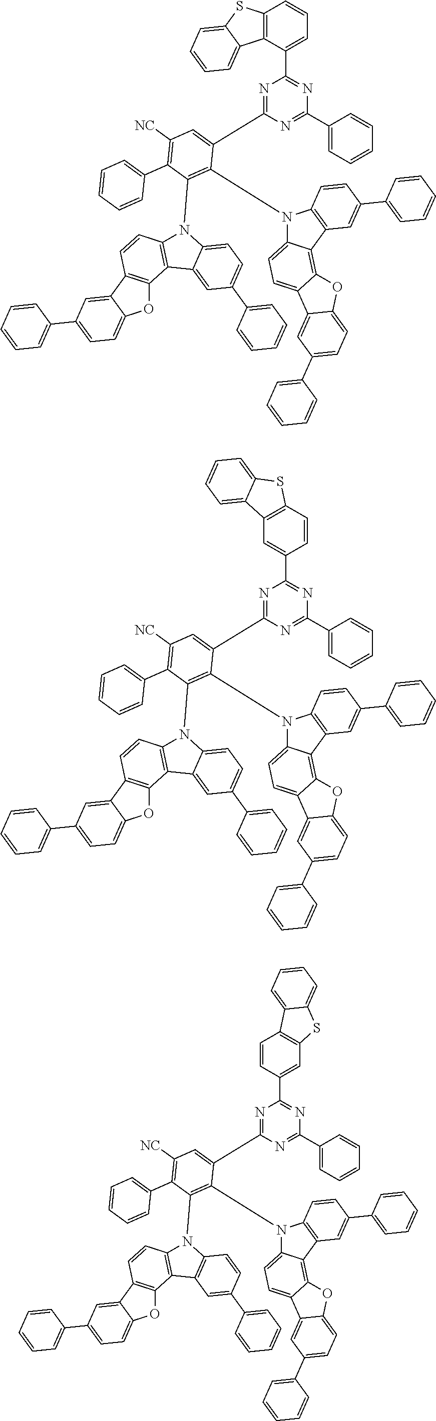

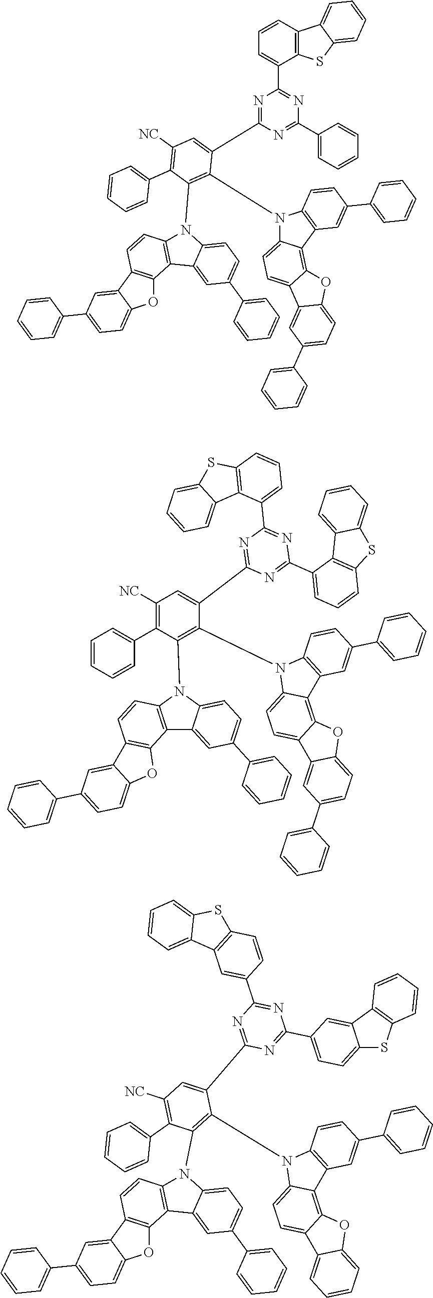

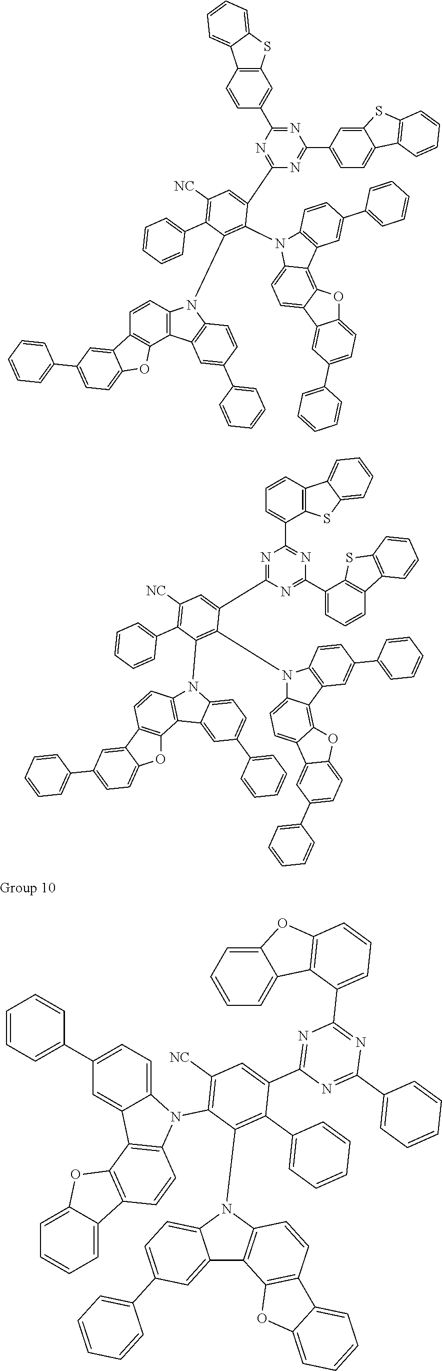

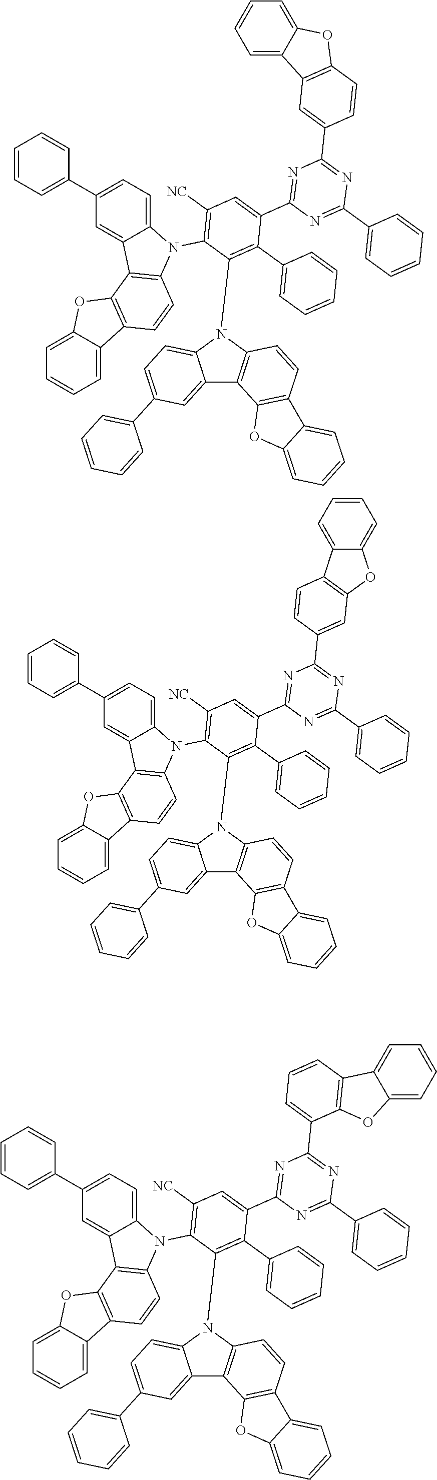

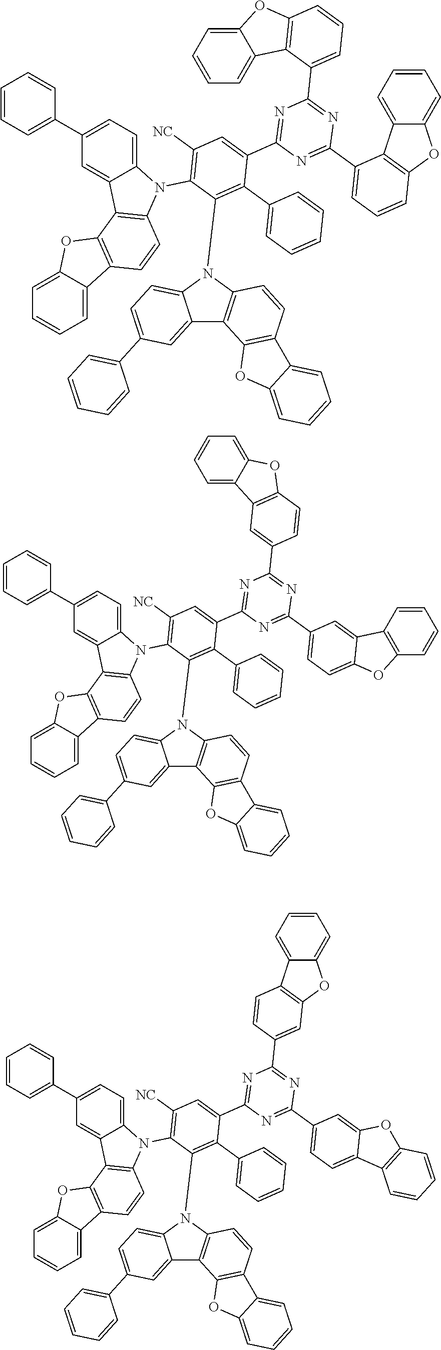 [SMILES: N#Cc1cc(-c2nc(-c3cccc4oc5ccccc5c34)nc(-c3cccc4oc5ccccc5c34)n2)c(-c2ccccc2)c(-n2c3ccc(-c4ccccc4)cc3c3c4oc5ccccc5c4ccc32)c1-n1c2ccc(-c3ccccc3)cc2c2c3oc4ccccc4c3ccc21 ; N#Cc1cc(-c2nc(-c3ccc4oc5ccccc5c4c3)nc(-c3ccc4oc5ccccc5c4c3)n2)c(-c2ccccc2)c(-n2c3ccc(-c4ccccc4)cc3c3c4oc5ccccc5c4ccc32)c1-n1c2ccc(-c3ccccc3)cc2c2c3oc4ccccc4c3ccc21 ; N#Cc1cc(-c2nc(-c3ccc4c(c3)oc3ccccc34)nc(-c3ccc4c(c3)oc3ccccc34)n2)c(-c2ccccc2)c(-n2c3ccc(-c4ccccc4)cc3c3c4oc5ccccc5c4ccc32)c1-n1c2ccc(-c3ccccc3)cc2c2c3oc4ccccc4c3ccc21]

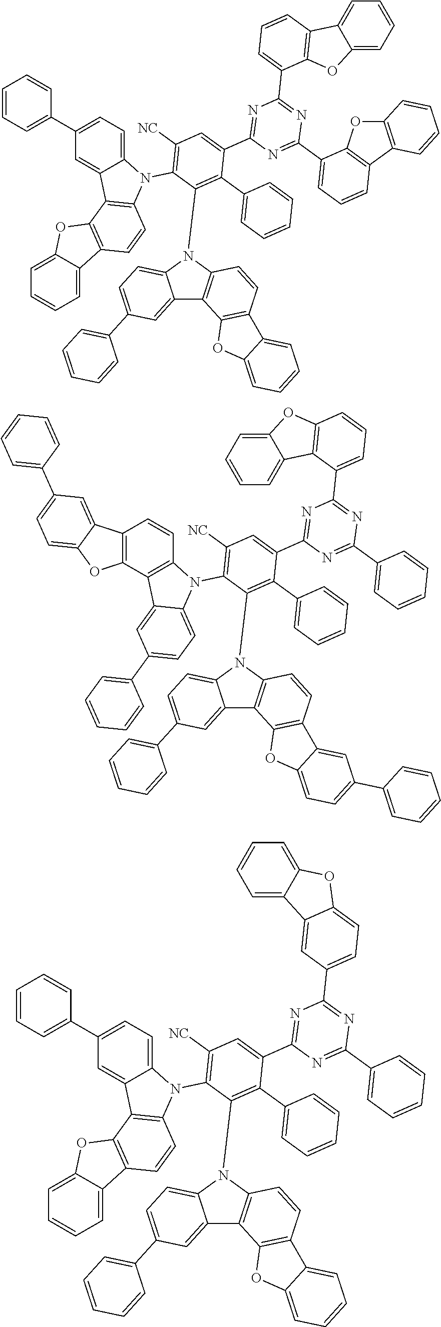

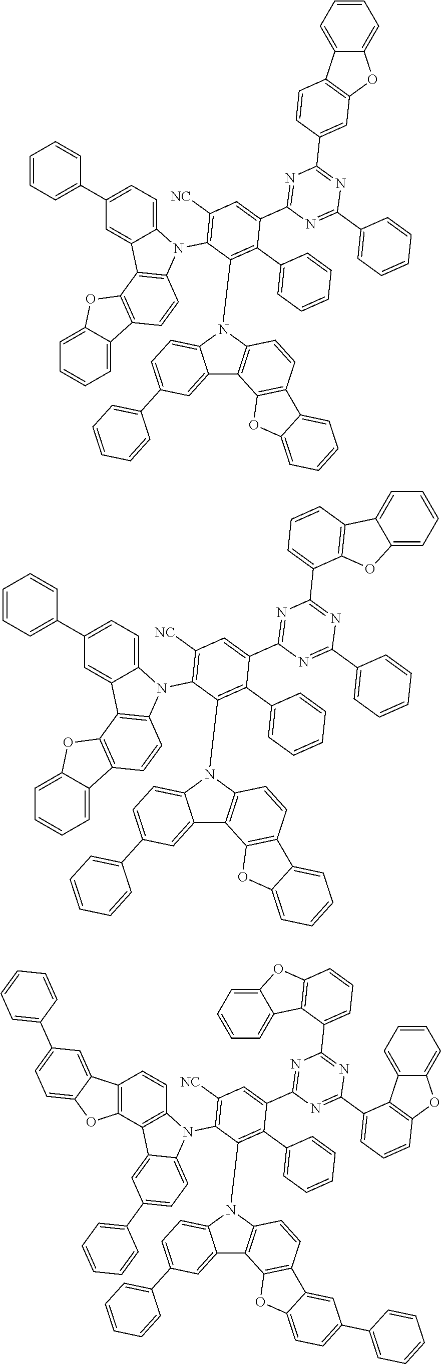

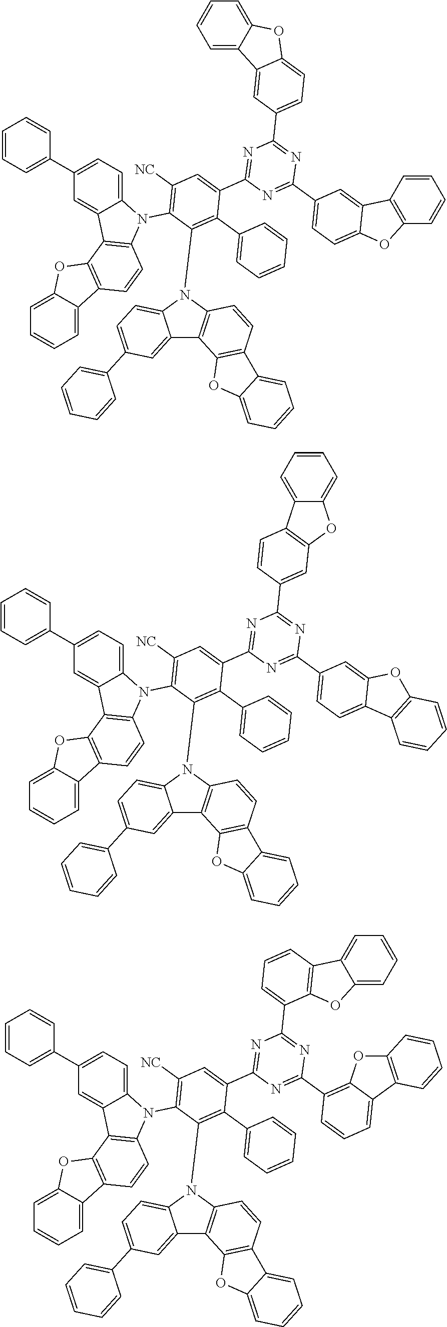

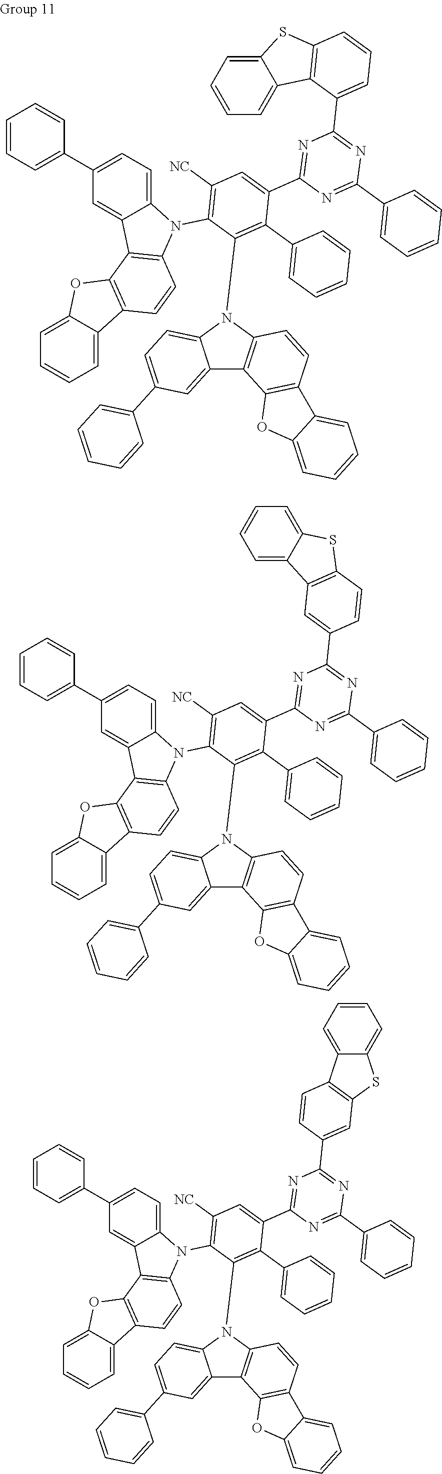

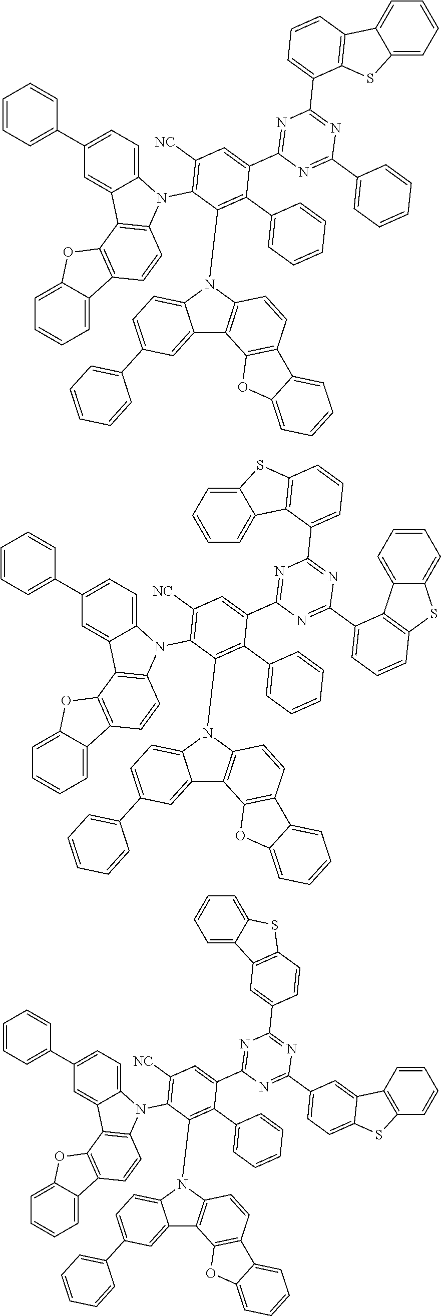

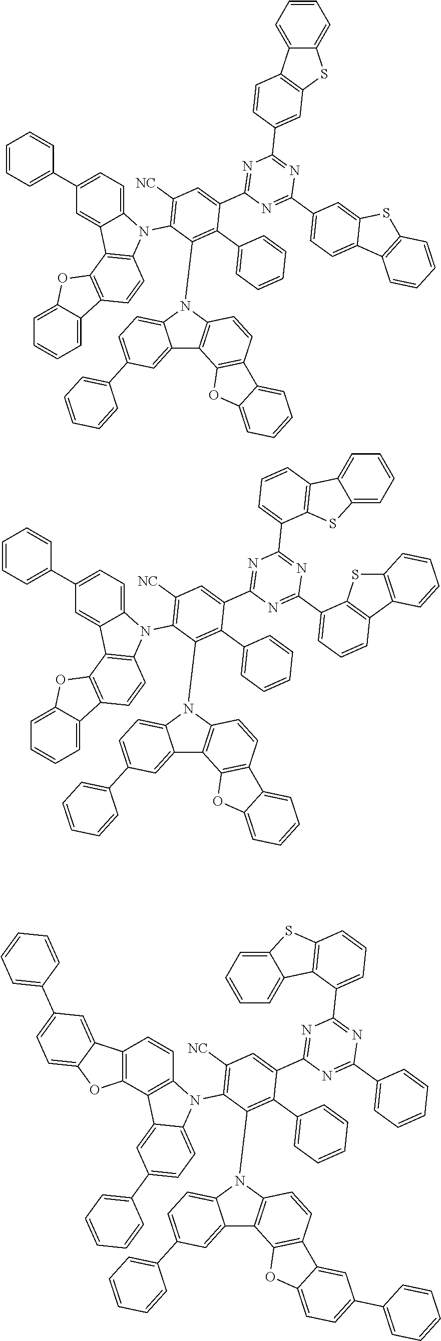

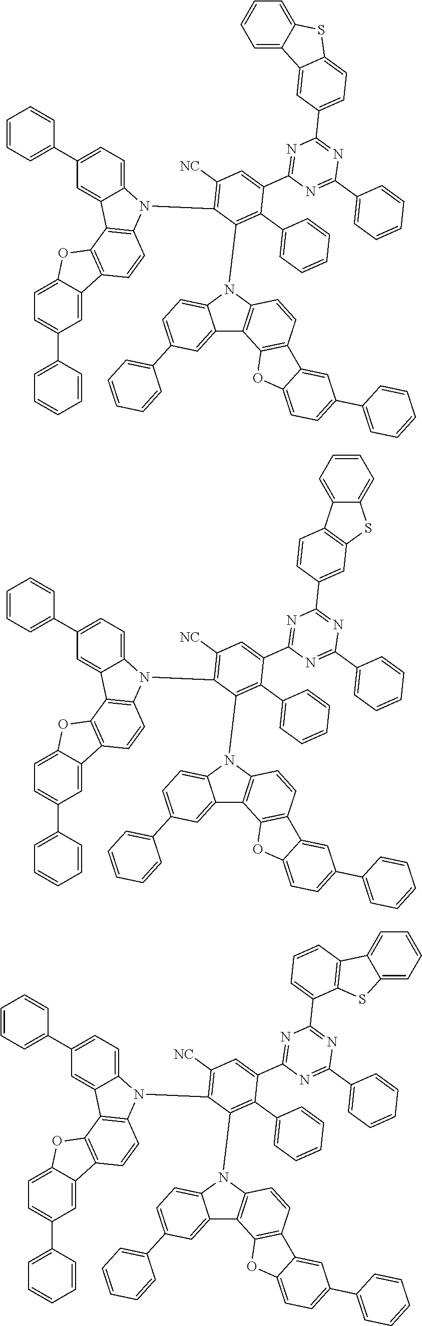

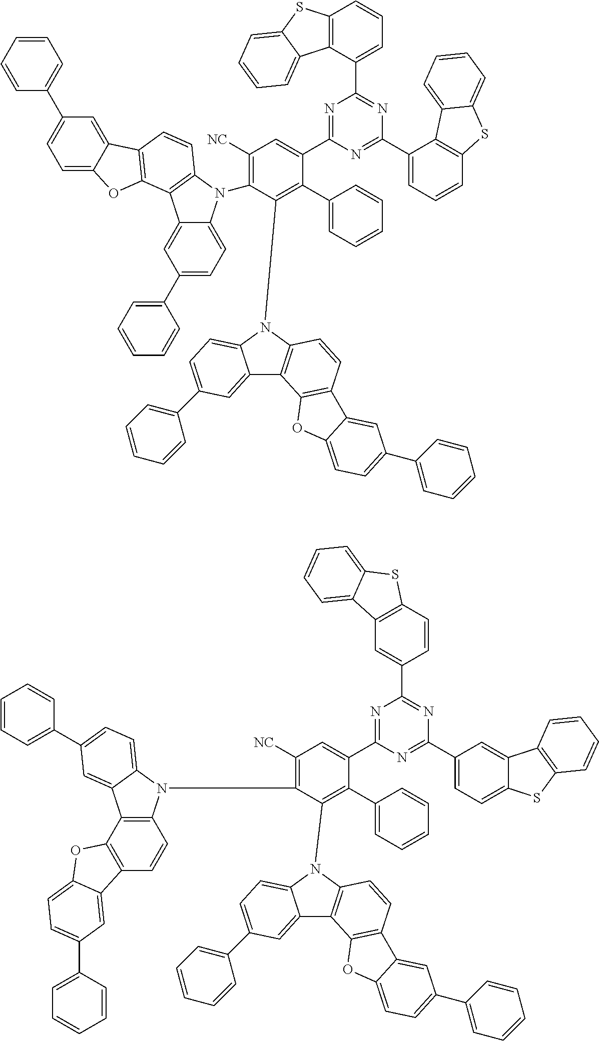 [SMILES: N#Cc1cc(-c2nc(-c3cccc4sc5ccccc5c34)nc(-c3cccc4sc5ccccc5c34)n2)c(-c2ccccc2)c(-n2c3ccc(-c4ccccc4)cc3c3c4oc5ccc(-c6ccccc6)cc5c4ccc32)c1-n1c2ccc(-c3ccccc3)cc2c2c3oc4ccc(-c5ccccc5)cc4c3ccc21 ; N#Cc1cc(-c2nc(-c3ccc4sc5ccccc5c4c3)nc(-c3ccc4sc5ccccc5c4c3)n2)c(-c2ccccc2)c(-n2c3ccc(-c4ccccc4)cc3c3c4oc5ccc(-c6ccccc6)cc5c4ccc32)c1-n1c2ccc(-c3ccccc3)cc2c2c3oc4ccc(-c5ccccc5)cc4c3ccc21]

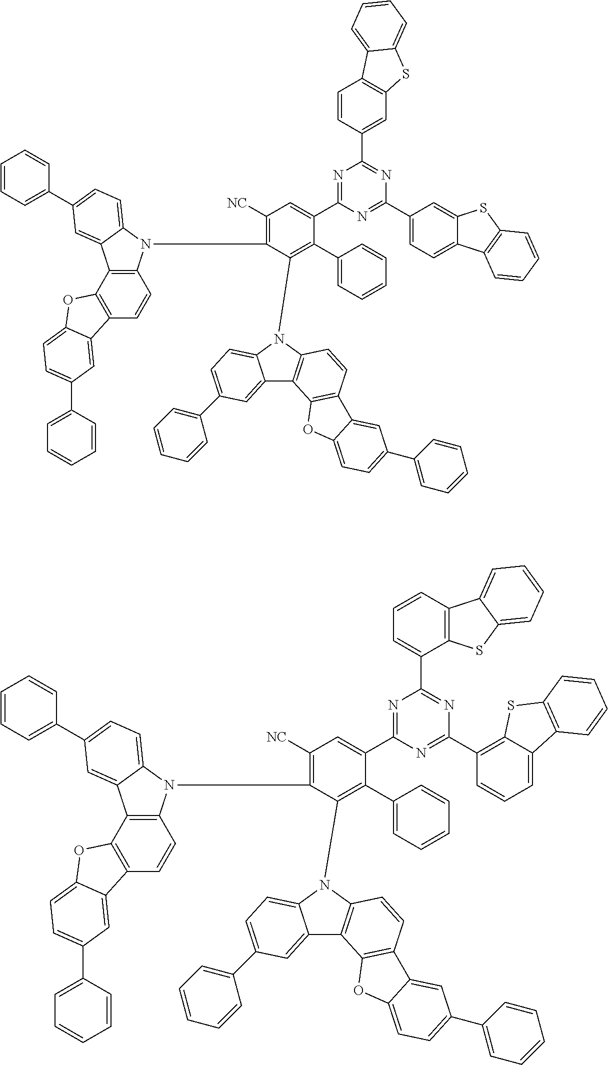 [SMILES: N#Cc1cc(-c2nc(-c3ccc4c(c3)sc3ccccc34)nc(-c3ccc4c(c3)sc3ccccc34)n2)c(-c2ccccc2)c(-n2c3ccc(-c4ccccc4)cc3c3c4oc5ccc(-c6ccccc6)cc5c4ccc32)c1-n1c2ccc(-c3ccccc3)cc2c2c3oc4ccc(-c5ccccc5)cc4c3ccc21 ; N#Cc1cc(-c2nc(-c3cccc4c3sc3ccccc34)nc(-c3cccc4c3sc3ccccc34)n2)c(-c2ccccc2)c(-n2c3ccc(-c4ccccc4)cc3c3c4oc5ccc(-c6ccccc6)cc5c4ccc32)c1-n1c2ccc(-c3ccccc3)cc2c2c3oc4ccc(-c5ccccc5)cc4c3ccc21]

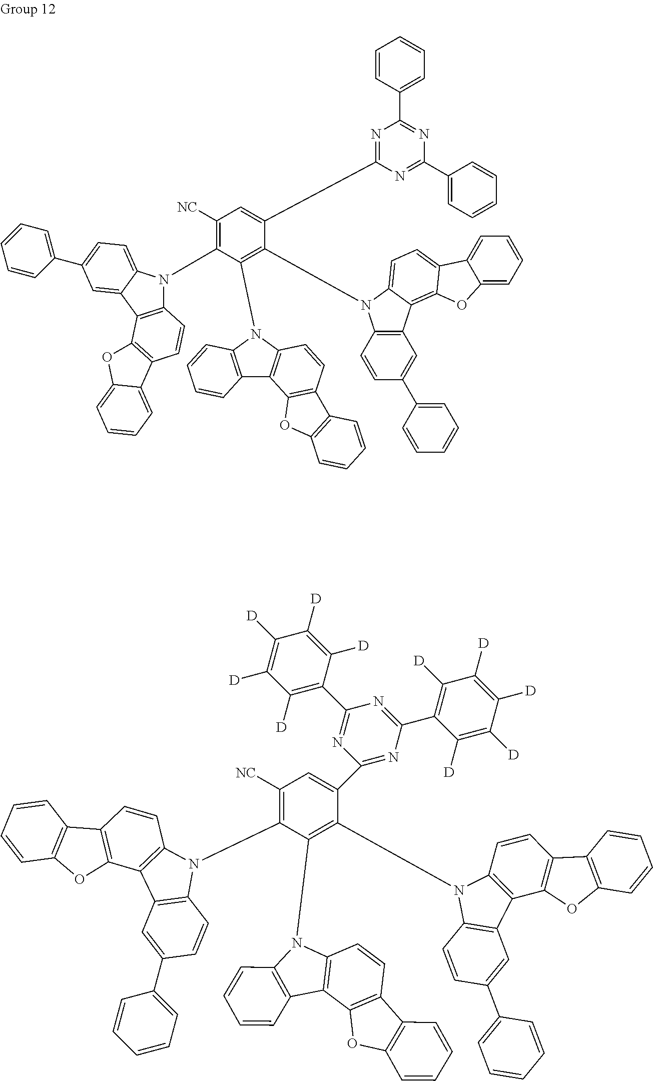 [SMILES: N#Cc1cc(-c2nc(-c3ccccc3)nc(-c3ccccc3)n2)c(-n2c3ccc(-c4ccccc4)cc3c3c4oc5ccccc5c4ccc32)c(-n2c3ccccc3c3c4oc5ccccc5c4ccc32)c1-n1c2ccc(-c3ccccc3)cc2c2c3oc4ccccc4c3ccc21 ; [2H]c1c([2H])c([2H])c(-c2nc(-c3cc(C#N)c(-n4c5ccc(-c6ccccc6)cc5c5c6oc7ccccc7c6ccc54)c(-n4c5ccccc5c5c6oc7ccccc7c6ccc54)c3-n3c4ccc(-c5ccccc5)cc4c4c5oc6ccccc6c5ccc43)nc(-c3c([2H])c([2H])c([2H])c([2H])c3[2H])n2)c([2H])c1[2H]]

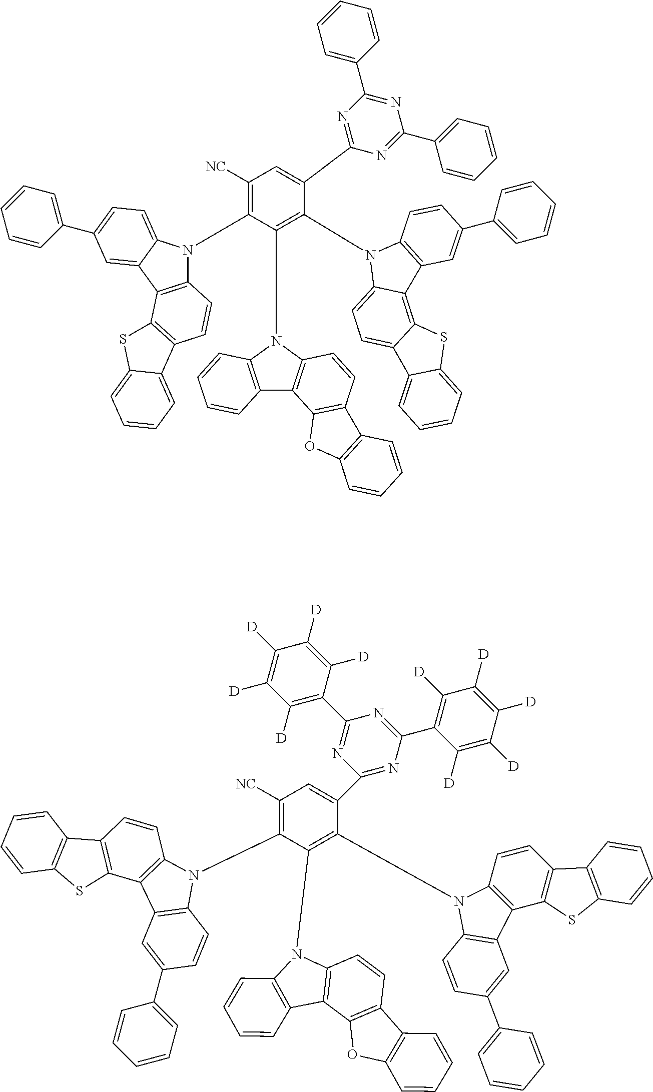 [SMILES: N#Cc1cc(-c2nc(-c3ccccc3)nc(-c3ccccc3)n2)c(-n2c3ccc(-c4ccccc4)cc3c3c4sc5ccccc5c4ccc32)c(-n2c3ccccc3c3c4oc5ccccc5c4ccc32)c1-n1c2ccc(-c3ccccc3)cc2c2c3sc4ccccc4c3ccc21 ; [2H]c1c([2H])c([2H])c(-c2nc(-c3cc(C#N)c(-n4c5ccc(-c6ccccc6)cc5c5c6sc7ccccc7c6ccc54)c(-n4c5ccccc5c5c6oc7ccccc7c6ccc54)c3-n3c4ccc(-c5ccccc5)cc4c4c5sc6ccccc6c5ccc43)nc(-c3c([2H])c([2H])c([2H])c([2H])c3[2H])n2)c([2H])c1[2H]]

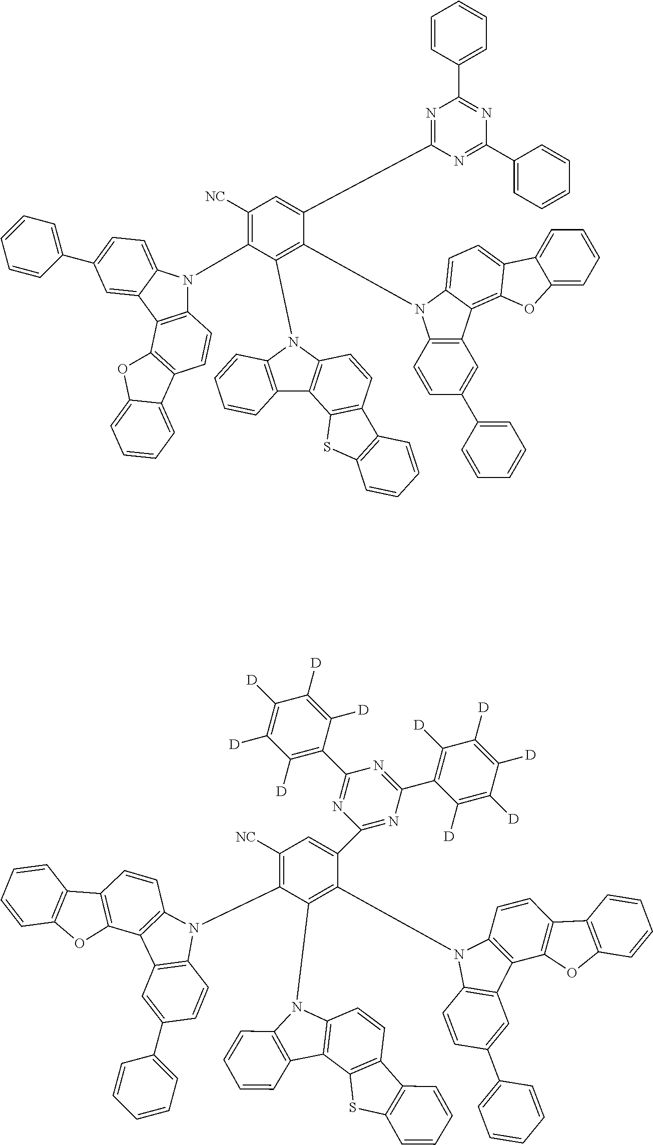 [SMILES: N#Cc1cc(-c2nc(-c3ccccc3)nc(-c3ccccc3)n2)c(-n2c3ccc(-c4ccccc4)cc3c3c4oc5ccccc5c4ccc32)c(-n2c3ccccc3c3c4sc5ccccc5c4ccc32)c1-n1c2ccc(-c3ccccc3)cc2c2c3oc4ccccc4c3ccc21 ; [2H]c1c([2H])c([2H])c(-c2nc(-c3cc(C#N)c(-n4c5ccc(-c6ccccc6)cc5c5c6oc7ccccc7c6ccc54)c(-n4c5ccccc5c5c6sc7ccccc7c6ccc54)c3-n3c4ccc(-c5ccccc5)cc4c4c5oc6ccccc6c5ccc43)nc(-c3c([2H])c([2H])c([2H])c([2H])c3[2H])n2)c([2H])c1[2H]]

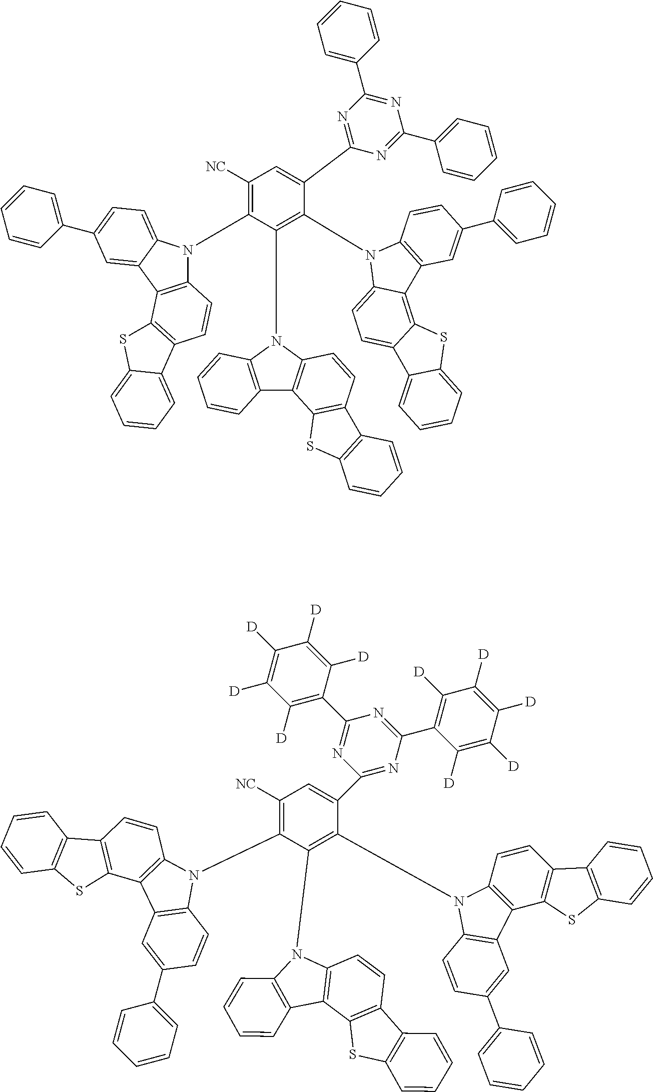 [SMILES: N#Cc1cc(-c2nc(-c3ccccc3)nc(-c3ccccc3)n2)c(-n2c3ccc(-c4ccccc4)cc3c3c4sc5ccccc5c4ccc32)c(-n2c3ccccc3c3c4sc5ccccc5c4ccc32)c1-n1c2ccc(-c3ccccc3)cc2c2c3sc4ccccc4c3ccc21 ; [2H]c1c([2H])c([2H])c(-c2nc(-c3cc(C#N)c(-n4c5ccc(-c6ccccc6)cc5c5c6sc7ccccc7c6ccc54)c(-n4c5ccccc5c5c6sc7ccccc7c6ccc54)c3-n3c4ccc(-c5ccccc5)cc4c4c5sc6ccccc6c5ccc43)nc(-c3c([2H])c([2H])c([2H])c([2H])c3[2H])n2)c([2H])c1[2H]]

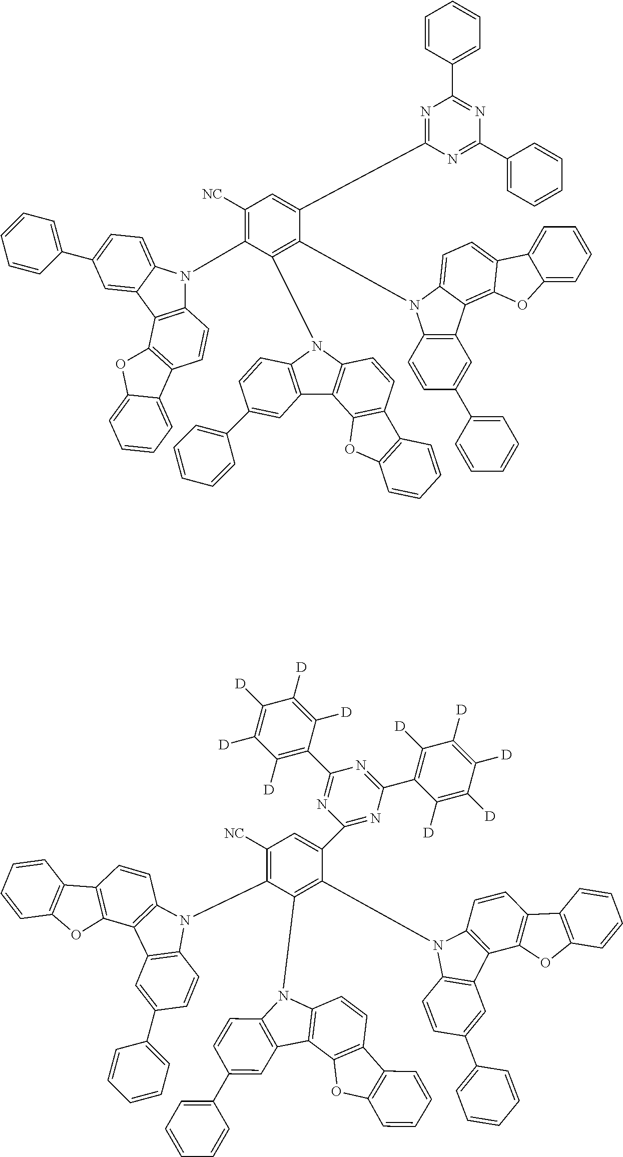 [SMILES: N#Cc1cc(-c2nc(-c3ccccc3)nc(-c3ccccc3)n2)c(-n2c3ccc(-c4ccccc4)cc3c3c4oc5ccccc5c4ccc32)c(-n2c3ccc(-c4ccccc4)cc3c3c4oc5ccccc5c4ccc32)c1-n1c2ccc(-c3ccccc3)cc2c2c3oc4ccccc4c3ccc21 ; [2H]c1c([2H])c([2H])c(-c2nc(-c3cc(C#N)c(-n4c5ccc(-c6ccccc6)cc5c5c6oc7ccccc7c6ccc54)c(-n4c5ccc(-c6ccccc6)cc5c5c6oc7ccccc7c6ccc54)c3-n3c4ccc(-c5ccccc5)cc4c4c5oc6ccccc6c5ccc43)nc(-c3c([2H])c([2H])c([2H])c([2H])c3[2H])n2)c([2H])c1[2H]]

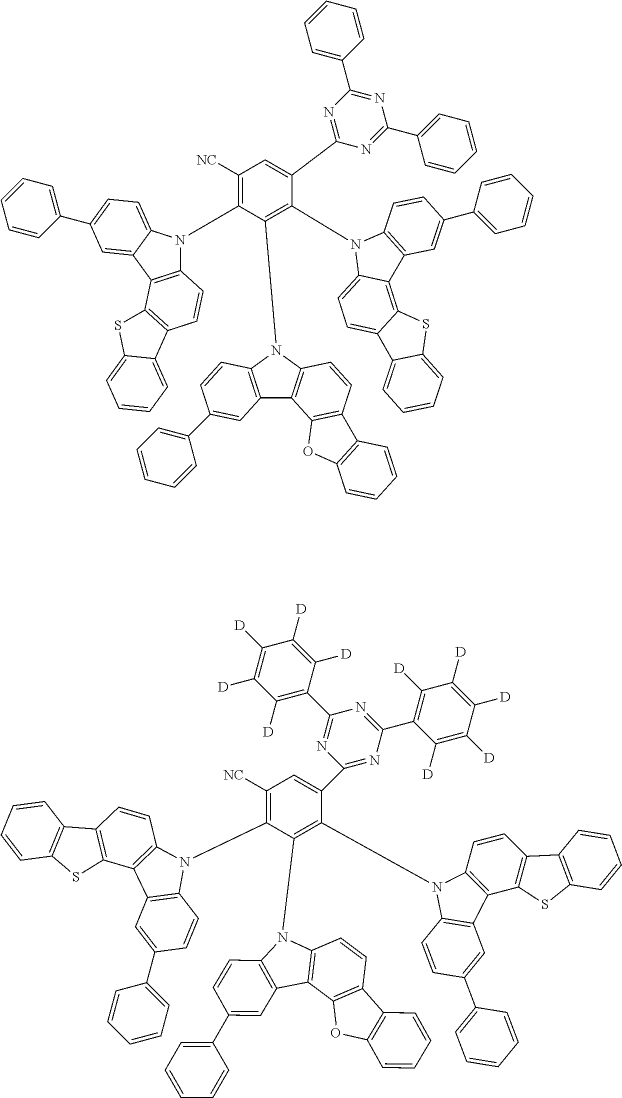 [SMILES: N#Cc1cc(-c2nc(-c3ccccc3)nc(-c3ccccc3)n2)c(-n2c3ccc(-c4ccccc4)cc3c3c4sc5ccccc5c4ccc32)c(-n2c3ccc(-c4ccccc4)cc3c3c4oc5ccccc5c4ccc32)c1-n1c2ccc(-c3ccccc3)cc2c2c3sc4ccccc4c3ccc21 ; [2H]c1c([2H])c([2H])c(-c2nc(-c3cc(C#N)c(-n4c5ccc(-c6ccccc6)cc5c5c6sc7ccccc7c6ccc54)c(-n4c5ccc(-c6ccccc6)cc5c5c6oc7ccccc7c6ccc54)c3-n3c4ccc(-c5ccccc5)cc4c4c5sc6ccccc6c5ccc43)nc(-c3c([2H])c([2H])c([2H])c([2H])c3[2H])n2)c([2H])c1[2H]]

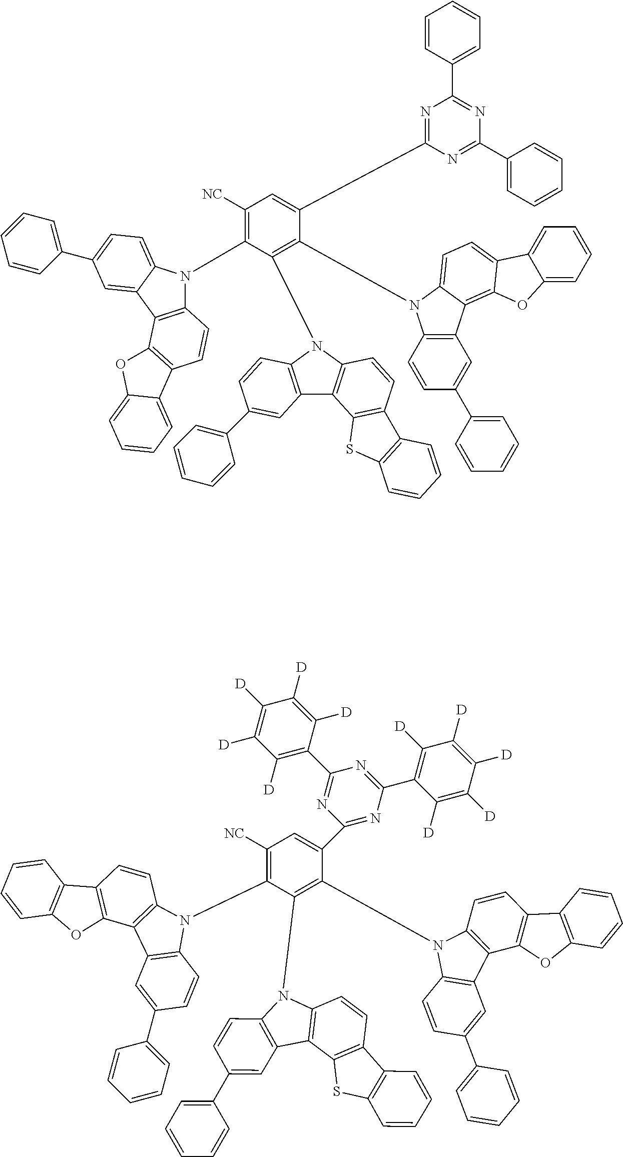 [SMILES: N#Cc1cc(-c2nc(-c3ccccc3)nc(-c3ccccc3)n2)c(-n2c3ccc(-c4ccccc4)cc3c3c4oc5ccccc5c4ccc32)c(-n2c3ccc(-c4ccccc4)cc3c3c4sc5ccccc5c4ccc32)c1-n1c2ccc(-c3ccccc3)cc2c2c3oc4ccccc4c3ccc21 ; [2H]c1c([2H])c([2H])c(-c2nc(-c3cc(C#N)c(-n4c5ccc(-c6ccccc6)cc5c5c6oc7ccccc7c6ccc54)c(-n4c5ccc(-c6ccccc6)cc5c5c6sc7ccccc7c6ccc54)c3-n3c4ccc(-c5ccccc5)cc4c4c5oc6ccccc6c5ccc43)nc(-c3c([2H])c([2H])c([2H])c([2H])c3[2H])n2)c([2H])c1[2H]]

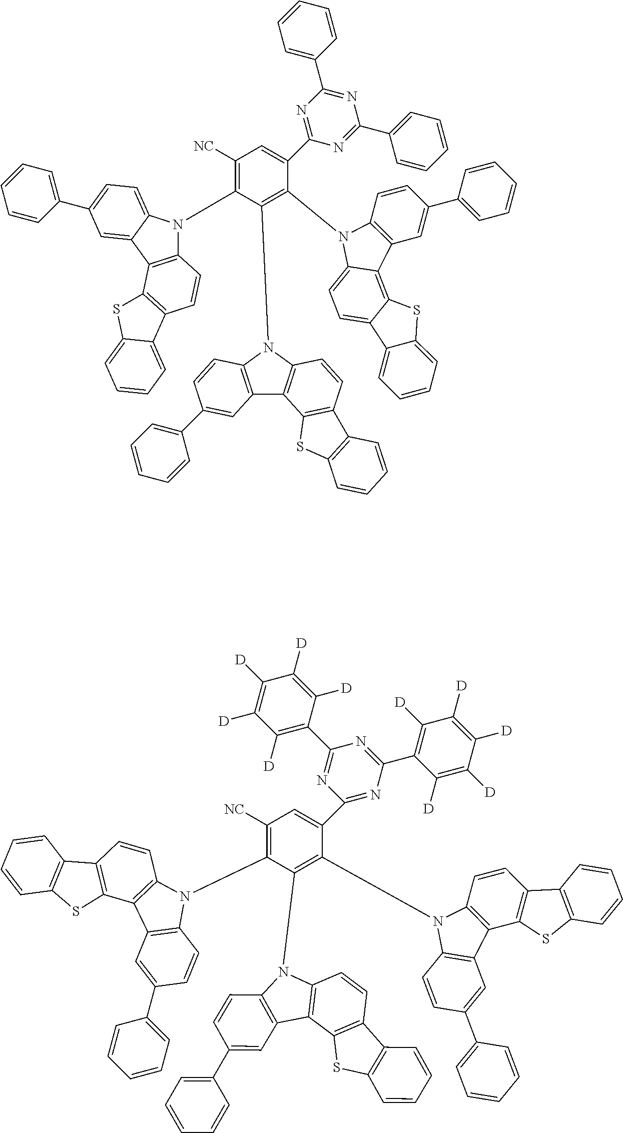 [SMILES: N#Cc1cc(-c2nc(-c3ccccc3)nc(-c3ccccc3)n2)c(-n2c3ccc(-c4ccccc4)cc3c3c4sc5ccccc5c4ccc32)c(-n2c3ccc(-c4ccccc4)cc3c3c4sc5ccccc5c4ccc32)c1-n1c2ccc(-c3ccccc3)cc2c2c3sc4ccccc4c3ccc21 ; [2H]c1c([2H])c([2H])c(-c2nc(-c3cc(C#N)c(-n4c5ccc(-c6ccccc6)cc5c5c6sc7ccccc7c6ccc54)c(-n4c5ccc(-c6ccccc6)cc5c5c6sc7ccccc7c6ccc54)c3-n3c4ccc(-c5ccccc5)cc4c4c5sc6ccccc6c5ccc43)nc(-c3c([2H])c([2H])c([2H])c([2H])c3[2H])n2)c([2H])c1[2H]]

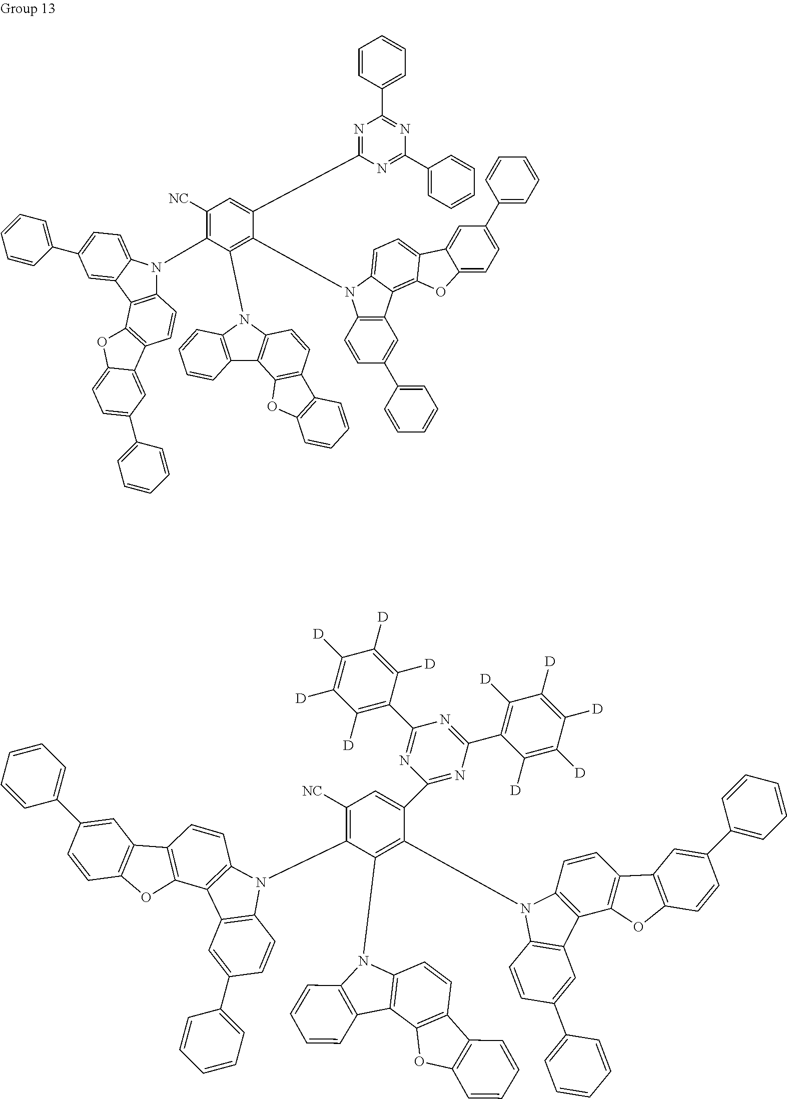 [SMILES: N#Cc1cc(-c2nc(-c3ccccc3)nc(-c3ccccc3)n2)c(-n2c3ccc(-c4ccccc4)cc3c3c4oc5ccc(-c6ccccc6)cc5c4ccc32)c(-n2c3ccccc3c3c4oc5ccccc5c4ccc32)c1-n1c2ccc(-c3ccccc3)cc2c2c3oc4ccc(-c5ccccc5)cc4c3ccc21 ; [2H]c1c([2H])c([2H])c(-c2nc(-c3cc(C#N)c(-n4c5ccc(-c6ccccc6)cc5c5c6oc7ccc(-c8ccccc8)cc7c6ccc54)c(-n4c5ccccc5c5c6oc7ccccc7c6ccc54)c3-n3c4ccc(-c5ccccc5)cc4c4c5oc6ccc(-c7ccccc7)cc6c5ccc43)nc(-c3c([2H])c([2H])c([2H])c([2H])c3[2H])n2)c([2H])c1[2H]]

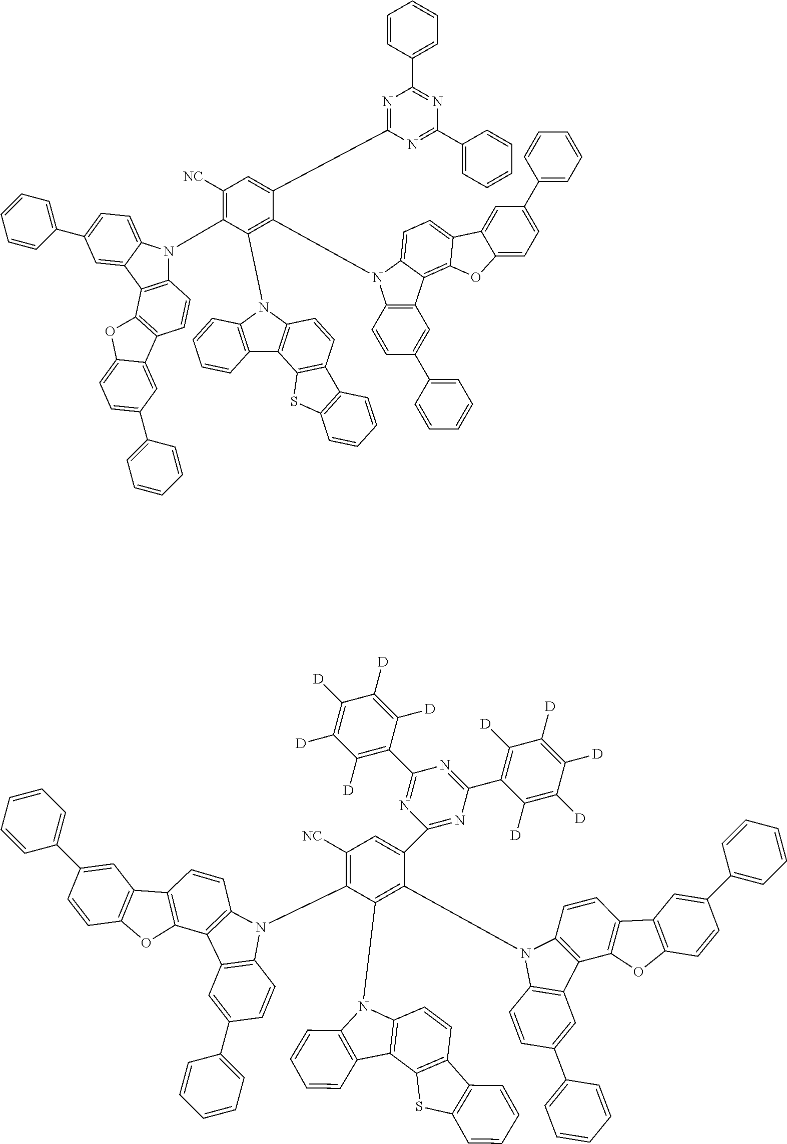 [SMILES: N#Cc1cc(-c2nc(-c3ccccc3)nc(-c3ccccc3)n2)c(-n2c3ccc(-c4ccccc4)cc3c3c4oc5ccc(-c6ccccc6)cc5c4ccc32)c(-n2c3ccccc3c3c4sc5ccccc5c4ccc32)c1-n1c2ccc(-c3ccccc3)cc2c2c3oc4ccc(-c5ccccc5)cc4c3ccc21 ; [2H]c1c([2H])c([2H])c(-c2nc(-c3cc(C#N)c(-n4c5ccc(-c6ccccc6)cc5c5c6oc7ccc(-c8ccccc8)cc7c6ccc54)c(-n4c5ccccc5c5c6sc7ccccc7c6ccc54)c3-n3c4ccc(-c5ccccc5)cc4c4c5oc6ccc(-c7ccccc7)cc6c5ccc43)nc(-c3c([2H])c([2H])c([2H])c([2H])c3[2H])n2)c([2H])c1[2H]]

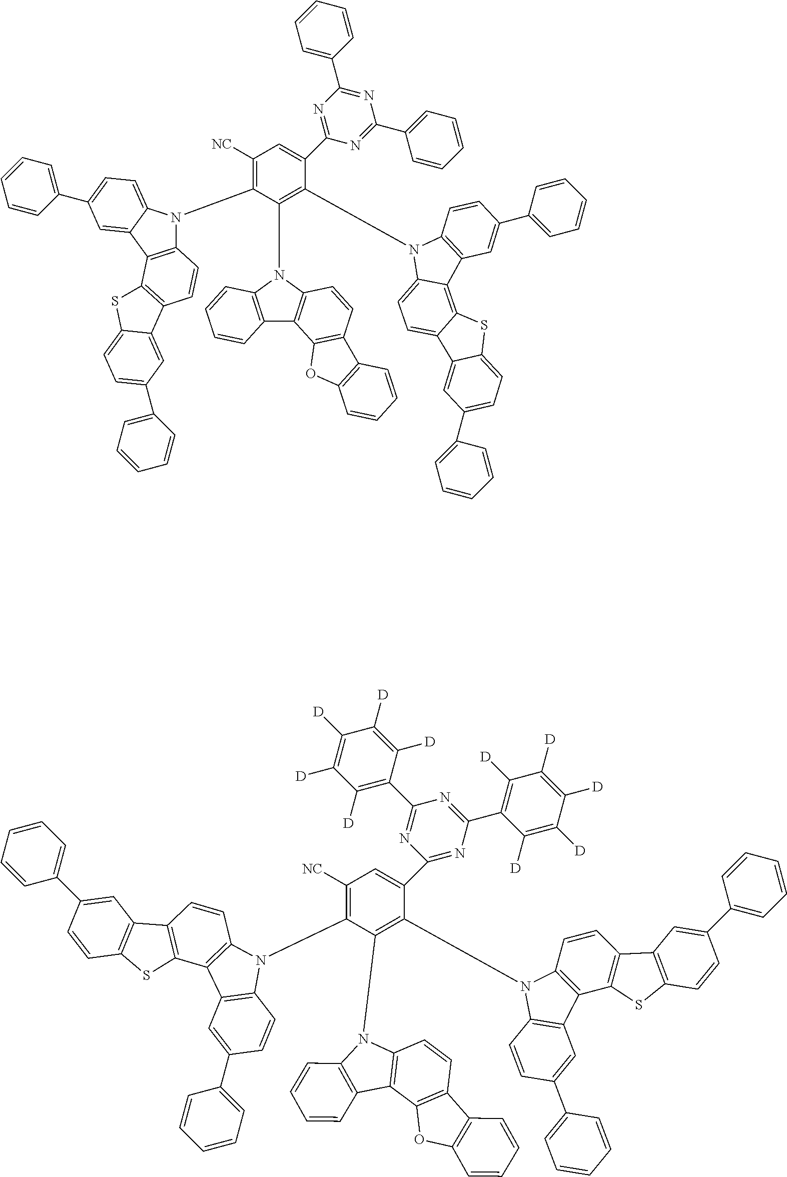 [SMILES: N#Cc1cc(-c2nc(-c3ccccc3)nc(-c3ccccc3)n2)c(-n2c3ccc(-c4ccccc4)cc3c3c4sc5ccc(-c6ccccc6)cc5c4ccc32)c(-n2c3ccccc3c3c4oc5ccccc5c4ccc32)c1-n1c2ccc(-c3ccccc3)cc2c2c3sc4ccc(-c5ccccc5)cc4c3ccc21 ; [2H]c1c([2H])c([2H])c(-c2nc(-c3cc(C#N)c(-n4c5ccc(-c6ccccc6)cc5c5c6sc7ccc(-c8ccccc8)cc7c6ccc54)c(-n4c5ccccc5c5c6oc7ccccc7c6ccc54)c3-n3c4ccc(-c5ccccc5)cc4c4c5sc6ccc(-c7ccccc7)cc6c5ccc43)nc(-c3c([2H])c([2H])c([2H])c([2H])c3[2H])n2)c([2H])c1[2H]]

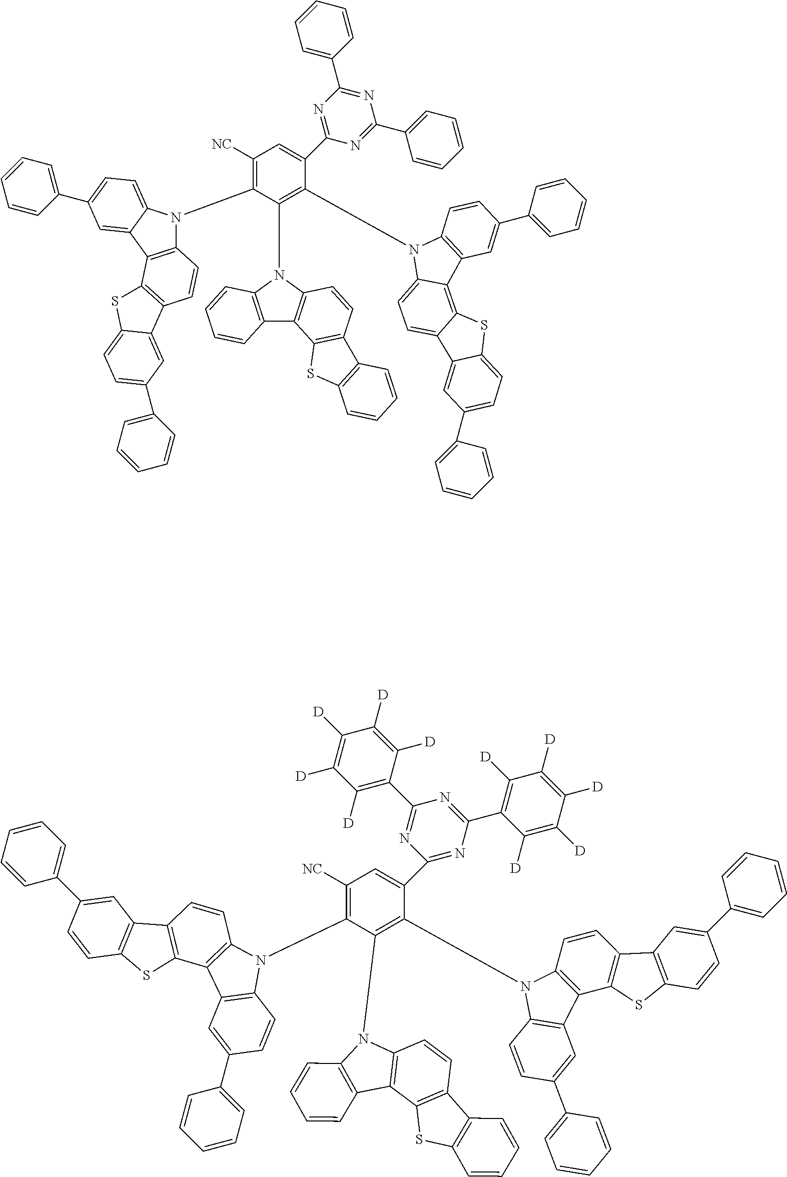 [SMILES: N#Cc1cc(-c2nc(-c3ccccc3)nc(-c3ccccc3)n2)c(-n2c3ccc(-c4ccccc4)cc3c3c4sc5ccc(-c6ccccc6)cc5c4ccc32)c(-n2c3ccccc3c3c4sc5ccccc5c4ccc32)c1-n1c2ccc(-c3ccccc3)cc2c2c3sc4ccc(-c5ccccc5)cc4c3ccc21 ; [2H]c1c([2H])c([2H])c(-c2nc(-c3cc(C#N)c(-n4c5ccc(-c6ccccc6)cc5c5c6sc7ccc(-c8ccccc8)cc7c6ccc54)c(-n4c5ccccc5c5c6sc7ccccc7c6ccc54)c3-n3c4ccc(-c5ccccc5)cc4c4c5sc6ccc(-c7ccccc7)cc6c5ccc43)nc(-c3c([2H])c([2H])c([2H])c([2H])c3[2H])n2)c([2H])c1[2H]]

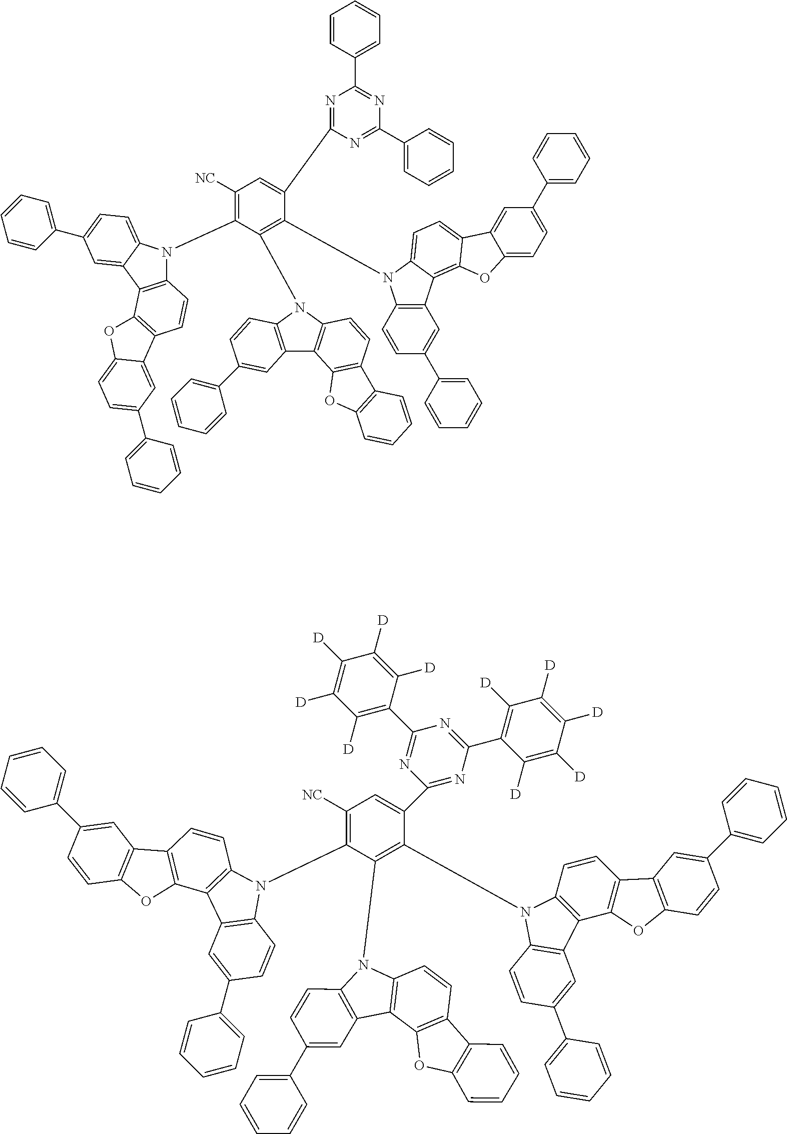 [SMILES: N#Cc1cc(-c2nc(-c3ccccc3)nc(-c3ccccc3)n2)c(-n2c3ccc(-c4ccccc4)cc3c3c4oc5ccc(-c6ccccc6)cc5c4ccc32)c(-n2c3ccc(-c4ccccc4)cc3c3c4oc5ccccc5c4ccc32)c1-n1c2ccc(-c3ccccc3)cc2c2c3oc4ccc(-c5ccccc5)cc4c3ccc21 ; [2H]c1c([2H])c([2H])c(-c2nc(-c3cc(C#N)c(-n4c5ccc(-c6ccccc6)cc5c5c6oc7ccc(-c8ccccc8)cc7c6ccc54)c(-n4c5ccc(-c6ccccc6)cc5c5c6oc7ccccc7c6ccc54)c3-n3c4ccc(-c5ccccc5)cc4c4c5oc6ccc(-c7ccccc7)cc6c5ccc43)nc(-c3c([2H])c([2H])c([2H])c([2H])c3[2H])n2)c([2H])c1[2H]]

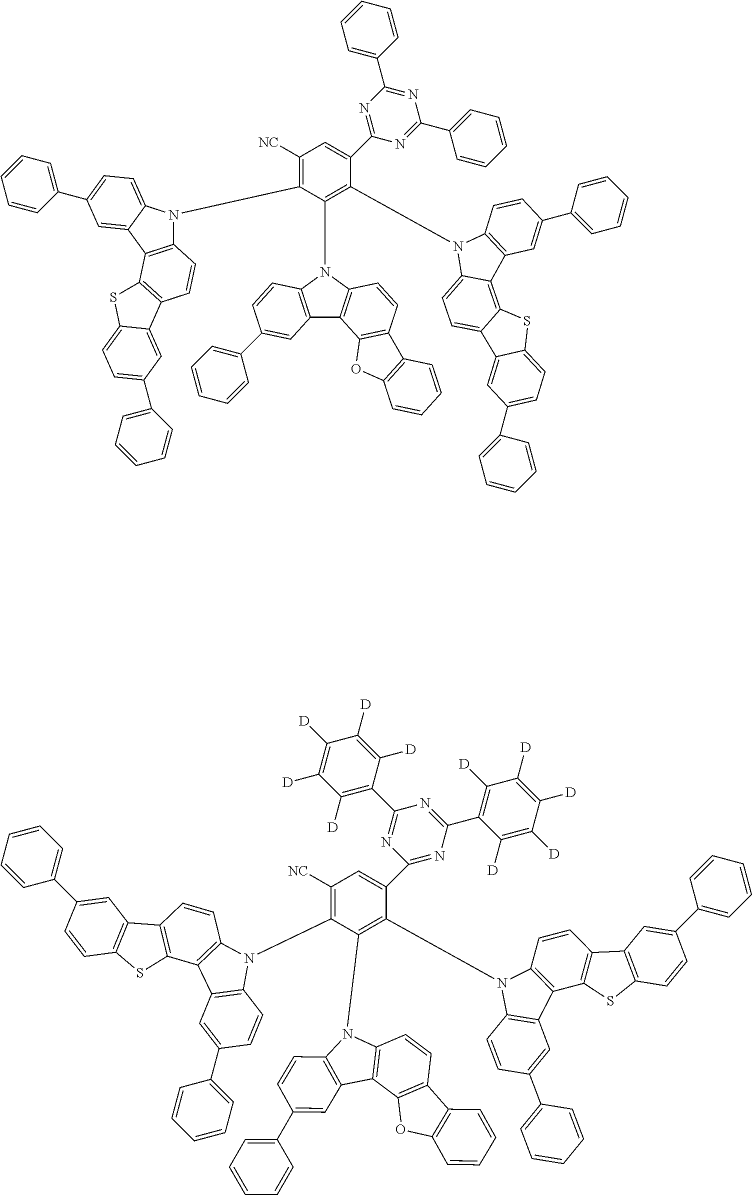 [SMILES: N#Cc1cc(-c2nc(-c3ccccc3)nc(-c3ccccc3)n2)c(-n2c3ccc(-c4ccccc4)cc3c3c4sc5ccc(-c6ccccc6)cc5c4ccc32)c(-n2c3ccc(-c4ccccc4)cc3c3c4oc5ccccc5c4ccc32)c1-n1c2ccc(-c3ccccc3)cc2c2c3sc4ccc(-c5ccccc5)cc4c3ccc21 ; [2H]c1c([2H])c([2H])c(-c2nc(-c3cc(C#N)c(-n4c5ccc(-c6ccccc6)cc5c5c6sc7ccc(-c8ccccc8)cc7c6ccc54)c(-n4c5ccc(-c6ccccc6)cc5c5c6oc7ccccc7c6ccc54)c3-n3c4ccc(-c5ccccc5)cc4c4c5sc6ccc(-c7ccccc7)cc6c5ccc43)nc(-c3c([2H])c([2H])c([2H])c([2H])c3[2H])n2)c([2H])c1[2H]]

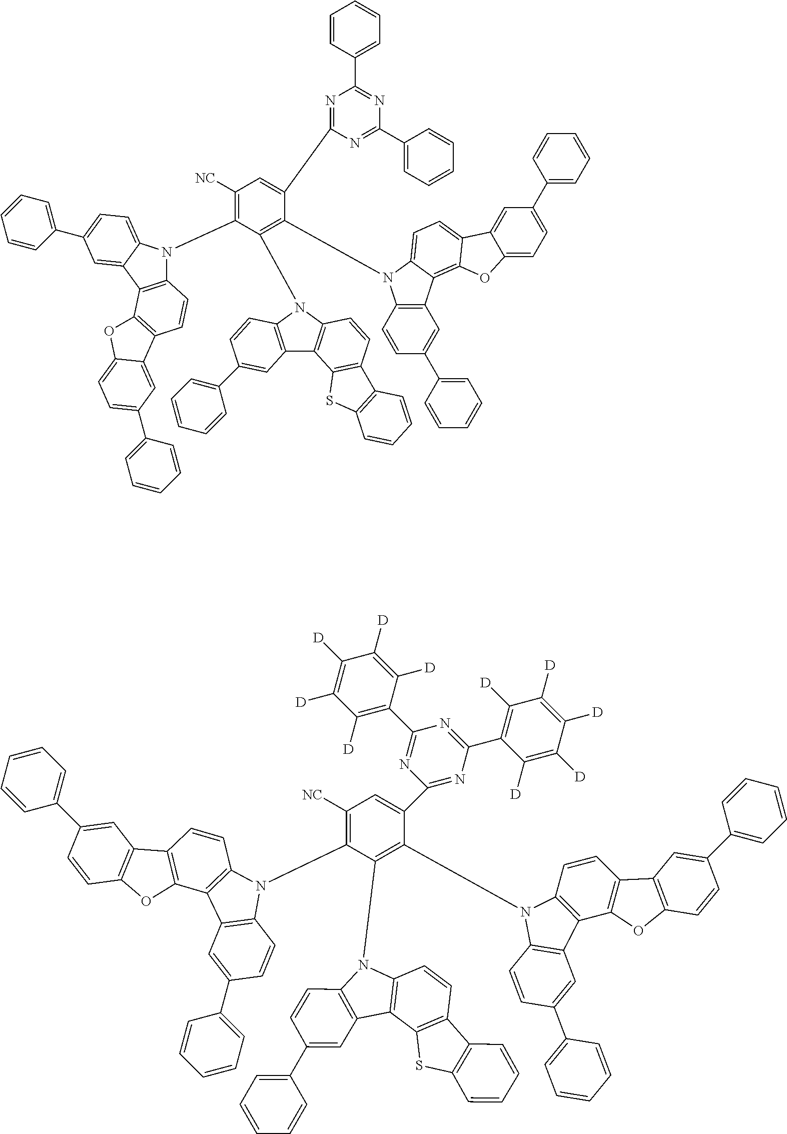 [SMILES: N#Cc1cc(-c2nc(-c3ccccc3)nc(-c3ccccc3)n2)c(-n2c3ccc(-c4ccccc4)cc3c3c4oc5ccc(-c6ccccc6)cc5c4ccc32)c(-n2c3ccc(-c4ccccc4)cc3c3c4sc5ccccc5c4ccc32)c1-n1c2ccc(-c3ccccc3)cc2c2c3oc4ccc(-c5ccccc5)cc4c3ccc21 ; [2H]c1c([2H])c([2H])c(-c2nc(-c3cc(C#N)c(-n4c5ccc(-c6ccccc6)cc5c5c6oc7ccc(-c8ccccc8)cc7c6ccc54)c(-n4c5ccc(-c6ccccc6)cc5c5c6sc7ccccc7c6ccc54)c3-n3c4ccc(-c5ccccc5)cc4c4c5oc6ccc(-c7ccccc7)cc6c5ccc43)nc(-c3c([2H])c([2H])c([2H])c([2H])c3[2H])n2)c([2H])c1[2H]]

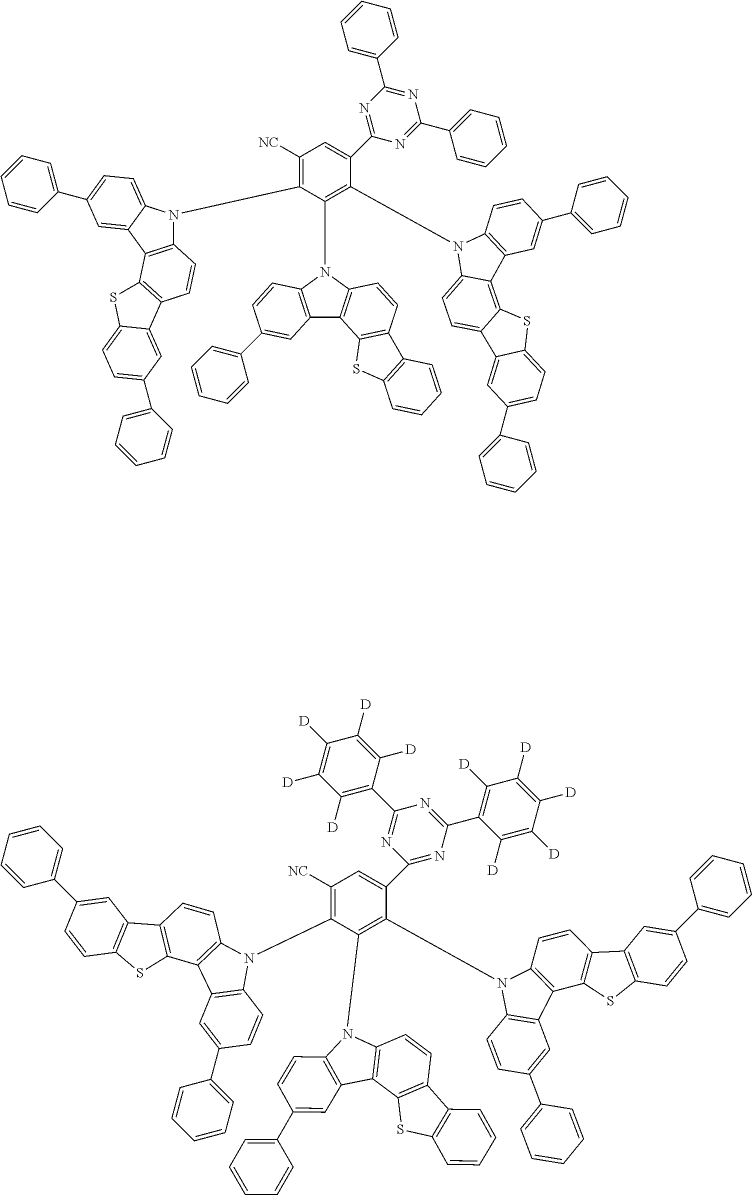 [SMILES: N#Cc1cc(-c2nc(-c3ccccc3)nc(-c3ccccc3)n2)c(-n2c3ccc(-c4ccccc4)cc3c3c4sc5ccc(-c6ccccc6)cc5c4ccc32)c(-n2c3ccc(-c4ccccc4)cc3c3c4sc5ccccc5c4ccc32)c1-n1c2ccc(-c3ccccc3)cc2c2c3sc4ccc(-c5ccccc5)cc4c3ccc21 ; [2H]c1c([2H])c([2H])c(-c2nc(-c3cc(C#N)c(-n4c5ccc(-c6ccccc6)cc5c5c6sc7ccc(-c8ccccc8)cc7c6ccc54)c(-n4c5ccc(-c6ccccc6)cc5c5c6sc7ccccc7c6ccc54)c3-n3c4ccc(-c5ccccc5)cc4c4c5sc6ccc(-c7ccccc7)cc6c5ccc43)nc(-c3c([2H])c([2H])c([2H])c([2H])c3[2H])n2)c([2H])c1[2H]]

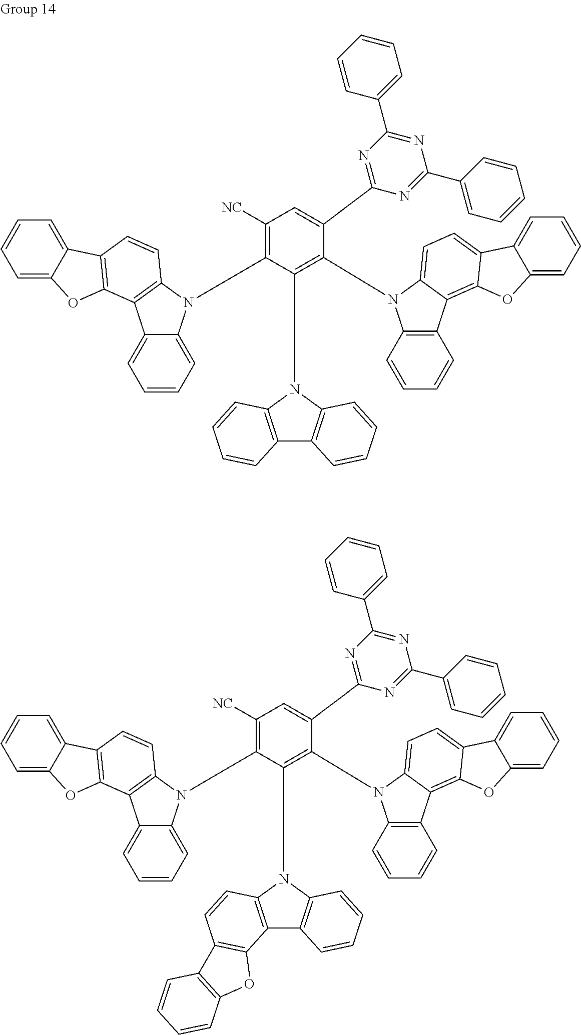 [SMILES: N#Cc1cc(-c2nc(-c3ccccc3)nc(-c3ccccc3)n2)c(-n2c3ccccc3c3c4oc5ccccc5c4ccc32)c(-n2c3ccccc3c3ccccc32)c1-n1c2ccccc2c2c3oc4ccccc4c3ccc21 ; N#Cc1cc(-c2nc(-c3ccccc3)nc(-c3ccccc3)n2)c(-n2c3ccccc3c3c4oc5ccccc5c4ccc32)c(-n2c3ccccc3c3c4oc5ccccc5c4ccc32)c1-n1c2ccccc2c2c3oc4ccccc4c3ccc21]

The molecular weight of the compound represented by the general formula (1) is preferably 1500 or less, more preferably 1200 or less, still more preferably 1000 or less, and even more preferably 900 or less, for example, in the case where an organic layer containing the compound represented by the general formula (1) is intended to be film-formed and used by a vapor deposition method. The lower limit of the molecular weight is the molecular weight of the minimum compound represented by the general formula (1).

The compound represented by the general formula (1) can be formed into a film by a coating method regardless of the molecular weight. When the coating method is used, even a compound having a relatively large molecular weight can be formed into a film. The compound represented by the general formula (1) has an advantage of being easily dissolved in an organic solvent. For this reason, the compound represented by the general formula (1) is easily applicable to a coating method and is easily purified to increase its purity.

It is also conceivable to use a compound containing a plurality of structures represented by the general formula (1) in a molecule as a light emitting material by applying the present invention.

For example, it is conceivable that a polymer obtained by allowing a polymerizable group to be present in the structure represented by the general formula (1) in advance and polymerizing the polymerizable group is used as the light emitting material. For example, it is conceivable that a polymer having a repeating unit is obtained by preparing a monomer containing a polymerizable functional group at any site of the general formula (1) and polymerizing the monomer alone or copolymerizing the monomer with another monomer, and the polymer is used as the light emitting material. Alternatively, it is also conceivable to obtain a dimer or a trimer by coupling compounds having a structure represented by the general formula (1) to each other and to use the dimer or the trimer as a light emitting material.

Examples of the polymer having a repeating unit containing a structure represented by the general formula (1) include polymers containing a structure represented by any one of the following two general formulae.

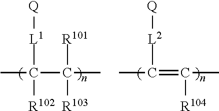

In the above general formulae, Q represents a group containing the structure represented by the general formula (1), and L¹ and L² represent a linking group. The linking group preferably has 0 to 20 carbon atoms, more preferably 1 to 15 carbon atoms, and still more preferably 2 to 10 carbon atoms. The linking group preferably has a structure represented by —X¹¹-L¹¹-. Here, X¹¹ represents an oxygen atom or a sulfur atom, and is preferably an oxygen atom. L¹¹ represents a linking group, and is preferably a substituted or unsubstituted alkylene group or a substituted or unsubstituted arylene group, and more preferably a substituted or unsubstituted alkylene group having 1 to 10 carbon atoms or a substituted or unsubstituted phenylene group.

In the above general formulae, R¹⁰¹, R¹⁰², R¹⁰³ and R¹⁰⁴ each independently represent a substituent. It is preferably a substituted or unsubstituted alkyl group having 1 to 6 carbon atoms, a substituted or unsubstituted alkoxy group having 1 to 6 carbon atoms, or a halogen atom, more preferably an unsubstituted alkyl group having 1 to 3 carbon atoms, an unsubstituted alkoxy group having 1 to 3 carbon atoms, a fluorine atom, or a chlorine atom, and still more preferably an unsubstituted alkyl group having 1 to 3 carbon atoms or an unsubstituted alkoxy group having 1 to 3 carbon atoms.

The linking group represented by L¹ and L² can bond to any site of the general formula (1) constituting Q. Two or more linking groups can be linked to one Q to form a cross-linked structure or a network structure.

Specific structural examples of the repeating unit include structures represented by the following formulae.

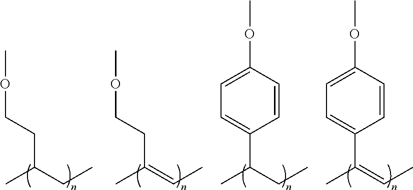

The polymer having a repeating unit including these formulae can be synthesized by introducing a hydroxy group into any site of the general formula (1), reacting the following compound using the hydroxy group as a linker to introduce a polymerizable group, and polymerizing the polymerizable group.

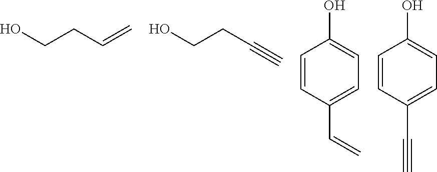

The polymer having a structure represented by the general formula (1) in the molecule can be a polymer having only a repeating unit that has the structure represented by the general formula (1), or can be a polymer containing a repeating unit that has any other structure. The repeating unit having the structure represented by the general formula (1) to be contained in the polymer can be a single kind or two or more kinds. The repeating unit not having the structure represented by the general formula (1) includes those derived from monomers used in general copolymerization. For example, it includes repeating units derived from monomers having an ethylenically unsaturated bond, such as ethylene or styrene.

In some embodiments, the compound represented by the general formula (1) is a light emitting material.

In some embodiments, the compound represented by the general formula (1) is a compound capable of emitting delayed fluorescence.

In some embodiments of the present disclosure, the compound represented by the general formula (1) is, when excited thermally or by an electronic means, able to emit light in a UV region, emit light of blue, green, yellow, orange, or red in a visible spectral region (e.g., about 420 nm to about 500 nm, about 500 nm to about 600 nm, or about 600 nm to about 700 nm) or emit light in a near IR region.

In some embodiments of the present disclosure, the compound represented by the general formula (1) is, when excited thermally or by an electronic means, able to emit light of red or orange in a visible spectral region (e.g., about 620 nm to about 780 nm, about 650 nm).

In some embodiments of the present disclosure, the compound represented by the general formula (1) is, when excited thermally or by an electronic means, able to emit light of orange or yellow in a visible spectral region (e.g., about 570 nm to about 620 nm, about 590 nm, about 570 nm).

In some embodiments of the present disclosure, the compound represented by the general formula (1) is, when excited thermally or by an electronic means, able to emit light of green in a visible spectral region (e.g., about 490 nm to about 575 nm, about 510 nm).

In some embodiments of the present disclosure, the compound represented by the general formula (1) is, when excited thermally or by an electronic means, able to emit light of blue in a visible spectral region (e.g., about 400 nm to about 490 nm, about 475 nm).

In some embodiments of the present disclosure, the compound represented by the general formula (1) is, when excited thermally or by an electronic means, able to emit light in a UV spectral region (e.g., about 280 to 400 nm).

In some embodiments of the present disclosure, the compound represented by the general formula (1) is, when excited thermally or by an electronic means, able to emit light in an IR spectral region (e.g., about 780 nm to 2 μm).

In some embodiments of the present disclosure, an organic semiconductor device using the compound represented by the general formula (1) can be produced. The organic semiconductor device referred to herein can be an organic optical device in which light is interposed or an organic device in which light is not interposed. The organic optical device can be an organic light emitting device in which the device emits light, an organic light receiving device in which the device receives light, or a device in which energy transfer by light occurs in the device. In some embodiments of the present disclosure, an organic optical device such as an organic electroluminescent device or a solid-state imaging device (for example, a CMOS image sensor) can be produced by using the compound represented by the general formula (1). In some embodiments of the present disclosure, a CMOS (complementary metal-oxide semiconductor) or the like using the compound represented by the general formula (1) can be produced.

Electronic characteristics of small-molecule chemical substance libraries can be calculated by known ab initio quantum chemistry calculation. For example, according to time-dependent density functional theory calculation using 6-31G* as a basis, and a functional group known as Becke's three parameters, Lee-Yang-Parr hybrid functionals, the Hartree-Fock equation (TD-DFT/B3LYP/6-31G*) is analyzed and molecular fractions (parts) having HOMO not lower than a specific threshold value and LUMO not higher than a specific threshold value can be screened.

With that, for example, in the presence of a HOMO energy (for example, ionizing potential) of −6.5 eV or more, a donor part (“D”) can be selected. On the other hand, for example, in the presence of a LUMO energy (for example, electron affinity) of −0.5 eV or less, an acceptor part (“A”) can be selected. A bridge part (“B”) is a strong conjugated system, for example, capable of strictly limiting the acceptor part and the donor part in a specific three-dimensional configuration, and therefore prevents the donor part and the acceptor part from overlapping in the n-conjugated system.

In some embodiments, a compound library is screened using at least one of the following characteristics.
1. Light emission around a specific wavelength
2. A triplet state over a calculated specific energy level
3. ΔE_{ST} value lower than a specific value
4. Quantum yield more than a specific value
5. HOMO level
6. LUMO level

In some embodiments, the difference (ΔE_{ST}) between the lowest singlet excited state and the lowest triplet excited state at 77 K is less than about 0.5 eV, less than about 0.4 eV, less than about 0.3 eV, less than about 0.2 eV, or less than about 0.1 eV. In some embodiments, ΔE_{ST} value is less than about 0.09 eV, less than about 0.08 eV, less than about 0.07 eV, less than about 0.06 eV, less than about 0.05 eV, less than about 0.04 eV, less than about 0.03 eV, less than about 0.02 eV, or less than about 0.01 eV.

In some embodiments, the compound represented by the general formula (1) shows a quantum yield of more than 25%, for example, about 30%, about 35%, about 40%, about 45%, about 50%, about 55%, about 60%, about 65%, about 70%, about 75%, about 80%, about 85%, about 90%, about 95% or more.

### [Method for Synthesizing Compound Represented by General Formula (1)]

The compound represented by the general formula (1) includes a novel compound.

The compound represented by the general formula (1) can be synthesized by combining known reactions. For example, by reacting a cyanobenzene having a substituted or unsubstituted aryl group (e.g., a phenyl group) and a halogen atom with a substituted ring-fused carbazole, the compound represented by the general formula (1) substituted with a substituted ring-fused carbazol-9-yl group can be synthesized. For details of the reaction conditions, Synthesis Examples described later can be referred to.

### [Structure Using Compound Represented by General Formula (1)]

In some embodiments, the compound represented by the general formula (1) is used along with one or more materials (e.g., small molecules, polymers, metals, metal complexes), by combining them, or by dispersing the compound, or by covalent-bonding with the compound, or by coating with the compound, or by carrying the compound, or by associating with the compound, and solid films or layers are formed. For example, by combining the compound represented by the general formula (1) with an electroactive material, a film can be formed. In some cases, the compound represented by the general formula (1) can be combined with a hole transporting polymer. In some cases, the compound represented by the general formula (1) can be combined with an electron transporting polymer. In some cases, the compound represented by the general formula (1) can be combined with a hole transporting polymer and an electron transporting polymer. In some cases, the compound represented by the general formula (1) can be combined with a copolymer having both a hole transporting moiety and an electron transporting moiety. In the embodiments mentioned above, the electrons and/or the holes formed in a solid film or layer can be interacted with the compound represented by the general formula (1).

### [Film Formation]

In some embodiments, a film containing the compound represented by the general formula (1) can be formed in a wet process. In a wet process, a solution prepared by dissolving a composition containing the compound of the present invention is applied onto a surface, and then the solvent is removed to form a film. The wet process includes a spin coating method, a slit coating method, an inkjet method (a spraying method), a gravure printing method, an offset printing method and flexographic printing method, which, however are not limitative. In the wet process, an appropriate organic solvent capable of dissolving a composition containing the compound of the present invention is selected and used. In some embodiments, a substituent (e.g., an alkyl group) capable of increasing the solubility in an organic solvent can be introduced into the compound contained in the composition.

In some embodiments, a film containing the compound of the present invention can be formed in a dry process. In some embodiments, a vacuum deposition method is employable as a dry process, which, however, is not limitative. In the case where a vacuum deposition method is employed, compounds to constitute a film can be vapor co-deposited from individual vapor deposition sources, or can be vapor co-deposited from a single vapor deposition source formed by mixing the compounds. In the case where a single vapor deposition source is used, a mixed powder prepared by mixing compound powders can be used, or a compression molded body prepared by compression-molding the mixed powder can be used, or a mixture prepared by heating and melting the compounds and cooling the resulting melt can be used. In some embodiments, by vapor co-deposition under the condition where the vapor deposition rate (weight reduction rate) of the plural compounds contained in a single vapor deposition source is the same or is nearly the same, a film having a compositional ratio corresponding to the compositional ratio of the plural compounds contained in the vapor deposition source can be formed. When plural compounds are mixed in the same compositional ratio as the compositional ratio of the film to be formed to prepare a vapor deposition source, a film having a desired compositional ratio can be formed in a simplified manner. In some embodiments, the temperature at which the compounds to be vapor co-deposited have the same weight reduction ratio is specifically defined, and the temperature can be employed as the temperature of vapor co-deposition.

### [Use Examples of Compound Represented by General Formula (1)]

The compound represented by the general formula (1) is useful as a material for an organic light emitting device. In particular, the compound is preferably used for an organic light emitting diode or the like.

### Organic Light Emitting Diode:

One embodiment of the present invention relates to use of the compound represented by the general formula (1) of the present invention as a light emitting material for organic light emitting devices. In some embodiments, the compound represented by the general formula (1) of the present invention can be effectively used as a light emitting material in a light emitting layer in an organic light emitting device. In some embodiments, the compound represented by the general formula (1) includes a delayed fluorescent material that emits delayed fluorescence. In some embodiments, the present invention provides a delayed fluorescent material having a structure represented by the general formula (1). In some embodiments, the present invention relates to use of the compound represented by the general formula (1) as a delayed fluorescent material. In some embodiments, the compound represented by the general formula (1) of the present invention can be used as a host material, and can be used along with one or more light emitting materials, and the light emitting material can be a fluorescent material, a phosphorescent material or a TADF. In some embodiments, the compound represented by the general formula (1) can be used as a hole transporting material. In some embodiments, the compound represented by the general formula (1) can be used as an electron transporting material. In some embodiments, the present invention relates to a method of generating delayed fluorescence from the compound represented by the general formula (1). In some embodiments, the organic light emitting device containing the compound as a light emitting material emits delayed fluorescence and shows a high light emission efficiency.

In some embodiments, the light emitting layer contains the compound represented by the general formula (1), and the compound represented by the general formula (1) is aligned in parallel to the substrate. In some embodiments, the substrate is a film-forming surface. In some embodiment, the alignment of the compound represented by the general formula (1) relative to the film-forming surface can have some influence on the propagation direction of light emitted by the aligned compounds, or can determine the direction. In some embodiments, by aligning the propagation direction of light emitted by the compound represented by the general formula (1), the light extraction efficiency from the light emitting layer can be improved.

One aspect of the present invention relates to an organic light emitting device. In some embodiments, the organic light emitting device includes a light emitting layer. In some embodiments, the light emitting layer contains, as a light emitting material, the compound represented by the general formula (1). In some embodiments, the organic light emitting device is an organic photoluminescent device (organic PL device). In some embodiments, the organic light emitting device is an organic electroluminescent device (organic EL device). In some embodiments, the compound represented by the general formula (1) assists light irradiation from the other light emitting materials contained in the light emitting layer (as a so-called assist dopant). In some embodiments, the compound represented by the general formula (1) contained in the light emitting layer is in a lowest excited singlet energy level, and is contained between the lowest excited singlet energy level of the host material contained in the light emitting layer and the lowest excited singlet energy level of the other light emitting materials contained in the light emitting layer.

In some embodiments, the organic photoluminescent device comprises at least one light emitting layer. In some embodiments, the organic electroluminescent device includes at least an anode, a cathode, and an organic layer between the anode and the cathode. In some embodiments, the organic layer includes at least a light emitting layer. In some embodiments, the organic layer includes only a light emitting layer. In some embodiments, the organic layer includes one or more organic layers in addition to the light emitting layer. Examples of the organic layer include a hole transporting layer, a hole injection layer, an electron barrier layer, a hole barrier layer, an electron injection layer, an electron transporting layer and an exciton barrier layer. In some embodiments, the hole transporting layer can be a hole injection and transporting layer having a hole injection function, and the electron transporting layer can be an electron injection and transporting layer having an electron injection function.

### Light Emitting Layer

In some embodiments, the light emitting layer is a layer where holes and electrons injected from the anode and the cathode, respectively, are recombined to form excitons. In some embodiments, the layer emits light.

In some embodiments, only a light emitting material is used as the light emitting layer. In some embodiments, the light emitting layer contains a light emitting material and a host material. In some embodiments, the light emitting material is one or more compounds represented by the general formula (1). In some embodiments, for improving luminous radiation efficiency of an organic electroluminescent device and an organic photoluminescent device, the singlet exciton and the triplet exciton generated in a light emitting material are confined inside the light emitting material. In some embodiments, a host material is used in the light emitting layer in addition to a light emitting material. In some embodiments, the host material is an organic compound. In some embodiments, the organic compound has an excited singlet energy and an excited triplet energy, and at least one of them is higher than those in the light emitting material of the present invention. In some embodiments, the singlet exciton and the triplet exciton generated in the light emitting material of the present invention are confined in the molecules of the light emitting material of the present invention. In some embodiments, the singlet and triplet excitons are fully confined for improving luminous radiation efficiency. In some embodiments, although high luminous radiation efficiency is still attained, singlet excitons and triplet excitons are not fully confined, that is, a host material capable of attaining high luminous radiation efficiency can be used in the present invention with no specific limitation. In some embodiments, in the light emitting material in the light emitting layer of the device of the present invention, luminous radiation occurs. In some embodiments, radiated light includes both fluorescence and delayed fluorescence. In some embodiments, radiated light includes radiated light from a host material. In some embodiments, radiated light is composed of radiated light from a host material. In some embodiments, radiated light includes radiated light from the compound represented by the general formula (1) and radiated light from a host material. In some embodiment, a TADF molecule and a host material are used. In some embodiments, TADF is an assist dopant and has a lower excited singlet energy than the host material in the light emitting layer and a higher excited singlet energy than the light emitting material in the light emitting layer.

In the case where the compound represented by the general formula (1) is used as an assist dopant, various compounds can be employed as a light emitting material (preferably a fluorescent material). As such light emitting materials, employable are an anthracene derivative, a tetracene derivative, a naphthacene derivative, a pyrene derivative, a perylene derivative, a chrysene derivative, a rubrene derivative, a coumarin derivative, a pyran derivative, a stilbene derivative, a fluorene derivative, an anthryl derivative, a pyrromethene derivative, a terphenyl derivative, a terphenylene derivative, a fluoranthene derivative, an amine derivative, a quinacridone derivative, an oxadiazole derivative, a malononitrile derivative, a pyran derivative, a carbazole derivative, a julolidine derivative, a thiazole derivative, and a derivative having a metal (Al, Zn). These exemplified skeletons can have a substituent, or may not have a substituent. These exemplified skeletons can be combined.

Light emitting materials that can be used in combination with the assist dopant having a structure represented by the general formula (1) are shown below.

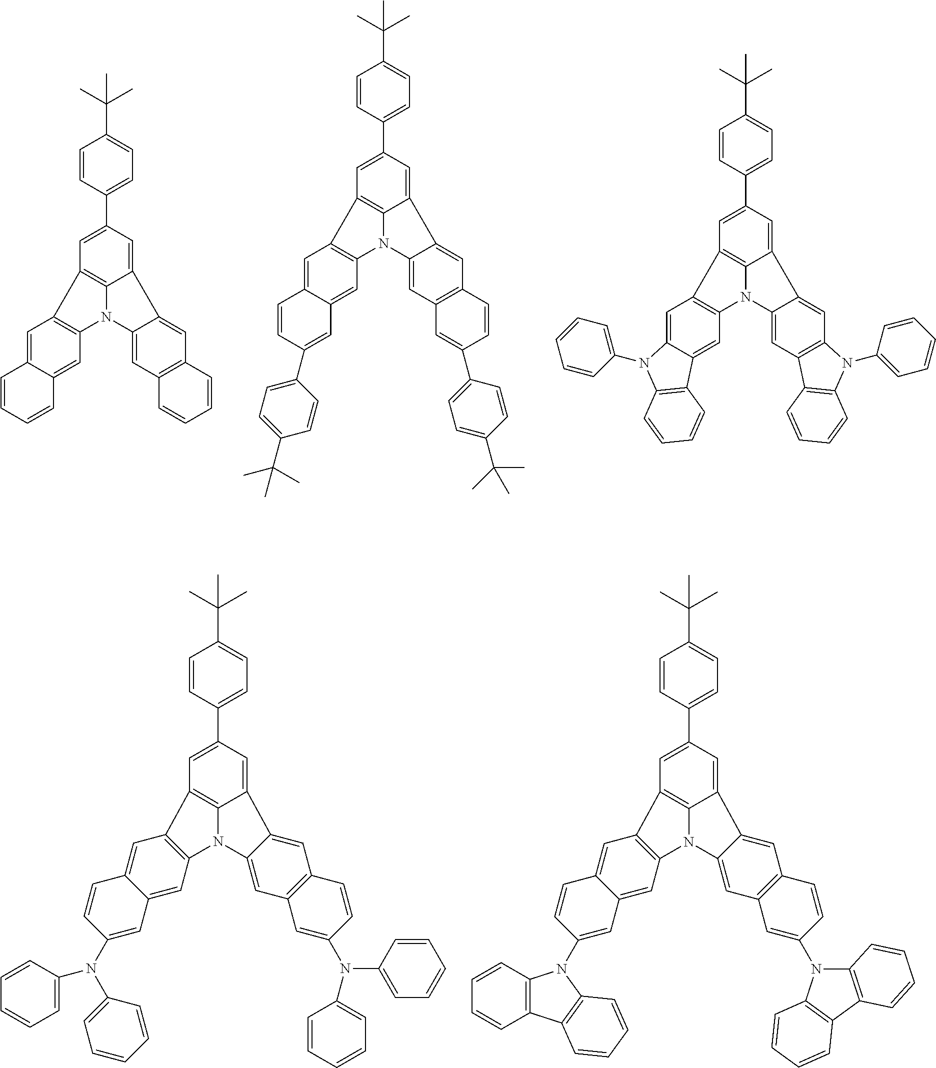

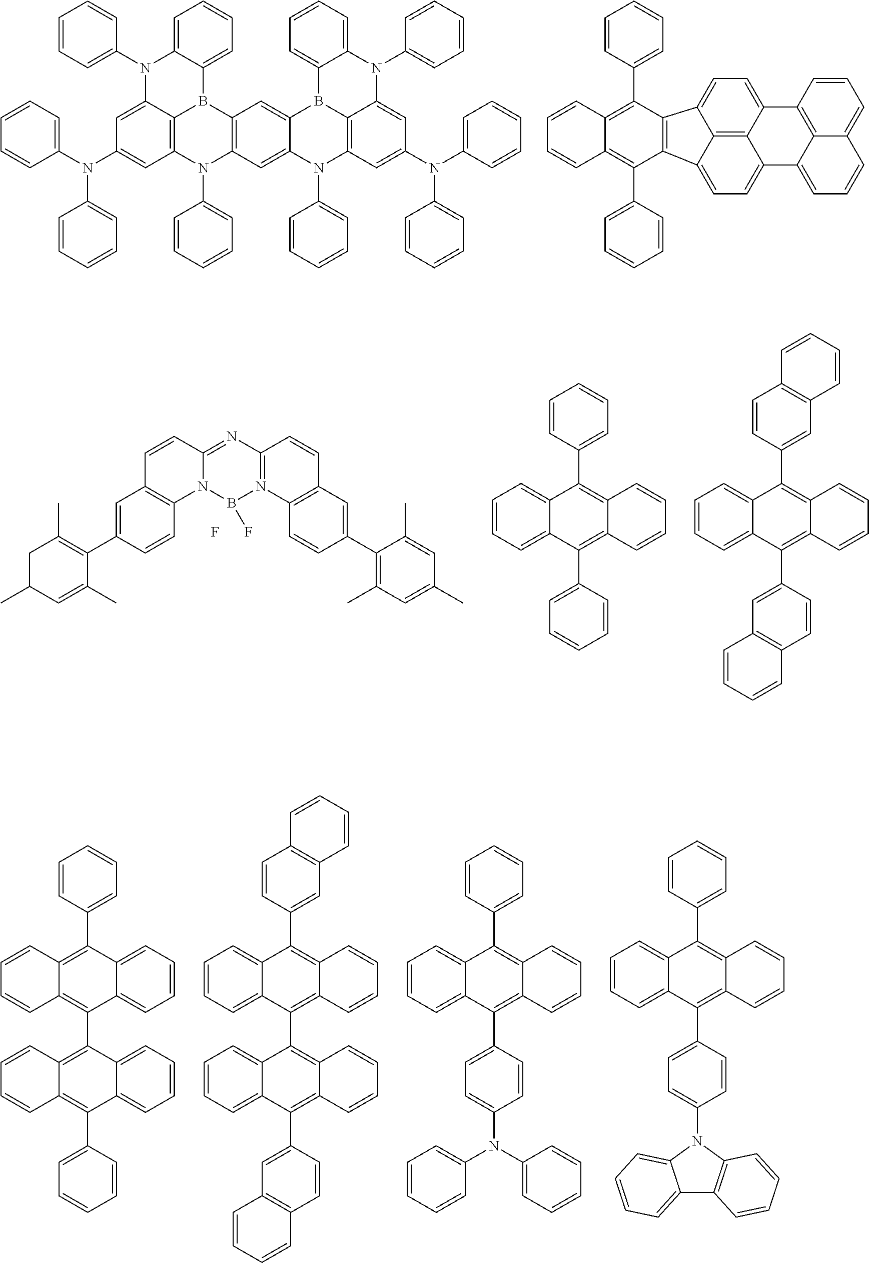

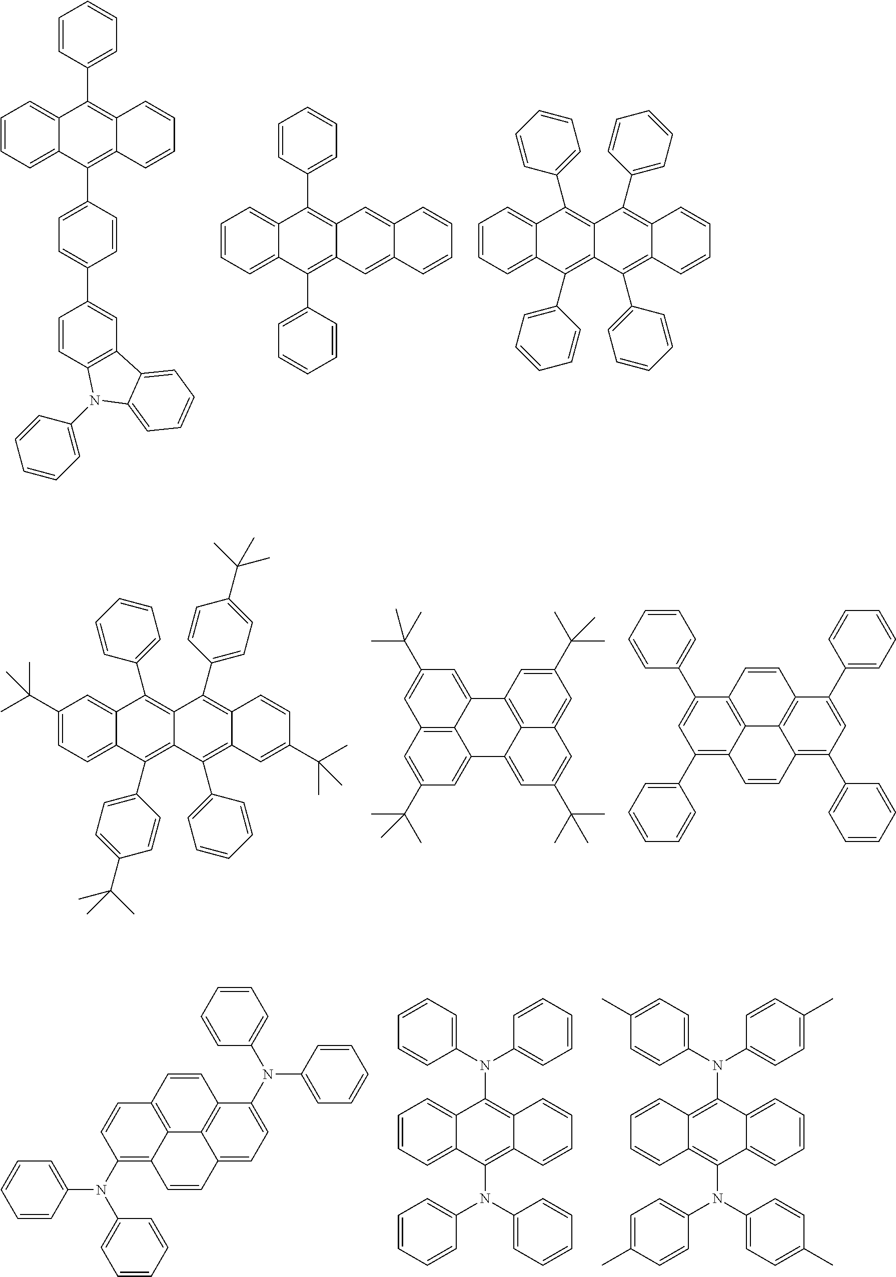

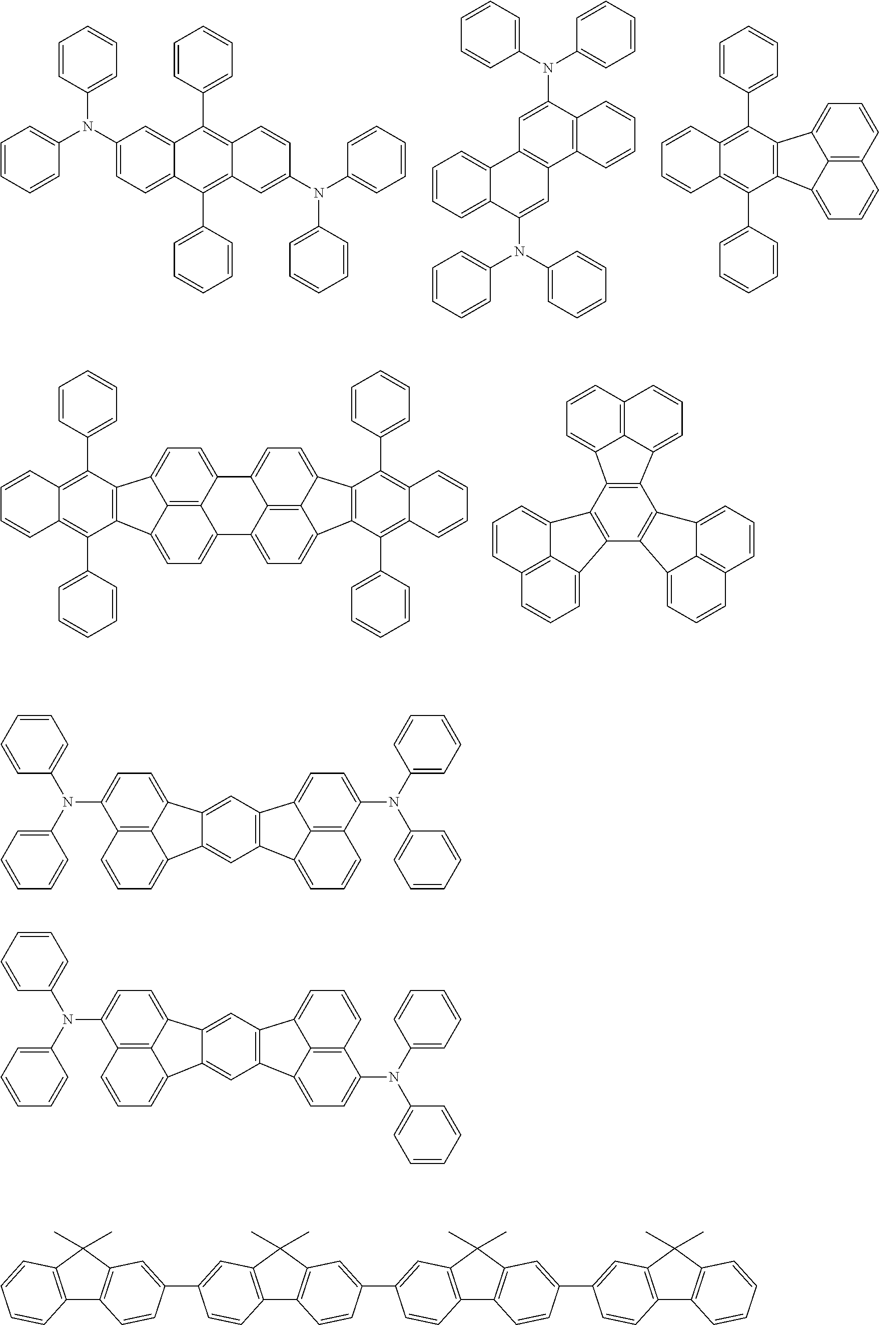

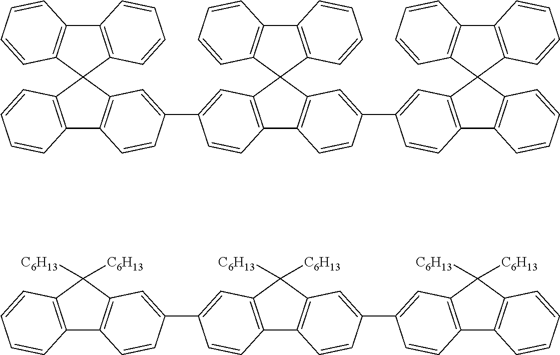

In addition, the compounds described in WO2015/022974, paragraphs 0220 to 0239 are also especially favorably employable as a light emitting material for use along with the assist dopant having a structure represented by the general formula (1).

Compounds represented by the following general formula (2) are further preferred light emitting materials.

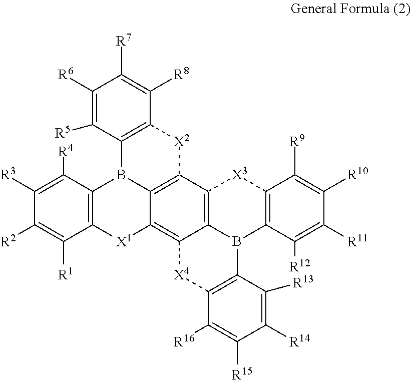

In the general formula (2), R¹, and R³ to R¹⁶ each independently represent a hydrogen atom, a deuterium atom, or a substituent.

R² represents an acceptor group, or R¹ and R² bond to each other to form an acceptor group, or R² and R³ bond to each other to form an acceptor group.

R³ and R⁴, R⁴ and R⁵, R⁵ and R⁶, R⁶ and R⁷, R⁷ and R⁸, R⁹ and R¹⁰, R¹⁰ and R¹¹, R¹¹ and R¹², R¹² and R¹³, R¹³ and R¹⁴, R¹⁴ and R¹⁵, and R¹⁵ and R¹⁶ each can bond to each other to form a cyclic structure.

X¹ represents O or NR, and R represents a substituent.

Of X² to X⁴, at least one of X³ and X⁴ is O or NR, and the remainder may be O or R, or unlinked. When not linked, both ends each independently represent a hydrogen atom, a deuterium atom or a substituent.
In the general formula (1), C—R¹, C—R³, C—R⁴, C—R⁵, C—R⁶, C—R⁷, C—R⁸, C—R⁹, C—R¹⁰, C—R¹¹, C—R¹², C—R³, C—R¹⁴, C—R¹⁵, and C—R¹⁶ can be substituted with N.

In one aspect of the present invention, when X² is O or NR, R⁷ is an acceptor group, R⁶ and R¹ bond to each other to form an acceptor group, or R⁷ and R⁸ bond to each other to form an acceptor group. In one aspect of the present invention, when X³ is O or NR, R¹⁰ is an acceptor group, R⁹ and R¹⁰ bond to each other to form an acceptor group, or R¹⁰ and R¹¹ bond to each other to form an acceptor group. In one aspect of the present invention, when X⁴ is O or NR, R¹⁵ is an acceptor group, R¹⁴ and R¹¹ bond to each other to form an acceptor group, or R¹⁵ and R¹⁶ bond to each other to form an acceptor group. In one aspect of the present invention, when X² is NR and when R is a substituted or unsubstituted phenyl group and forms a carbazole ring by directly bonding to the carbon atom to which R⁸ bonds, at least one of the 3-position and the 6-position of the carbazole ring is substituted with an acceptor group. In one aspect of the present invention, when X³ is NR and when R is a substituted or unsubstituted phenyl group and forms a carbazole ring by directly bonding to the carbon atom to which R⁹ bonds, at least one of the 3-position and the 6-position of the carbazole ring is substituted with an acceptor group. In one aspect of the present invention, when X⁴ is NR and when R is a substituted or unsubstituted phenyl group and forms a carbazole ring by directly bonding to the carbon atom to which R¹⁶ bonds, at least one of the 3-position and the 6-position of the carbazole ring is substituted with an acceptor group. In one aspect of the present invention, when X¹ is NR and when R is a substituted or unsubstituted phenyl group and forms a carbazole ring by directly bonding to the carbon atom to which R¹ bonds, the 3-position of the carbazole ring is substituted with an acceptor group (here, the 3-position is on the phenyl group). One aspect of the present invention is a compound represented by the following general formula (2a).

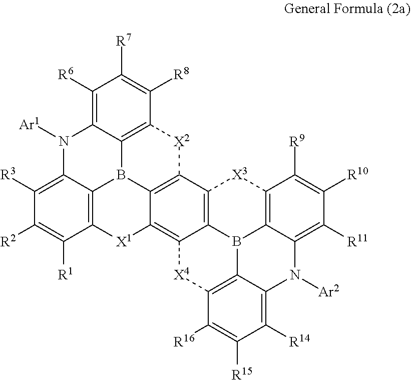

In the general formula (2a), R¹, R³, R⁶ to R¹¹, and R¹⁴ to R¹⁶ each independently represent a hydrogen atom, a deuterium atom, or a substituent.

R² represents an acceptor group, R¹ and R² bond to each other to form an acceptor group, or R² and R³ bond to each other to form an acceptor group.

R⁶ and R⁷, R⁷ and R⁸, R⁹ and R¹⁰, R¹⁰ and R¹¹, R¹⁴ and R¹⁵, and R¹⁵ and R¹⁶ each can bond to each other to form a cyclic structure.

X¹ represents O or NR, and R represents a substituent.

Of X² to X⁴, at least one of X³ and X⁴ is O or NR, and the remainder may be O or NR, or unlinked. When not linked, both ends each independently represent a hydrogen atom, a deuterium atom or a substituent.

Ar¹ and Ar2 each independently represent a substituted or unsubstituted aryl group, or a substituted or unsubstituted heteroaryl group.

In the general formula (1), C—R¹, C—R³, C—R⁶, C—R⁷, C—R⁸, C—R⁹, C—R¹⁰, C—R¹¹, C—R¹⁴, C—R¹⁵, and C—R¹⁶ can be substituted with N.

Compounds represented by the following general formula (3) are further preferred light emitting materials.

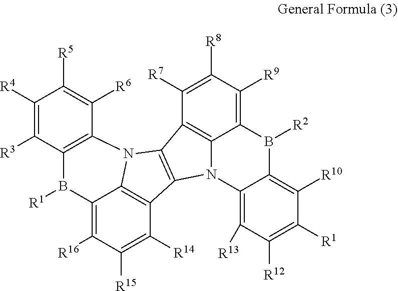

In the general formula (3), R¹ and R² each independently represent a substituted or unsubstituted alkyl group, a substituted or unsubstituted aryl group, or a substituted or unsubstituted heteroaryl group, and R³ to R¹⁶ each independently represent a hydrogen atom, a deuterium atom or a substituent.

R¹ and R³, R³ and R⁴, R⁴ and R⁵, R⁵ and R⁶, R⁶ and R⁷, R⁷ and R⁸, R⁸ and R⁹, R⁹ and R², R² and R¹⁰, R¹⁰ and R¹¹, R¹¹ and R¹², R¹² and R³, R¹³ and R¹⁴, R¹⁴ and R¹⁵, R¹⁵ and R¹⁶, and R¹⁶ and R¹ each can bond to each other to form a cyclic structure. In the general formula (1), C—R³, C—R⁴, C—R⁵, C—R⁶, C—R⁷, C—R⁸, C—R⁹, C—R¹⁰, C—R¹¹, C—R¹², C—R¹³, C—R¹⁴, C—R¹⁵, and C—R¹⁶ can be substituted with N.

In one aspect of the present invention, R¹ and R² are each independently a substituted or unsubstituted phenyl group optionally fused with any other ring. In one aspect of the present invention, R³ and R¹⁰ are each independently a substituted amino group. In one aspect of the present invention, at least one pair of R¹ and R³ and R² and R¹⁰ bonds to each other to form a cyclic structure. In one aspect of the present invention, the cyclic structure includes a benzazaborine ring.

Compounds represented by the following general formula (4) are further preferred light emitting materials.

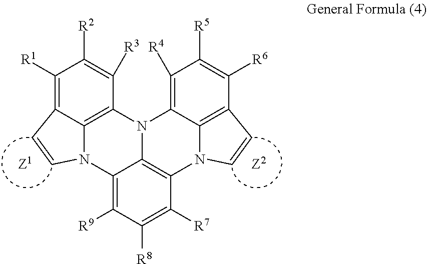

In the general formula (4), Z¹ and Z² each independently represent a substituted or unsubstituted aromatic ring, or a substituted or unsubstituted heteroaromatic ring, R¹ to R⁹ each independently represent a hydrogen atom, a deuterium atom or a substituent.

R¹ and R², R² and R³, R³ and R⁴, R⁴ and R⁵, R⁵ and R⁶, R⁷ and R⁸, and R⁸ and R⁹ each can bond to each other to form a cyclic structure.

However, at least one of the ring formed by Z¹, Z², R¹ and R² bonding to each other, the ring formed by R² and R³ bonding to each other, the ring formed by R⁴ and R⁵ bonding to each other, and the ring formed by R⁵ and R⁶ bonding to each other is a furan ring of a substituted or unsubstituted benzofuran, a thiophene ring of a substituted or unsubstituted benzothiophene, or a pyrrole ring of a substituted or unsubstituted indole, and
at least one of R¹ to R⁹ is a substituted or unsubstituted aryl group or an acceptor group, or at least one of Z¹ and Z² is a ring having an aryl group or an acceptor group as a substituent.

Of the benzene ring skeleton-constituting carbon atoms to constitute the benzofuran ring, the benzothiophene ring, and the indole ring, a substitutable carbon atom can be substituted with a nitrogen atom. In the general formula (1), C—R¹, C—R², C—R³, C—R⁴, C—R⁵, C—R⁶, C—R⁷, C—R⁸, and C—R⁹ can be substituted with N.

In one aspect of the present invention, Z1 and Z2 are each independently a substituted or unsubstituted non-fused benzene ring, a furan ring fused with a substituted or unsubstituted benzene ring, a thiophene ring fused with a substituted or unsubstituted benzene ring, or a pyrrole ring fused with a substituted or unsubstituted benzene ring. In one aspect of the present invention, R¹ to R⁹ are each independently substituted or unsubstituted aryl group or an acceptor group, or at least one ring selected from the group consisting of the ring formed by R¹ and R² bonding to each other, the ring formed by R² and R³ bonding to each other, the ring formed by R⁴ and R⁵ bonding to each other, and the ring formed by R⁵ and R⁶ bonding to each other is a furan ring fused with a substituted or unsubstituted benzene ring, a thiophene ring fused with a substituted or unsubstituted benzene ring, or a pyrrole ring fused with a substituted or unsubstituted benzene ring. In one aspect of the present invention, R¹ is a substituted or unsubstituted aryl group, or an acceptor group. One aspect of the present invention contains two or more rings selected from the group consisting of the benzofuran ring, the benzothiophene ring, and the indole ring.

Further more preferred light emitting materials include compounds having a ring-fused structure A, in which the carbon-carbon bond a in the following structure a is fused with a furan ring constituting a substituted or unsubstituted benzofuran ring, a thiophene ring constituting a substituted or unsubstituted benzothiophene ring, or a pyrrole ring constituting a substituted or unsubstituted indole ring, or the carbon-carbon bond b is fused with a benzene ring constituting a substituted or unsubstituted dibenzofuran ring, a benzene ring constituting a substituted or unsubstituted dibenzothiophene ring, a benzene ring constituting a substituted or unsubstituted carbazole ring, or a benzene ring constituting a substituted or unsubstituted dibenzodioxane ring (the hydrogen atom in the structure can be substituted with a deuterium atom or a substituent).

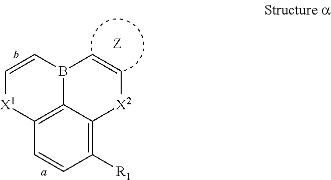

In the structure a, X¹ and X² each independently represent a nitrogen atom to which a substituted or unsubstituted aryl group or a substituted or unsubstituted aryl group bonds, or an oxygen atom,
Z represents a substituted or unsubstituted aromatic ring, or a substituted or unsubstituted heteroaromatic ring,
R¹ represents a hydrogen atom, a deuterium atom or a substituent, and
Z and X² can bond to each other to form a cyclic structure.

In the ring-fused structure A, the structure fused to b and X¹, the structure fused to b and Z, and Z and X² each can bond to each other to form a cyclic structure.

Compounds represented by the following general formula (5) are further preferred light emitting materials.

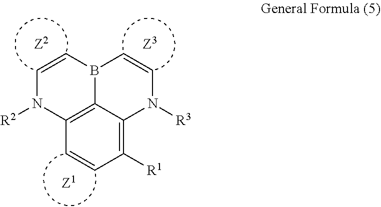

In the general formula (5), Z¹ represents a furan ring fused with a substituted or unsubstituted benzene ring, a thiophene ring fused with a substituted or unsubstituted benzene ring, or an N-substituted pyrrole ring fused with a substituted or unsubstituted benzene ring,
Z² and Z³ each independently represent a substituted or unsubstituted aromatic ring, or a substituted or unsubstituted heteroaromatic ring,
R¹ represents a hydrogen atom, a deuterium atom, or a substituent, and
R² and R³ each independently represent a substituted or unsubstituted aryl group, or a substituted or unsubstituted heteroaryl group.
Z¹ and R¹, R² and Z², Z² and Z³, and Z³ and R³ each can bond to each other to form a cyclic structure. However, at least one pair of R² and Z², Z² and Z³, and Z³ and R³ bonds to each other to form a cyclic structure.

Compounds represented by the following general formula (6) are further

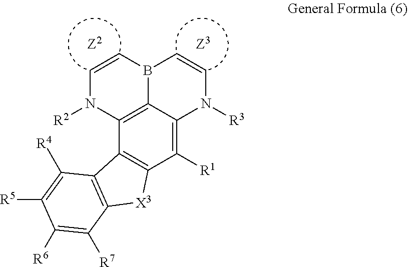

In the general formula (6), X³ represents an oxygen atom or a sulfur atom,
Z² and Z³ each independently represent a substituted or unsubstituted aromatic ring, or a substituted or unsubstituted heteroaromatic ring,
R¹ and R⁴ to R⁷ each represent a hydrogen atom, a deuterium atom or a substituent,
R² and R³ each independently represent a substituted or unsubstituted aryl group, or a substituted or unsubstituted heteroaryl group.
R² and Z², Z² and Z³, Z³ and R³, R⁴ and R⁵, R⁵ and R⁶, and R⁶ and R⁷ each can bond to each other to form a cyclic structure. However, at least one pair of R² and Z², Z² and Z³, and Z³ and R³ bonds to each other to form a cyclic structure.

Compounds represented by the following general formula (7) are further preferred light emitting materials.

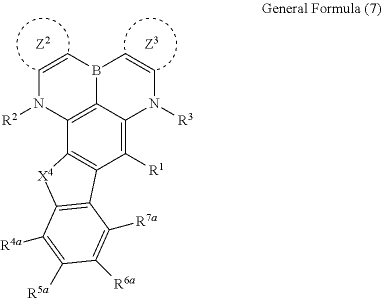

In the general formula (7), X⁴ represents an oxygen atom or a sulfur atom,
Z² and Z³ each independently represent a substituted or unsubstituted aromatic ring, or a substituted or unsubstituted heteroaromatic ring,
R¹ and R^{4a} to R^{7a} each represent a hydrogen atom, a deuterium atom or a substituent, and
R² and R³ each independently represent a substituted or unsubstituted aryl group, or a substituted or unsubstituted heteroaryl group.
R² and Z², Z² and Z³, Z³ and R³, R^{4a} and R^{5a}, R^{5a} and R^{6a}, R^{6a} and R^{7a}, and R^{7a} and R¹ each can bond to each other to forma cyclic structure. However, at least one pair of R² and Z², Z² and Z³, and Z³ and R³ bonds to each other to form a cyclic structure.

Compounds represented by the following general formula (8) are further

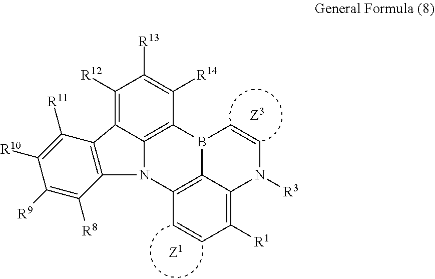

In the general formula (8), Z¹ represents a furan ring fused with a substituted or unsubstituted benzene ring, a thiophene ring fused with a substituted or unsubstituted benzene ring, or an N-substituted pyrrole ring fused with a substituted or unsubstituted benzene ring,
Z³ represents a substituted or unsubstituted aromatic ring, or a substituted or unsubstituted heteroaromatic ring,
R¹ and R⁸ to R¹¹ each independently represent a hydrogen atom, a deuterium atom, or a substituent, and
R³ represents a substituted or unsubstituted aryl group, or a substituted or unsubstituted heteroaryl group.
Z¹ and R¹, R⁸ and R⁹, R⁹ and R¹⁰, R¹⁰ and R¹¹, R¹¹ and R¹², R¹¹ and R¹³, R¹³ and R¹⁴, R¹⁴ and Z³, and Z³ and R³ each can bond to each other to form a cyclic structure.

Compounds represented by the following general formula (9) are further preferred light emitting materials.

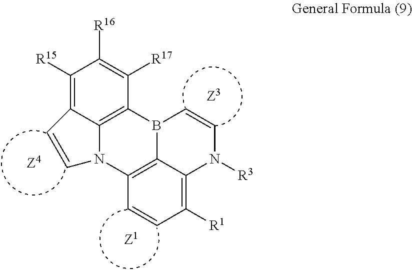

In the general formula (9), Z¹ and Z⁴ each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring,
Z³ represents a substituted or unsubstituted aromatic ring, or a substituted or unsubstituted heteroaromatic ring,
R¹ and R⁵ to R¹⁷ each independently represent a hydrogen atom, a deuterium atom, or a substituent, and
R³ represents a substituted or unsubstituted aryl group, or a substituted or unsubstituted heteroaryl group.
Z¹ and R¹, Z⁴ and R¹⁵, R¹⁵ and R¹⁶, R¹⁶ and R¹⁷, R¹⁷ and Z³, and Z³ and R³ each can bond to each other to form a cyclic structure.

Compounds represented by the following general formula (10) are further preferred light emitting materials.

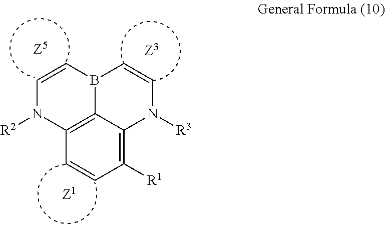

In the general formula (10), Z¹ and Z⁵ each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring,
Z³ represents a substituted or unsubstituted aromatic ring, or a substituted or unsubstituted heteroaromatic ring,
R¹ represents a hydrogen atom, a deuterium atom, or a substituent,
R² and R³ each independently represent a substituted or unsubstituted aryl group, or a substituted or unsubstituted heteroaryl group.
Z¹ and R¹, R² and Z⁵, Z⁵ and Z³, Z³ and R³ each can bond to each other to form a cyclic structure. However, at least one pair of R² and Z², Z² and Z³, and Z³ and R³ bonds to each other to form a cyclic structure.

Compounds represented by the following general formula (11) are further preferred light emitting materials.

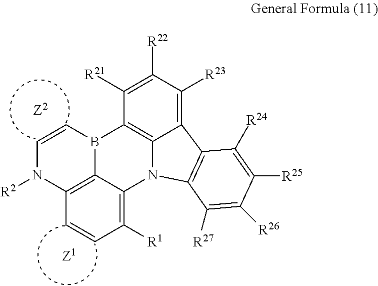

In the general formula (11), Z¹ represents a furan ring fused with a substituted or unsubstituted benzene ring, a thiophene ring fused with a substituted or unsubstituted benzene ring, or an N-substituted pyrrole ring fused with a substituted or unsubstituted benzene ring,
Z² represents a substituted or unsubstituted aromatic ring, or a substituted or unsubstituted heteroaromatic ring,
R¹ and R²¹ to R²⁷ each independently represent a hydrogen atom, a deuterium atom, or a substituent, and
R² represents a substituted or unsubstituted aryl group, or a substituted or unsubstituted heteroaryl group.
R¹ and Z¹, R² and Z², Z² and R²¹, R²¹ and R²², R²² and R²³, R²³ and R²⁴, R²⁴ and R²¹, R²⁵ and R²⁶, and R²⁶ and R²⁷ each can bond to each other to form a cyclic structure.

Compounds represented by the following general formula (12) are further preferred light emitting materials.

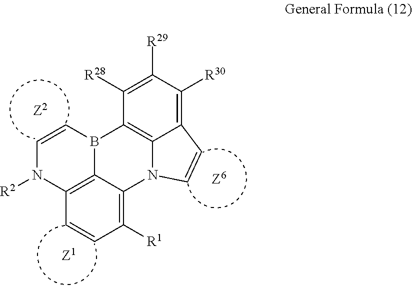

In the general formula (12), Z¹ and Z⁶ each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring,
Z² represents a substituted or unsubstituted aromatic ring, or a substituted or unsubstituted heteroaromatic ring,
R¹ and R²⁸ to R³⁰ each independently represent a hydrogen atom, a deuterium atom, or a substituent, and
R² represents a substituted or unsubstituted aryl group, or a substituted or unsubstituted heteroaryl group.
R¹ and Z¹, R² and Z², Z² and R²⁸, R²⁸ and R²⁹, R²⁹ and R³⁰, and R³⁰ and Z⁶ each can bond to each other to form a cyclic structure.

Compounds represented by the following general formula (13) are further preferred light emitting materials.

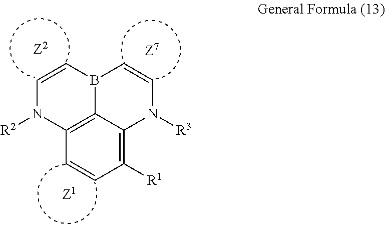

In the general formula (13), Z¹ and Z⁷ each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring,
Z² represents a substituted or unsubstituted aromatic ring, or a substituted or unsubstituted heteroaromatic ring,
R¹ represents a hydrogen atom, a deuterium atom, or a substituent, and
R² and R³ each independently represent a substituted or unsubstituted aryl group, or a substituted or unsubstituted heteroaryl group.

R¹ and Z¹, R² and Z², Z² and Z⁷, and Z⁷ and R³ each can bond to each other to form a cyclic structure. However, at least one pair of R² and Z², Z² and Z⁷, and Z⁷ and R³ bonds to each other to form a cyclic structure.

Compounds represented by the following general formula (14) are further preferred light emitting materials.

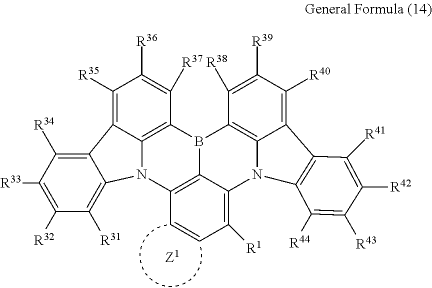

In the general formula (14), Z¹ represents a furan ring fused with a substituted or unsubstituted benzene ring, a thiophene ring fused with a substituted or unsubstituted benzene ring, or an N-substituted pyrrole ring fused with a substituted or unsubstituted benzene ring, and
R¹ and R³¹ to R¹¹ each independently represent a substituted or unsubstituted aryl group, or a substituted or unsubstituted heteroaryl group.
R¹ and Z¹, R³¹ and R³², R³² and R³³, R³³ and R³⁴, R³⁴ and R³⁵, R³⁵ and R³⁶, R³⁶ and R³⁷, R³⁷ and R³⁸, R³⁸ and R³⁹, R³⁹ and R⁴⁰, R⁴⁰ and R⁴¹, R⁴¹ and R⁴², R⁴² and R⁴³, and R⁴³ and R⁴⁴ each can bond to each other to form a cyclic structure.

Compounds represented by the following general formula (15) are further preferred light emitting materials.

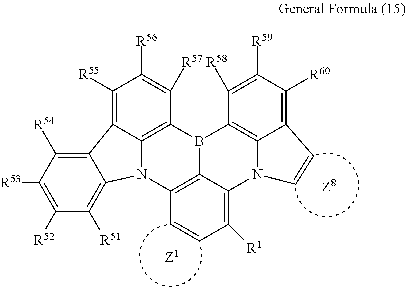

In the general formula (15), Z¹ and Z⁸ each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring, and
R¹ and R⁵¹ to R⁶⁰ each independently represent a hydrogen atom, a deuterium atom, or a substituent.

R¹ and Z¹, R⁵¹ and R⁵², R⁵² and R⁵¹, R⁵³ and R⁵⁴, R⁵⁴ and R⁵⁵, R⁵⁵ and R⁵⁶, R⁵⁶ and R⁵⁷, R⁵⁷ and R⁵⁸, R⁵⁸ and R⁵⁹, R⁵⁹ and R⁶⁰, and R⁶⁰ and Z⁸ each can bond to each other to form a cyclic structure.

Compounds represented by the following general formula (16) are further preferred light emitting materials.

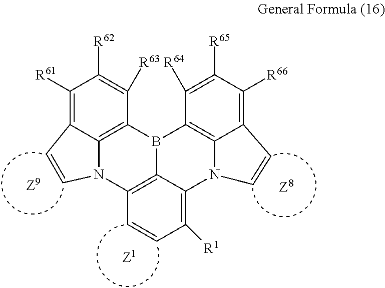

In the general formula (16), Z¹, Z⁸ and Z⁹ each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring, and
R¹ and R⁶¹ to R¹¹ each independently represent a hydrogen atom, a deuterium atom, or a substituent.

R¹ and Z¹, Z⁹ and R⁶¹, R⁶¹ and R⁶², R⁶¹ and R⁶³, R⁶³ and R⁶⁴, R⁶⁴ and R⁶⁵, R⁶⁵ and R⁶⁶, and R⁶⁶ and Z¹ each can bond to each other to form a cyclic structure.

Compounds represented by the following general formula (17) are further preferred light emitting materials.

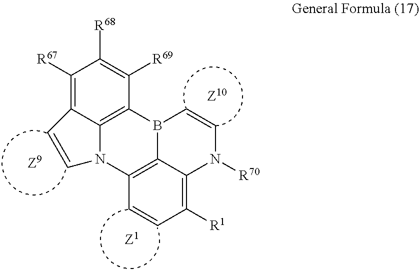

In the general formula (17), Z¹, Z⁹ and Z¹⁰ each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring,
R¹ and R⁶⁷ to R⁶⁹ each independently represent a hydrogen atom, a deuterium atom, or a substituent, and
R⁷⁰ represents a substituted or unsubstituted aryl group, or a substituted or unsubstituted heteroaryl group.

R¹ and Z¹, Z⁹ and R⁶⁷, R⁶⁷ and R⁶⁸, R⁶⁸ and R⁶⁹, R⁶⁹ and Z¹⁰, and Z¹⁰ and R⁷⁰ each can bond to each other to form a cyclic structure.

Compounds represented by the following general formula (18) are further preferred light emitting materials.

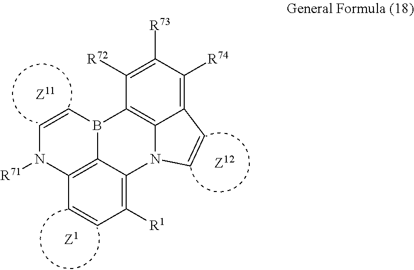

In the general formula (18), Z¹, Z¹¹ and Z¹² each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring,
R¹ and R⁷² to R⁷⁴ each independently represent a hydrogen atom, a deuterium atom, or a substituent, and
R⁷¹ represents a substituted or unsubstituted aryl group, or a substituted or unsubstituted heteroaryl group.

R¹ and Z¹, R⁷¹ and Z¹¹, Z¹¹ and R⁷², R⁷² and R⁷³, R⁷³ and Z⁷⁴, and R⁷⁴ and Z¹² each can bond to each other to form a cyclic structure.

Compounds represented by the following general formula (19) are further preferred light emitting materials.

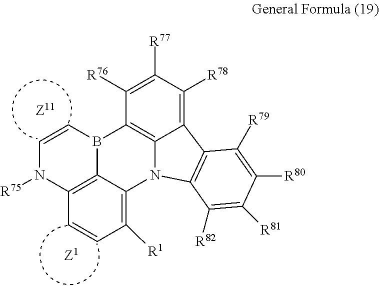

In the general formula (19), Z¹ and Z¹¹ each independently represent a furan ring fused with a substituted or unsubstituted benzene ring, a thiophene ring fused with a substituted or unsubstituted benzene ring, or an N-substituted pyrrole ring fused with a substituted or unsubstituted benzene ring,
R¹ and R⁷⁶ to R⁸² each independently represent a substituted or unsubstituted aryl group, or a substituted or unsubstituted heteroaryl group, and
R⁷⁵ represents a substituted or unsubstituted aryl group, or a substituted or unsubstituted heteroaryl group.
R¹ and Z¹, R⁷⁵ and Z¹¹, Z¹¹ and R⁷⁶, R⁷⁶ and R⁷⁷, R⁷⁷ and R⁷⁸, R⁷⁸ and R⁷⁹, R⁷⁹ and R⁸⁰, R⁸⁰ and R⁸¹, and R⁸¹ and R⁸² each can bond to each other to form a cyclic structure.

Compounds represented by the following general formula (20) are further preferred light emitting materials.

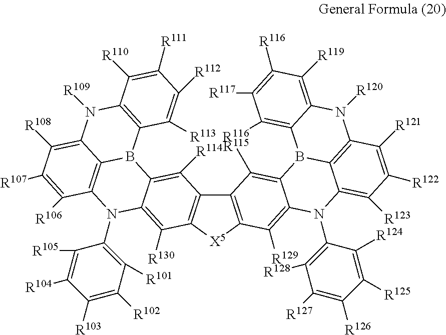

In the general formula (20), X⁵ represents an oxygen atom, a sulfur atom, or a nitrogen atom to which a substituted or unsubstituted aryl group or a substituted or unsubstituted heteroaryl group bonds,
R¹⁰¹ to R¹³⁰ each independently represents a hydrogen atom, a deuterium atom or a substituent, and
R¹⁰¹ and R¹⁰², R¹⁰² and R¹⁰³, R¹⁰³ and R¹⁰⁴, R¹⁰⁴ and R¹⁰⁵, R¹⁰⁵ and R¹⁰⁶, R¹⁰⁶ and R¹⁰⁷, R¹⁰⁷ and R¹⁰⁸, R¹⁰⁸ and R¹⁰⁹, R¹⁰⁹ and R¹⁰⁰, R¹¹⁰ and R¹¹¹, R¹¹¹ and R¹¹², R¹¹² and R¹¹³, R¹¹³ and R¹¹⁴, R¹¹⁴ and R¹¹⁵, R¹¹⁵ and R¹¹⁶, R¹¹⁶ and R¹¹⁷, R¹¹⁷ and R¹¹⁸, R¹¹⁸ and R¹¹⁹, R¹¹⁹ and R¹²⁰, R¹²⁰ and R¹²¹, R¹²¹ and R¹²², R¹²² and R¹²³, R¹²³ and R¹²⁴, R¹²⁴ and R¹²⁵, R¹²⁵ and R¹²⁶, R¹²⁶ and R¹²⁷, R¹²⁷ and R¹²⁸, R¹²⁸ and R¹²⁹, R¹²⁹ and R¹³⁰, and R¹³⁰ and R¹⁰¹ each can bond to each other to form a cyclic structure.

Compounds represented by the following general formula (21) are further preferred light emitting materials.

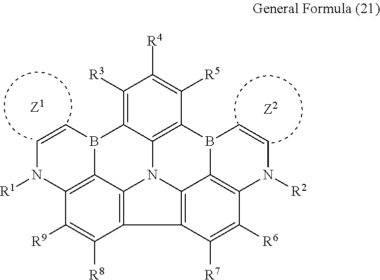

In the general formula (21), R¹ and R² each independently represent a substituted or unsubstituted alkyl group, a substituted or unsubstituted aryl group, or a substituted or unsubstituted heteroaryl group,
Z¹ and Z² each independently represent a substituted or unsubstituted aromatic ring, or a substituted or unsubstituted heteroaromatic ring, and
R³ to R⁹ each independently represent a hydrogen atom, a deuterium atom or a substituent.

However, at least one of R¹, R², Z¹ and Z² includes a substituted or unsubstituted benzofuran ring, a substituted or unsubstituted benzothiophene ring, or a substituted or unsubstituted indole ring.

R¹ and Z¹, Z¹ and R³, R³ and R⁴, R⁴ and R¹, R¹ and Z², Z² and R², R² and R⁶, R⁶ and R⁷, R¹ and R⁸, R⁸ and R⁹, and R⁹ and R¹ each can bond to each other to form a cyclic structure.

Of the benzene ring skeleton-constituting carbon atoms to constitute the benzofuran ring, the benzothiophene ring, and the indole ring, a substitutable carbon atom can be substituted with a nitrogen atom. In the general formula (1), C—R³, C—R⁴, C—R¹, C—R⁶, C—R⁷, C—R¹¹, and C—R⁹ can be substituted with N.

In one aspect of the present invention, R¹ and R² are each independently a substituted or unsubstituted alkyl group, a substituted or unsubstituted phenyl group, or a group containing at least one ring structure selected from the group consisting of a substituted or unsubstituted benzofuran ring, a substituted or unsubstituted benzothiophene ring and a substituted or unsubstituted indole ring. In one aspect of the present invention, Z¹ and Z² are each independently a substituted or unsubstituted non-fused benzene ring, a furan ring fused with a substituted or unsubstituted benzene ring, a thiophene ring fused with a substituted or unsubstituted benzene ring, a pyrrole ring fused with a substituted or unsubstituted benzene ring, a benzene ring fused with a substituted or unsubstituted benzofuran ring, a benzene ring fused with a substituted or unsubstituted benzothiophene ring, or a benzene ring fused with a substituted or unsubstituted indole ring. In one aspect of the present invention, R¹ and Z¹ bond to each other to form a cyclic structure. In one aspect of the present invention, R¹ and Z¹ bond to each other to form a pyrrole ring.

Compounds represented by the following general formula (22) are further preferred light emitting materials.

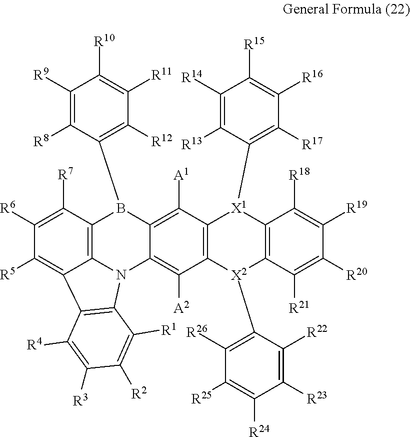

In the general formula (22), one of X¹ and X² is a nitrogen atom, and the other is a boron atom. R¹ to R²⁶, A¹ and A² each independently represent a hydrogen atom, a deuterium atom, or a substituent. R¹ and R², R² and R³, R³ and R⁴, R⁴ and R⁵, R⁵ and R⁶, R⁶ and R⁷, R⁷ and R⁸, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁷ and R¹⁸, R¹⁸ and R¹⁹, R¹⁹ and R²⁰, R²⁰ and R²¹, R²¹ and R²², R²² and R²³, R²³ and R²⁴, R²⁴ and R²⁵, and R²⁵ and R²⁶ each can bond to each other to form a cyclic structure. However, when X¹ is a nitrogen atom, R¹⁷ and R¹⁸ bond to each other to be a single bond to form a pyrrole ring, and when X² is a nitrogen atom, R²¹ and R²² bond to each other to be a single bond to form a pyrrole ring. However, in the case where X¹ is a nitrogen atom, and where R⁷ and R⁸ and R²¹ and R²² each bond to each other via a nitrogen atom to form a 6-membered ring, and R¹⁷ and R¹⁸ bond to each other to form a single bond, at least one of R¹ to R⁶ is a substituted or unsubstituted aryl group, or any of R¹ and R², R² and R³, R³ and R⁴, R⁴ and R⁵, and R⁵ and R⁶ bond to each other to form an aromatic ring or a heteroaromatic ring.

In one aspect of the present invention, at least one of R³ and R⁶ is a substituent. In one aspect of the present invention, both R³ and R⁶ are substituents. In one aspect of the present invention, the substituent represented by R³ and R⁶ is one group selected from the group consisting of an alkyl group and an aryl group, or a group obtained by combining two or more of the groups. In one aspect of the present invention, both R⁸ and R¹² are substituents. In one aspect of the present invention, the compounds are represented by the following general formula (22a).

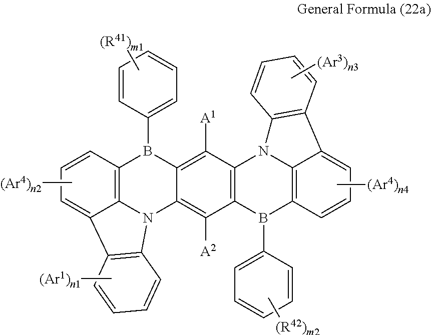

In the general formula (22a), Ar¹ to Ar⁴ each independently represent a substituted or unsubstituted aryl group, or a substituted or unsubstituted heteroaryl group. R⁴¹ and R⁴² each independently represent a substituted or unsubstituted alkyl group. m1 and m2 each independently represent an integer of 0 to 5, n1 and n3 each independently represent an integer of 0 to 4, n2 and n4 each independently represent an integer of 0 to 3. A¹ and A² each independently represent a hydrogen atom, a deuterium atom, or a substituent.

In one aspect of the present invention, A¹ and A² each are independently a group having a Hammett' σp value of more than 0.2. In one aspect of the present invention, both A¹ and A² are cyano groups. In one aspect of the present invention, both A¹ and A² are halogen atoms. One aspect of the present invention has a rotationally symmetrical structure.

Preferred examples of compounds having the above-mentioned ring-fused structure A, and compounds represented by any of the general formulae (5) to (22) are shown below.

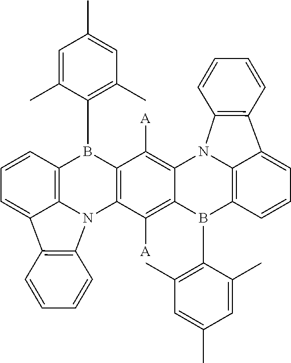

1a: A = A1
1b: A = A2
1c: A = A3
1d: A = A4
1e: A = A7
1f: A = A10
1g: A = A11
1h: A = A16
1i: A = A35
1j: A = A39
1k: A = A40
1l: A = A41
1m: A = A42

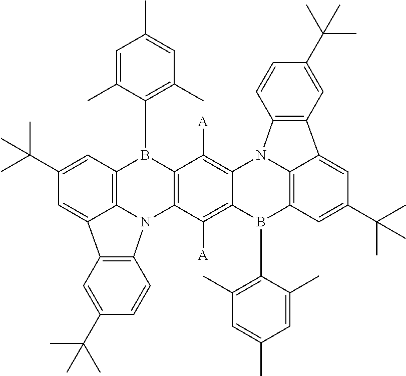

2a: A = A1
2b: A = A2
2c: A = A3
2d: A = A4
2e: A = A7
2f: A = A10
2g: A = A11
2h: A = A16
2i: A = A35
2j: A = A39
2k: A = A40
2l: A = A41
2m: A = A42

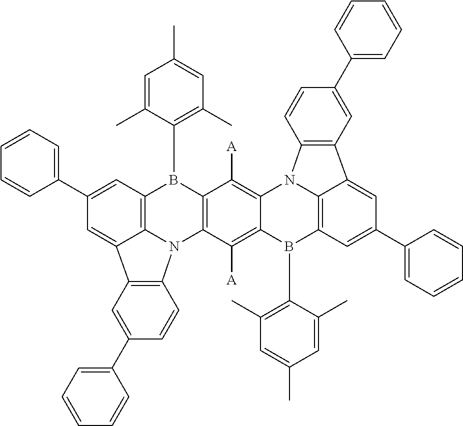

3a: A = A1
3b: A = A2
3c: A = A3
3d: A = A4
3e: A = A7
3f: A = A10
3g: A = A11
3h: A = A16
3i: A = A35
3j: A = A39
3k: A = A40
3l: A = A41
3m: A = A42

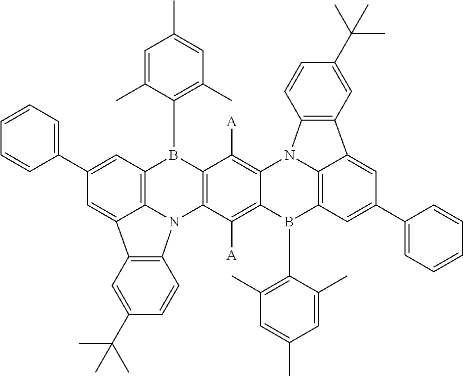

4a: A = A1
4b: A = A2
4c: A = A3
4d: A = A4
4e: A = A7
4f: A = A10
4g: A = A11
4h: A = A16
4i: A = A35
4j: A = A39
4k: A = A40
4l: A = A41
4m: A = A42

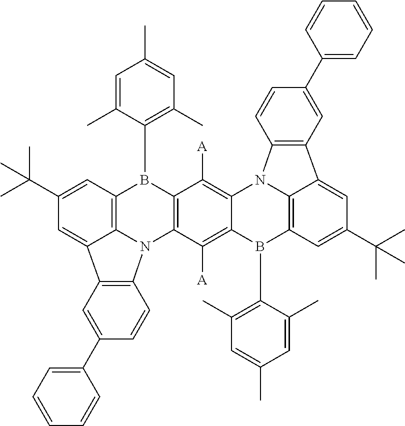

5a: A = A1
5b: A = A2
5c: A = A3
5d: A = A4
5e: A = A7
5f: A = A10
5g: A = A11
5h: A = A16
5i: A = A35
5j: A = A 39
5k: A = A40
5l: A = A41
5m: A = A42

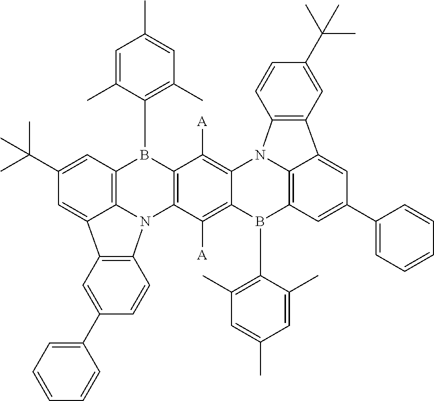

6a: A = A1
6b: A = A2
6c: A = A3
6d: A = A4
6e: A = A7
6f: A = A10
6g: A = A11
6h: A = A16
6i: A = A 35
6j: A = A39
6k: A = A40
6l: A = A41
6m: A = A42

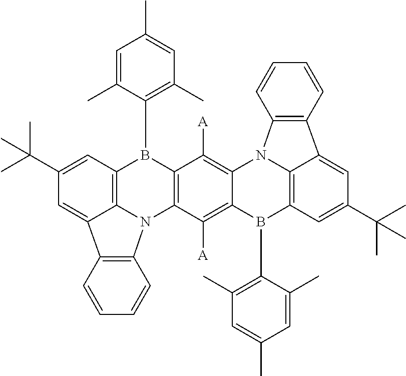

7a: A = A1
7b: A = A2
7c: A = A3
7d: A = A4
7e: A = A7
7f: A = A10
7g: A = A11
7h: A = A16
7i: A = A35
7j: A = A39
7k: A = A40
7l: A = A41
7m: A = A42

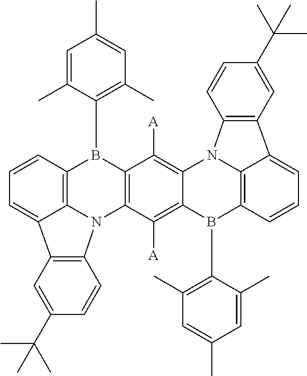

8a: A = A1
8b: A = A2
8c: A = A3
8d: A = A4
8e: A = A7
8f: A = A10
8g: A = A11
8h: A = A16
8i: A = A35
8j: A = A39
8k: A = A40
8l: A = A41
8m: A = A42

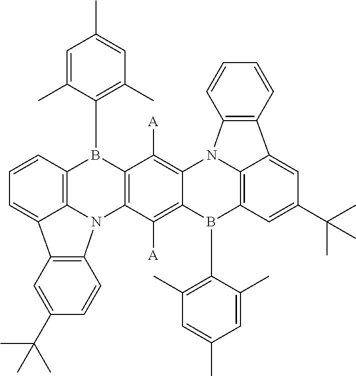

9a: A = A1
9b: A = A2
9c: A = A3
9d: A = A4
9e: A = A7
9f: A = A10
9g: A = A11
9h: A = A16
9i: A = A35
9j: A = A39
9k: A = A40
9l: A = A41
9m: A = A42

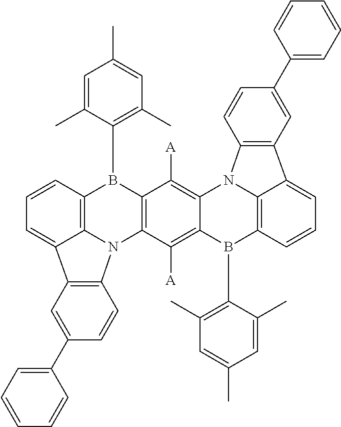

10a: A = A1
10b: A = A2
10c: A = A3
10d: A = A4
10e: A = A7
10f: A = A10
10g: A = A11
10h: A = A16
10i: A = A35
10j: A = A39
10k: A = A40
10l: A = A41
10m: A = A42

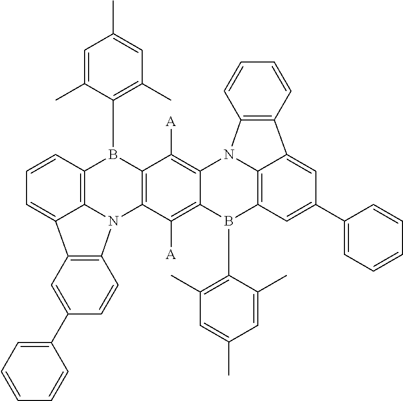

11a: A = A1
11b: A = A2
11c: A = A3
11d: A = A4
11e: A = A7
11f: A = A10
11g: A = A11
11h: A = A16
11i: A = A35
11j: A = A39
11k: A = A40
11l: A = A41
11m: A = A42

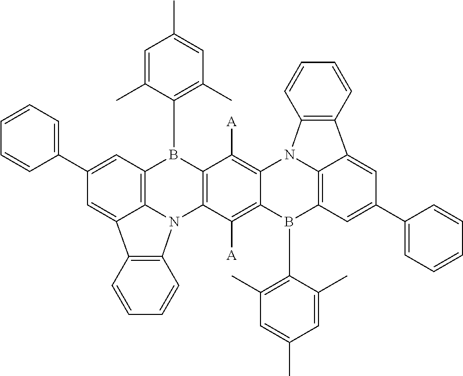

12a: A = A1
12b: A = A2
12c: A = A3
12d: A = A4
12e: A = A7
12f: A = A10
12g: A = A11
12h: A = A16
12i: A = A35
12j: A = A39
12k: A = A40
12l: A = A41
12m: A = A42

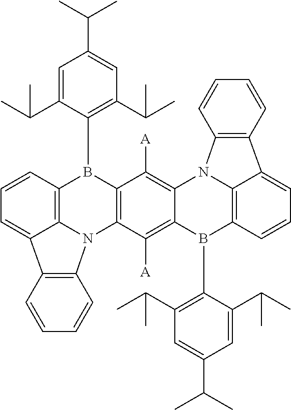

13a: A = A1
13b: A = A2
13c: A = A3
13d: A = A4
13e: A = A7
13f: A = A10
13g: A = A11
13h: A = A16
13i: A = A35
13j: A = A39
13k: A = A40
13l: A = A41
13m: A = A42

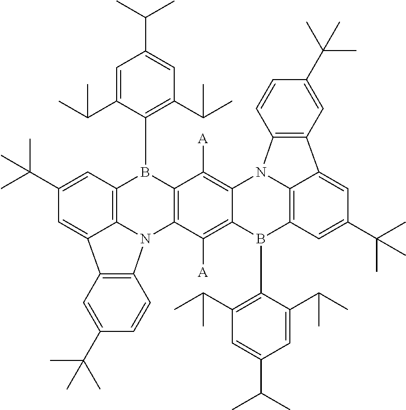

14a: A = A1
14b: A = A2
14c: A = A3
14d: A = A4
14e: A = A7
14f: A = A10
14g: A = A11
14h: A = A16
14i: A = A35
14j: A = A39
14k: A = A40
14l: A = A41
14m: A = A42

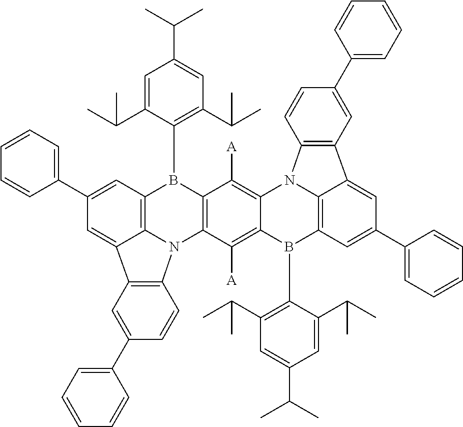

15a: A = A1
15b: A = A2
15c: A = A3
15d: A = A4
15e: A = A7
15f: A = A10
15g: A = A11
15h: A = A16
15i: A = A35
15j: A = A39
15k: A = A40
15l: A = A41
15m: A = A42

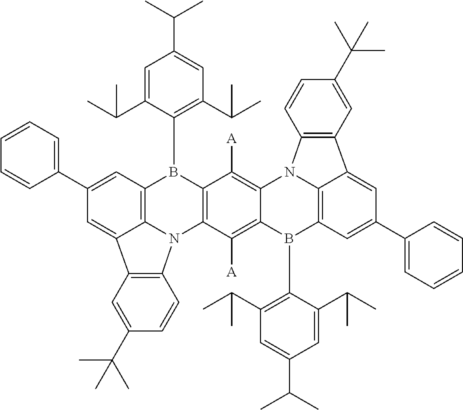

16a: A = A1
16b: A = A2
16c: A = A3
16d: A = A4
16e: A = A7
16f: A = A10
16g: A = A11
16h: A = A16
16i: A = A35
16j: A = A39
16k: A = A40
16l: A = A41
16m: A = A42

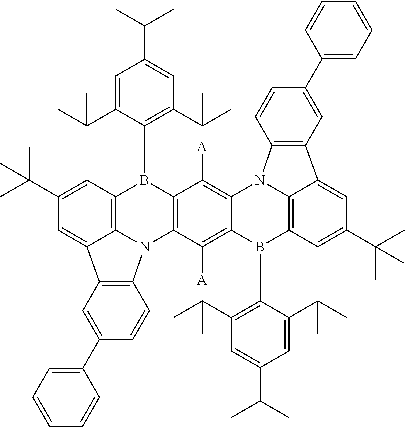

17a: A = A1
17b: A = A2
17c: A = A3
17d: A = A4
17e: A = A7
17f: A = A10
17g: A = A11
17h: A = A16
17i: A = A35
17j: A = A39
17k: A = A40
17l: A = A41
17m: A = A42

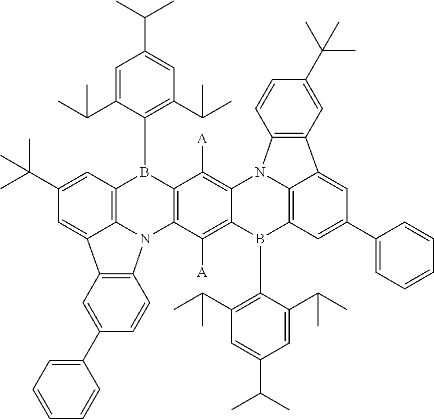

18a: A = A1
18b: A = A2
18c: A = A3
18d: A = A4
18e: A = A7
18f: A = A10
18g: A = A11
18h: A = A16
18i: A = A35
18j: A = A39
18k: A = A40
18l: A = A41
18m: A = A42

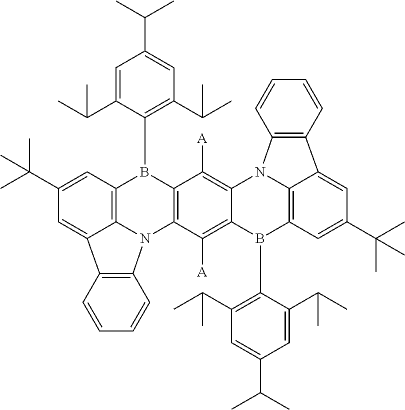

19a: A = A1
19b: A = A2
19c: A = A3
19d: A = A4
19e: A = A7
19f: A = A10
19g: A = A11
19h: A = A16
19i: A = A35
19j: A = A39
19k: A = A40
19l: A = A41
19m: A = A42

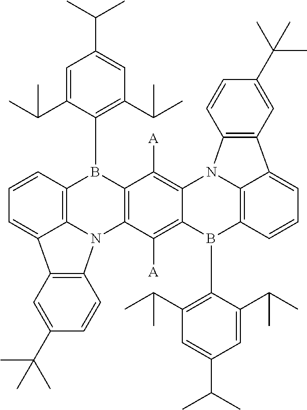

20a: A = A1
20b: A = A2
20c: A = A3
20d: A = A4
20e: A = A7
20f: A = A10
20g: A = A11
20h: A = A16
20i: A = A35
20j: A = A39
20k: A = A40
20l: A = A41
20m: A = A42

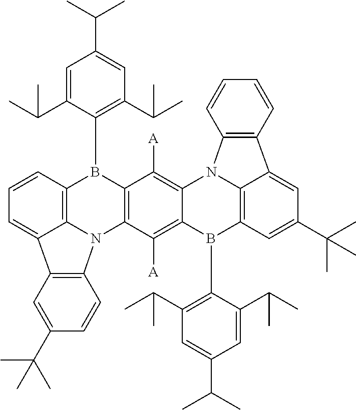

21a: A = A1
21b: A = A2
21c: A = A3
21d: A = A4
21e: A = A7
21f: A = A10
21g: A = A11
21h: A = A16
21i: A = A35
21j: A = A39
21k: A = A40
21l: A = A41
21m: A = A42

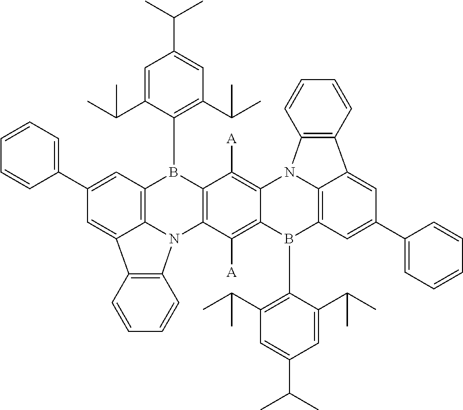

22a: A = A1
22b: A = A2
22c: A = A3
22d: A = A4
22e: A = A7
22f: A = A10
22g: A = A11
22h: A = A16
22i: A = A35
22j: A = A39
22k: A = A40
22l: A = A41
22m: A = A42

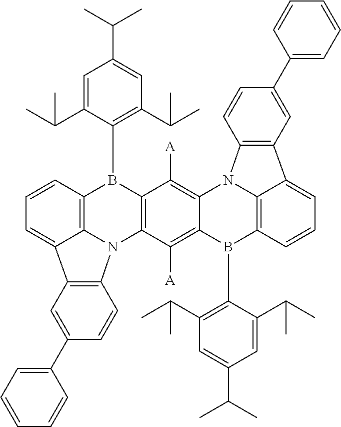

23a: A = A1
23b: A = A2
23c: A = A3
23d: A = A4
23e: A = A7
23f: A = A10
23g: A = A11
23h: A = A16
23i: A = A35
23j: A = A39
23k: A = A40
23l: A = A41
23m: A = A42

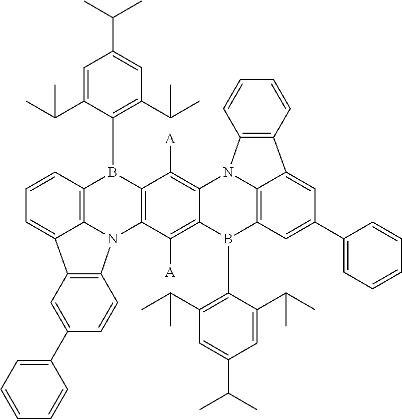

24a: A = A1
24b: A = A2
24c: A = A3
24d: A = A4
24e: A = A7
24f: A = A10
24g: A = A11
24h: A = A16
24i: A = A35
24j: A = A39
24k: A = A40
24l: A = A41
24m: A = A42

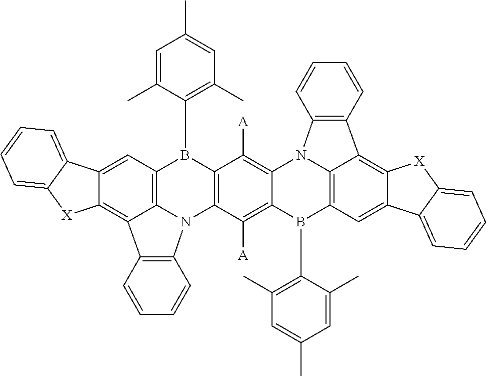

25a: A = A1
25b: A = A2
25c: A = A3
25d: A = A4
25e: A = A7
25f: A = A10
25g: A = A11
25h: A = A16
25i: A = A35
25j: A = A39
25k: A = A40
25l: A = A41
25m: A = A42

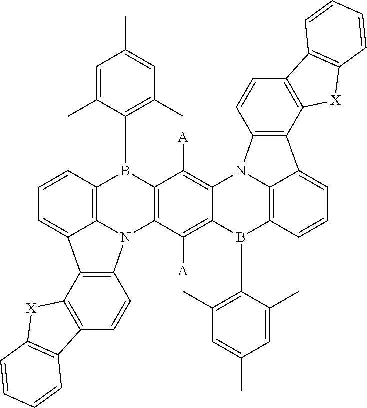

26a: A = A1
26b: A = A2
26c: A = A3
26d: A = A4
26e: A = A7
26f: A = A10
26g: A = A11
26h: A = A16
26i: A = A35
26j: A = A39
26k: A = A40
26l: A = A41
26m: A = A42

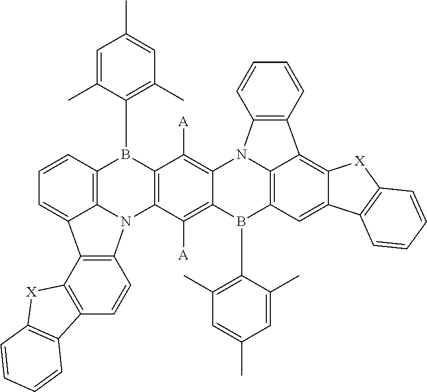

27a: A = A1
27b: A = A2
27c: A = A3
27d: A = A4
27e: A = A7
27f: A = A10
27g: A = A11
27h: A = A16
27i: A = A35
27j: A = A39
27k: A = A40
27l: A = A41
27m: A = A42

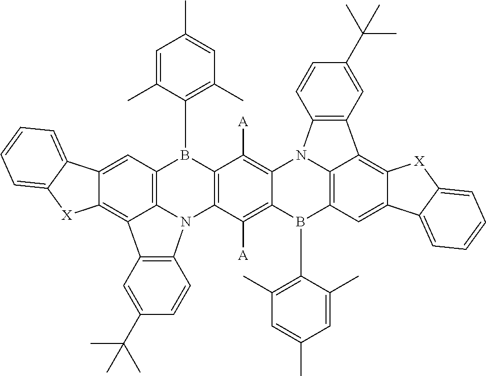

28a: A = A1
28b: A = A2
28c: A = A3
28d: A = A4
28e: A = A7
28f: A = A10
28g: A = A11
28h: A = A16
28i: A = A35
28j: A = A39
28k: A = A40
28l: A = A41
28m: A = A42

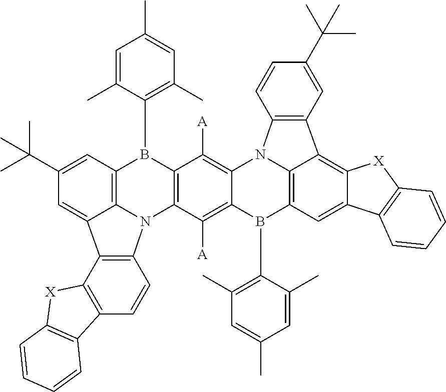

29a: A = A1
29b: A = A2
29c: A = A3
29d: A = A4
29e: A = A7
29f: A = A10
29g: A = A11
29h: A = A16
29i: A = A35
29j: A = A39
29k: A = A40
29l: A = A41
29m: A = A42

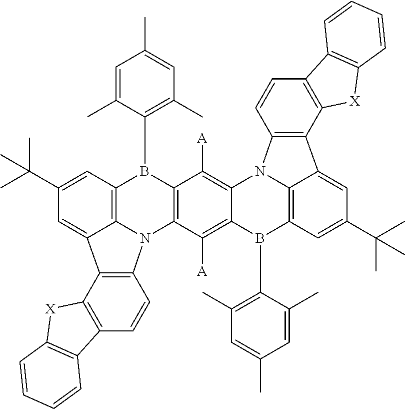

30a: A = A1
30b: A = A2
30c: A = A3
30d: A = A4
30e: A = A7
30f: A = A10
30g: A = A11
30h: A = A16
30i: A = A35
30j: A = A39
30k: A = A40
30l: A = A41
30m: A = A42

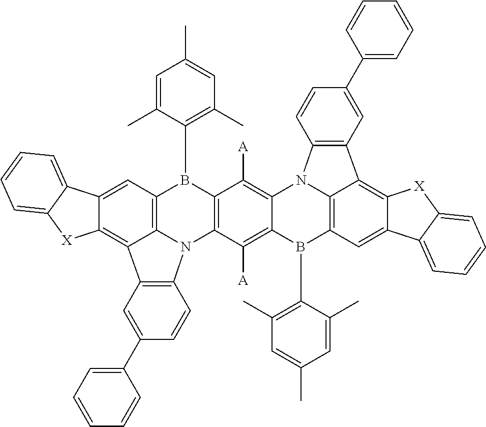

31a: A = A1
31b: A = A2
31c: A = A3
31d: A = A4
31e: A = A7
31f: A = A10
31g: A = A11
31h: A = A16
31i: A = A35
31j: A = A39
31k: A = A40
31l: A = A41
31m: A = A42

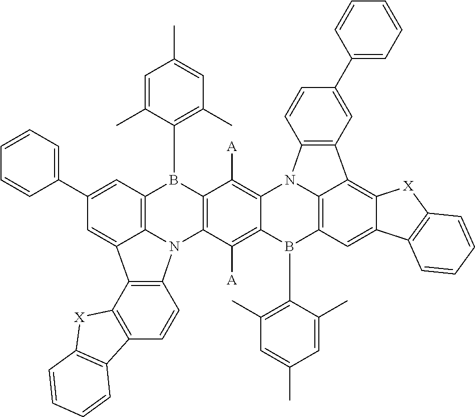

32a: A = A1
32b: A = A2
32c: A = A3
32d: A = A4
32e: A = A7
32f: A = A10
32g: A = A11
32h: A = A16
32i: A = A35
32j: A = A39
32k: A = A40
32l: A = A41
32m: A = A42

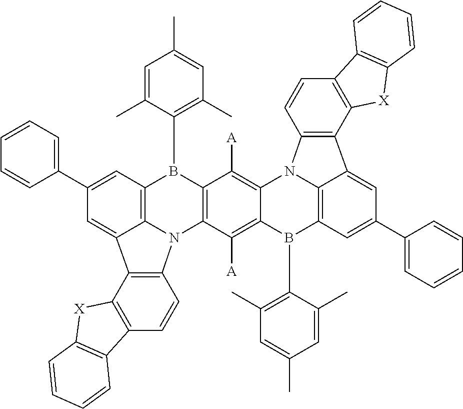

33a: A = A1
33b: A = A2
33c: A = A3
33d: A = A4
33e: A = A7
33f: A = A10
33g: A = A11
33h: A = A16
33i: A = A35
33j: A = A39
33k: A = A40
33l: A = A41
33m: A = A42

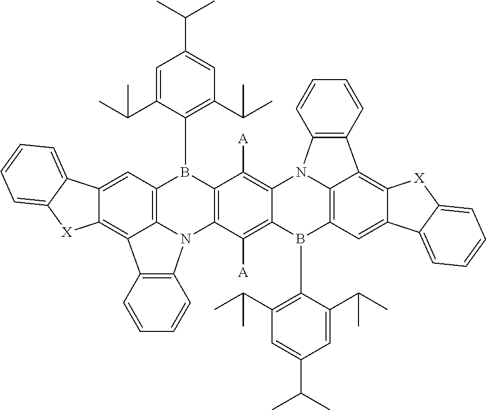

34a: A = A1
34b: A = A2
34c: A = A3
34d: A = A4
34e: A = A7
34f: A = A10
34g: A = A11
34h: A = A16
34i: A = A35
34j: A = A39
34k: A = A40
34l: A = A41
34m: A = A42

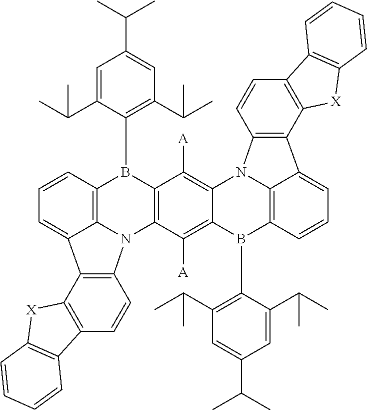

35a: A = A1
35b: A = A2
35c: A = A3
35d: A = A4
35e: A = A7
35f: A = A10
35g: A = A11
35h: A = A16
35i: A = A35
35j: A = A39
35k: A = A40
35l: A = A41
35m: A = A42

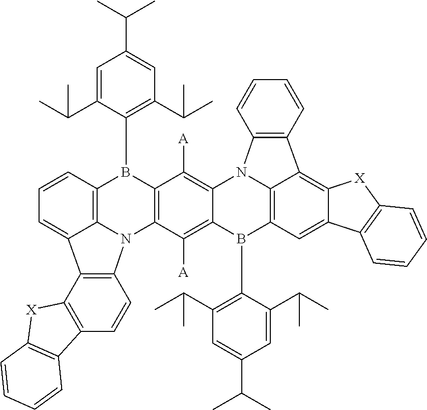

36a: A = A1
36b: A = A2
36c: A = A3
36d: A = A4
36e: A = A7
36f: A = A10
36g: A = A11
36h: A = A16
36i: A = A35
36j: A = A39
36k: A = A40
36l: A = A41
36m: A = A42

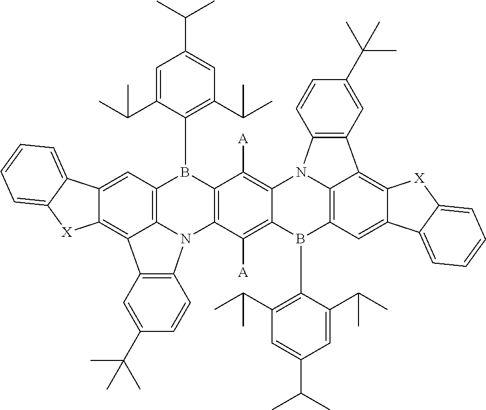

37a: A = A1
37b: A = A2
37c: A = A3
37d: A = A4
37e: A = A7
37f: A = A10
37g: A = A11
37h: A = A16
37i: A = A35
37j: A = A39
37k: A = A40
37l: A = A41
37m: A = A42

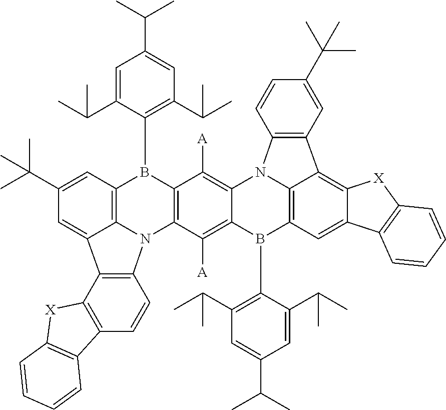

38a: A = A1
38b: A = A2
38c: A = A3
38d: A = A4
38e: A = A7
38f: A = A10
38g: A = A11
38h: A = A16
38i: A = A35
38j: A = A39
38k: A = A40
38l: A = A41
38m: A = A42

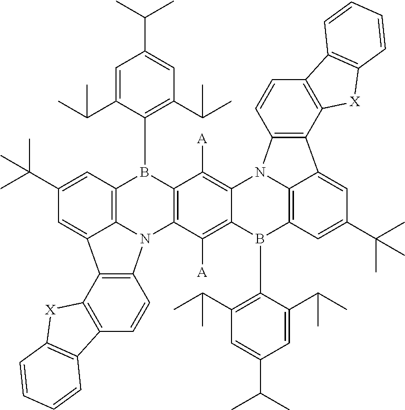

39a: A = A1
39b: A = A2
39c: A = A3
39d: A = A4
39e: A = A7
39f: A = A10
39g: A = A11
39h: A = A16
39i: A = A35
39j: A = A39
39k: A = A40
39l: A = A41
39m: A = A42

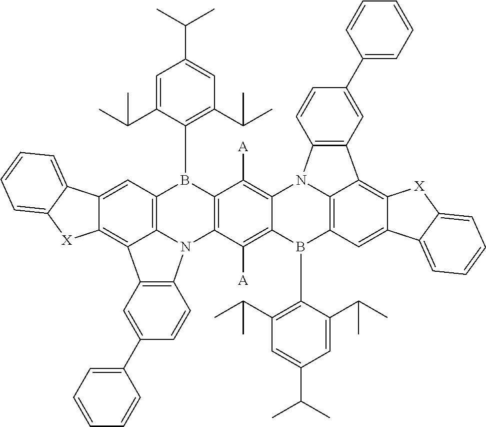

40a: A = A1
40b: A = A2
40c: A = A3
40d: A = A4
40e: A = A7
40f: A = A10
40g: A = A11
40h: A = A16
40i: A = A35
40j: A = A39
40k: A = A40
40l: A = A41
40m: A = A42

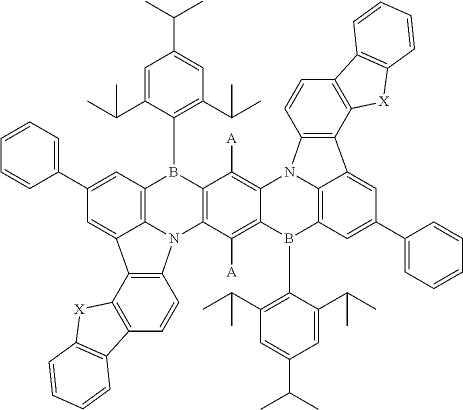

41a: A = A1
41b: A = A2
41c: A = A3
41d: A = A4
41e: A = A7
41f: A = A10
41g: A = A11
41h: A = A16
41i: A = A35
41j: A = A39
41k: A = A40
41l: A = A41
41m: A = A 42

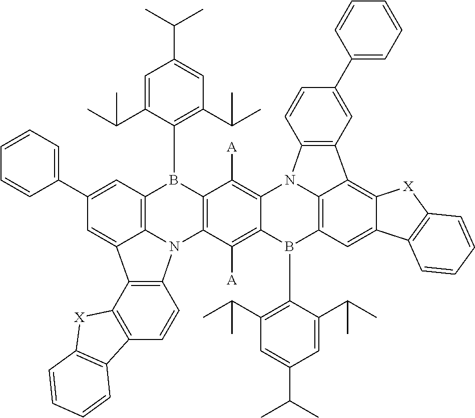

42a: A = A1
42b: A = A2
42c: A = A3
42d: A = A4
42e: A = A7
42f: A = A10
42g: A = A11
42h: A = A16
42i: A = A35
42j: A = A39
42k: A = A40
42l: A = A41
42m: A = A42

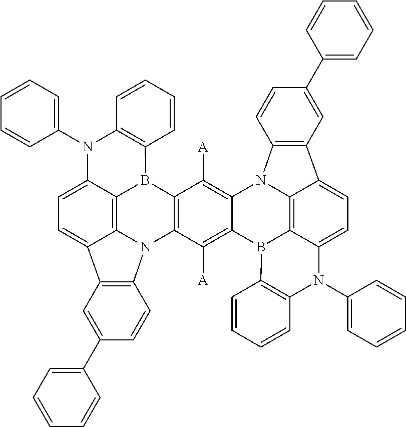

43a: A = A1
43b: A = A2
43c: A = A3
43d: A = A4
43e: A = A7
43f: A = A10
43g: A = A11
43h: A = A16
43i: A = A39
43j: A = A35
43k: A = A40
43l: A = A41
43m: A = A42

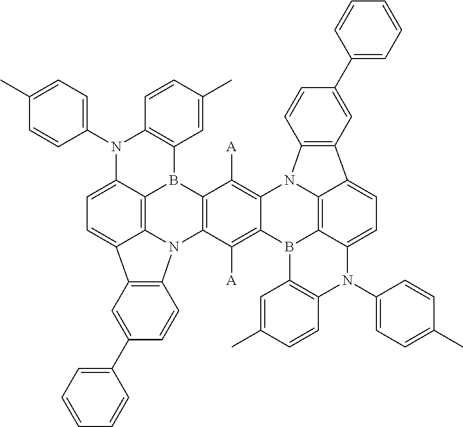

44a: A = A1
44b: A = A2
44c: A = A3
44d: A = A4
44e: A = A7
44f: A = A10
44g: A = A11
44h: A = A16
44i: A = A35
44j: A = A39
44k: A = A40
44l: A = A41
44m: A = A42

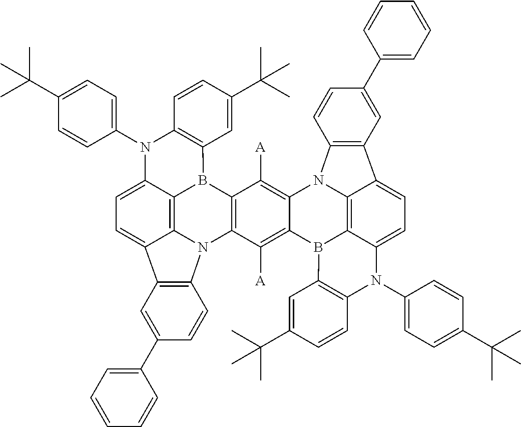

45a: A = A1
45b: A = A2
45c: A = A3
45d: A = A4
45e: A = A7
45f: A = A10
45g: A = A11
45h: A = A16
45i: A = A35
45j: A = A39
45k: A = A40
45l: A = A41
45m: A = A42

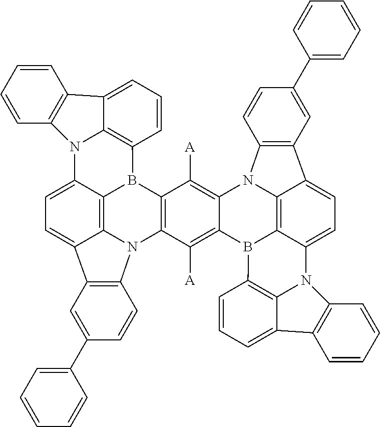

46a: A = A1
46b: A = A2
46c: A = A3
46d: A = A4
46e: A = A7
46f: A = A10
46g: A = A11
46h: A = A16
46i: A = A35
46j: A = A39
46k: A = A40
46l: A = A41
46m: A = A42

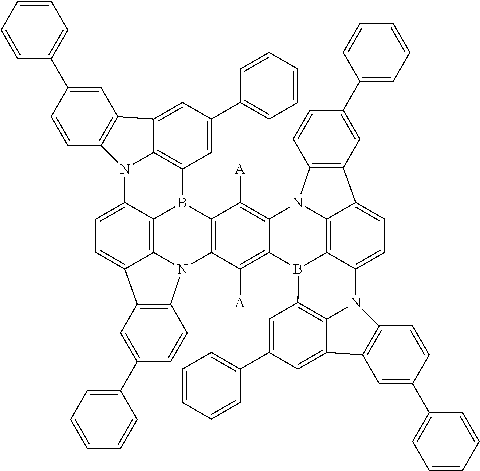

47a: A = A1
47b: A = A2
47c: A = A3
47d: A = A4
47e: A = A7
47f: A = A10
47g: A = A11
47h: A = A16
47i: A = A35
47j: A = A39
47k: A = A40
47l: A = A41
47m: A = A42

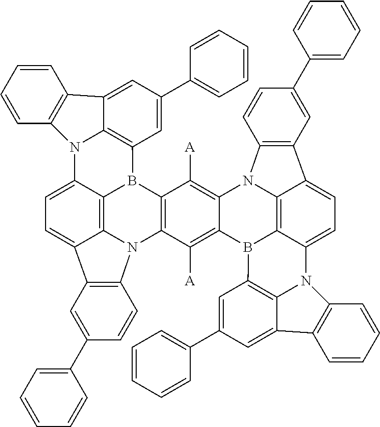

48a: A = A1
48b: A = A2
48c: A = A3
48d: A = A4
48e: A = A7
48f: A = A10
48g: A = A11
48h: A = A16
48i: A = A35
48j: A = A39
48k: A = A40
48l: A = A41
48m: A = A42

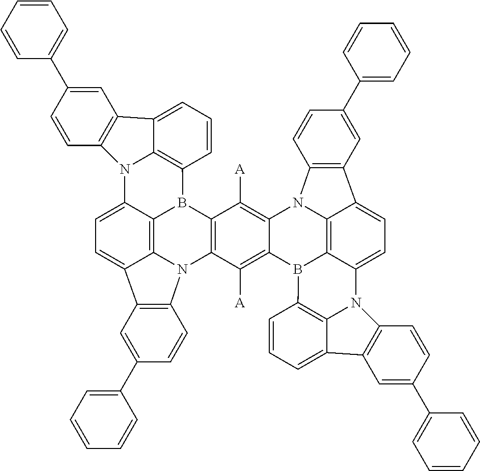

49a: A = A1
49b: A = A2
49c: A = A3
49d: A = A4
49e: A = A7
49f: A = A10
49g: A = A11
49h: A = A16
49i: A = A35
49j: A = A39
49k: A = A40
49l: A = A41
49m: A = A42

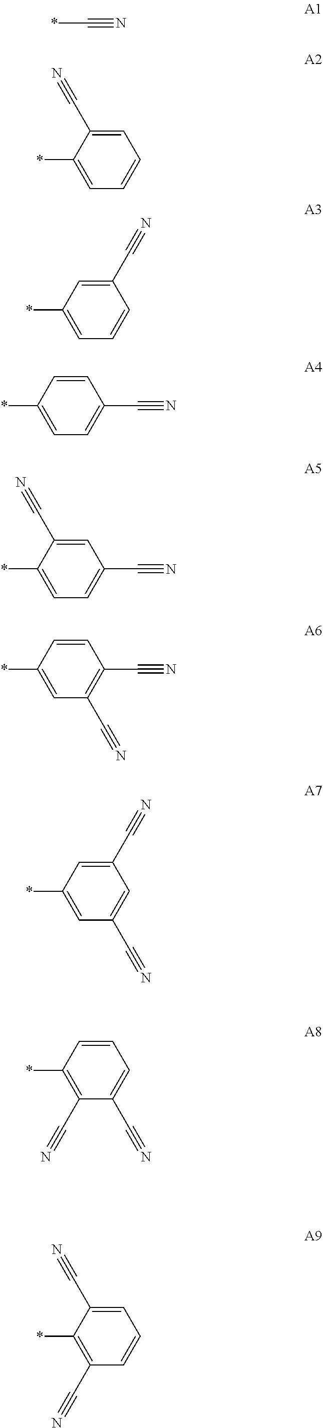

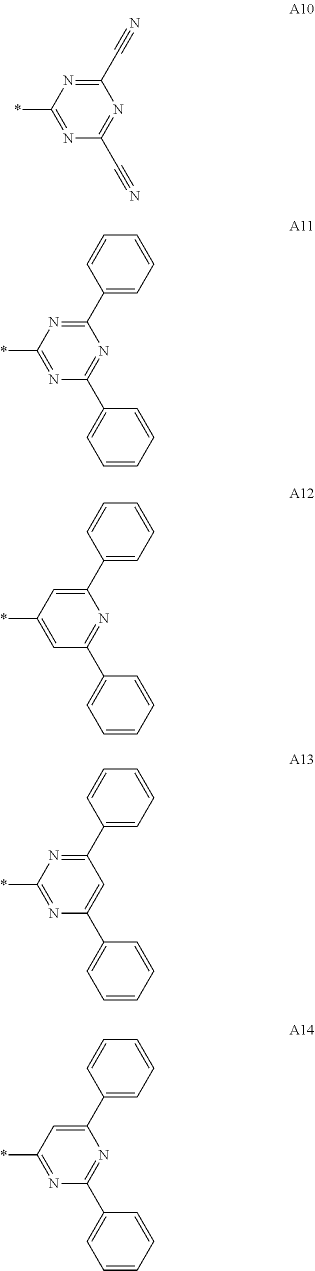

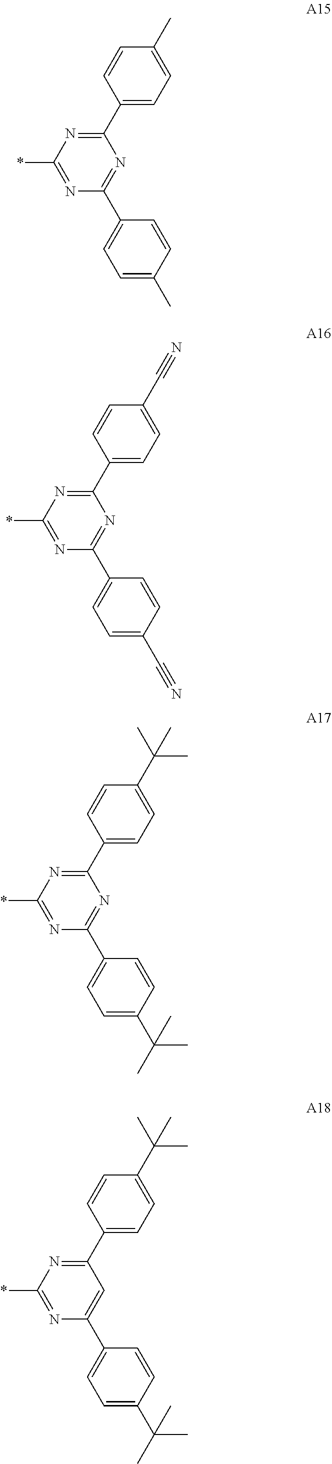

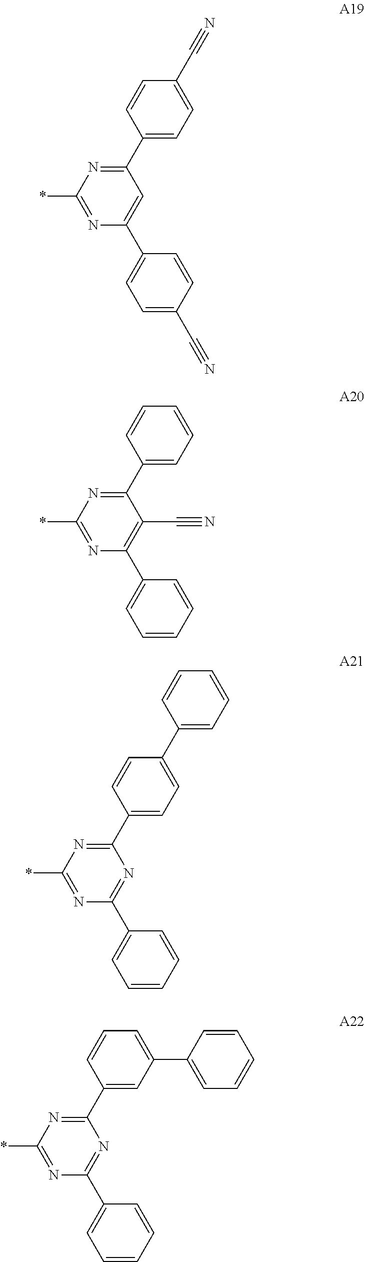

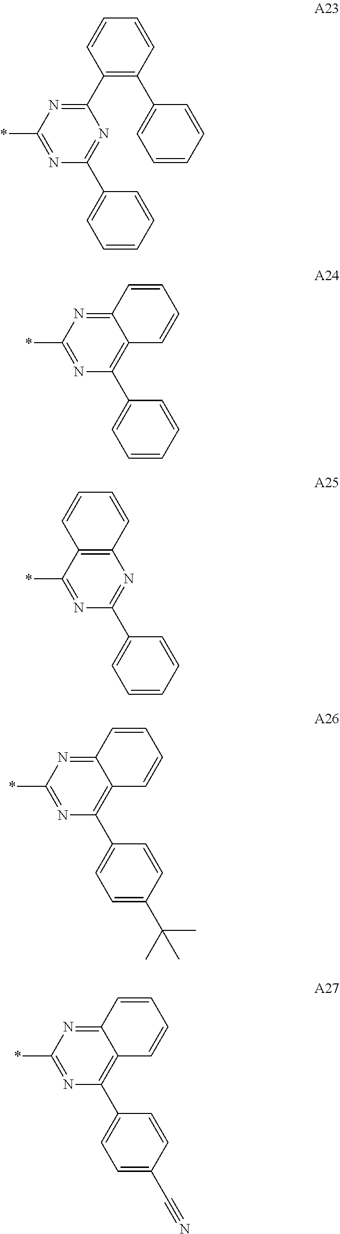

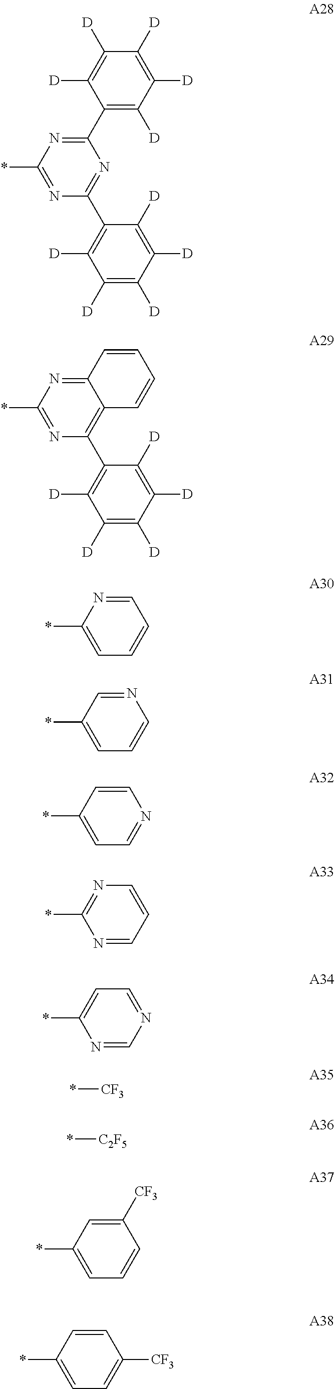

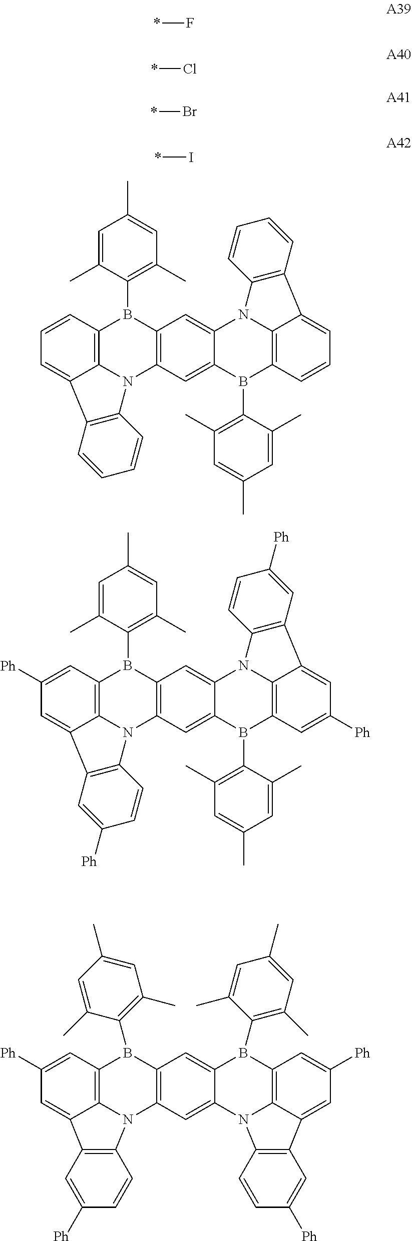

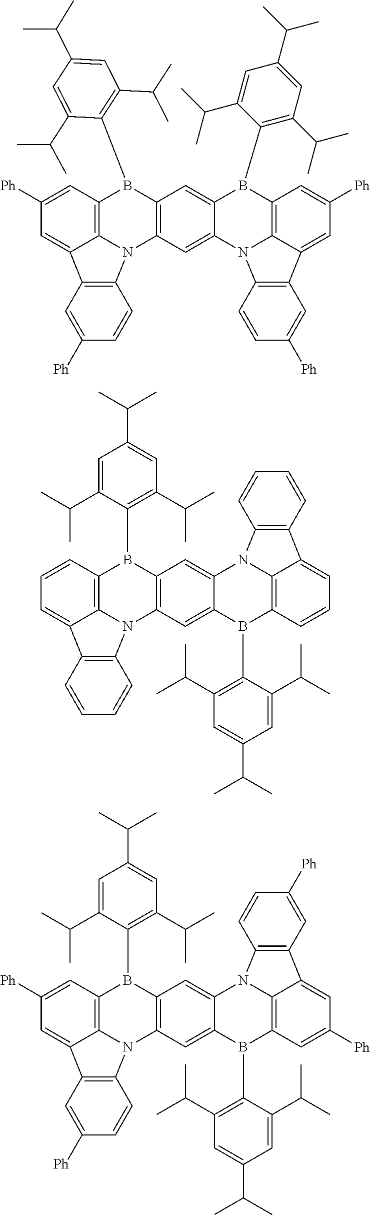

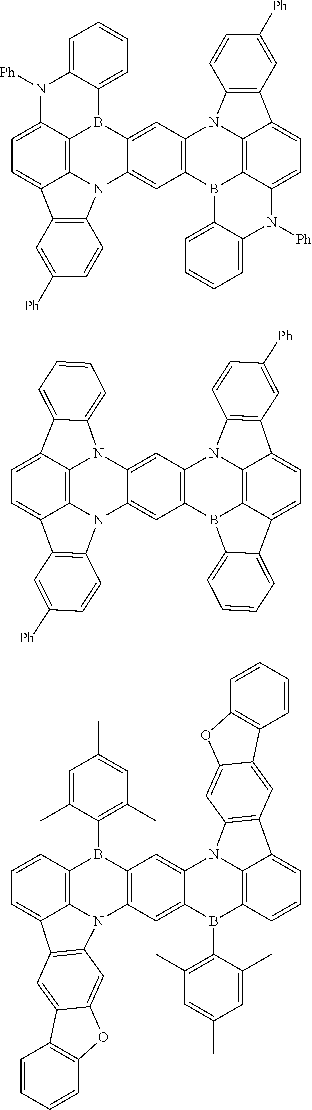 [SMILES: c1ccc(-c2ccc3c(c2)c2ccc4c5c2n3-c2cc3c(cc2B5c2ccccc2N4c2ccccc2)-n2c4ccc(-c5ccccc5)cc4c4ccc5c(c42)B3c2ccccc2N5c2ccccc2)cc1 ; c1ccc(-c2ccc3c(c2)c2ccc4c5c2n3-c2cc3c(cc2B5c2ccccc2-4)n2c4ccc(-c5ccccc5)cc4c4ccc5c6ccccc6n3c5c42)cc1 ; Cc1cc(C)c(B2c3cc4c(cc3-n3c5cc6oc7ccccc7c6cc5c5cccc2c53)B(c2c(C)cc(C)cc2C)c2cccc3c5cc6c(cc5n-4c23)oc2ccccc26)c(C)c1]

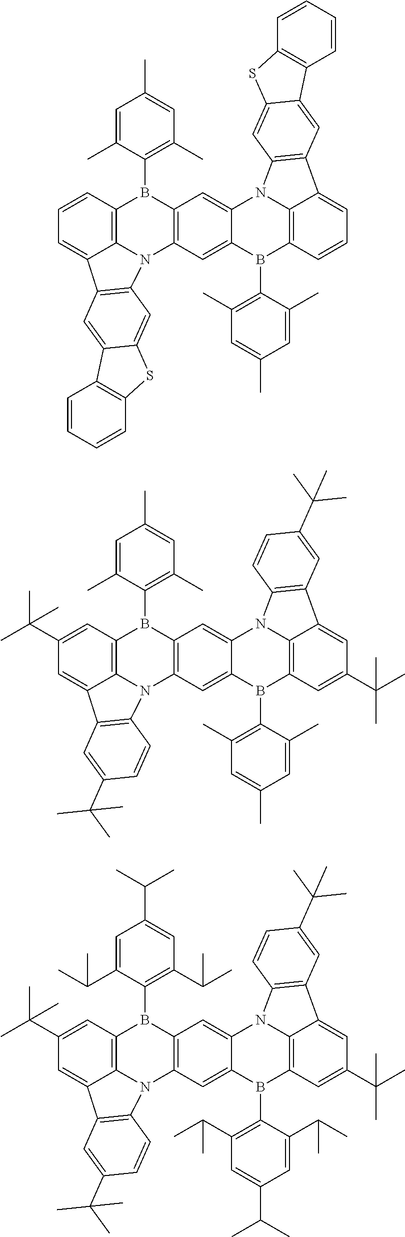

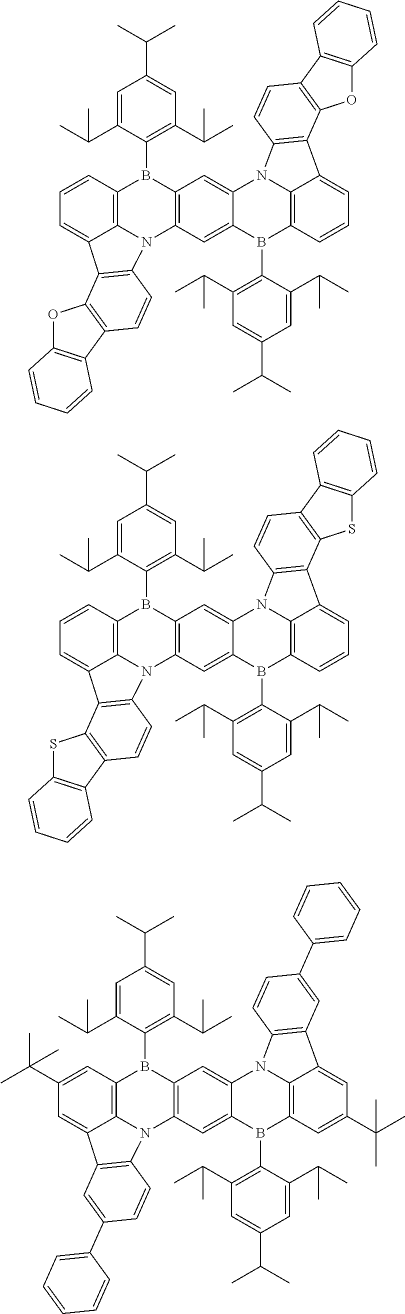 [SMILES: CC(C)c1cc(C(C)C)c(B2c3cc4c(cc3-n3c5ccc6c7ccccc7oc6c5c5cccc2c53)B(c2c(C(C)C)cc(C(C)C)cc2C(C)C)c2cccc3c5c6oc7ccccc7c6ccc5n-4c23)c(C(C)C)c1 ; CC(C)c1cc(C(C)C)c(B2c3cc4c(cc3-n3c5ccc6c7ccccc7sc6c5c5cccc2c53)B(c2c(C(C)C)cc(C(C)C)cc2C(C)C)c2cccc3c5c6sc7ccccc7c6ccc5n-4c23)c(C(C)C)c1 ; CC(C)c1cc(C(C)C)c(B2c3cc4c(cc3-n3c5ccc(-c6ccccc6)cc5c5cc(C(C)(C)C)cc2c53)B(c2c(C(C)C)cc(C(C)C)cc2C(C)C)c2cc(C(C)(C)C)cc3c5cc(-c6ccccc6)ccc5n-4c23)c(C(C)C)c1]

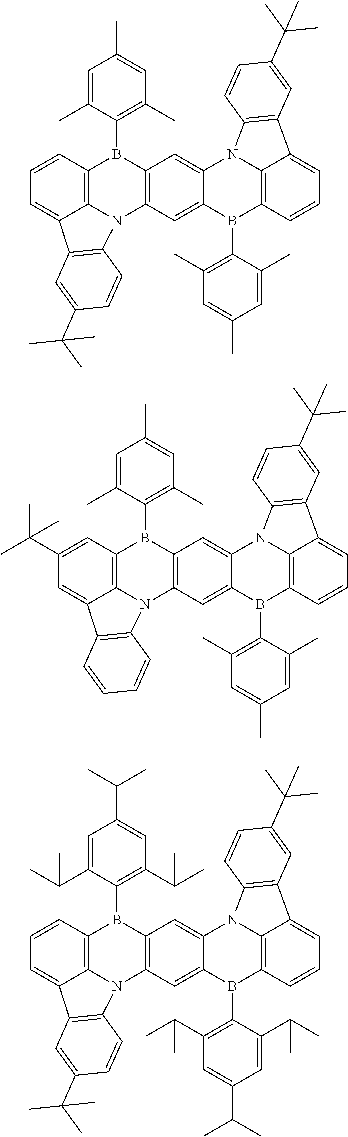

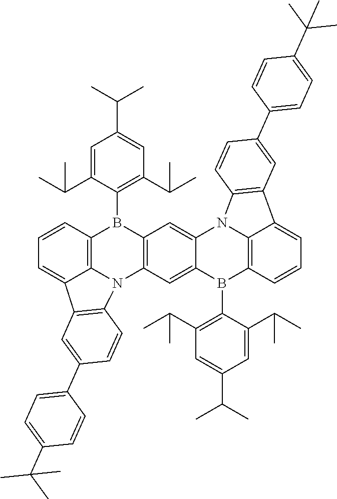 [SMILES: CC(C)c1cc(C(C)C)c(B2c3cc4c(cc3-n3c5ccc(-c6ccc(C(C)(C)C)cc6)cc5c5cccc2c53)B(c2c(C(C)C)cc(C(C)C)cc2C(C)C)c2cccc3c5cc(-c6ccc(C(C)(C)C)cc6)ccc5n-4c23)c(C(C)C)c1]

In some embodiments where a host material is used, the amount of the compound of the present invention contained in a light emitting layer as a light emitting material is 0.1% by weight or more. In some embodiments where a host material is used, the amount of the compound of the present invention contained in a light emitting layer as a light emitting material is 1% by weight or more. In some embodiments where a host material is used, the amount of the compound of the present invention contained in a light emitting layer as a light emitting material is 50% by weight or less. In some embodiments where a host material is used, the amount of the compound of the present invention contained in a light emitting layer as a light emitting material is 20% by weight or less. In some embodiments where a host material is used, the amount of the compound of the present invention contained in a light emitting layer as a light emitting material is 10% by weight or less.

In some embodiments, the host material in a light emitting layer is an organic compound having a hole transporting capability and an electron transporting capability. In some embodiments, the host material in a light emitting layer is an organic compound that prevents increase in the wavelength of emitted light. In some embodiments, the host material in a light emitting layer is an organic compound having a high glass transition temperature.

In some embodiments, the host material is selected from the group consisting of the followings:

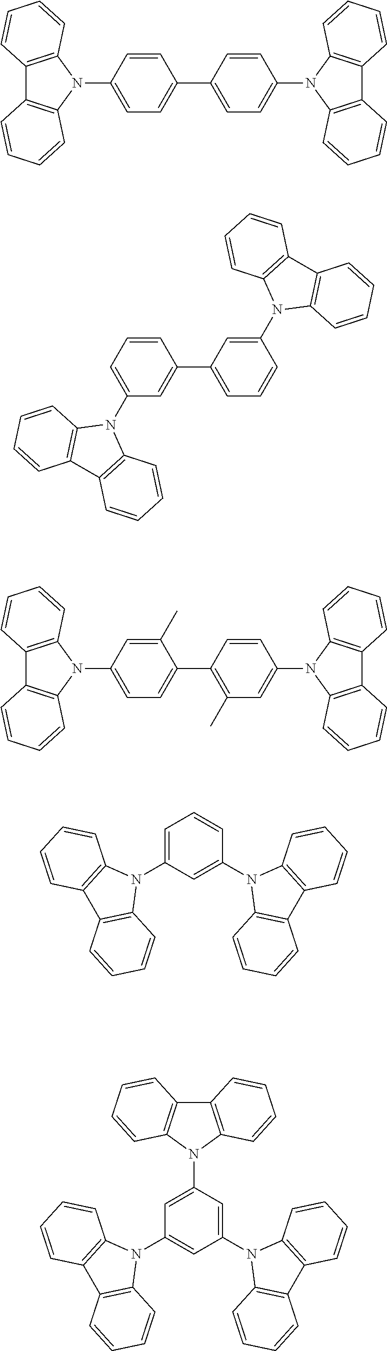 [SMILES: c1ccc2c(c1)c1ccccc1n2-c1ccc(-c2ccc(-n3c4ccccc4c4ccccc43)cc2)cc1 ; c1cc(-c2cccc(-n3c4ccccc4c4ccccc43)c2)cc(-n2c3ccccc3c3ccccc32)c1 ; Cc1cc(-n2c3ccccc3c3ccccc32)ccc1-c1ccc(-n2c3ccccc3c3ccccc32)cc1C ; c1cc(-n2c3ccccc3c3ccccc32)cc(-n2c3ccccc3c3ccccc32)c1 ; c1ccc2c(c1)c1ccccc1n2-c1cc(-n2c3ccccc3c3ccccc32)cc(-n2c3ccccc3c3ccccc32)c1]

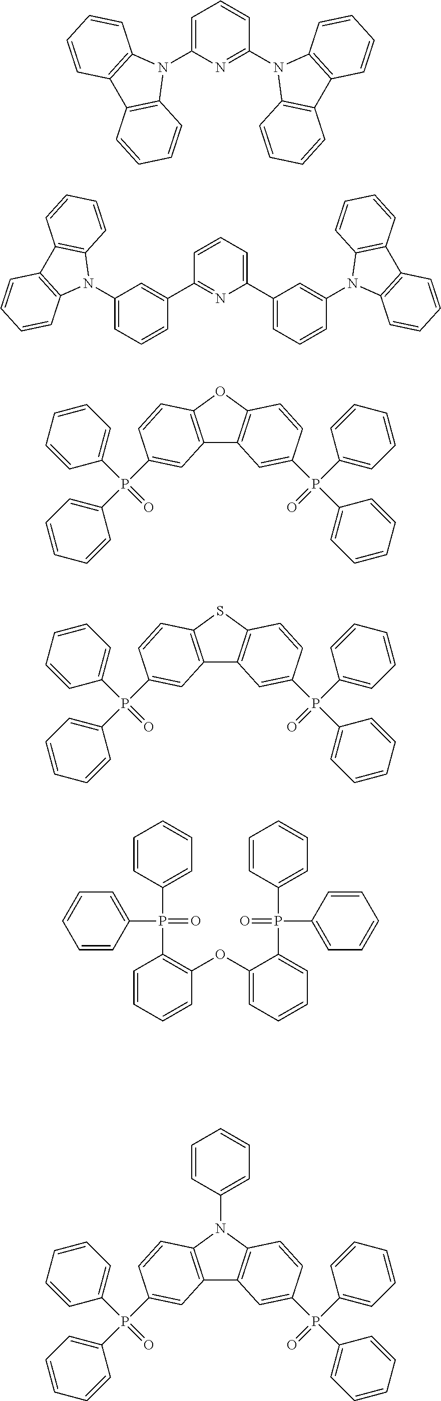 [SMILES: c1cc(-n2c3ccccc3c3ccccc32)nc(-n2c3ccccc3c3ccccc32)c1 ; c1cc(-c2cccc(-c3cccc(-n4c5ccccc5c5ccccc54)c3)n2)cc(-n2c3ccccc3c3ccccc32)c1 ; O=P(c1ccccc1)(c1ccccc1)c1ccc2oc3ccc(P(=O)(c4ccccc4)c4ccccc4)cc3c2c1 ; O=P(c1ccccc1)(c1ccccc1)c1ccc2sc3ccc(P(=O)(c4ccccc4)c4ccccc4)cc3c2c1 ; O=P(c1ccccc1)(c1ccccc1)c1ccccc1Oc1ccccc1P(=O)(c1ccccc1)c1ccccc1 ; O=P(c1ccccc1)(c1ccccc1)c1ccc2c(c1)c1cc(P(=O)(c3ccccc3)c3ccccc3)ccc1n2-c1ccccc1]

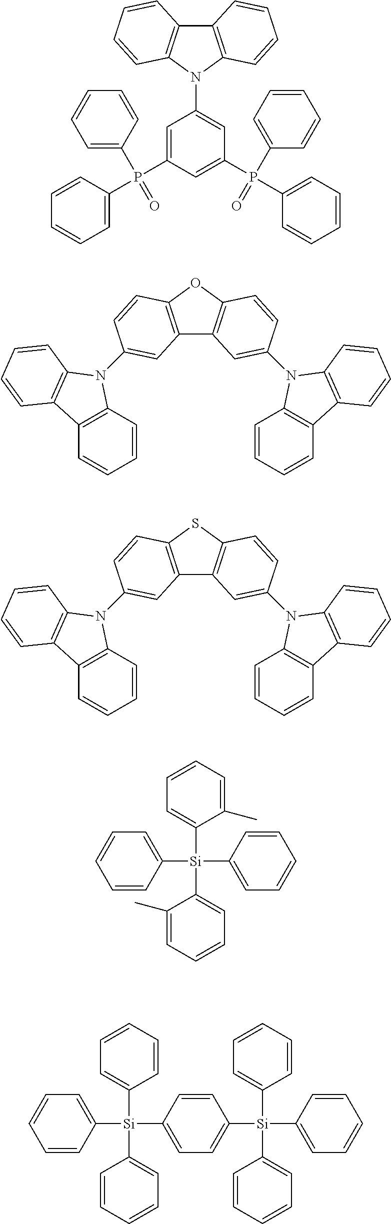 [SMILES: O=P(c1ccccc1)(c1ccccc1)c1cc(-n2c3ccccc3c3ccccc32)cc(P(=O)(c2ccccc2)c2ccccc2)c1 ; c1ccc2c(c1)c1ccccc1n2-c1ccc2oc3ccc(-n4c5ccccc5c5ccccc54)cc3c2c1 ; c1ccc2c(c1)c1ccccc1n2-c1ccc2sc3ccc(-n4c5ccccc5c5ccccc54)cc3c2c1 ; Cc1ccccc1[Si](c1ccccc1)(c1ccccc1)c1ccccc1C ; c1ccc([Si](c2ccccc2)(c2ccccc2)c2ccc([Si](c3ccccc3)(c3ccccc3)c3ccccc3)cc2)cc1]

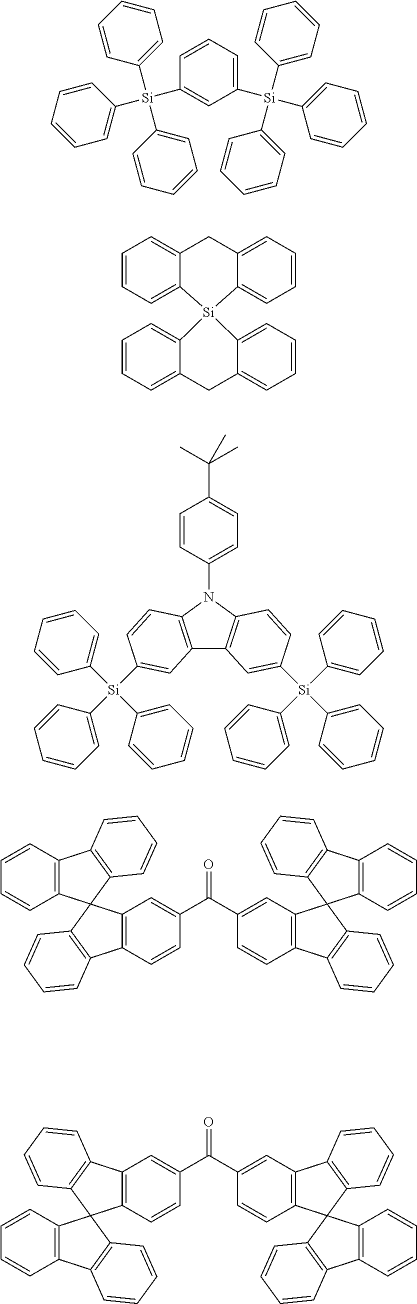 [SMILES: c1ccc([Si](c2ccccc2)(c2ccccc2)c2cccc([Si](c3ccccc3)(c3ccccc3)c3ccccc3)c2)cc1 ; c1ccc2c(c1)Cc1ccccc1[Si]21c2ccccc2Cc2ccccc21 ; CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1 ; O=C(c1ccc2c(c1)C1(c3ccccc3-c3ccccc31)c1ccccc1-2)c1ccc2c(c1)C1(c3ccccc3-c3ccccc31)c1ccccc1-2 ; O=C(c1ccc2c(c1)-c1ccccc1C21c2ccccc2-c2ccccc21)c1ccc2c(c1)-c1ccccc1C21c2ccccc2-c2ccccc21]

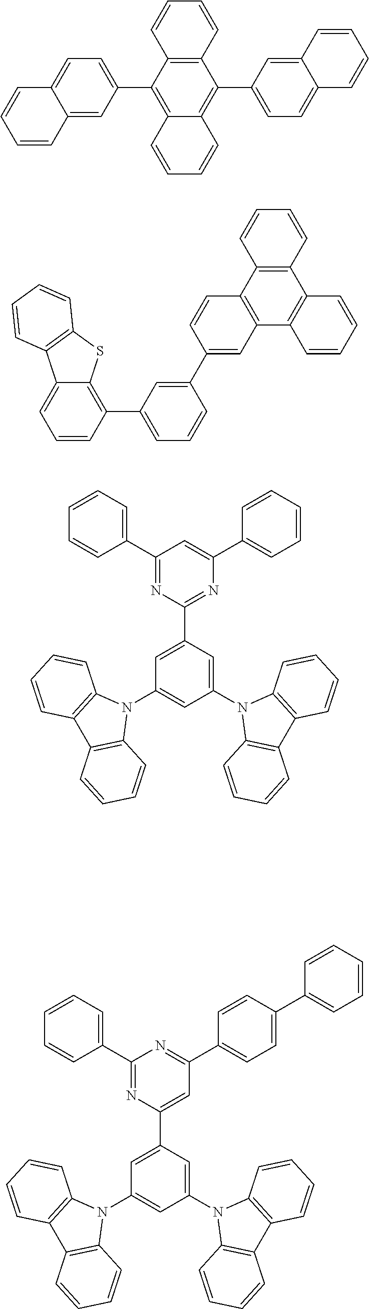

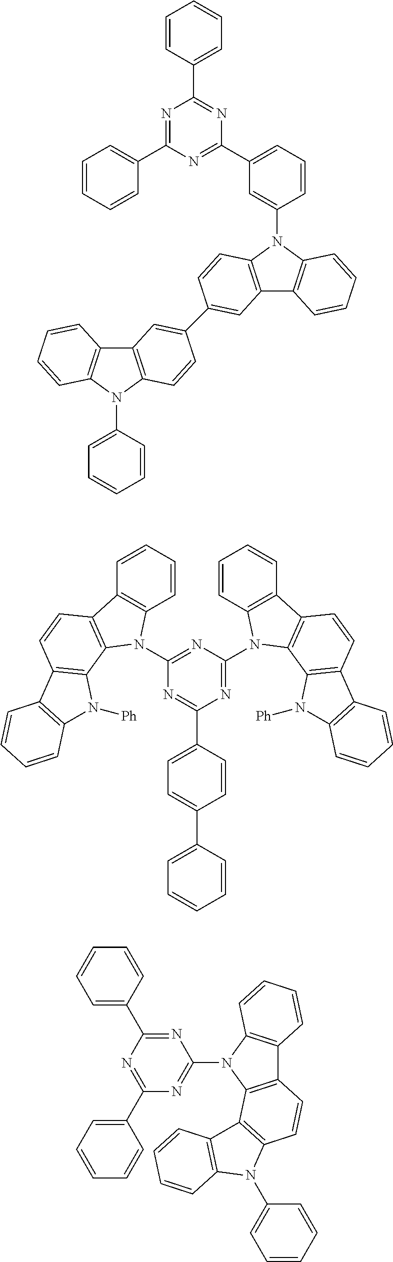 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1]

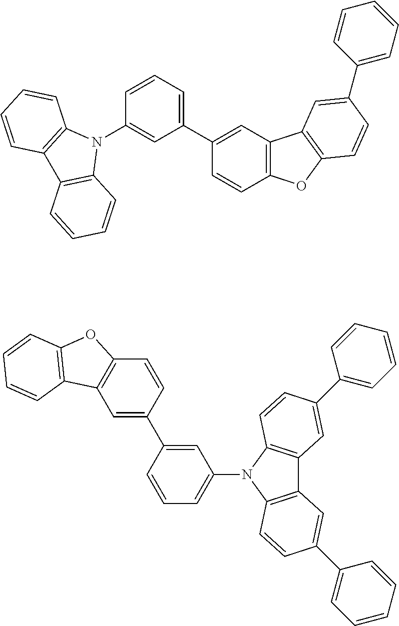 [SMILES: c1ccc(-c2ccc3oc4ccc(-c5cccc(-n6c7ccccc7c7ccccc76)c5)cc4c3c2)cc1 ; c1ccc(-c2ccc3c(c2)c2cc(-c4ccccc4)ccc2n3-c2cccc(-c3ccc4oc5ccccc5c4c3)c2)cc1]

In some embodiments, the light emitting layer contains two or more kinds of TADF molecules differing in the structure. For example, the light emitting layer can contain three kinds of materials of a host material, a first TADF molecule and a second TADF molecule whose excited singlet energy level is higher in that order. In that case, both the first TADF molecule and the second TADF molecule are preferably such that the difference ΔE_{ST} between the lowest excited singlet energy level and the lowest excited triplet energy level at 77 K is 0.3 eV or less, more preferably 0.25 eV or less, even more preferably 0.2 eV or less, further more preferably 0.15 eV or less, further more preferably 0.1 eV or less, further more preferably 0.07 eV or less, further more preferably 0.05 eV or less, further more preferably 0.03 eV or less, and particularly preferably 0.01 eV or less. The content of the first TADF molecule in the light emitting layer is preferably larger than the content of the second TADF molecule therein. The content of the host material in the light emitting layer is preferably larger than the content of the second TADF molecule therein. The content of the first TADF molecule in the light emitting layer can be larger than or can be smaller than or can be the same as the content of the host material therein. In some embodiments, the composition in the light emitting layer can be 10 to 70% by weight of a host material, 10 to 80% by weight of a first TADF molecule, and 0.1 to 30% by weight of a second TADF molecule. In some embodiments, the composition in the light emitting layer can be 20 to 45% by weight of a host material, 50 to 75% by weight of a first TADF molecule, and 5 to 20% by weight of a second TADF molecule. In some embodiments, the emission quantum yield φPL1(A) by photo-excitation of a vapor co-deposited film of a first TADF molecule and a host material (the content of the first TADF molecule in the vapor co-deposited film=A % by weight) and the emission quantum yield φPL2(A) by photo-excitation of a vapor co-deposited film of a second TADF molecule and a host material (the content of the second TADF molecule in the vapor co-deposited film=A % by weight) satisfy a relational formula φPL1(A)>φPL2(A). In some embodiments, the emission quantum yield φPL2(B) by photo-excitation of a vapor co-deposited film of a second TADF molecule and a host material (the content of the second TADF molecule in the vapor co-deposited film=B % by weight) and the emission quantum yield φPL2(100) by photo-excitation of a single film of a second TADF molecule satisfy a relational formula φPL2(B)>φPL2(100). In some embodiments, the light emitting layer can contain three kinds of TADF molecules differing in the structure. The compound of the present invention can be any of the plural TADF compounds contained in the light emitting layer.

In some embodiments, the light emitting layer can be composed of materials selected from the group consisting of a host material, an assist dopant and a light emitting material. In some embodiments, the light emitting layer does not contain a metal element. In some embodiments, the light emitting layer can be formed of a material composed of atoms alone selected from the group consisting of a carbon atom, a hydrogen atom, a deuterium atom, a nitrogen atom, an oxygen atom and a sulfur atom. Or the light emitting layer can be formed of a material composed of atoms alone selected from the group consisting of a carbon atom, a hydrogen atom, a deuterium atom, a nitrogen atom and an oxygen atom. Or the light emitting layer can be formed of a material composed of atoms alone selected from the group consisting of a carbon atom, a hydrogen atom, a nitrogen atom and an oxygen atom.

In the case where the light emitting layer contains any other TADF material than the compound of the present invention, the TADF material can be a known delayed fluorescent material. As preferred delayed fluorescent materials, there can be mentioned compounds included in the general formulae described in WO2013/154064, paragraphs 0008 to 0048 and 0095 to 0133; WO2013/011954, paragraphs 0007 to 0047 and 0073 to 0085; WO2013/011955, paragraphs 0007 to 0033 and 0059 to 0066; WO2013/081088, paragraphs 0008 to 0071 and 0118 to 0133; JP 2013-256490 A, paragraphs 0009 to 0046 and 0093 to 0134; JP 2013-116975 A, paragraphs 0008 to 0020 and 0038 to 0040; WO2013/133359, paragraphs 0007 to 0032 and 0079 to 0084; WO2013/161437, paragraphs 0008 to 0054 and 0101 to 0121; JP 2014-9352 A, paragraphs 0007 to 0041 and 0060 to 0069; JP 2014-9224 A, paragraphs 0008 to 0048 and 0067 to 0076; JP 2017-119663 A, paragraphs 0013 to 0025; JP 2017-119664 A, paragraphs 0013 to 0026; JP 2017-222623 A, paragraphs 0012 to 0025; JP 2017-226838 A, paragraphs 0010 to 0050; JP 2018-100411 A, paragraphs 0012 to 0043; WO2018/047853, paragraphs 0016 to 0044; and especially, exemplary compounds therein capable of emitting delayed fluorescence. In addition, also preferably employable here are light emitting materials capable of emitting delayed fluorescence, as described in JP 2013-253121 A, WO2013/133359, WO2014/034535, WO2014/115743, WO2014/122895, WO2014/126200, WO2014/136758, WO2014/133121, WO2014/136860, WO2014/196585, WO2014/189122, WO2014/168101, WO2015/008580, WO2014/203840, WO2015/002213, WO2015/016200, WO2015/019725, WO2015/072470, WO2015/108049, WO2015/080182, WO2015/072537, WO2015/080183, JP 2015-129240 A, WO2015/129714, WO2015/129715, WO2015/133501, WO2015/136880, WO2015/137244, WO2015/137202, WO2015/137136, WO2015/146541, and WO2015/159541. These patent publications described in this paragraph are hereby incorporated as a part of this description by reference.

In the following, the constituent members and the other layers than the light emitting layer of the organic electroluminescent device are described.

### Substrate:

In some embodiments, the organic electroluminescent device of the invention is supported by a substrate, wherein the substrate is not particularly limited and can be any of those that have been commonly used in an organic electroluminescent device, for example those formed of glass, transparent plastics, quartz and silicon.

### Anode:

In some embodiments, the anode of the organic electroluminescent device is made of a metal, an alloy, an electroconductive compound, or a combination thereof. In some embodiments, the metal, alloy, or electroconductive compound has a large work function (4 eV or more). In some embodiments, the metal is Au. In some embodiments, the electroconductive transparent material is selected from CuI, indium tin oxide (ITO), SnO₂, and ZnO. In some embodiments, an amorphous material capable of forming a transparent electroconductive film, such as IDIXO (In₂O₃—ZnO), is be used. In some embodiments, the anode is a thin film. In some embodiments, the thin film is made by vapor deposition or sputtering. In some embodiments, the film is patterned by a photolithography method. In some embodiments, where the pattern may not require high accuracy (for example, approximately 100 μm or more), the pattern can be formed with a mask having a desired shape on vapor deposition or sputtering of the electrode material. In some embodiments, when a material can be applied as a coating, such as an organic electroconductive compound, a wet film forming method, such as a printing method or a coating method is used. In some embodiments, when the emitted light goes through the anode, the anode has a transmittance of more than 10%, and the anode has a sheet resistance of several hundred Ohm per unit area or less. In some embodiments, the thickness of the anode is from 10 to 1,000 nm. In some embodiments, the thickness of the anode is from 10 to 200 nm. In some embodiments, the thickness of the anode varies depending on the material used.

### Cathode:

In some embodiments, the cathode is made of an electrode material such as a metal having a small work function (4 eV or less) (referred to as an electron injection metal), an alloy, an electroconductive compound, or a combination thereof. In some embodiments, the electrode material is selected from sodium, a sodium-potassium alloy, magnesium, lithium, a magnesium-copper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth element. In some embodiments, a mixture of an electron injection metal and a second metal that is a stable metal having a larger work function than the electron injection metal is used. In some embodiments, the mixture is selected from a magnesium-silver mixture, a magnesium-aluminum mixture, a magnesium-indium mixture, an aluminum-aluminum oxide (Al₂O₃) mixture, a lithium-aluminum mixture, and aluminum. In some embodiments, the mixture increases the electron injection property and the durability against oxidation. In some embodiments, the cathode is produced by forming the electrode material into a thin film by vapor deposition or sputtering. In some embodiments, the cathode has a sheet resistance of several hundred Ohm per unit area or less. In some embodiments, the thickness of the cathode ranges from 10 nm to 5 μm. In some embodiments, the thickness of the cathode ranges from 50 to 200 nm. In some embodiments, for transmitting the emitted light, any one of the anode and the cathode of the organic electroluminescent device is transparent or translucent. In some embodiments, the transparent or translucent electroluminescent devices enhances the light emission luminance.

In some embodiments, the cathode is formed with an electroconductive transparent material, as described for the anode, to form a transparent or translucent cathode. In some embodiments, a device comprises an anode and a cathode, both being transparent or translucent.

### Injection Layer:

An injection layer is a layer between the electrode and the organic layer. In some embodiments, the injection layer decreases the drive voltage and enhances the light emission luminance. In some embodiments, the injection layer includes a hole injection layer and an electron injection layer. The injection layer can be positioned between the anode and the light emitting layer or the hole transporting layer, and between the cathode and the light emitting layer or the electron transporting layer. In some embodiments, an injection layer is present. In some embodiments, no injection layer is present.

Preferred compound examples for use as a hole injection material are shown below.

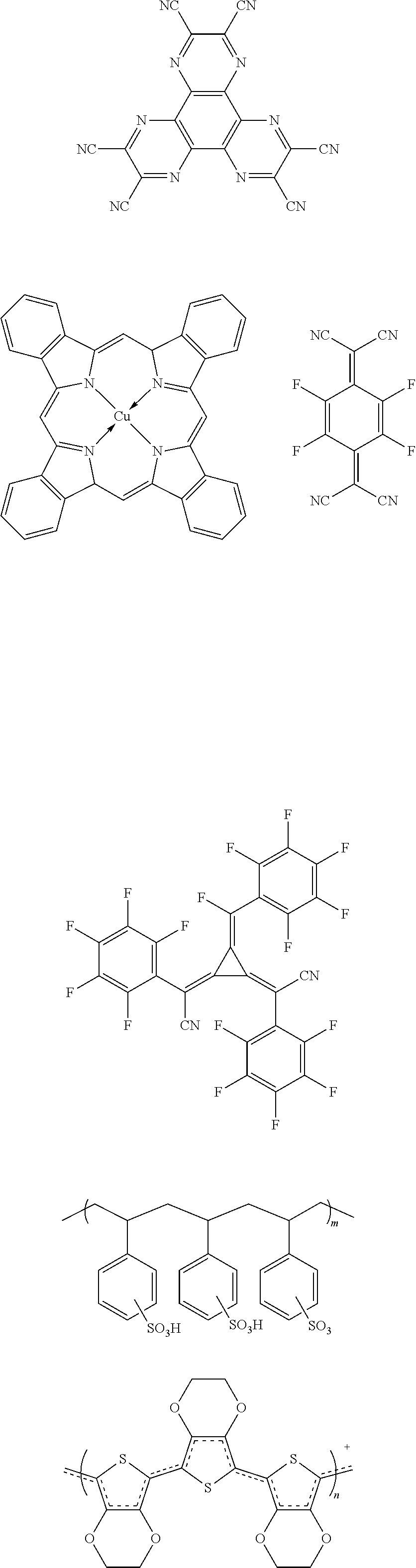

Next, preferred compound examples for use as an electron injection material are shown below.

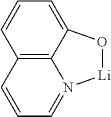

### Barrier Layer:

A barrier layer is a layer capable of inhibiting charges (electrons or holes) and/or excitons present in the light emitting layer from being diffused outside the light emitting layer. In some embodiments, the electron barrier layer is between the light emitting layer and the hole transporting layer, and inhibits electrons from passing through the light emitting layer toward the hole transporting layer. In some embodiments, the hole barrier layer is between the light emitting layer and the electron transporting layer, and inhibits holes from passing through the light emitting layer toward the electron transporting layer. In some embodiments, the barrier layer inhibits excitons from being diffused outside the light emitting layer. In some embodiments, the electron barrier layer and the hole barrier layer are exciton barrier layers. As used herein, the term “electron barrier layer” or “exciton barrier layer” includes a layer that has both the function of an electron barrier layer and the function of an exciton barrier layer.

### Hole Barrier Layer:

A hole barrier layer acts as an electron transporting layer. In some embodiments, the hole barrier layer inhibits holes from reaching the electron transporting layer while transporting electrons. In some embodiments, the hole barrier layer enhances the recombination probability of electrons and holes in the light emitting layer. The material used for the hole barrier layer can be the same materials as the ones described for the electron transporting layer.

Preferred compound examples for use for the hole barrier layer are shown below.

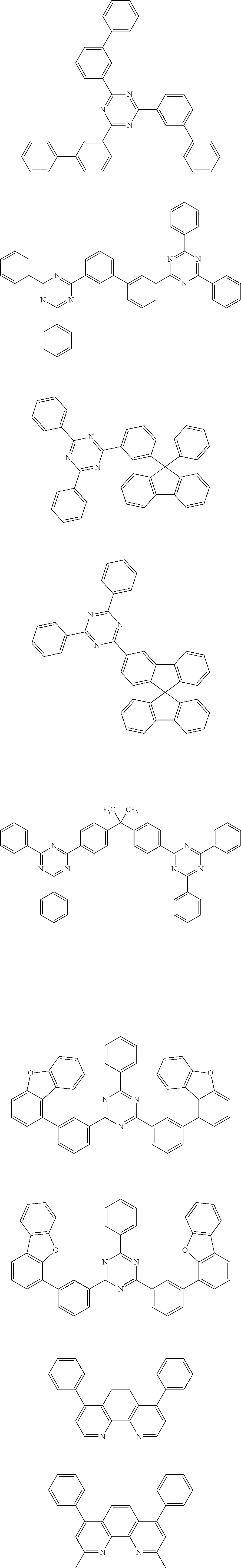

### Electron Barrier Layer:

An electron barrier layer transports holes. In some embodiments, the electron barrier layer inhibits electrons from reaching the hole transporting layer while transporting holes. In some embodiments, the electron barrier layer enhances the recombination probability of electrons and holes in the light emitting layer. The material used for the electron barrier layer can be the same material as the ones described above for the hole transporting layer.

Preferred compound examples for use as the electron barrier material are shown below.

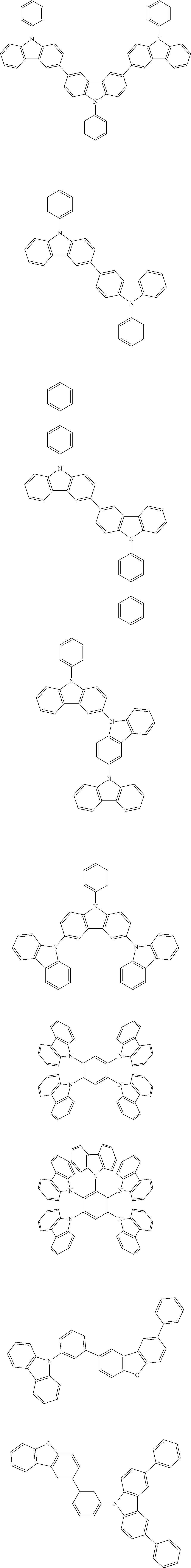

### Exciton Barrier Layer:

An exciton barrier layer inhibits excitons generated through recombination of holes and electrons in the light emitting layer from being diffused to the charge transporting layer. In some embodiments, the exciton barrier layer enables effective confinement of excitons in the light emitting layer. In some embodiments, the light emission efficiency of the device is enhanced. In some embodiments, the exciton barrier layer is adjacent to the light emitting layer on any of the side of the anode and the side of the cathode, and on both the sides. In some embodiments, where the exciton barrier layer is on the side of the anode, the layer can be between the hole transporting layer and the light emitting layer and adjacent to the light emitting layer. In some embodiments, where the exciton barrier layer is on the side of the cathode, the layer can be between the light emitting layer and the cathode and adjacent to the light emitting layer. In some embodiments, a hole injection layer, an electron barrier layer, or a similar layer is between the anode and the exciton barrier layer that is adjacent to the light emitting layer on the side of the anode. In some embodiments, a hole injection layer, an electron barrier layer, a hole barrier layer, or a similar layer is between the cathode and the exciton barrier layer that is adjacent to the light emitting layer on the side of the cathode. In some embodiments, the exciton barrier layer comprises excited singlet energy and excited triplet energy, at least one of which is higher than the excited singlet energy and the excited triplet energy of the light emitting material, respectively.

### Hole Transporting Layer:

The hole transporting layer comprises a hole transporting material. In some embodiments, the hole transporting layer is a single layer. In some embodiments, the hole transporting layer comprises a plurality of layers.

In some embodiments, the hole transporting material has one of injection or transporting property of holes and barrier property of electrons. In some embodiments, the hole transporting material is an organic material. In some embodiments, the hole transporting material is an inorganic material. Examples of known hole transporting materials that can be used in the present invention include but are not limited to a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an allylamine derivative, an amino-substituted chalcone derivative, an oxazole derivative, a styrylanthracene derivative, a fluorenone derivative, a hydrazone derivative, a stilbene derivative, a silazane derivative, an aniline copolymer and an electroconductive polymer oligomer, (particularly, a thiophene oligomer), or a combination thereof. In some embodiments, the hole transporting material is selected from a porphyrin compound, an aromatic tertiary amine compound, and a styrylamine compound. In some embodiments, the hole transporting material is an aromatic tertiary amine compound. Preferred compound examples for use as the hole transporting material are shown below.

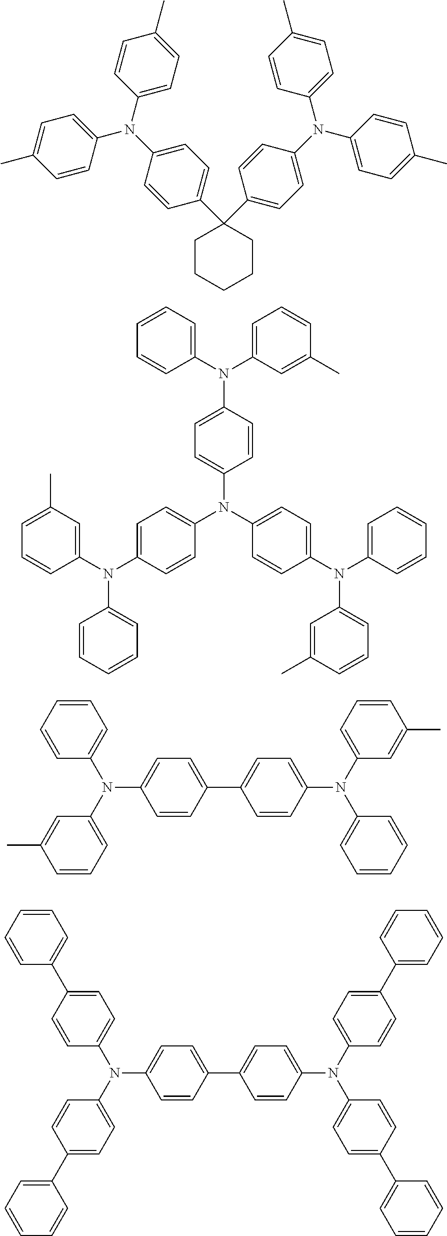

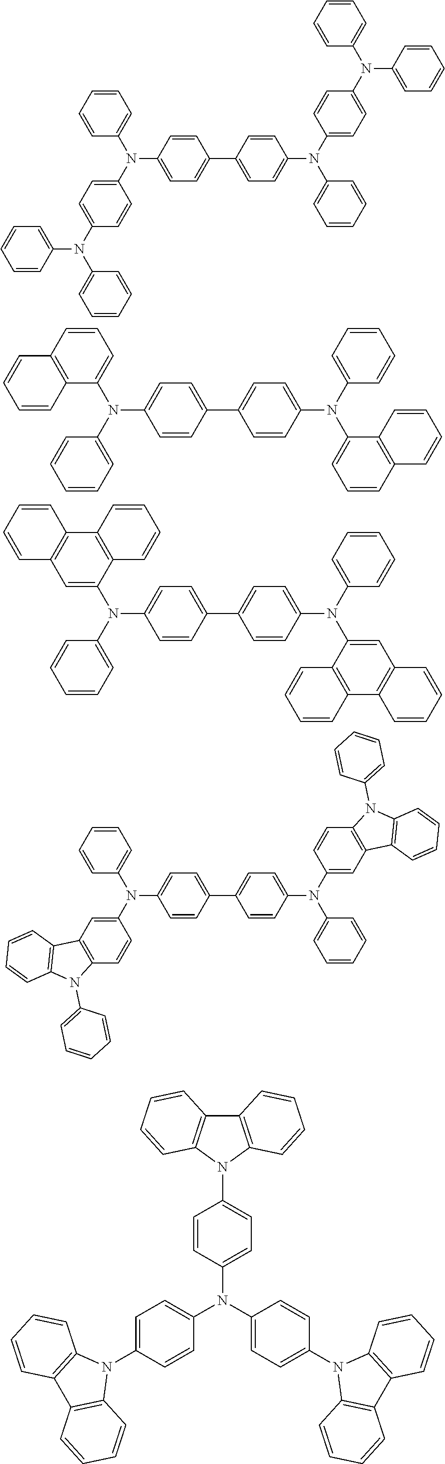

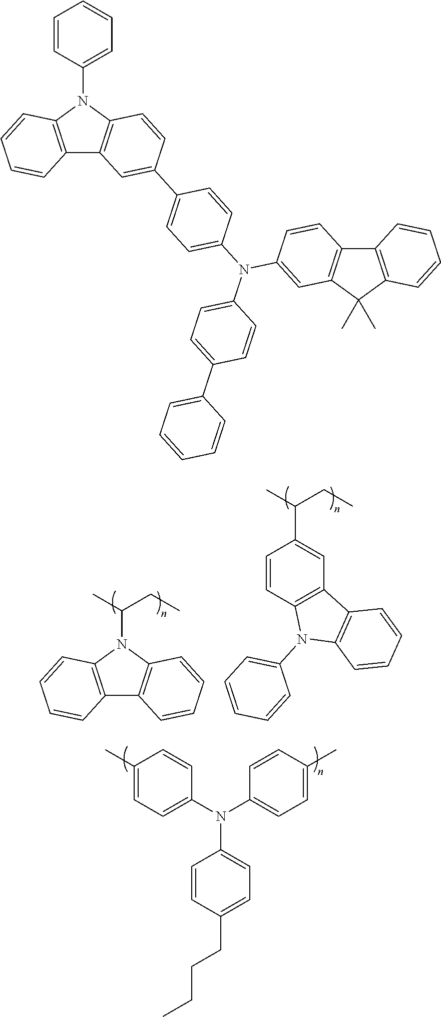

### Electron Transporting Layer:

The electron transporting layer comprises an electron transporting material. In some embodiments, the electron transporting layer is a single layer. In some embodiments, the electron transporting layer comprises a plurality of layers.

In some embodiments, the electron transporting material needs only to have a function of transporting electrons, which are injected from the cathode, to the light emitting layer. In some embodiments, the electron transporting material also function as a hole barrier material. Examples of the electron transporting layer that can be used in the present invention include but are not limited to a nitro-substituted fluorene derivative, a diphenylquinone derivative, a thiopyran dioxide derivative, carbodiimide, a fluorenylidene methane derivative, anthraquinodimethane, an anthrone derivatives, an oxadiazole derivative, an azole derivative, an azine derivative, or a combination thereof, or a polymer thereof. In some embodiments, the electron transporting material is a thiadiazole derivative, or a quinoxaline derivative. In some embodiments, the electron transporting material is a polymer material. Preferred compound examples for use as the electron transporting material are shown below.

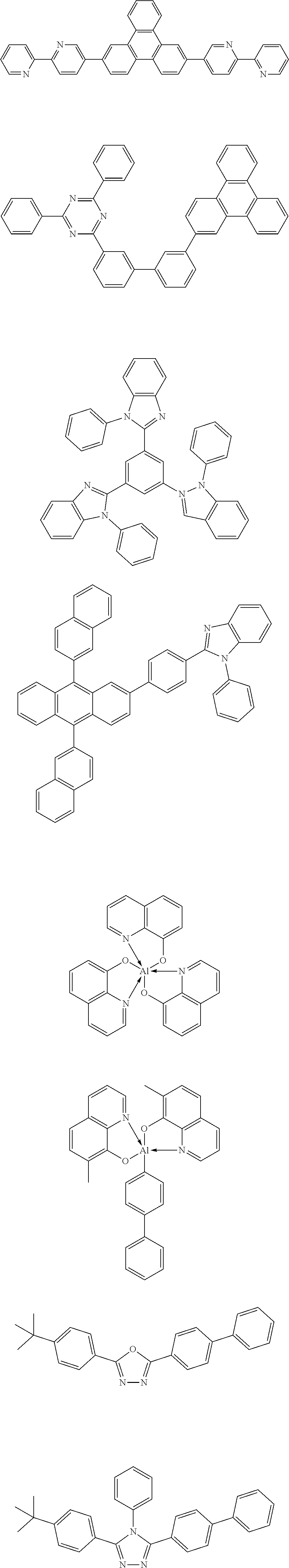

Hereinunder, compound examples preferred as a material that can be added to the organic layers are shown. For example, it is conceivable to add these as a stabilization material.

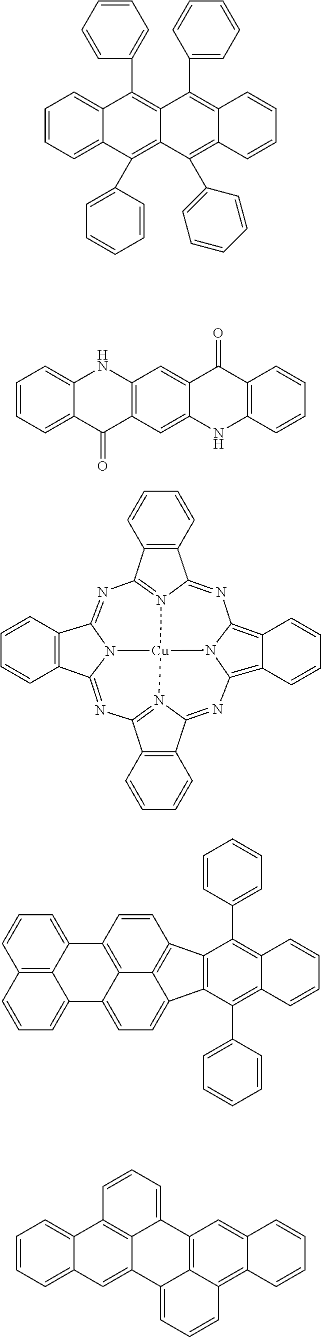

Preferred materials for use in the organic electroluminescent device are specifically shown. However, the materials usable in the invention should not be limitatively interpreted by the following exemplary compounds. Compounds that are exemplified as materials having a specific function can also be used as materials having any other function.

### Devices:

In some embodiments, an light emitting layer is incorporated into a device. For example, the device includes, but is not limited to an OLED bulb, an OLED lamp, a television screen, a computer monitor, a mobile phone, and a tablet.

In some embodiments, an electronic device comprises an OLED comprising an anode, a cathode, and at least one organic layer comprising a light emitting layer between the anode and the cathode.

In some embodiments, compositions described herein can be incorporated into various light-sensitive or light-activated devices, such as OLEDs or opto-electronic devices. In some embodiments, the composition can be useful in facilitating charge transfer or energy transfer within a device and/or as a hole-transport material. The device can be, for example, an organic light emitting diode (OLED), an organic integrated circuit (O-IC), an organic field-effect transistor (O-FET), an organic thin-film transistor (O-TFT), an organic light emitting transistor (O-LET), an organic solar cell (O-SC), an organic optical detector, an organic photoreceptor, an organic field-quench device (O-FQD), a light emitting electrochemical cell (LEC) or an organic laser diode (O-laser).

### Bulbs or Lamps:

In some embodiments, an electronic device comprises an OLED comprising an anode, a cathode, and at least one organic layer comprising a light emitting layer between the anode and the cathode.

In some embodiments, a device comprises OLEDs that differ in color. In some embodiments, a device comprises an array comprising a combination of OLEDs. In some embodiments, the combination of OLEDs is a combination of three colors (e.g., RGB). In some embodiments, the combination of OLEDs is a combination of colors that are not red, green, or blue (for example, orange and yellow green). In some embodiments, the combination of OLEDs is a combination of two, four, or more colors.

In some embodiments, a device is an OLED light comprising:
a circuit board having a first surface with a mounting surface and an opposing second surface, and defining at least one aperture;
at least one OLED on the mounting surface, the at least one OLED configured to emanate light, the OLED comprising: an anode, a cathode, and at least one organic layer comprising a light emitting layer between the anode and the cathode;
a housing for the circuit board; and
at least one connector arranged at an end of the housing, the housing and the connector defining a package adapted for installation in a light fixture.

In some embodiments, the OLED light comprises a plurality of OLEDs mounted on a circuit board such that light emanates in a plurality of directions. In some embodiments, a portion of the light emanated in a first direction is deflected to emanate in a second direction. In some embodiments, a reflector is used to deflect the light emanated in a first direction.

### Displays or Screens:

In some embodiments, the light emitting layer of the invention can be used in a screen or a display. In some embodiments, the compounds of the invention are deposited onto a substrate using a process including, but not limited to, vacuum evaporation, deposition, vapor deposition, or chemical vapor deposition (CVD). In some embodiments, the substrate is a photoplate structure useful in a two-sided etching that provides a unique aspect ratio pixel. The screen (which can also be referred to as a mask) is used in a process in the manufacturing of OLED displays. The corresponding artwork pattern design facilitates a very steep and narrow tie-bar between the pixels in the vertical direction and a large, sweeping bevel opening in the horizontal direction. This allows the fine patterning of pixels needed for high definition displays while optimizing the chemical deposition onto a TFT backplane.

The internal patterning of the pixel allows the construction of a three-dimensional pixel opening with varying aspect ratios in the horizontal and vertical directions. Additionally, the use of imaged “stripes” or halftone circles within the pixel area inhibits etching in specific areas until these specific patterns are undercut and fall off the substrate. At that point, the entire pixel area is subjected to a similar etching rate but the depths are varying depending on the halftone pattern. Varying the size and spacing of the halftone pattern allows etching to be inhibited at different rates within the pixel and allows a localized deeper etch needed to create steep vertical bevels.

A preferred material for the deposition mask is invar. Invar is a metal alloy that is cold rolled into a long thin sheet in a steel mill. Invar cannot be electrodeposited onto a rotating mandrel as the nickel mask. A preferred and more cost feasible method for forming the open areas in the mask used for deposition is through a wet chemical etching.

In some embodiments, a screen or display pattern is a pixel matrix on a substrate. In some embodiments, a screen or display pattern is fabricated using lithography (e.g., photolithography and e-beam lithography). In some embodiments, a screen or display pattern is fabricated using a wet chemical etching. In further embodiments, a screen or display pattern is fabricated using plasma etching.

### Methods of Manufacturing Devices:

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light emitting layer, a counter electrode, and an encapsulation layer, and then is cut from the mother panel.

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light emitting layer, a counter electrode, and an encapsulation layer, and then is cut from the mother panel.

In another aspect, provided herein is a method of manufacturing an organic light emitting diode (OLED) display, the method comprising:
forming a barrier layer on a base substrate of a mother panel;
forming a plurality of display units in units of cell panels on the barrier layer;
forming an encapsulation layer on each of the display units of the cell panels;
applying an organic film to an interface portion between the cell panels.

In some embodiments, the barrier layer is an inorganic film formed of, for example, SiNx, and an edge portion of the barrier layer is covered with an organic film formed of polyimide or acryl. In some embodiments, the organic film helps the mother panel to be softly cut in units of the cell panel.

In some embodiments, the thin film transistor (TFT) layer includes a light emitting layer, a gate electrode, and a source/drain electrode. Each of the plurality of display units may include a thin film transistor (TFT) layer, a planarization film formed on the TFT layer, and a light emitting unit formed on the planarization film, wherein the organic film applied to the interface portion is formed of a same material as a material of the planarization film and is formed at a same time as the planarization film is formed. In some embodiments, a light emitting unit is connected to the TFT layer with a passivation layer and a planarization film therebetween and an encapsulation layer that covers and protects the light emitting unit. In some embodiments of the method of manufacturing, the organic film contacts neither the display units nor the encapsulation layer.

Each of the organic film and the planarization film can include any one of polyimide and acryl. In some embodiments, the barrier layer can be an inorganic film. In some embodiments, the base substrate can be formed of polyimide. The method can further include, before the forming of the barrier layer on one surface of the base substrate formed of polyimide, attaching a carrier substrate formed of a glass material to another surface of the base substrate, and before the cutting along the interface portion, separating the carrier substrate from the base substrate. In some embodiments, the OLED display is a flexible display.

In some embodiments, the passivation layer is an organic film disposed on the TFT layer to cover the TFT layer. In some embodiments, the planarization film is an organic film formed on the passivation layer. In some embodiments, the planarization film is formed of polyimide or acryl, like the organic film formed on the edge portion of the barrier layer. In some embodiments, the planarization film and the organic film are simultaneously formed when the OLED display is manufactured. In some embodiments, the organic film can be formed on the edge portion of the barrier layer such that a portion of the organic film directly contacts the base substrate and a remaining portion of the organic film contacts the barrier layer while surrounding the edge portion of the barrier layer.

In some embodiments, the light emitting layer includes a pixel electrode, a counter electrode, and an organic light emitting layer disposed between the pixel electrode and the counter electrode. In some embodiments, the pixel electrode is connected to the source/drain electrode of the TFT layer.

In some embodiments, when a voltage is applied to the pixel electrode through the TFT layer, an appropriate voltage is formed between the pixel electrode and the counter electrode, and thus the organic light emitting layer emits light, thereby forming an image. Hereinafter, an image forming unit including the TFT layer and the light emitting unit is referred to as a display unit.

In some embodiments, the encapsulation layer that covers the display unit and prevents penetration of external moisture can be formed to have a thin film encapsulation structure in which an organic film and an inorganic film are alternately stacked. In some embodiments, the encapsulation layer has a thin film encapsulation structure in which a plurality of thin films are stacked. In some embodiments, the organic film applied to the interface portion is spaced apart from each of the plurality of display units. In some embodiments, the organic film is formed such that a portion of the organic film directly contacts the base substrate and a remaining portion of the organic film contacts the barrier layer while surrounding an edge portion of the barrier layer.

In one embodiment, the OLED display is flexible and uses the soft base substrate formed of polyimide. In some embodiments, the base substrate is formed on a carrier substrate formed of a glass material, and then the carrier substrate is separated.

In some embodiments, the barrier layer is formed on a surface of the base substrate opposite to the carrier substrate. In one embodiment, the barrier layer is patterned according to a size of each of the cell panels. For example, while the base substrate is formed over the entire surface of a mother panel, the barrier layer is formed according to a size of each of the cell panels, and thus a groove is formed at an interface portion between the barrier layers of the cell panels. Each of the cell panels can be cut along the groove.

In some embodiments, the manufacturing method further comprises cutting along the interface portion, wherein a groove is formed in the barrier layer, wherein at least a portion of the organic film is formed in the groove, and wherein the groove does not penetrate into the base substrate. In some embodiments, the TFT layer of each of the cell panels is formed, and the passivation layer which is an inorganic film and the planarization film which is an organic film are disposed on the TFT layer to cover the TFT layer. At the same time as the planarization film formed of, for example, polyimide or acryl is formed, the groove at the interface portion is covered with the organic film formed of, for example, polyimide or acryl. This is to prevent cracks from occurring by allowing the organic film to absorb an impact generated when each of the cell panels is cut along the groove at the interface portion. That is, if the entire barrier layer is entirely exposed without the organic film, an impact generated when each of the cell panels is cut along the groove at the interface portion is transferred to the barrier layer, thereby increasing the risk of cracks. However, in one embodiment, since the groove at the interface portion between the barrier layers is covered with the organic film and the organic film absorbs an impact that would otherwise be transferred to the barrier layer, each of the cell panels can be softly cut and cracks can be prevented from occurring in the barrier layer. In one embodiment, the organic film covering the groove at the interface portion and the planarization film are spaced apart from each other. For example, if the organic film and the planarization film are connected to each other as one layer, since external moisture may penetrate into the display unit through the planarization film and a portion where the organic film remains, the organic film and the planarization film are spaced apart from each other such that the organic film is spaced apart from the display unit.

In some embodiments, the display unit is formed by forming the light emitting unit, and the encapsulation layer is disposed on the display unit to cover the display unit. As such, once the mother panel is completely manufactured, the carrier substrate that supports the base substrate is separated from the base substrate. In some embodiments, when a laser beam is emitted toward the carrier substrate, the carrier substrate is separated from the base substrate due to a difference in a thermal expansion coefficient between the carrier substrate and the base substrate.

In some embodiments, the mother panel is cut in units of the cell panels. In some embodiments, the mother panel is cut along an interface portion between the cell panels by using a cutter. In some embodiments, since the groove at the interface portion along which the mother panel is cut is covered with the organic film, the organic film absorbs an impact during the cutting. In some embodiments, cracks can be prevented from occurring in the barrier layer during the cutting.

In some embodiments, the methods reduce a defect rate of a product and stabilize its quality.

Another aspect is an OLED display including: a barrier layer that is formed on a base substrate; a display unit that is formed on the barrier layer; an encapsulation layer that is formed on the display unit; and an organic film that is applied to an edge portion of the barrier layer.

### EXAMPLES

The features of the present invention will be described more specifically with reference to Synthesis Examples and Examples given below. The materials, processes, procedures and the like shown below can be appropriately modified unless they deviate from the substance of the invention. Accordingly, the scope of the invention is not construed as being limited to the specific examples shown below. Hereinunder, the light emission characteristics were evaluated using a source meter (available from Keithley Instruments, Inc., Keithley 2400), a semiconductor parameter analyzer (available from Agilent Technologies, Inc., E5273A), a light power meter apparatus (available from Newport Corporation, 1930C), an optical spectroscope (available from Ocean Optics Corporation, USB 2000), a spectroradiometer (available from Topcon Corporation, SR-3) and a streak camera (available from Hamamatsu Photonics K.K., C4334). The energies of HOMO and LUMO were measured by atmospheric photoelectron spectroscopy (such as AC-3 manufactured by Riken Keiki Co., Ltd.). In the following Synthesis Examples, compounds included in the general formula (1) were synthesized.

### (Synthesis Example 1) Synthesis of Compound 1

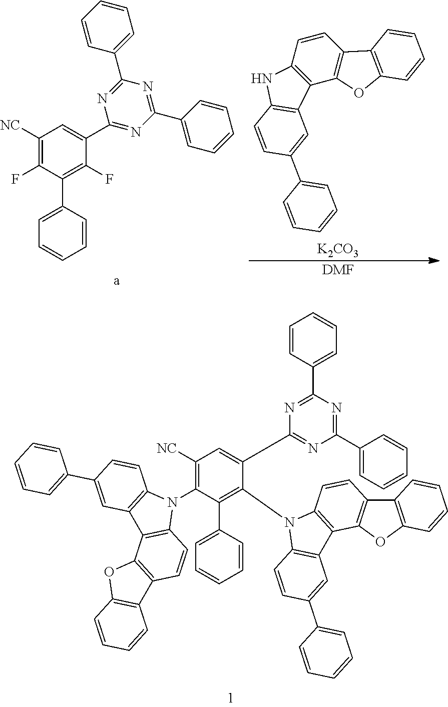

Compound a (0.56 g, 1.25 mmol) was added to a dimethylformamide solution (25 mL) of 2-phenyl-5H-benzofuro[3,2-c]carbazole (1.05 g, 3.1 mmol) and potassium carbonate (0.86 g, 3.76 mmol) under a nitrogen stream, and the mixture was stirred at 100° C. for 15 hours. The mixture was returned to room temperature, quenched by the addition of methanol, and the precipitated solid was filtered and washed with water and methanol. The resultant solid was purified by silica gel column chromatography to give Compound 1 (1.10 g, 1.02 mmol, yield 81.9%).

¹H NMR (400 MHz, CDCl₃) δ 9.06 (s, 1H), 8.65 (dd, J=4.4, 0.8 Hz, 1H), 8.45 (d, J=2.0 Hz, 1H), 8.00-7.94 (m, 6H), 7.91 (d, J=7.6 Hz, 1H), 7.86 (dd, J=8.4, 0.8 Hz, 1H), 7.80-7.60 (m, 8H), 7.54-7.30 (m, 12H), 7.24-7.16 (m, 8H), 6.78-6.76 (m, 2H), 6.54-6.45 (m, 3H),

ASAP Mass Spectrometry: theoretical value 1072.4, observed value 1072.8

### (Synthesis Example 2) Synthesis of Compound 1(Db)

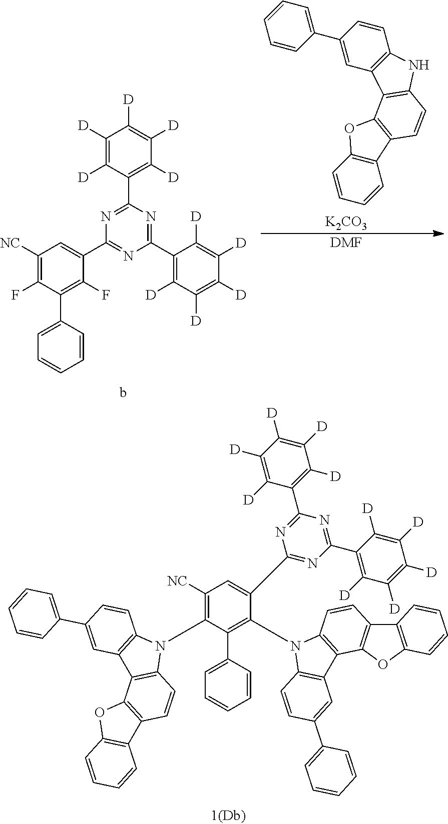

Under a nitrogen stream, a dimethylformamide (32 mL) solution of Compound b (0.75 g, 1.64 mmol), 2-phenyl-5H-benzofuro[3,2-c]carbazole (1.09 g, 3.28 mmol) and potassium carbonate (0.56 g, 4.10 mmol) was stirred at room temperature for 14 hours. Water was added, and the mixture was washed with methanol, filtered and dried. After solvent removal by evaporation, this was purified by silica gel column chromatography (toluene/hexane=1/1) and then recrystallized with toluene to give 0.75 g of a yellow solid, Compound 1(Db) (0.69 mmol, yield 42.1%).

¹H NMR (400 MHz, CDCl₃): δ 9.05 (s, 1H), 8.66 (d, J=5.2 Hz, 1H) 8.44 (s, 1H), 7.99-7.59 (m, 12H), 7.52-7.17 (m, 16H), 6.77 (d, J=7.2 Hz, 2H), 6.49 (dd, J=21.2 Hz, 7.2 Hz, 2H)

ASAP MS Spectrometry: C₇₆H₃₄D₁₀N₆O₂: theoretical value 1082.42, observed value 1083.50

### (Synthesis Example 3) Synthesis of Compound 1(Dg)

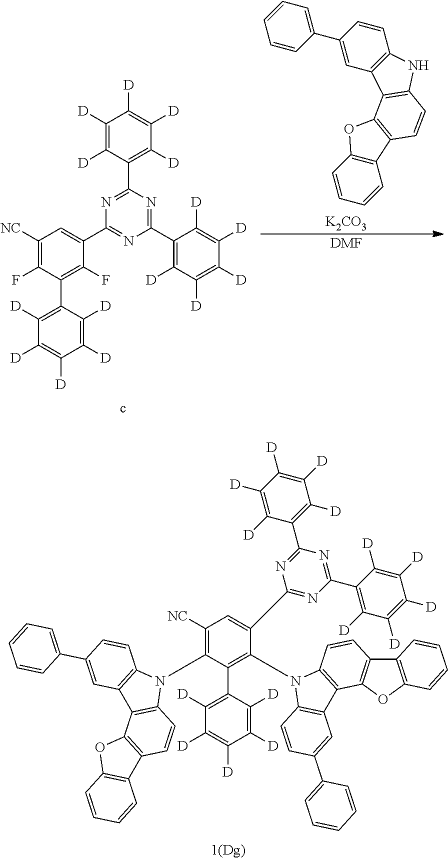

Under a nitrogen stream, a dimethylformamide (85 mL) solution of Compound c (2.0 g, 4.33 mmol), 2-phenyl-5H-benzofuro[3,2-c]carbazole (2.89 g, 8.66 mmol) and potassium carbonate (1.5 g, 10.83 mmol) was stirred at room temperature for 20 hours. Water was added, and the mixture was washed with methanol, filtered and dried. After solvent removal by evaporation, this was purified by silica gel column chromatography (toluene/hexane/chloroform=4/5/1) and then recrystallized with toluene to give 2.5 g of a yellow solid, Compound 1(Dg) (2.29 mmol, yield 53.0%).

¹H NMR (400 MHz, CDCl₃): δ 9.06 (s, 1H), 8.66 (dd, J=1.6 Hz, 4.5 Hz, 1H) 8.45 (d, J=1.6 Hz, 1H), 8.00-7.90 (m, 3H), 7.86 (dd, J=8.4 Hz, 6.4 Hz, 1H), 7.80-7.77 (m, 2H), 7.75-7.66 (m, 4H), 7.65-7.60 (m, 2H), 7.54-7.46 (m, 6H), 7.44-7.30 (m, 6H), 7.22-7.19 (m, 2H)

ASAP MS Spectrometry: C₇₆H₂₉D₁₅N₆O₂: theoretical value 1087.45, observed value 1088.42

### (Synthesis Example 4) Synthesis of Compound 3

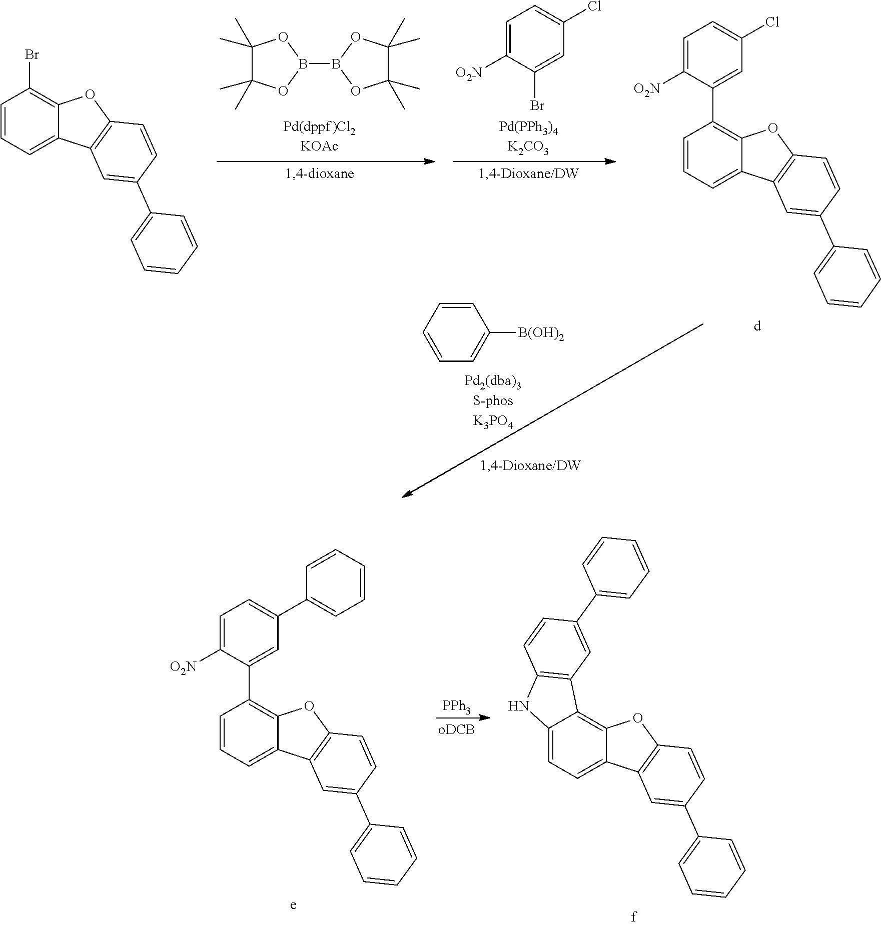

### Compound d

Under a nitrogen stream, [1,1′-bis(diphenylphosphino)ferrocene]dichloropalladium(II) (5.43 g, 7.40 mmol) was added to a 1,4-dioxane (800 mL) solution of 6-bromo-2-phenyldibenzofuran (60.7 g, 185 mmol), bis(piconalate)diborane (70.5 g, 278 mmol) and potassium acetate (54.5 g, 555 mmol) and stirred at 110° C. for 20 hours. The reaction solution was restored to room temperature, and potassium carbonate (51.1 g, 370 mmol), 2-bromo-4-chloro-nitrobenze (52.5 g, 222 mmol), tetrakis(triphenylphosphine)palladium(0) (10.7 g, 9.25 mmol) and deionized water (100 mL) were added and stirred at 80° C. for 12 hours. The reaction solution was restored to room temperature, and after celite filtration, the crude product was purified by silica gel column chromatography (tetrahydrofuran) to give 33.3 g of a yellow solid, compound d (83.3 mmol, yield 45%).

¹H NMR (400 MHz, CDCl₃): δ 8.16 (s, 1H), 8.09 (d, J=8.7 Hz, 1H), 8.06 (dd, J=8.7 Hz, 1.8 Hz, 1H), 7.69-7.64 (m, 4H), 7.59-7.64 (m, 2H), 7.51-7.44 (m, 4H), 7.38 (t, J=8.7 Hz, 1H).

ASAP MS Spectrometry: C₂₄H₁₄ClNO₃: theoretical value 399.07, observed value 400.16

### Compound e

Under a nitrogen stream, tris(dibenzylideneacetone)dipalladium(0) (1.57 g, 1.71 mmol) was added to a mixed solution of 1,4-dioxane/water (210/70 mL) of Compound d (34.3 g, 85.7 mmol), phenylboronic acid (12.5 g, 103 mmol), 2-dicyclohexylphosphino-2′,6′-dimethoxybiphenyl (1.41 g, 3.43 mmol), and tripotassium phosphate (36.4 g, 171 mmol), and stirred at 110° C. for 12 hours. The reaction solution was restored to room temperature, washed with water, and the aqueous layer was extracted with chloroform and dried over anhydrous magnesium sulfate. The crude product was purified by silica gel column chromatography (hexane/dichloromethane=3/2) to give 33.1 g of a yellow solid, Compound e (0.85 mmol, yield 88%).

¹H NMR (400 MHz, CDCl₃: δ 8.23 (d, J=8.2 Hz, 1H), 8.17 (s, 1H), 8.06 (d, J=8.2 Hz, 1H), 7.84-7.79 (m, 2H), 7.69-7.66 (m, 5H), 7.57-7.43 (m, 8H), 7.38 (t, J=8.2 Hz, 1H).

ASAP MS Spectrometry: C₃₀H₁₉NO₃: theoretical value 441.14, observed value 442.33

### Compound f

Under a nitrogen stream, Compound e (733.1 g, 32.7 mmol) was added to an o-dichlorobenzene (74.0 mL) solution of triphenylphosphine (58.2 g, 222 mmol), and stirred at 170° C. for 18 hours. The reaction mixture was returned to room temperature, water was added thereto, and the precipitate was filtered off. The crude product was purified by silica gel column chromatography (hexane/dichloromethane=2/1), and then recrystallized with toluene to give 21.5 g of a brown solid, Compound f (52.6 mmol, yield 71%).

¹H-NMR (400 MHz, CDCl₃): δ 8.73 (s, 1H), 7.38 (s, 1H), 8.18 (s, 1H), 8.02 (d, J=8.2 Hz, 2H), 7.81-7.71 (m, 6H), 7.66 (d, J=8.2 Hz, 1H), 7.59 (d, J=8.2 Hz, 1H), 7.55-7.47 (m, 5H), 7.38 (s, 2H).

ASAP MS Spectrometry: C₃₀H₁₉NO: theoretical value 409.15, observed value 410.15

### Compound 3

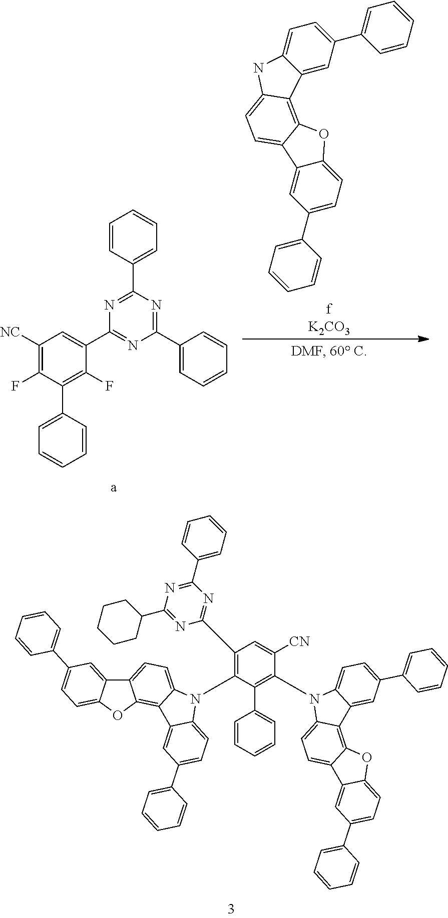 [SMILES: N#Cc1cc(-c2nc(-c3ccccc3)nc(-c3ccccc3)n2)c(F)c(-c2ccccc2)c1F ; c1ccc(-c2ccc3oc4c(ccc5[nH]c6ccc(-c7ccccc7)cc6c54)c3c2)cc1 ; "K2CO3" ; N#Cc1cc(-c2nc(-c3ccccc3)nc(C3CCCCC3)n2)c(-n2c3ccc(-c4ccccc4)cc3c3c4oc5ccc(-c6ccccc6)cc5c4ccc32)c(-c2ccccc2)c1-n1c2ccc(-c3ccccc3)cc2c2c3oc4ccc(-c5ccccc5)cc4c3ccc21]

Under a nitrogen stream, a dimethylformamide (36 mL) solution of Compound a (0.8 g, 1.79 mmol), Compound f (1.32 g, 3.22 mmol) and potassium carbonate (0.57 g, 4.12 mmol) was stirred at room temperature for 14 hours. Water was added, and the mixture was washed with methanol, filtered and dried. After solvent removal by evaporation, this was purified by silica gel column chromatography (toluene/hexane/chloroform=10/9/1) and then recrystallized with toluene to give 0.9 g of a yellow solid, Compound 3 (0.73 mmol, yield 41.0%).

¹H NMR (400 MHz, CDCl₃): δ 9.06 (s, 1H), 8.66 (dd, J=4.8 Hz, 1.2 Hz, 1H), 8.45 (d, J=1.6 Hz, 1H), 8.15 (dd, J=6.0 Hz, 2.0 Hz, 1H), 8.09 (d, J=2.0 Hz, 1H), 8.02-7.95 (m, 5H), 7.88 (dd, J=8.4 Hz, 2.4 Hz, 1H), 7.80-7.60 (m, 14H), 7.53-7.30 (m, 16H), 7.26-7.13 (m, 6H), 6.78 (d, J=6.4 Hz, 2H), 6.56-6.44 (m, 3H)

ASAP MS Spectrometry: C₈₈H₅₂N₆O₂: theoretical value 1225.42, observed value 1225.51

### (Example 1) Preparation and Evaluation of Thin Film

Compound 1 was vapor-deposited on a quartz substrate by a vacuum deposition method under conditions of a vacuum degree of less than 1×10⁻³ Pa to form a neat thin film of Compound 1 having a thickness of 100 nm.

Separately, Compound 1 and mCBP were vapor-deposited from different vapor deposition sources on a quartz substrate by a vacuum deposition method under conditions of a vacuum degree of less than 1×10³ Pa to form a doped thin film having a content of Compound 1 of 20% by weight and a thickness of 100 nm.

In the same manner but using Compound 1(Db), Compound 1(Dg), Compound 3 and Comparative Compound 1 in place of Compound 1, neat thin films and doped thin films were formed.

The maximum emission wavelength (λmax) and the photoluminescence quantum yield (PLQY) were measured when the formed doped thin films were irradiated with excitation light of 300 nm. Also, using the formed neat thin films, the HOMO energy (E_{HOMO}) and the LUMO energy (E_{LUMO}) were measured. The results are shown in Table 4.

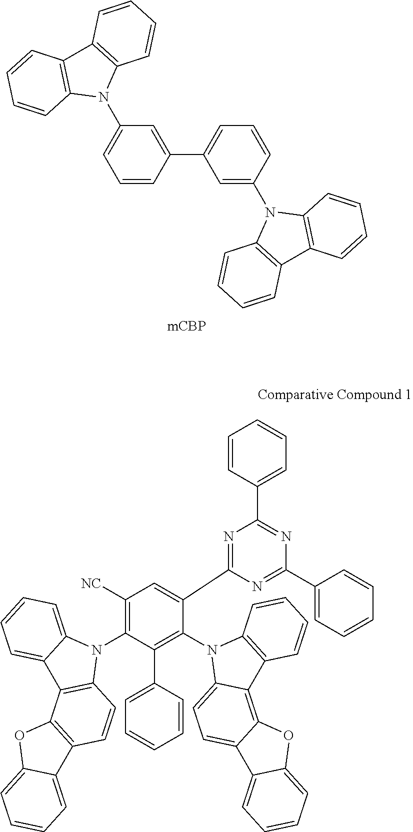 [SMILES: c1cc(-c2cccc(-n3c4ccccc4c4ccccc43)c2)cc(-n2c3ccccc3c3ccccc32)c1 ; N#Cc1cc(-c2nc(-c3ccccc3)nc(-c3ccccc3)n2)c(-n2c3ccccc3c3c4oc5ccccc5c4ccc32)c(-c2ccccc2)c1-n1c2ccccc2c2c3oc4ccccc4c3ccc21]

### (Example 2) Production and Evaluation of Organic Electroluminescent Device

On a glass substrate on which an anode made of indium-tin oxide (ITO) having a film thickness of 50 nm was formed, each thin film was laminated by a vacuum deposition method at a vacuum degree of 5.0×10⁵ Pa. First, HAT-CN was formed to a thickness of 10 nm on the ITO, NPD was formed to a thickness of 35 nm on the HAT-CN, and further PTCz was formed to a thickness of 10 nm on the NPD. Next, H1 and Compound 1 were vapor co-deposited from different vapor deposition sources to form a layer with a thickness of 40 nm as a light emitting layer. The content of Compound 1 in the light emitting layer was 30% by mass. Next, after ET 1 was formed to a thickness of 10 nm, Liq and SF3-TRZ were vapor co-deposited from different vapor deposition sources to form a layer with a thickness of 20 nm. The contents of Liq and SF3-TRZ in this layer were 30% by mass and 70% by mass, respectively. Furthermore, Liq was formed to a thickness of 2 nm, and aluminum (Al) was vapor-deposited to a thickness of 100 nm to form a cathode, thereby obtaining an organic electroluminescent device.

Organic electroluminescent devices were produced in the same manner except that Compound 1(Db), Compound 1(Dg), Compound 3 and Comparative Compound 1 were used in place of Compound 1.

Of each organic electroluminescent device, the external quantum efficiency (EQE) at 6.3 mA, the drive voltage (Vᵢₙᵢₜ), the lifetime of delayed fluorescence (τ2), and the time taken until the emission intensity at 12.6 mA/cm² reached 95% at the start of the test (LT95) were measured, and the results are shown in Table 4. LT95 is expressed as a relative value when the device using Comparative Compound 1 is defined as 1.

TABLE 4

| | Compound | Compound | Compound | Compound | Comparative |
|---|---|---|---|---|---|
| | 1 | 1(Db) | 1(Dg) | 3 | Compound 1 |
| λmax (nm) | 525 | 525 | 527 | 524 | 519 |
| PLQY (%) | 73.5 | 74.0 | 73.5 | 71.2 | 66.8 |
| E_{HOMO} (eV) | 6.01 | 5.96 | 5.93 | 6.04 | 5.93 |
| E_{LUMO} (eV) | 3.53 | 3.44 | 3.38 | 3.49 | 3.35 |
| EQE (%) | 14.7 | 14.6 | 15.1 | 14.6 | 12.7 |
| Vᵢₙᵢₜ (V) | 3.7 | 3.6 | 3.6 | 3.8 | 3.9 |
| LT95 | 1.30 | 1.32 | 1.47 | — | 1 |

It was confirmed that the use of the compound represented by the general formula (1) can improve the light emission efficiency and shorten the delayed fluorescence lifetime. Also it was confirmed that the use of the compound represented by the general formula (1) can prolong the device lifetime and improve the durability.

### (Example 3) Production and Evaluation of Organic Electroluminescent Device Using Compound 1 as Assist Dopant

An organic electroluminescent device was produced in the same manner as in Example 2 only except that a light emitting layer having a thickness of 40 nm was formed by depositing H1, Compound 1 and EM1 as a light emitting material in order of 69.5% by weight, 30.0% by weight and 0.5% by weight from different evaporation sources in place of the light emitting layer in Example 2.

Of the formed organic electroluminescent device, the maximum emission wavelength (λmax) was 528 nm, and the external quantum efficiency (EQE) at 6.3 mA was 20.9%. The durability of the device was also good.

The above confirms that when the compound represented by the general formula (1) is used as an assist dopant, an organic electroluminescent device having a high light emission efficiency and a good durability can also be provided.

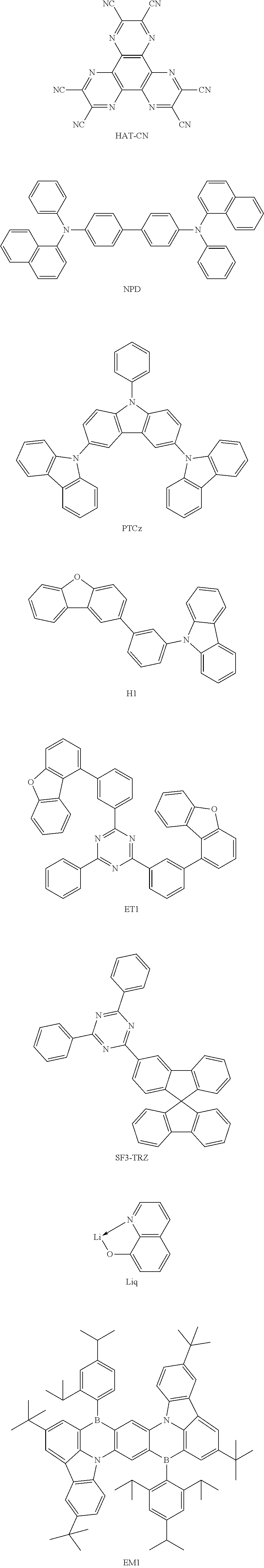

### INDUSTRIAL APPLICABILITY

By using a compound represented by the general formula (1), there can be provided an excellent organic light emitting device having a high light emission efficiency. Accordingly, the industrial applicability of the present invention is great.

1. A compound represented by the following general formula (1): 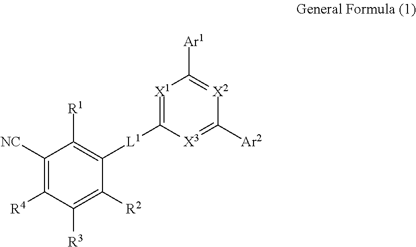
wherein R¹ to R⁴ each independently represent a hydrogen atom, a deuterium atom, a substituted or unsubstituted alkyl group, a substituted or unsubstituted aryl group, or a donor group;
two or more of R¹ to R⁴ are donor groups, and at least one of the two or more donor groups is a substituted ring-fused carbazol-9-yl group;
X¹ to X³ each independently represent N or C(R), and at least one of X¹ to X³ is N;
R represents a hydrogen atom, a deuterium atom or a substituent;
Ar¹ and Ar² each independently represent a substituted or unsubstituted aryl group; and
L¹ represents a single bond or a divalent linking group.

2. The compound according to claim 1, wherein the ring-fused carbazol-9-yl group is substituted with a substituted or unsubstituted alkyl group, or a substituted or unsubstituted aryl group.

3. The compound according to claim 1, wherein the ring-fused carbazol-9-yl group is a carbazol-9-yl group in which a ring having one or more atoms selected from the group consisting of an oxygen atom, a sulfur atom and a nitrogen atom as a ring skeleton-constituting atom is fused.

4. The compound according to claim 1, wherein X¹ to X³ are N.

5. The compound according to claim 1, wherein Ar¹ and Ar² each are an aryl group optionally substituted with a deuterium atom.

6. The compound according to claim 1, wherein L¹ is a single bond.

7. The compound according to claim 1, wherein R¹ is a hydrogen atom.

8. The compound according to claim 7, wherein R³ is a phenyl group.

9. The compound according to claim 8, wherein R² and R⁴ are the same substituted ring-fused carbazol-9-yl group.

10. The compound according to claim 1, wherein the compound has at least one deuterium atom.

11-12. (canceled)

13. A film comprising the compound according to claim 1.

14. An organic semiconductor device comprising the compound according to claim 1.

15. An organic light emitting device comprising the compound according to claim 1.

16. The organic light emitting device according to claim 15, wherein the device has a layer containing the compound, and the layer also contains a host material.

17. The organic light emitting device according to claim 16, wherein the layer containing the compound further contains a delayed fluorescent material in addition to the compound and the host material, and the delayed fluorescent material has a lowest excited singlet energy lower than that of the host material and higher than that of the compound.

18. The organic light emitting device according to claim 16, wherein the device has a layer containing the compound, and the layer also contains a light emitting material having a structure different from that of the compound.

19. The organic light emitting device according to claim 16, wherein the amount of light emitted from the compound is the largest among materials contained in the device.

20. The organic light emitting device according to claim 18, wherein the amount of light emitted from the light emitting material is larger than the amount of light emitted from the compound.

21. The organic light emitting device according to claim 15, which is an organic electroluminescent device.

22. The organic light emitting device according to claim 15, which emits delayed fluorescence.
